SEQUENCE LISTING (93 sequences)

SEQ ID NO: 1 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 2 - protein, 525 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 3 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 4 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 5 - protein, 133 aa, Homo sapiens [sequence omitted]

SEQ ID NO: 6 - protein, 214 aa, Artificial Sequence
  Description of Artificial Sequence Synthetic polypeptide
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDT

SEQ ID NO: 7 - protein, 524 aa, Artificial Sequence
  Description of Artificial Sequence Synthetic polypeptide
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHL

SEQ ID NO: 8 - protein, 24 aa, Artificial Sequence
  Description of Artificial Sequence Synthetic peptide
LLLGVSVSCIVILAVCLLCYVSIT

SEQ ID NO: 9 - protein, 569 aa, Artificial Sequence
  Description of Artificial Sequence Synthetic polypeptide
KIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 10 - protein, 25 aa, Artificial Sequence
  Description of Artificial Sequence Synthetic peptide
PILLTISILSFFSVALLVILACVLW

SEQ ID NO: 11 - protein, 195 aa, Artificial Sequence
  Description of Artificial Sequence Synthetic polypeptide
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 12 - protein, 21 aa, Artificial Sequence
  Description of Artificial Sequence Synthetic peptide
GNTLVAVSIFLLLTGPTYLLF

SEQ ID NO: 13 - protein, 230 aa, Artificial Sequence
  Description of Artificial Sequence Synthetic polypeptide
KLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 14 - protein, 21 aa, Artificial Sequence
  Description of Artificial Sequence Synthetic peptide
GWNPHLLLLLLLVIVFIPAFW

SEQ ID NO: 15 - protein, 285 aa, Artificial Sequence
  Description of Artificial Sequence Synthetic polypeptide
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 16 - protein, 4 aa, Artificial Sequence
  Description of Artificial Sequence Synthetic peptide
YLPQ

SEQ ID NO: 17 - protein, 4 aa, Artificial Sequence
  Description of Artificial Sequence Synthetic peptide
YMPQ

SEQ ID NO: 18 - protein, 5 aa, Artificial Sequence
  Description of Artificial Sequence Synthetic peptide
YDKPH

SEQ ID NO: 19 - protein, 4 aa, Artificial Sequence
  Description of Artificial Sequence Synthetic peptide
  MOD_RES (2)..(3): Any natural amino acid
  See specification as filed for detailed description of substitutions and preferred embodiments
YXXL

SEQ ID NO: 20 - protein, 4 aa, Artificial Sequence
  Description of Artificial Sequence Synthetic peptide
YLSL

SEQ ID NO: 21 - protein, 4 aa, Artificial Sequence
  Description of Artificial Sequence Synthetic peptide
  MOD_RES (2)..(3): Any amino acid
  See specification as filed for detailed description of substitutions and preferred embodiments
YXXQ

SEQ ID NO: 22 - protein, 11 aa, Artificial Sequence
  Description of Artificial Sequence Synthetic peptide
DICLPRWGCLW

SEQ ID NO: 23 - protein, 132 aa, Artificial Sequence
  Description of Artificial Sequence Synthetic polypeptide
PTSSSTKKTQLQLSQLLVLLKAILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 24 - protein, 4 aa, Artificial Sequence
  Description of Artificial Sequence Synthetic peptide
YLRQ

SEQ ID NO: 25 - protein, 4 aa, Artificial Sequence
  Description of Artificial Sequence Synthetic peptide
YLKQ

SEQ ID NO: 26 - protein, 4 aa, Artificial Sequence
  Description of Artificial Sequence Synthetic peptide
YRHQ

SEQ ID NO: 27 - DNA, 50 bp, Artificial Sequence
  Description of Artificial Sequence Synthetic primer
ccattcaaaatcatctgtaagagcaccagtaactggctcagttgtagctg

SEQ ID NO: 28 - protein, 4 aa, Artificial Sequence
  Description of Artificial Sequence Synthetic peptide
YFKQ

SEQ ID NO: 29 - protein, 525 aa, Artificial Sequence
  Description of Artificial Sequence Synthetic polypeptide
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 30 - protein, 489 aa, Artificial Sequence
  Description of Artificial Sequence Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 1060 aa, Artificial Sequence
  Description of Artificial Sequence Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPGSGEGRGSLLTCGDVEENPGPMAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 32 - protein, 488 aa, Artificial Sequence
  Description of Artificial Sequence Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 33 - protein, 486 aa, Artificial Sequence
  Description of Artificial Sequence Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 490 aa, Artificial Sequence
  Description of Artificial Sequence Synthetic polypeptide
MALPVTALLLPLALLLHAARPQVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSSASGGGGSGGRASGGGGSDIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 1062 aa, Artificial Sequence
  Description of Artificial Sequence Synthetic polypeptide
MALPVTALLLPLALLLHAARPQVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSSASGGGGSGGRASGGGGSDIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 36 - protein, 488 aa, Artificial Sequence
  Description of Artificial Sequence Synthetic polypeptide
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 1060 aa, Artificial Sequence
  Description of Artificial Sequence Synthetic polypeptide
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 38 - protein, 493 aa, Artificial Sequence
  Description of Artificial Sequence Synthetic polypeptide
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 483 aa, Artificial Sequence
  Description of Artificial Sequence Synthetic polypeptide
MALPVTALLLPLALLLHAARPEVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 1055 aa, Artificial Sequence
  Description of Artificial Sequence Synthetic polypeptide
MALPVTALLLPLALLLHAARPEVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 41 - protein, 483 aa, Artificial Sequence
  Description of Artificial Sequence Synthetic polypeptide
MALPVTALLLPLALLLHAARPDIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKGSTSGGGSGGGSGGGGSSEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 1055 aa, Artificial Sequence
  Description of Artificial Sequence Synthetic polypeptide
MALPVTALLLPLALLLHAARPDIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKGSTSGGGSGGGSGGGGSSEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 43 - protein, 482 aa, Artificial Sequence
  Description of Artificial Sequence Synthetic polypeptide
MALPVTALLLPLALLLHAARPDIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKGSTSGGGSGGGSGGGGSSEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 44 - protein, 1054 aa, Artificial Sequence
  Description of Artificial Sequence Synthetic polypeptide
MALPVTALLLPLALLLHAARPDIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKGSTSGGGSGGGSGGGGSSEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPMAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 45 - protein, 469 aa, Artificial Sequence
  Description of Artificial Sequence Synthetic polypeptide
DVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 46 - protein, 464 aa, Artificial Sequence
  Description of Artificial Sequence Synthetic polypeptide
DVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 47 - protein, 603 aa, Artificial Sequence
  Description of Artificial Sequence Synthetic polypeptide
MALEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGHPSSNSGYALNFGKGTSLLVTPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSRAKRSGSGATNFSLLKQAGDVEENPGPMGTSLLCWVVLGFLGTDHTGAGVSQSPRYKVTKRGQDVALRCDPISGHVSLYWYRQALGQGPEFLTYFNYEAQQDKSGLPNDRFSAERPEGSISTLTIQRTEQRDSAMYRCASSSQTGARTDTQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 48 - protein, 598 aa, Artificial Sequence
  Description of Artificial Sequence Synthetic polypeptide
MAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 49 - protein, 245 aa, Artificial Sequence
  Description of Artificial Sequence Synthetic polypeptide
DVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELGGGGSGGGGSGGGGSGGGGSEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSS

SEQ ID NO: 50 - protein, 133 aa, Artificial Sequence
  Description of Artificial Sequence Synthetic polypeptide
  MOD_RES (1)..(1): A or not present
  MOD_RES (2)..(2): P or not present
  MOD_RES (3)..(3): T, C, A, G, Q, E, N, D, R, K, P, or not present
  MOD_RES (4)..(4): S or not present
  MOD_RES (5)..(5): S or not present
  MOD_RES (6)..(6): S or not present
  MOD_RES (7)..(7): T or not present
  MOD_RES (8)..(8): K or not present
  MOD_RES (9)..(9): K or not present
  MOD_RES (13)..(13): Q, W, or not present
  MOD_RES (14)..(14): L, M, W, or not present
  MOD_RES (15)..(15): E, K, D, T, A, S, Q, H, or not present
  MOD_RES (16)..(16): H, N, Q, or not present
  MOD_RES (18)..(18): L, R, G, M, F, E, H, W, K, Q, S, V, I, Y, H, D, or T
  MOD_RES (19)..(19): L, A, V, I, or not present
  MOD_RES (20)..(20): D, T, S, M, L, or not present
  MOD_RES (22)..(22): Q, F, E, G, A, L, M, F, W, K, S, V, I, Y, H, R, N, D, T, F, or not present
  MOD_RES (23)..(23): M, A, W, H, Y, F, Q, S, V, L, T, or not present
  MOD_RES (27)..(27): G, K, S, or not present
  MOD_RES (38)..(38): R, W, or G
  MOD_RES (39)..(39): M, L, or V
  MOD_RES (42)..(42): F or K
  MOD_RES (51)..(51): T, I, or not present
  MOD_RES (55)..(55): H or Y
  MOD_RES (74)..(74): Q, N, H, or S
  MOD_RES (80)..(80): L, F, or V
  MOD_RES (81)..(81): R, I, D, Y, T, or not present
  MOD_RES (85)..(85): L or V
  MOD_RES (86)..(86): I or V
  MOD_RES (89)..(89): I or V
  MOD_RES (91)..(91): V, R, or K
  MOD_RES (92)..(92): I or F
  MOD_RES (97)..(97): K or Q
  MOD_RES (104)..(104): M or A
  MOD_RES (109)..(109): D, C, or a non-natural amino acid with an activated side chain
  MOD_RES (113)..(113): T or N
  MOD_RES (125)..(125): C, A, or S
  MOD_RES (126)..(126): Q, H, M, K, C, D, E, G, I, R, S, or T
  MOD_RES (130)..(130): S, T, or R
XXXXXXXXXTQLXXXXLXXXLXXILNXINNYKNPKLTXXLTXKFYMPKKAXELKXLQCLEEELKPLEEVLNLAXSKNFHXXPRDXXSNXNXXVLELXGSETTFXCEYAXETAXIVEFLNRWITFXXSIIXTLT

SEQ ID NO: 51 - protein, 6 aa, Artificial Sequence
  Description of Artificial Sequence Synthetic His tag
  SITE (1)..(6): This sequence may encompass 3-6 residues
HHHHHH

SEQ ID NO: 52 - protein, 6 aa, Artificial Sequence
  Description of Artificial Sequence Synthetic 6xHis tag
HHHHHH

SEQ ID NO: 53 - protein, 214 aa, Artificial Sequence
  Description of Artificial Sequence Synthetic polypeptide
  MOD_RES (70)..(70): Q or Y
  MOD_RES (73)..(73): T, D, or Y
  MOD_RES (133)..(133): H, D, E, or K
  MOD_RES (134)..(134): Y, F, E, or R
AVNGTSQFTCFYNSRANISCVWSQDGALQDTSCQVHAWPDRRRWNQTCELLPVSQASWACNLILGAPDSXKLXTVDIVTLRVLCREGVRWRVMAIQDFKPFENLRLMAPISLQVVHVETHRCNISWEISQASXXFERHLEFEARTLSPGHTWEEAPLLTLKQKQEWICLETLTPDTQYEFQVRVKPLQGEFTTWSPWSQPLAFRTKPAALGKDT

SEQ ID NO: 54 - protein, 6 aa, Artificial Sequence
  Description of Artificial Sequence Synthetic peptide
GGYLRQ

SEQ ID NO: 55 - protein, 6 aa, Artificial Sequence
  Description of Artificial Sequence Synthetic peptide
GGYLKQ

SEQ ID NO: 56 - protein, 6 aa, Artificial Sequence
  Description of Artificial Sequence Synthetic peptide
GGYRHQ

SEQ ID NO: 57 - DNA, 60 bp, Artificial Sequence
  Description of Artificial Sequence Synthetic primer
caactgagccagttactggtgctcttaaaggccattttgaatggaattaataattacaag

SEQ ID NO: 58 - protein, 6 aa, Artificial Sequence
  Description of Artificial Sequence Synthetic peptide
GGYFKQ

SEQ ID NO: 59 - protein, 6 aa, Artificial Sequence
  Description of Artificial Sequence Synthetic peptide
GGYLPQ

SEQ ID NO: 60 - protein, 6 aa, Artificial Sequence
  Description of Artificial Sequence Synthetic peptide
GGYMPQ

SEQ ID NO: 61 - protein, 7 aa, Artificial Sequence
  Description of Artificial Sequence Synthetic peptide
GGYDKPH

SEQ ID NO: 62 - protein, 10 aa, Homo sapiens
YFTYDPYSEE

SEQ ID NO: 63 - protein, 4 aa, Homo sapiens
YFTY

SEQ ID NO: 64 - protein, 4 aa, Homo sapiens
YDPY

SEQ ID NO: 65 - protein, 4 aa, Homo sapiens
YSEE

SEQ ID NO: 66 - DNA, 40 bp, Artificial Sequence
  Description of Artificial Sequence Synthetic primer
tatagtcagcgccacccatgtacaggatgcaactcctgtc

SEQ ID NO: 67 - DNA, 31 bp, Artificial Sequence
  Description of Artificial Sequence Synthetic primer
tatagggccctatcaagtcagtgttgagatg

SEQ ID NO: 68 - DNA, 49 bp, Artificial Sequence
  Description of Artificial Sequence Synthetic primer
caaagaaaacacagctacaactggagcagttactggtgctcttaaaggc

SEQ ID NO: 69 - DNA, 49 bp, Artificial Sequence
  Description of Artificial Sequence Synthetic primer
gcctttaagagcaccagtaactgctccagttgtagctgtgttttctttg

SEQ ID NO: 70 - DNA, 35 bp, Artificial Sequence
  Description of Artificial Sequence Synthetic primer
cagctacaactgagccatttactggtgctcttaaa

SEQ ID NO: 71 - DNA, 35 bp, Artificial Sequence
  Description of Artificial Sequence Synthetic primer
tttaagagcaccagtaaatggctcagttgtagctg

SEQ ID NO: 72 - DNA, 30 bp, Artificial Sequence
  Description of Artificial Sequence Synthetic primer
ctgagccagttactgctgctcttaaaggcc

SEQ ID NO: 73 - DNA, 30 bp, Artificial Sequence
  Description of Artificial Sequence Synthetic primer
ggcctttaagagcagcagtaactggctcag

SEQ ID NO: 74 - DNA, 42 bp, Artificial Sequence
  Description of Artificial Sequence Synthetic primer
aactgagccagttactggtggatttaaaggccattttgaatg

SEQ ID NO: 75 - DNA, 42 bp, Artificial Sequence
  Description of Artificial Sequence Synthetic primer
cattcaaaatggcctttaaatccaccagtaactggctcagtt

SEQ ID NO: 76 - DNA, 36 bp, Artificial Sequence
  Description of Artificial Sequence Synthetic primer
tgagccagttactggtgctcttacaggccattttga

SEQ ID NO: 77 - DNA, 36 bp, Artificial Sequence
  Description of Artificial Sequence Synthetic primer
tcaaaatggcctgtaagagcaccagtaactggctca

SEQ ID NO: 78 - DNA, 35 bp, Artificial Sequence
  Description of Artificial Sequence Synthetic primer
ctggtgctcttaaagatgattttgaatggaattaa

SEQ ID NO: 79 - DNA, 35 bp, Artificial Sequence
  Description of Artificial Sequence Synthetic primer
ttaattccattcaaaatcatctttaagagcaccag

SEQ ID NO: 80 - DNA, 30 bp, Artificial Sequence
  Description of Artificial Sequence Synthetic primer
cacagctacaactgtcgcatttactgctgg

SEQ ID NO: 81 - DNA, 30 bp, Artificial Sequence
  Description of Artificial Sequence Synthetic primer
ccagcagtaaatgcgacagttgtagctgtg

SEQ ID NO: 82 - DNA, 32 bp, Artificial Sequence
  Description of Artificial Sequence Synthetic primer
gctacaactggagcagttactgctggatttac

SEQ ID NO: 83 - DNA, 32 bp, Artificial Sequence
  Description of Artificial Sequence Synthetic primer
gtaaatccagcagtaactgctccagttgtagc

SEQ ID NO: 84 - DNA, 36 bp, Artificial Sequence
  Description of Artificial Sequence Synthetic primer
ctggagcatttactggtggatttacagatgattttg

SEQ ID NO: 85 - DNA, 36 bp, Artificial Sequence
  Description of Artificial Sequence Synthetic primer
caaaatcatctgtaaatccaccagtaaatgctccag

SEQ ID NO: 86 - DNA, 33 bp, Artificial Sequence
  Description of Artificial Sequence Synthetic primer
gagcatttactgctgctattacagatgattttg

SEQ ID NO: 87 - DNA, 33 bp, Artificial Sequence
  Description of Artificial Sequence Synthetic primer
caaaatcatctgtaatagcagcagtaaatgctc

SEQ ID NO: 88 - DNA, 35 bp, Artificial Sequence
  Description of Artificial Sequence Synthetic primer
catttactgctggatttaaagatgattttgaatgg

SEQ ID NO: 89 - DNA, 35 bp, Artificial Sequence
  Description of Artificial Sequence Synthetic primer
ccattcaaaatcatctttaaatccagcagtaaatg

SEQ ID NO: 90 - DNA, 55 bp, Artificial Sequence
  Description of Artificial Sequence Synthetic primer
ctggagcatttactgctggatttacaggcgattttgaatggaattaataattaca

SEQ ID NO: 91 - DNA, 55 bp, Artificial Sequence
  Description of Artificial Sequence Synthetic primer
tgtaattattaattccattcaaaatcgcctgtaaatccagcagtaaatgctccag

SEQ ID NO: 92 - DNA, 50 bp, Artificial Sequence
  Description of Artificial Sequence Synthetic primer
caaagaaaacacagctacaactgagccagttactgctggatttacagatg

SEQ ID NO: 93 - DNA, 50 bp, Artificial Sequence
  Description of Artificial Sequence Synthetic primer
catctgtaaatccagcagtaactggctcagttgtagctgtgttttctttg

(19) United States
(12) Patent Application Publication
(10) Pub. No.: US 2023/0076768 A1
(43) Pub. Date: Mar. 9, 2023

(54) IL2 Orthologs and Methods of Use

(71) Applicant: Synthekine, Inc., Menlo Park, CA (US)

(72) Inventors: Paul-Joseph Penaflor Aspuria, Menlo Park, CA (US); Patrick J. Lupardus, Menlo Park, CA (US); Richard B. Murphy, Menlo Park, CA (US); Martin Oft, Menlo Park, CA (US)

(21) Appl. No.: 17/758,830

(22) PCT Filed: Jan. 14, 2021
(86) PCT No.: PCT/US2021/013521

§ 371 Date: Jul. 14, 2022

Related U.S. Application Data
(60) Provisional application No. 62/961,200, filed on Jan. 14, 2020.
(60) Provisional application No. 63/015,476, filed on Apr. 24, 2020.
(60) Provisional application No. 63/016,256, filed on Apr. 27, 2020.

(51) Int. Cl. C07K 14/715; C12N 15/86; A61P 35/00

(52) U.S. Cl. CPC C07K 14/7155; C12N 15/86; A61P 35/00

(57) ABSTRACT

The present disclosure provides orthogonal receptors. In some embodiments, the orthogonal receptor is an orthogonal CD122. In some embodiments, the orthogonal receptor is an orthogonal human CD122 (hCD122). In some embodiments, the orthogonal receptor is an orthogonal CD122 comprising at least one STAT3 binding motifs.

### CROSS-REFERENCE TO RELATED PATENT APPLICATIONS

The present application claims benefit of priority to each of U.S. Provisional Patent Application No. 62/961,200, filed Jan. 14, 2020; U.S. Provisional Patent Application No. 63/015,476, filed Apr. 24, 2020; and U.S. Provisional Patent Application No. 63/016,256, filed Apr. 27, 2020, each of which are incorporated by reference for all purposes.

### SEQUENCE LISTING

The instant application contains a Sequence Listing which has been submitted electronically in ASCII format and is hereby incorporated by reference in its entirety. Said ASCII copy, created on Jun. 14, 2022, is named 1218671-Sequence-Listing.txt and is 171,176 bytes in size.

### BACKGROUND OF THE INVENTION

Adoptive cell therapy has been documented as a therapeutic modality having efficacy in the treatment of disease in human subjects. In some instances, cell therapy involves the administration of a cell product comprising ex vivo expanded tumor infiltrating lymphocytes (TILs) obtained from resected tumor tissue from the patient. See, e.g., Rosenberg (U.S. Pat. No. 5,126,132A issued Jun. 30, 1992 and Spiess, et al (1987) J Natl Cancer Inst 79:1067-1075. Subjects suffering from metastatic melanoma treated with adoptive TIL therapy in substantial accordance with this regimen obtained objective tumor responses of around 50% in several phase I/II clinical trials. See, e.g. Rosenberg, et al. (2011) Clin Cancer Res 17:4550-4557; Andersen, et al. (2016) Clin Cancer Res 22:3734-3745; and Besser, et al (2013) Clin Cancer Res 19:4792-4800. Building on the success of TIL therapy observed in melanoma patients, others demonstrated that it is possible to obtain TILs from a wide variety of other tumor types including but not limited to cervical cancer (Stevanovic, et al (2015) J Clin Oncol 33:1543-1550), renal cell cancer (Andersen, et al (2018) Cancer Immunol Res 6:222-235), breast cancer (Lee, et al (2017) Oncotarget 8:113345-113359), non-small cell lung cancer (Ben-Avi, et al (2018) Cancer Immunol Immunother 67:1221-1230) gastrointestinal cancers (Turcotte (2013) J Immunol 191:2217-2225 and Turcotte, et al (2014) Clin Cancer Res 20:331-343), cholangiocarcinoma (Tran, et al (2014) Science 344:641-645), pancreatic cancer (Hall, et al (2016) J Immunotherapy Cancer 4:61) head and neck cancer (Junker, et al (2011) Cytotherapy 13:822-834) and ovarian cancer (Fujita, et al (1995) Clin Cancer Res 1: 501-507).

A wide variety of engineered immune cells (e.g. T cells, NK cells, and TILs) have been developed for the immunotherapeutic treatment of human disease. Human immune cells have been engineered for use in therapeutic applications such as the recognition and killing of cancer cells, intracellular pathogens and cells involved in autoimmunity. The use of engineered cell therapies in the treatment of cancer is facilitated by the selective activation and expansion of engineered cells (such as T cells) to provide specific functions and are directed to selectively attack cancer cells. In some examples of adoptive immunotherapy, T cells are isolated from the blood or tumor tissue of a subject, processed ex vivo, and reinfused into the subject. Compositions and methods that enable selective activation of such a targeted engineered cell population are therefore desirable.

A challenge to the clinical application of adoptive cell therapies is the maintenance of the viability of the adoptively transferred cells following administration to a subject to maintain and maximize their therapeutic effectiveness. Successful maintenance of the viability of the adoptively transferred cells following administration to the subject facilitates the clinical response to such adoptive cell therapy. Although the cells administered to a subject an adoptive cell therapy regimen may be detectable in the subject for months or even years following the administration, a significant fraction (typically the majority) of the administered cells lapse into a quiescent state in which they lose therapeutic anti-tumor efficacy. Such loss of activity of the adoptively transferred cells frequently correlates with a loss of clinical efficacy including relapse or recurrence of the neoplastic disease.

In clinical practice in human subjects, to support the adoptively transferred cells (e.g., TIL therapy and engineered T cells such as CAR-T cells, a commonly employed means to support the viability of the adoptively transferred cells following administration to the subject is the systemic administration of the pluripotent cytokine, interleukin-2. Human interleukin 2 (IL2) is a 4 alpha-helix bundle cytokine of 133 amino acids. IL2 is member of the IL2 family of cytokines which includes IL2, IL-4, IL-7, IL 9, IL-15 and IL21. The amino acid sequence of a hIL2 (SEQ ID NO: 1) is found in Genbank under accession locator NP 000577.2. IL2 is produced by antigen activated T cells and exerts a wide spectrum of effects on the immune system and plays important roles in regulating both immune activation, suppression and homeostasis. IL2 promotes the proliferation and expansion of activated T lymphocytes, induces proliferation and activation of naïve T cells, potentiates B cell growth, and promotes the proliferation and expansion of NK cells. Clinical experience demonstrates that HD-IL2 treatment activates naïve T cells and NK cells. Preclinical experiments have implicated NK cells as the dominant mechanism for IL2 mediated acute toxicity. Assier, et al. (2004) J Immunol 172:7661-7668. The administration of HD-IL2 also stimulates the expansion of CD25+ regulatory T cells (Tregs) which mediate the activity of CD8+ T cells. The expansion of NK and Tregs are conventionally believed to result in additional toxicities such as CRS.

In the typical current clinical practice of adoptive cell therapy with engineered tumor infiltrating lymphocytes (“TILs”) or CAR-T cells, in conjunction with or shortly after infusion of the TILs or CAR-T cells, the subject receives intravenous high-dose hIL2 (HD-hIL2) 720,000 IU/kg) hIL2 every 8 hours for as long as the subject is able to tolerate the treatment. This administration of HD-hIL2 in conjunction with adoptive cell therapy is thought to further enhance the survival and clinical efficacy of the engineered cell product. Anderson, et al (2016) Clinical Cancer Research 22:3734-3745. The commonly used form human IL2 (hIL2) is aldesleukin (Proleukin®), a hIL2 analog having a substitution of the cysteine at position 125 the mature hIL2 molecule with a serine (C125S).

However, the systemic administration of hIL2 is associated with non-specific stimulatory effects beyond the population of adoptively transferred cells and is associated, particularly in high doses, with significant toxicity in human subjects. The effect of high dose hIL2 such as that used in support of adoptive cell therapy regimens is documented to result in significant toxicities in human subjects. The most prevalent side effects observed from the administration of HD-hIL2 in conjunction with adoptive cell transfer (ACT) include chills, high fever, hypotension, oliguria, and edema due to the systemic inflammatory and capillary leak syndrome as well as reports of autoimmune phenomena such as vitiligo or uveitis. The toxicities associated with HD-hIL2 require expert management and is therefore typically applied in the hospital setting and frequently requires admission to an intensive care unit. Dutcher, et al. (2014) J Immunother Cancer 2(1): 26. HD-hIL2 treatment activates most lymphatic cells, including naïve T cells and NK cells, which predominantly express the intermediate affinity receptor (CD122/CD132) and CD25+ regulatory T cells (Tregs), which express the high affinity trimeric receptor (CD25/CD122/CD132). HD-hIL2 monotherapy may also induce generalized capillary leak syndrome which can lead to death. This limits the use of HD-IL2 therapy to mostly younger, very healthy patients with normal cardiac and pulmonary function. HD-IL2 therapy is typically applied in the hospital setting and frequently requires admission to an intensive care unit. Although nitric oxide synthase inhibitors have been suggested to ameliorate the symptoms of VLS, the common practice when VLS is observed is the withdrawal of IL2 therapy. To mitigate the VLS associated with HD IL2 treatment, low-dose IL2 regimens have been tested in patients. While low dose IL2 treatment regimens do partially mitigate the VLS toxicity, this lower toxicity was achieved at the expense of optimal therapeutic results in the treatment of neoplasms.

Furthermore, the clinically approved forms of hIL2 (e.g. Proleukin®) possess a comparatively short lifespan in vivo (on the order of hours) necessitating that frequent dosing of the IL2 to maintain sufficient exposure of the engineered T cells to the IL2 to maintain the cells in an activated state. Although modification of IL2 to extend its in vivo lifespan have been described in the literature (e.g. PEGylation, Katre, et al, U.S. Pat. No. 5,206,344 issued Apr. 27, 1993, Meyers, et al. (1991) Clinical Pharmacology and Therapeutics 13(1):307-313) the administration of such long acting forms of wild type hIL2 nevertheless present similar toxicity concerns as the parent molecule and the prolonged exposure of such agents may serve to exacerbate such toxicities. Consequently, a significant challenge to cell-based therapies is to confer the engineered cell with a desired regulatable proliferative behavioral signal that is independent from modulation by from endogenous signaling pathways, that exhibits minimal cross reactivity with non-targeted endogenous cells, and that can be controlled selectively following administration of the engineered cell population to a subject.

In current practice, prior to the administration of the adoptive cell therapy product, the subject is subjected to a lymphodepleting preparative regimen and subsequent support of interleukin-2 (IL-2). As the doses of cells in a cell therapy regimen are typically very high (typically a single administration in the range of 10⁶-10⁸ cells for current CD19 CAR T products), the lymphodepleting preparative regimen depletes Tregs and removes cellular “sinks” and is believed to provide “room” for the adoptively transferred cells. However, lymphodepletion significantly compromises the patient by leaving them vulnerable to environmental factors. Consequently, avoiding lymphodepleting regimens in conjunction with cell therapy would be of significant benefit to the patient.

A challenge to the manufacture of cell therapy products is that such ‘living drugs’ require close control of their environment to preserve viability and functionality. In practice, isolated cells, whether derived from a patient (autologous) or from a single donor source (allogeneic), begin to lose function rapidly following removal from a subject or the controlled culture conditions. Successful maintenance of the viability of isolated cells while outside the subject or controlled culture conditions enables the isolated cells to return to functionality for reinsertion into the cell product manufacturing workflow or into patients. During the ex vivo preparation of cells for use in adoptive cell therapy, the cells are frequently cultured in the presence of exogenous hIL2. As the effect of hIL2 is non-specific to the engineered cell in a mixed cell population comprising both engineered and non-engineered immune cells, this exposure to IL2 leads to the expansion of not just the desired therapeutically useful cells (e.g. CAR-T cells or antigen experienced TILs) in the cell population but also the expansion of a variety of undesired background cells from the isolated tissue (e.g. neoplasm or blood) sample which provide no clinical benefit, complicate the dosing of the engineered cell product, and may contribute to toxicity. Consequently, current ex vivo expansion methods for the preparation of cells useful in autologous cell transfer result in cell products in which the percentage of desired therapeutically useful cells is contaminated with undesired cells resulting in a suboptimal cell product. As significant toxicity remains a significant issue with currently ACT protocols, methods that enable the preparation of a cell product comprising a more homogeneous cell population enriched the desired efficacious cells (e.g. CAR-T cells or antigen experienced TILs) product for use in ACT therapy is desirable.

Additionally, current cell therapies are very expensive due to their complex nature and the management of associated toxicities often requiring the patient to remain at or near a major medical center for prolonged periods of time. The manufacture of large doses of cell therapeutics is also complex and time consuming. Many efforts are being directed at reducing the time from extraction of the patient cells to the reinfusion of their engineered version (so called “vein-to-vein” time) of autologous cell therapies which are currently approximately three weeks. Allogenic or “off-the-shelf” engineered T cells are currently being investigated to avoid this delay in therapy and to avoid the expenses associated with such individualized cell therapies. The lack of persistence of current cell therapies also leads to requiring large doses of the engineered cells which further increases costs and further delay in vein-to-vein times. Despite their demonstrated therapeutic utility and promise, the cost of cell therapies puts significant strains on healthcare systems of limited resources and consequently may have the effect of limiting their broader availability to patients in need. The compositions and methods of the present disclosure address many of these issues.

CD122 is a component of the intermediate and high affinity IL2 receptor complexes. Sockolosky, et al. (Science (2018) 359: 1037-1042) and Garcia, et al. (United States Patent Application Publication US2018/0228841A1 published Aug. 16, 2018) describe an orthogonal IL2/CD122 ligand/receptor system to facilitate selective stimulation of cells engineered to express an orthogonal receptor, especially an orthogonal CD122. The present patent application incorporates by reference the disclosures of WO 2019/104092 and US 2018-0228842 A1) in their entireties. The contact of engineered T cells that express the orthogonal CD122 with a corresponding orthogonal ligand cognate for such orthogonal CD122 (“IL2 ortholog”) facilitates specific activation of such engineered T cells that express the orthogonal CD122. In particular this orthogonal IL2 receptor ligand complex provides for selective expansion of cells engineered to express the orthogonal receptor in a mixed population of cells, in particular a mixed population of T cells.

### SUMMARY OF THE INVENTION

The present disclosure provides methods and compositions useful in the practice of adoptive cell therapy.

In some embodiments, the present disclosure provides orthogonal receptors. In some embodiments, the orthogonal receptor is an orthogonal CD122. In some embodiments, the orthogonal receptor is an orthogonal human CD122 (hCD122). In some embodiments, the orthogonal receptor is an orthogonal CD122 comprising at least one STAT3 binding motifs.

In some embodiments, the present disclosure provides a recombinant vector comprising a nucleic acid sequence encoding an orthogonal hCD122 receptor.

In some embodiments, the present disclosure provides a recombinant vector comprising a nucleic acid sequence encoding an orthogonal hCD122 receptor and a CAR. In some embodiments the CAR.

In some embodiments, the present disclosure provides recombinant mammalian immune cell comprising a nucleic acid sequence encoding an orthogonal hCD122 receptor and a nucleic acid sequence encoding a CAR.

In some embodiments, the present disclosure provides orthologs that are cognate ligands for an orthogonal receptor. In some embodiments, the orthologs are IL2 orthologs. In some embodiments, the IL2 orthologs are ligands for an orthogonal CD122 receptor. In some embodiments, the IL2 orthologs are cognate ligands for a transmembrane receptor protein comprising the extracellular domain of an orthogonal hCD122 receptor. In some embodiments, the IL2 orthologs are cognate ligands for a transmembrane receptor protein comprising the extracellular domain of an orthogonal hCD122 receptor comprising amino acid substitutions at positions H133 and Y134. In some embodiments, the IL2 orthologs are ligands for an orthogonal hCD122 comprising amino acid substitutions at positions H133 and Y134. In some embodiments, the IL2 orthologs are ligands for an orthogonal hCD122 comprising the amino acid substitutions H133D and Y134F.

In some embodiments, the present disclosure provides pharmaceutically acceptable formulations comprising a hIL2 ortholog of Formula 1.

In some embodiments, the present disclosure provides a nucleic acid sequence encoding a hIL2 ortholog of Formula 1.

In some embodiments, the present disclosure provides a recombinant vector comprising a nucleic acid sequence encoding a hIL2 ortholog of Formula 1.

In some embodiments, the present disclosure provides a pharmaceutically acceptable formulation of a recombinant vector comprising a nucleic acid sequence encoding a hIL2 ortholog of Formula 1.

In some embodiments, the present disclosure provides a recombinantly modified mammalian cell comprising a recombinant vector, the vector comprising a nucleic acid sequence encoding a hIL2 ortholog of Formula 1.

In some embodiments, the present disclosure provides orthogonal mammalian immune cells that are recombinantly modified to express an orthogonal receptor (orthogonal immune cells). In some embodiments, the orthogonal immune cells immune cell is a T cell. In In some embodiments the T cell is selected from naïve CD8⁺ T cells, cytotoxic CD8⁺ T cells, naïve CD4⁺ T cells, helper T cells, e.g. T_{H}1, T_{H}2, T_{H}9, T_{H}11, T_{H}22, T_{FH}; regulatory T cells, e.g. T_{R}1, Tregs, inducible Tregs; memory T cells, e.g. central memory T cells, effector memory T cells, NK cells, tumor infiltrating lymphocytes (TILs) and engineered variants of such T-cells including but not limited to CAR-T cells, recombinantly modified TILs and TCR engineered cells.

In some embodiments, the present disclosure provides an orthogonal Treg cell. The present invention further provides a method of induce immune suppression in a subject by the administration a therapeutically effective amount of an orthogonal Treg in combination with the administration of an orthogonal ligand sufficient to cause the proliferation and/or activation of the orthogonal Treg in the subject. In some embodiments, the orthogonal Treg optionally provides a targeting domain (e.g., an engineered TCR or CAR). In some embodiments, the orthogonal Treg is an orthogonal CAR-Treg (oCAR-Treg). In some embodiments the ABD of the CAR of the oCAR-Treg specifically binds to human Factor VIII. In some embodiments, the ABD of the CAR of the oCAR-Treg specifically binds to an alloantigen. In some embodiments, the ABD of the CAR of the oCAR-Treg specifically binds to HLA-A2. In some embodiments, the oCAR-Treg is a CD19-oCAR-Treg. Orthogonal Treys are useful in the treatment of diseases associated with inflammatory diseases.

In some embodiments, the present disclosure provides an orthogonal NK cell. The present invention further provides a method of treating neoplastic disease in a subject by the administration a therapeutically effective amount of an orthogonal NK (oNK) cell in combination with the administration of an orthogonal ligand sufficient to cause the proliferation and/or activation of the orthogonal Treg in the subject. In some embodiments, the oNK cell optionally provides a targeting domain (e.g., an engineered TCR or CAR). In some embodiments, the orthogonal NK cell is an orthogonal CAR-NK cell (oCAR-NK cell).

In some embodiments, the present disclosure provides mammalian immune cells that are recombinantly modified to express an orthogonal CD122 polypeptide. In some embodiments, the present disclosure provides mammalian cells that are recombinantly modified to express an orthogonal receptor comprising the extracellular domain of an orthogonal hCD122. In some embodiments, the present disclosure provides mammalian cells that are recombinantly modified to express an orthogonal receptor comprising the extracellular domain of CD122 comprising amino acid substitutions at positions H133 and Y134. In some embodiments, the present disclosure provides mammalian cells that are recombinantly modified to express an orthogonal hCD122 comprising amino acid substitutions at positions H133 and Y134. In some embodiments, the present disclosure provides mammalian cells that are recombinantly modified to express an orthogonal hCD122 comprising the amino acid substitutions H133D and Y134F.

In some embodiments, the present disclosure provides methods of use of an orthogonal receptor and its cognate orthogonal ligand (ortholog) to induce a signal in a mammalian cell expressing the orthogonal receptor. In some embodiments, the present disclosure provides a method of causing a response in a mammalian cell, the cell engineered to express an orthogonal receptor, the method comprising contacting the engineered mammalian cell expressing an orthogonal receptor with an effective amount of a cognate orthogonal ligand (ortholog) wherein the response caused by such contacting is the is the activation of the engineered cell, maintenance of an activated state of the engineered cell and/or proliferation of the engineered cell. In some embodiments, the present disclosure provides a method of causing a response in a mammalian cell, the cell engineered to express an orthogonal receptor, the method comprising contacting the engineered mammalian cell expressing an orthogonal receptor with an effective amount of a cognate orthogonal ligand (ortholog) wherein the response caused by such contacting is an increase in STAT5 phosphorylation and/or STAT3 phosphorylation in the engineered cell.

In some embodiments, the present disclosure provides a method of causing a response in a mammalian immune cell, the cell engineered to express an orthogonal receptor, the method comprising contacting the engineered mammalian cell expressing an orthogonal receptor with an effective amount of a cognate orthogonal ligand (ortholog) wherein the contacting is performed ex vivo (in vitro) and the response caused by such contacting is the is the activation of the engineered cell, maintenance of an activated state of the engineered cell and/or causing proliferation of the engineered cell.

In some embodiments, the present disclosure provides a method of preparing an engineered immune cell product substantially enriched for engineered cells the method comprising the steps of: (a) isolating a mixed population of immune cells from a subject; (b) transfecting a fraction of the population of said isolated immune cells with a recombinant vector capable of effecting the expression of an orthogonal receptor in the transfected cells; (c) culturing said mixed immune cell population in the presence of an orthogonal ligand that specifically binds to the ECD of the orthogonal receptor such that the cells expressing the orthogonal receptor selectively proliferate enriching the population of cells for cells expressing the orthogonal receptor.

In some embodiments, the present disclosure provides a method of causing a response in a mammalian immune cell, the cell engineered to express an orthogonal receptor, the method comprising contacting the engineered mammalian cell expressing an orthogonal receptor with an effective amount of a cognate orthogonal ligand (ortholog) and the response caused by such contacting is the activation of the engineered cell, maintenance of an activated state of the engineered cell and/or proliferation of the engineered cell.

In some embodiments, the present disclosure provides a method of causing a response in a mammalian cell expressing an orthogonal receptor the method comprising contacting the mammalian cell expressing an orthogonal receptor ex vivo (in vitro) with a cognate orthogonal ligand (ortholog) in an amount sufficient to cause a response.

In some embodiments, the present disclosure provides a method of causing a response in a mammalian cell expressing an orthogonal receptor the method comprising contacting the mammalian cell expressing an orthogonal receptor in vivo with a cognate ortholog ligand in an amount sufficient to cause a response.

In some embodiments, the present disclosure provides methods of use comprising the use a first ortholog (i.e., cognate ligand) ex vivo and a second ortholog in vivo. In some embodiments, the present disclosure provides methods of use comprising the use a first ortholog ex vivo and a second ortholog in vivo, wherein the first ortholog and the second ortholog are the same orthologs or different orthologs. In some embodiments, the present disclosure provides methods of use of orthologs to cause the proliferation of a mammalian cell expressing an orthogonal receptor.

In some embodiments, the present disclosure provides methods of use of orthologs to cause the activation of a mammalian cell expressing an orthogonal receptor. In some embodiments, the present disclosure provides methods of use of orthologs ex vivo and/or in vivo to cause the proliferation of a mammalian cell expressing an orthogonal receptor. In some embodiments, the present disclosure provides methods of use of IL2 orthologs to cause the proliferation of a mammalian cell expressing an orthogonal CD122.

In some embodiments, the present disclosure provides methods of use of IL2 orthologs to cause the activation of a mammalian cell expressing an orthogonal CD122. In some embodiments, the present disclosure provides methods of use of orthologs to cause the activation of a mammalian cell recombinantly modified to express an orthogonal receptor comprising the extracellular domain of CD122 comprising amino acid substitutions at positions H133 and Y134.

In some embodiments, the present disclosure provides methods of use of orthologs to cause the activation of a mammalian cell recombinantly modified to express an orthogonal CD122 comprising amino acid substitutions at positions H133 and Y134. In some embodiments, the present disclosure provides methods of use of orthologs to cause the activation of a mammalian cell recombinantly modified to express an orthogonal CD122 comprising the amino acid substitutions H133D and Y134F.

In some embodiments, the present disclosure provides methods for the preparation of a population of cells enriched for mammalian cells recombinantly modified to express an orthogonal receptor. In some embodiments, the present disclosure provides a population of mammalian cells enriched for mammalian cells recombinantly modified to express an orthogonal receptor.

In some embodiments, the present disclosure provides the use of orthologs that are cognate ligands for orthogonal receptors. In some embodiments, the ortholog is an ortholog of Formula 1. In some embodiments, the ortholog is STK-007. In some embodiments the ortholog is STK-009.

In some embodiments, the present disclosure provides IL2 orthologs that exhibit diminished affinity for the non-engineered intermediate affinity (CD122/CD132) IL2 receptor complex or high-affinity (CD25/CD122/CD132) IL2 receptor complex are also useful to selectively bias the activity of ortholog IL2 towards cells which constitutively express CD25 (e.g. Tregs) or inducibly express CD25 (e.g. activated CD8+ T cells). IL2 orthologs with significantly diminished affinity for the extracellular domain (ECD) of native wild-type CD122 but retain binding to the ECD of CD25 may also be used as competitive antagonists of wild-type IL2 by interfering with the high-affinity IL2 receptor complex formation and consequently may be employed in the treatment of autoimmune diseases or graft-versus-host (GVH) disease.

In some embodiments, the present disclosure provides IL2 orthologs that specifically and selectively bind to the extracellular domain (ECD) of a transmembrane polypeptide comprising of a modified CD122 polypeptide (orthogonal CD122). The binding of the IL2 ortholog to the orthogonal CD122 participates in the transduction pathway of intracellular signaling resulting in the activation of native intracellular signaling patterns associated with IL2 binding to either the intermediate or high affinity IL2 receptor but which exhibits selectivity to an engineered cell expressing an orthogonal CD122. The IL2 orthologs can exhibit significantly reduced binding to the extracellular domain of wild type CD122, either alone or when CD122 is present in the form of the endogenous high or intermediate affinity IL2 receptor, relative to the IL2 ortholog's binding to the CD122 orthogonal receptor. In some embodiments, the affinity of the IL2 ortholog for the extracellular domain of the orthogonal CD122 is comparable to the affinity of wild-type IL2 for wild-type CD122. In some embodiments, the affinity of the IL2 ortholog for the extracellular domain of the orthogonal CD122 is greater than to the affinity of wild-type IL2 for extracellular domain of wild-type CD122. In some embodiments, the affinity of the IL2 ortholog for the extracellular domain of the orthogonal CD122 is less than to the affinity of wild-type IL2 for the extracellular domain of the wild-type CD122.

The present disclosure further provides methods of making the IL2 orthologs of the present invention. In particular, the present disclosure provides recombinant expression vectors comprising a nucleic acid sequence encoding the IL2 orthologs operably linked to control elements to provide for expression of the nucleic acid sequence encoding the IL2 ortholog in a host cell.

The present invention further provides engineered mammalian cells expressing an orthogonal receptor. The present invention further provides pharmaceutically acceptable formulations of an engineered mammalian cells expressing an orthogonal receptor.

The disclosure further provides a method of generating a pharmaceutically acceptable dosage form of an engineered cell therapy product the dosage form comprising a population of T cells wherein the population of T cells is substantially enriched for one or more species of engineered T cells, the engineered T cells expressing a receptor comprising the extracellular domain of an CD122 orthogonal polypeptide, the method comprising the steps culturing the population of T cells comprising engineered T cells expressing a receptor comprising the extracellular domain of an CD122 orthogonal polypeptide ex vivo in the presence of an IL2 ortholog of the present invention for a period of time sufficient to enrich the cell population in of one or more such engineered T cells.

In some embodiments, a recombinant vector comprising a nucleic acid sequence encoding the IL2 ortholog described herein operably linked to control elements to facilitate expression and secretion of the IL2 ortholog from a mammalian cell is administered to the subject to provide for in situ expression of the IL2 ortholog. In some embodiments, the recombinant vector is administered intratumorally to a subject suffering from cancer. In some embodiments, the recombinant vector is a recombinant viral vector. In some embodiments the recombinant viral vector is a recombinant adeno-associated virus (rAAV) or recombinant adenovirus (rAd), for example in some embodiments, a replication deficient adenovirus derived from human adenovirus serotypes 3 and/or 5. In some embodiments, the replication deficient adenovirus has one or more modifications to the E1 region which interfere with the ability of the virus to initiate the cell cycle and/or apoptotic pathways. The replication deficient adenoviral vector may optionally comprise deletions in the E3 domain. In some embodiments the adenovirus is a replication competent adenovirus. In some embodiments the adenovirus is a replication competent recombinant virus engineered to selectively replicate in neoplastic cells.

The present disclosure further provides methods of preparing a pharmaceutically acceptable dosage form of a cell therapy product comprising at least one (alternatively 2, 3, 4 or more) species of engineered T cells that express a transmembrane receptor protein wherein the extracellular domain of such transmembrane receptor protein comprises the extracellular domain of an CD122 orthogonal polypeptide wherein the fraction of engineered cells in the cell therapy product comprises at least 30%, alternatively at least 40%, alternatively at least 50%, alternatively at least 60%, alternatively at least 70%, alternatively at least 80%, or alternatively at least 90% of the total number of cells in the cell therapy product.

In some embodiments a therapeutic method is provided, the method comprising introducing into a subject in need thereof of population of cells, said cell population comprising engineered human immune cells comprising a nucleic acid sequence encoding a cell membrane spanning polypeptide comprising an ECD of a human orthogonal CD122, a transmembrane domain and an intracellular signaling domain that provides in an intracellular signal in response to the binding of a ligand to the ECD of said cell membrane spanning polypeptide, said nucleic acid sequence operably linked to expression control elements to facilitate transcription and translation and cell surface presentation of said membrane spanning polypeptide. Such cell population may comprise cells which have been modified ex vivo and are autologous or allogeneic with respect to the subject. In some embodiments, the therapeutic method comprises (1) contacting a cell population ex vivo an amount of a cognate IL2 ortholog at a concentration and for a duration of time sufficient to activate said engineered cells comprising a receptor comprising an extracellular domain that is an orthogonal CD122 ECD; and (2) administering the cell population to the subject; and (3) administering the cognate IL2 ortholog in vivo following administration of the engineered cells. In some embodiments, the introduced cell population is contacted with the cognate orthogonal cytokine in vivo, following administration of the engineered cells.

The present disclosure further provides a method of extending of an active form (“persistence”) of an orthogonal engineered immune cell (e.g., an orthogonal CAR-T cell) in vivo in a mammalian subject the administration to the subject of an effective amount of an orthogonal ligand.

The present disclosure further provides a method a method of specifically and selectively activating and/or inducing the proliferation of an orthogonal immune cells (e.g. a orthogonal CAR-T cell) in vivo in a mammalian subject by administering to the mammalian subject an effective amount of an orthogonal immune cell in conjunction with the administration of an effective amount of an orthogonal ligand.

The present disclosure further provides a method of treating a mammalian subject suffering from neoplastic disease by administering to the mammalian subject an effective amount of an orthogonal CAR-T cell in combination with the administration of an effective amount of an cognate orthogonal ligand for the receptor expressed on the orthogonal CAR-T cell.

The present disclosure further provides a method of treating a mammalian subject suffering from a diffuse (non-solid tumor) neoplastic disease by administering to the mammalian subject an effective amount of an orthogonal CAR-T cell in conjunction with the administration of an effective amount of an cognate orthogonal ligand for the receptor expressed on the orthogonal CAR-T cell.

The present disclosure further provides a method of treating a mammalian subject suffering from neoplastic disease characterized by the presence of a solid tumor (e.g. a lymphoma, prostate cancer, lung cancer, bladder cancer, HPV related cancers such as cervical cancer, neuroblastoma) by administering to the mammalian subject an effective amount of an orthogonal T cell in conjunction with the administration of an effective amount of an cognate orthogonal ligand for the receptor expressed on the orthogonal T cell.

The present disclosure further provides a method of restoring the activity of an exhausted orthogonal immune cell in a subject by the administration of an effective amount of an orthogonal ligand to the subject.

The present disclosure further provides a method of treating a mammalian subject suffering from relapse of a neoplastic disease in a treatment regimen characterized by the administration of orthogonal CAR-T cell product, the method comprising the steps: (i) of administering to the subject an effective amount of a cognate orthogonal ligand for the receptor expressed on the orthogonal CAR-T cell previously administered sufficient to restore the activity of the previously administered of orthogonal CAR-T cells; (ii) periodically administering to the subject an effective amount of a cognate orthogonal ligand for the receptor expressed on the orthogonal CAR-T cell previously administered to maintain the activity of orthogonal CAR-T cells for a period of time sufficient; (iii) evaluating the subject for the presence of the neoplastic disease and upon the lack of evidence of neoplastic disease either discontinuing the administration of the orthogonal ligand or continuing to administer the orthogonal ligand periodically in accordance with a maintenance dosing protocol sufficient to maintain a quantity of orthogonal CAR-T cells sufficient for immune surveillance of the neoplastic cells.

The present disclosure further provides a method of preventing and/or treating relapse in the treatment of a neoplastic disease with a CAR-T cell therapy by the administration of orthogonal CAR-T cell followed by the periodic administration of an effective amount of an cognate orthogonal ligand for the receptor expressed on the orthogonal CAR-T cell with a maintenance dosing protocol sufficient to maintain a quantity of orthogonal CAR-T cells sufficient for immune surveillance of the neoplastic cells.

The present disclosure further provides a method of treating a mammalian subject suffering from a relapsed or refractory neoplastic disease in a treatment regimen characterized by the prior administration of orthogonal CAR-T cell product, the method comprising the steps: (i) of administering to the subject an effective amount of a cognate orthogonal ligand for the receptor expressed on the orthogonal CAR-T cell previously administered sufficient to restore the activity of the previously administered of orthogonal CAR-T cells; (ii) periodically administering to the subject an effective amount of a cognate orthogonal ligand for the receptor expressed on the orthogonal CAR-T cell previously administered to maintain the activity of orthogonal CAR-T cells for a period of time sufficient to effect a therapeutic response; (iii) evaluating the subject for the presence of the neoplastic disease and upon the lack of evidence of neoplastic disease either discontinuing the administration of the orthogonal ligand or continuing to administer the orthogonal ligand periodically in accordance with a maintenance dosing protocol sufficient to maintain a quantity of orthogonal CAR-T cells sufficient for immune surveillance of the neoplastic cells.

The present disclosure further provides a method of treating a mammalian subject suffering from a neoplastic disease with an orthogonal hCD122 cell and an orthogonal hIL2 ligand therefor, the method comprising the steps: (i) within a period of two weeks to one day prior to the administration of the orthogonal hCD122 cell, administering to the subject a therapeutically effective dose of an orthogonal ligand for the receptor expressed on the orthogonal CAR-T (i.e. priming); (ii) administering to the subject a therapeutically effective amount of orthogonal hCD122 cell in combination with an orthogonal ligand that is a cognate hIL2 ligand for the orthogonal CD122 receptor of the orthogonal hCD122 cell, and optionally (iii) evaluating the subject for the presence of the neoplastic disease and upon the lack of evidence of neoplastic disease either discontinuing the administration of the orthogonal ligand or continuing to administer the orthogonal ligand subsequently at a dose sufficient to maintain a level of circulating orthogonal CAR-T cells sufficient to maintain immune surveillance of the neoplastic cells, and optionally, in the event of relapse, administration of a therapeutically effective amount of an orthogonal ligand that is a cognate hIL2 ligand for the orthogonal CD122 receptor of the orthogonal hCD122 cell previously administered.

In another aspect, the present disclosure provides a method of treating chronic viral infections by the administration of an orthogonal cell comprising a targeting domain specific for antigens expressed on the surface of virally infected cell. Examples of chronic viral infections amenable to treatment with the compositions and methods of the present disclosure include but are not limited to cytomegalovirus (CMV), HTLV1, herpes simplex virus type 2 (HSV-2), Epstein-Barr Virus, human herpesvirus 6, human herpesvirus 7, hepatitis C virus (HCV), human immunodeficiency virus (HIV1 and HIV2).

In some embodiments, the engineered T cell is genomically modified to eliminate checkpoint expression (i.e. a checkpoint knock-out”). Examples of such checkpoint knockout cells include PD1 knock-out (“PD1KO”) T cells. Strategies for to create PD1KO T cells are well known in the art. PD1KO T cells are well known in the art. McGowan, et al (121PD1 disrupted Car-T Cells In The Treatment of Solid Tumors: Promises and Challenges) Biomedicine and Pharmacotherapy Biomedicine & Pharmacotherapy, Volume 121 (2020, available online 13 Nov. 2019) 109625, https://doi.org/10.1016/j.biopha.2019.109625) provides an extensive review of engineered and isolated PD1KO T cells being explored in a variety of clinical applications (see Table 1).

Alternative to completely eliminating PD1 function in the orthogonal immune cell, in some embodiments the orthogonal immune cells of the present disclosure provide a mechanism for the downregulation of PD1 activity in the orthogonal immune cell. The expression of PDL1 on tumor cells and chronically virally infected cells enables such cells to avoid immune surveillance by binding the PD1 expressed on human immune cells. Although groups have demonstrated that engineered T cells that knock out PD1 expression are effective, they also are indiscriminate and lead to significant toxicity as they are capable of significant target toxicity typically mediated by self-tolerance mechanism of PD1/PDL1 interaction. In some embodiments, the downregulation is conditional in response to signaling initiated by binding of the immune cell to its target, either engineered in the case of cells engineered to express a specific targeting/activation domain such as a CAR (e.g., oCAR-T cells, oCAR NK cells, oTCR engineered T cells) or the endogenous binding domain (e.g. an ortho TIL). Factors upregulated by the binding of the immune cell can be employed to selectively downregulate PD1 expression control sequences in the orthogonal immune cell resulting in down-regulation of PD1 expression only once the orthogonal cell interacts with its proper target. In some embodiments, domains may be engineered into the CAR ICD such that binding of the CAR to the target antigen results in intracellular signaling that suppresses PD1 expression following interaction of the targeting domain with the target thereby enabling the activated orthogonal immune cell to avoid downregulation and potential the immune evasion caused by interaction of PD1 with the PDL1 expressing cell, e.g., chronically virally infected or tumor cells. In addition to the foregoing, other mechanisms for mitigating PDL1 immunomodulation of engineered immune cells which may be incorporated into the orthogonal immune cells of the present disclosure. See, e.g. Busser, et al United States Patent Application Publication US2020/0407694A1 published Dec. 31, 2020, Li, et al., United States Patent Application Publication US20180327470A1 published Nov. 15, 2018.

As previously discussed, a significant issue with current cell therapies is the lack of non-toxic means to support the proliferation of the transferred cells once they have been administered to a patient.

The present disclosure provides a human orthogonal cell. cell mammalian immune cell comprising a nucleic acid sequence encoding an orthogonal hCD122 receptor operably linked to one or more expression control elements such that the mammalian immune cell expresses the orthogonal hCD122 receptor.

The present disclosure provides a mammalian immune cell comprising (a) a nucleic acid sequence encoding an orthogonal hCD122 receptor operably linked to one or more expression control elements such that the mammalian immune cell expresses the orthogonal hCD122 receptor, and (b) a nucleic acid sequence encoding a chimeric antigen receptor (CAR) operably linked to one or more expression control elements such that the mammalian immune cell expresses the CAR.

The present disclosure provides a recombinant expression vector, the vector comprising: (a) a nucleic acid sequence encoding an orthogonal hCD122 receptor operably linked to one or more expression control elements such that the mammalian immune cell expresses the orthogonal hCD122 receptor, and (b) a nucleic acid sequence encoding a chimeric antigen receptor (CAR) operably linked to one or more expression control elements such that the mammalian immune cell expresses the CAR.

The present disclosure provides a method of treating a neoplastic disease by the administration of mammalian immune cell comprising a nucleic acid sequence encoding an orthogonal CD122 receptor.

The present disclosure provides a method of treating a neoplastic disease in a human subject comprising the administration of a mammalian immune cell comprising a nucleic acid sequence encoding an orthogonal hCD122 receptor operably linked to one or more expression control elements such that the mammalian immune cell expresses the orthogonal hCD122 receptor and the administration of an orthogonal ligand that binds to the ECD of the hCD122 receptor and results in intracellular signaling. In some embodiments, the ligand is administered prior to the administration of the mammalian immune cell

The present invention provides significant issue with current cell therapies is the lack of non-toxic means to support the proliferation of the transferred cells once they have been administered to a patient without

The present disclosure provides a human orthogonal cell. cell mammalian immune cell comprising a nucleic acid sequence encoding an orthogonal hCD122 receptor operably linked to one or more expression control elements such that the mammalian immune cell expresses the orthogonal hCD122 receptor.

The present disclosure provides a mammalian immune cell comprising (a) a nucleic acid sequence encoding an orthogonal hCD122 receptor operably linked to one or more expression control elements such that the mammalian immune cell expresses the orthogonal hCD122 receptor, and (b) a nucleic acid sequence encoding a chimeric antigen receptor (CAR) operably linked to one or more expression control elements such that the mammalian immune cell expresses the CAR.

The present disclosure provides a recombinant expression vector, the vector comprising: (a) a nucleic acid sequence encoding an orthogonal hCD122 receptor operably linked to one or more expression control elements such that the mammalian immune cell expresses the orthogonal hCD122 receptor, and (b) a nucleic acid sequence encoding a chimeric antigen receptor (CAR) operably linked to one or more expression control elements such that the mammalian immune cell expresses the CAR.

The present disclosure provides a method of treating a neoplastic disease by the administration of mammalian immune cell comprising a nucleic acid sequence encoding an orthogonal CD122 receptor.

The present disclosure provides a method of treating a neoplastic disease in a human subject comprising the administration of a mammalian immune cell comprising a nucleic acid sequence encoding an orthogonal hCD122 receptor operably linked to one or more expression control elements such that the mammalian immune cell expresses the orthogonal hCD122 receptor and the administration of an orthogonal ligand that binds to the ECD of the hCD122 receptor and results in intracellular signaling. In some embodiments, the ligand is administered prior to the administration of the mammalian immune cell.

In some embodiments, a method of treating or preventing a disease, disorder, or condition in a mammalian subject in need of treatment or prevention is provided, the method comprising the steps of:

(a) Isolating a quantity of immune cells from the subject;
(b) Contacting said isolated quantity of isolated immune cells with a nucleic acid sequence under conditions for the uptake of said nucleic acid sequence by the isolated immune cells, said nucleic acid sequence encoding a transmembrane receptor, said transmembrane receptor comprising an intracellular signaling domain in operable communication with an extracellular domain, said extracellular domain of said receptor comprising the ECD of a an orthogonal hCD122 or a functional fragment thereof;
(c) Contacting the isolated quantity of cells from step (b) ex vivo with a quantity of a orthogonal ligand sufficient to induce proliferation of cells transduced by the contacting of step (b), said contacting being applied for a period of time to such that the transduced cells comprise at least 20% of the cells of the population;
(d) Administering a therapeutically effective quantity of the cells of the cell population produced from step (c) to the mammalian subject in combination with the administration of a therapeutically effective dose of an orthogonal ligand.

In some embodiments, the population comprises one or more of species human immune cells selected from the group consisting myeloid cells, lymphocytes, peripheral blood mononuclear cells (PBMCs), tumor infiltrating lymphocytes (TILs), T cells, CD8+ T cells, CD25+CD8+ T cells, CAR-T cells, NK cells, CD4+ T cells, and Tregs.

In some embodiments, after step (a) but prior to step (b), the population of cells is manipulated ex vivo to enrich said population for activated immune cells or antigen experienced T cells.

In some embodiments, the orthogonal hCD122 or functional fragment thereof comprises an amino acid sequence with an amino acid substitution at position 133 and/or 134 numbered in accordance with wild-type hCD122.

In some embodiments, the contacting of step (b) further comprises the uptake of a nucleic acid sequence encoding a chimeric antigen receptor (CAR).

In some embodiments, the nucleic acid sequence encoding the CAR and the nucleic acid sequence encoding the receptor are provided on separate vectors, each nucleic acid sequence operably linked to an expression control sequence operatable in a mammalian immune cell.

In some embodiments, the nucleic acid sequence encoding the CAR and the nucleic acid sequence encoding the receptor are provided on a single vector.

In some embodiments, the nucleic acid sequences are operably linked to the same expression control element.

In some embodiments, the vector comprises the two nucleic acid sequences are separated by an IRES element of T2A coding sequence.

In some embodiments, the vector is a viral vector.

In some embodiments, the vector is a lentiviral vector or retroviral vector.

In some embodiments, the orthogonal ligand employed ex vivo in step (b) is different than the orthogonal ligand used in vivo in step (c).

In some embodiments, wherein prior to step (d) the subject is treated with a lymphodepeting regiment.

In some embodiments, the initial dose administered in step (d) is between 100,000 and 1,000,000 activate immune cells per kg of bodyweight of the subject.

In some embodiments, the administration of the orthogonal ligand is administered periodically to the subject to maintain a level of between 100,000 and 1,000,000 activate immune cells per kg of bodyweight of the subject for a period of time of at least two weeks

In some embodiments, the orthogonal ligand is administered until a point where there is no substantial sign of remaining tumor at which time the dose of the orthogonal ligand is reduced to a level sufficient to maintain a low circulating level of orthogonal immune cells of approximately 10,000 to 100,000 cells per kg of bodyweight for a period of time of at least 3 months following the observation

In some embodiments, the orthogonal ligand is administered until a point where there is no substantial sign of remaining tumor at which time the dose of the orthogonal ligand terminated.

In some embodiments, if the patient relapses from the initial course of immune cell therapy, the method further comprising the step of administering to the patient in relapse a therapeutically effective amount of an orthogonal ligand in the absence of additional dose of the orthogonal engineered cell such that the orthogonal ligand induces the activation and/or proliferation of the previously administered orthogonal cell, the orthogonal ligand being applied to the subject for a period of time until remission of the relapsed tumor is observed.

In some embodiments, the disease, disorder of condition is a neoplastic disease.

In some embodiments, the disease, disorder of condition is a chronic viral disease.

In some embodiments, the disease, disorder of condition is a inflammatory disease.

Also provided is a cell product substantially enriched for a population of activated orthogonal immune cells the product obtained by a process comprising the steps of:

(a) Isolating a quantity of immune cells from a mammalian subject;
(b) Contacting said isolated quantity of isolated immune cells with a nucleic acid sequence under conditions for the uptake of said nucleic acid sequence by the isolated immune cells, said nucleic acid sequence encoding a transmembrane receptor, said transmembrane receptor comprising an intracellular signaling domain in operable communication with an extracellular domain, said extracellular domain of said receptor comprising the ECD of a an orthogonal hCD122 or a functional fragment thereof;
(c) Contacting the isolated quantity of cells from step (b) ex vivo with a quantity of a orthogonal ligand sufficient to induce proliferation of cells transduced by the contacting of step (b), said contacting being applied for a period of time to such that the transduced cells comprise at least 20% of the cells of the population.

In some embodiments, the cell product comprises one or more of species human immune cells selected from the group consisting myeloid cells, lymphocytes, peripheral blood mononuclear cells (PBMCs), tumor infiltrating lymphocytes (TILs), T cells, CD8+ T cells, CD25+CD8+ T cells, CAR-T cells, NK cells, CD4+ T cells, and Tregs.

In some embodiments, the cell product is further manipulated to deleted the endogenous TCR domain of said cell.

### BRIEF DESCRIPTION OF THE FIGURES

The invention is best understood from the following detailed description when read in conjunction with the accompanying figures. It is emphasized that, according to common practice, the various features of the drawings are not to-scale. On the contrary, the dimensions of the various features are arbitrarily expanded or reduced for clarity. Included in the drawings are the following figures.

FIG. 1 of the attached drawing provides Celltiterglo values for NKL cells treated with 293T transfection supernatant from experiments as described in Example 6. For NKL cells receiving the indicated dilution of each supernatant, indicated in bold, duplicate celltiterglo values are shown in side-by-side columns.

FIG. 2 of the attached drawing provides Celltiterglo values for NKL hoRB cells treated with 293T transfection supernatants from experiments as described in Example 6. For NKL hoRB cells receiving the indicated dilution of each supernatant, indicated in bold, duplicate Celltiterglo values are shown in side-by-side columns.

FIG. 3A-B provides the results of a disseminated in vivo RAJI tumor study as more fully described in Example 7 below demonstrating the effect of the treatment of the CAR-T orthogonal cells with an orthogonal ligand as compared to CAR-T or PBS alone.

FIG. 4A-B provides the results of the rechallenge study as more fully described below in Example 8 demonstrating that the administration of the orthogonal ligand alone (without the need to provide additional cells) is capable of restoring the anti-tumor activity of CAR T cells even a prolonged period of no antigen or tumor ligand exposure.

FIG. 5A-B provides the results of redosing in a sub cutaneous RAJI-luc lymphoma relapse mouse model more fully described in Example 8. This data demonstrates that the administration of STK-009 alone is capable of effectuating anti-tumor activity of CAR-T cells in animal that have relapsed from a prior course of therapy.

FIG. 6 is a histogram of the results of FACS analysis the data as more fully described in Example 8 demonstrating that an orthogonal ligand (STK-009) is capable of expanding the orthogonal CAR-T cells and retains the stem cell memory CAR-T cell population.

FIG. 7A-C provides caliper measured data generated from a subcutaneous Raji solid tumor model as more fully described in Example 11 below the efficacy of the orthogonal CAR-T cells in combination with an orthogonal ligand in the in vivo treatment of a solid tumor model.

FIG. 8 provides a graphical representation of physical measurement of tumor response data generated from an experiments as described in Example 11 herein showing that the administration of an CD19 orthogonal CAR-T cell in combination with an orthogonal ligand (STK-009) is efficacious in the treatment of solid tumors.

FIG. 9 provides a Kaplan-Meier survival plot relating to the subcutaneous solid tumor model study as described in Example 8. The data provided demonstrate that the administration of a CD19 orthogonal CAR-T cell in combination with an orthogonal ligand (STK-009) is efficacious in the treatment of solid tumors and confers a significant survival advantage.

FIG. 10 provides immunohistochemistry of tissues isolated from mice sacrificed following the conclusion of subcutaneous solid tumor model study as described in Example 8.

### DETAILED DESCRIPTION

### Introduction

In order for the present disclosure to be more readily understood, certain terms and phrases are defined below as well as throughout the specification. The definitions provided herein are non-limiting and should be read in view of the knowledge of one of skill in the art would know.

Before the present methods and compositions are described, it is to be understood that this disclosure is not limited to particular method or composition described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless indicated otherwise the following abbreviation are used herein: parts are parts by weight, molecular weight is weight average molecular weight, temperature is in degrees Celsius (° C.), and pressure is at or near atmospheric. Standard abbreviations are used, including the following: bp=base pair(s); kb=kilobase(s); pl=picoliter(s); s or sec=second(s); min=minute(s); h or hr=hour(s); AA or aa=amino acid(s); kb=kilobase(s); nt=nucleotide(s); pg=picogram; ng=nanogram; μg=microgram; mg=milligram; g=gram; kg=kilogram; dl or dL=deciliter; μl or μL=microliter; ml or mL=milliliter; 1 or L=liter; μM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=once weekly; QM=once monthly; HPLC=high performance liquid chromatography; BW=body weight; U=unit; ns=not statistically significant; PBS=phosphate-buffered saline; PCR=polymerase chain reaction; HSA=human serum albumin; MSA=mouse serum albumin; DMEM=Dulbeco's Modification of Eagle's Medium; EDTA=ethylenediaminetetraacetic acid.

It will be appreciated that throughout this disclosure reference is made to amino acids according to the single letter or three letter codes. For the reader's convenience, the single and three letter amino acid codes are provided in Table 1 below:

TABLE 1
Amino Acid Abbreviations

| | | |
|---|---|---|
| G | Glycine | Gly |
| P | Proline | Pro |
| A | Alanine | Ala |
| V | Valine | Val |
| L | Leucine | Leu |
| I | Isoleucine | Ile |
| M | Methionine | Met |
| C | Cysteine | Cys |
| F | Phenylalanine | Phe |
| Y | Tyrosine | Tyr |
| W | Tryptophan | Trp |
| H | Histidine | His |
| K | Lysine | Lys |
| R | Arginine | Arg |
| Q | Glutamine | Gln |
| N | Asparagine | Asn |
| E | Glutamic Acid | Glu |
| D | Aspartic Acid | Asp |
| S | Serine | Ser |
| T | Threonine | Thr |

Standard methods in molecular biology are described in the scientific literature (see, e.g., Sambrook and Russell (2001) Molecular Cloning, 3rd ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)). The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., NY).

### B. Definitions

Unless otherwise indicated, the following terms are intended to have the meaning set forth below. Other terms are defined elsewhere throughout the specification.

Activate: As used herein the term “activate” is used in reference to a receptor or receptor complex to reflect a biological effect, directly and/or by participation in a multicomponent signaling cascade, arising from the binding of an agonist ligand to a receptor responsive to the binding of the ligand. For example, it is said that the binding of an IL2 agonist (an IL2 agonist ligand) to the IL2 receptor “activates” the signaling of the receptor to produce one or more intracellular biological effects (e.g. phosphorylation of STAT5).

Activity: As used herein, the term “activity” is used with respect to a molecule to describe a property of the molecule with respect to a test system (e.g. an assay) or biological or chemical property (e.g. the degree of binding of the molecule to another molecule) or of a physical property of a material or cell (e.g. modification of cell membrane potential). Examples of such biological functions include but are not limited to catalytic activity of a biological agent, the ability to stimulate intracellular signaling, gene expression, cell proliferation, the ability to modulate immunological activity such as inflammatory response. “Activity” is typically expressed as a level of a biological activity per unit of agent tested such as [catalytic activity]/[mg protein], [immunological activity]/[mg protein], international units (IU) of activity, [STAT5 phosphorylation]/[mg protein], [T-cell proliferation]/[mg protein], plaque forming units (pfu), etc. As used herein, the term “proliferative activity” refers to an activity that promotes cell proliferation and replication, including dysregulated cell division such as that observed in neoplastic diseases, inflammatory diseases, fibrosis, dysplasia, cell transformation, metastasis, and angiogenesis.

Administer/Administration: The terms “administration” and “administer” are used interchangeably herein to refer the act of contacting a subject, including contacting a cell, tissue, organ, or biological fluid of the subject in vitro, in vivo or ex vivo with an agent (e.g. an ortholog, an IL2 ortholog, an engineered cell expressing an orthogonal receptor, an engineered cell expressing an orthogonal IL2 receptor (e.g. a CAR-T cell expressing an orthogonal IL2 receptor) a chemotherapeutic agent, an antibody) or a pharmaceutical formulation comprising one or more of the foregoing. Administration of an agent may be achieved through any of a variety of art recognized methods including but not limited to the topical administration, intravascular injection (including intravenous or intraarterial infusion), intradermal injection, subcutaneous injection, intramuscular injection, intraperitoneal injection, intracranial injection, intratumoral injection, transdermal, transmucosal, iontophoretic delivery, intralymphatic injection, intragastric infusion, intraprostatic injection, intravesical infusion (e.g., bladder), inhalation (e.g respiratory inhalers including dry-powder inhalers), intraocular injection, intraabdominal injection, intralesional injection, intraovarian injection, intracerebral infusion or injection, intracerebroventricular injection (ICVI), and the like. The term “administration” includes contact of an agent to the cell, tissue or organ as well as the contact of an agent to a fluid, where the fluid is in contact with the cell, tissue or organ.

Adverse Event: As used herein, the term “adverse event” refers to any undesirable experience associated with the use of a therapeutic or prophylactic agent in a subject. Adverse events do not have to be caused by the administration of the therapeutic or prophylactic agent (e.g. the IL2 ortholog) but may arise from unrelated circumstances. Adverse events are typically categorized as mild, moderate, or severe. As used herein, the classification of adverse events as used herein is in accordance with the Common Terminology Criteria for Adverse Events v5.0 (CTCAE) dated published Nov. 27, 2017 published by the United States Department of Health and Human Services, the National Institutes of Health and the National Cancer Institute.

Affinity: As used herein the term “affinity” refers to the degree of specific binding of a first molecule (e.g. a ligand) to a second molecule (e.g. a receptor) and is measured by the binding kinetics expressed as K_{d}, a ratio of the dissociation constant between the molecule and the its target (K_{off}) and the association constant between the molecule and its target (Kₒₙ).

Agonist: As used herein, the term “agonist” refers an first agent that specifically binds a second agent (“target”) and interacts with the target to cause or promote an increase in the activation of the target. In some instances, agonists are activators of receptor proteins that modulate cell activation, enhance activation, sensitize cells to activation by a second agent, or up-regulate the expression of one or more genes, proteins, ligands, receptors, biological pathways, that may result in cell proliferation or pathways that result in cell cycle arrest or cell death such as by apoptosis. In some embodiments, an agonist is an agent that binds to a receptor and alters the receptor state, resulting in a biological response. The response mimics the effect of the endogenous activator of the receptor. The term “agonist” includes partial agonists, full agonists and superagonists. An agonist may be described as a “full agonist” when such agonist which leads to a substantially full biological response (i.e. the response associated with the naturally occurring ligand/receptor binding interaction) induced by receptor under study, or a partial agonist. In contrast to agonists, antagonists may specifically bind to a receptor but do not result in the signal cascade typically initiated by the receptor and may to modify the actions of an agonist at that receptor. Inverse agonists are agents that produce a pharmacological response that is opposite in direction to that of an agonist. A “superagonist” is a type of agonist that is capable of producing a maximal response greater than the endogenous agonist for the target receptor, and thus has an activity of more than 100% of the native ligand. A super agonist is typically a synthetic molecule that exhibits greater than 110%, alternatively greater than 120%, alternatively greater than 130%, alternatively greater than 140%, alternatively greater than 150%, alternatively greater than 160%, or alternatively greater than 170% of the response in an evaluable quantitative or qualitative parameter of the naturally occurring form of the molecule when evaluated at similar concentrations in a comparable assay. It should be noted that the biological effects associated with the full agonist may differ in degree and/or in kind from those biological effects of partial or superagonists.

Antagonist: As used herein, the term “antagonist” or “inhibitor” refers a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist. Inhibitors are molecules that decrease, block, prevent, delay activation, inactivate, desensitize, or down-regulate, e.g., a gene, protein, ligand, receptor, biological pathway including an immune checkpoint pathway, or cell.

Antibody: As used herein, the term “antibody” refers collectively to: (a) glycosylated and non-glycosylated the immunoglobulins (including but not limited to mammalian immunoglobulin classes IgG1, IgG2, IgG3 and IgG4) that specifically binds to target molecule and (b) immunoglobulin derivatives including but not limited to IgG(1-4)deltaC_{H}2, F(ab′)₂, Fab, ScFv, V_{H}, V_{L}, tetrabodies, triabodies, diabodies, dsFv, F(ab′)₃, scFv-Fc and (scFv)₂ that competes with the immunoglobulin from which it was derived for binding to the target molecule. The term antibody is not restricted to immunoglobulins derived from any particular mammalian species and includes murine, human, equine, camelids, antibodies, human antibodies. The term antibody includes so called “heavy chain antibodies” or “VHHs” or “Nanobodies®” as typically obtained from immunization of camelids (including camels, llamas and alpacas (see, e.g. Hamers-Casterman, et al. (1993) Nature 363:446-448). Antibodies having a given specificity may also be derived from non-mammalian sources such as VHHs obtained from immunization of cartilaginous fishes including, but not limited to, sharks. The term “antibody” encompasses antibodies isolatable from natural sources or from animals following immunization with an antigen and as well as engineered antibodies including monoclonal antibodies, bispecific antibodies, tri-specific, chimeric antibodies, humanized antibodies, human antibodies, CDR-grafted, veneered, or deimmunized (e.g., to remove T-cell epitopes) antibodies. The term “human antibody” includes antibodies obtained from human beings as well as antibodies obtained from transgenic mammals comprising human immunoglobulin genes such that, upon stimulation with an antigen the transgenic animal produces antibodies comprising amino acid sequences characteristic of antibodies produced by human beings. The term antibody includes both the parent antibody and its derivatives such as affinity matured, veneered, CDR grafted, humanized, camelized (in the case of VHHs), or binding molecules comprising binding domains of antibodies (e.g. CDRs) in non-immunoglobulin scaffolds. The term “antibody” should not be construed as limited to any particular means of synthesis and includes naturally occurring antibodies isolatable from natural sources and as well as engineered antibodies molecules that are prepared by “recombinant” means including antibodies isolated from transgenic animals that are transgenic for human immunoglobulin genes or a hybridoma prepared therefrom, antibodies isolated from a host cell transformed with a nucleic acid construct that results in expression of an antibody, antibodies isolated from a combinatorial antibody library including phage display libraries. In one embodiment, an “antibody” is a mammalian immunoglobulin. In some embodiments, the antibody is a “full length antibody” comprising variable and constant domains providing binding and effector functions. In most instances, a full-length antibody comprises two light chains and two heavy chains, each light chain comprising a variable region and a constant region. In some embodiments the term “full length antibody” is used to refer to conventional IgG immunoglobulin structures comprising two light chains and two heavy chains, each light chain comprising a variable region and a constant region providing binding and effector functions. The term antibody includes antibody conjugates comprising modifications to prolong duration of action such as fusion proteins (e.g., Fc fusions) or conjugation to polymers (e.g. polyethylene glycol) as described in more detail below.

Biological Sample: As used herein, the term “biological sample” or “sample” refers to a sample obtained (or derived) from a subject. By way of example, a biological sample comprises a material selected from the group consisting of body fluids, blood, whole blood, plasma, serum, mucus secretions, saliva, cerebrospinal fluid (CSF), bronchoalveolar lavage fluid (BALF), fluids of the eye (e.g., vitreous fluid, aqueous humor), lymph fluid, lymph node tissue, spleen tissue, bone marrow, and an immunoglobulin enriched fraction derived from one or more of these tissues. In some embodiments, the sample is obtained from a subject who has been exposed to a therapeutic treatment regimen such as repeated exposure to one or more therapeutic agents, the agent including a pharmaceutical formulation of an IL2 ortholog. In other embodiments, the sample is obtained from a subject who has not recently been exposed to the IL2 ortholog or obtained from the subject prior to the planned administration of the IL2 ortholog or treatment regimen comprising an IL2 ortholog.

CAR” or “Chimeric Antigen Receptor”: As used herein, the terms “chimeric antigen receptor” and “CAR” are used interchangeably to refer to a chimeric polypeptide comprising multiple functional domains arranged from amino to carboxy terminus in the sequence: (a) an extracellular domain (ECD) comprising an antigen binding domain (ABD), and optionally comprising a “hinge” domain, (b) a transmembrane domain (TD); and (c) one or more cytoplasmic signaling domains (CSDs) wherein the foregoing domains may optionally be linked by one or more spacer domains. The CAR may also further comprise a signal peptide sequence which is conventionally removed during post-translational processing and presentation of the CAR on the cell surface of a cell transformed with an expression vector comprising a nucleic acid sequence encoding the CAR. CARs may be prepared in accordance with principles well known in the art. See e.g., Eshhar, et al. (U.S. Pat. No. 7,741,465 B1 issued Jun. 22, 2010); Sadelain, et al. (2013) Cancer Discovery 3(4):388-398; Campana and Imai (U.S. Pat. No. 8,399,645 issued Mar. 19, 2013) Jensen and Riddell (2015) Current Opinions in Immunology 33:9-15; Gross, et al. (1989) PNAS (USA) 86(24):10024-10028; Curran, et al. (2012) J Gene Med 14(6):405-15; Brogdon, et al. (U.S. Pat. No. 10,174,095 issued Jan. 8, 2019) Guedan, et al. (2019) Engineering and Design of Chimeric Antigen Receptors Molecular Therapy: Methods & Clinical Development Vol. 12: 145-156. From a nomenclature perspective, in common practice CARs are referred to in reference to the target of the antigen binding domain (ABD) of the CAR such that a “CD19 CAR” refers to a CAR the ABD of which specifically binds to CD19, a “BCMA CAR” refers to a CAR the ABD of which CAR specifically binds to BCMA, and so forth. In some embodiments the ABD of the CAR is bivalent (or multivalent) such that the ABD specifically binds to more than one target antigen, for example the CD19 and CD20 tumor antigens. In such instances, the CAR would commonly be referred to as a “CD19/CD20” CAR referencing the multivalent nature of its ABD.

CAR-T Cell: As used herein, the terms “chimeric antigen receptor T-cell” and “CAR-T cell” are used interchangeably to refer to a T-cell that has been recombinantly modified to express a chimeric antigen receptor. As used herein, a CAR-T cell may be engineered to express a modified receptor comprising the extracellular domain of an orthogonal CD122 polypeptide (orthogonal CAR-T cells). From a nomenclature perspective, in common practice CAR-T cells are referred to in reference to the target of the antigen binding domain of the CAR such that a “CD19 CAR-T cell” would refer a CAR-T cell comprising a CAR wherein the ABD of CAR selectively binds to CD19, a “BCMA CAR-T cell” would refer to a CAR-T cell comprising a CAR wherein the ABD of CAR selectively binds to BCMA, and so forth. Examples of commercially available CD19 CAR-T cell products that may be modified to incorporate an orthogonal receptor of the present invention include axicabtagene ciloleucel (marketed as Yescarta® commercially available from Gilead Pharmaceuticals) and tisagenlecleucel (marketed as Kymriah® commercially available from Novartis). In some embodiments the ABD of the CAR is bivalent (or multivalent) such that the ABD specifically binds to more than one target antigen, for example the CD19 and CD20 tumor antigens. In such instances, the CAR-T cell comprising such a CAR would commonly be referred to as a “CD19/CD20” CAR referencing the multivalent nature of its ABD.

CD25: As used herein, the terms “CD25”, “IL2 receptor alpha”, “IL2Rα”, “IL2Ra” and “p55” are used interchangeably to refer to the 55 kD polypeptide that is constitutively expressed in Treg cells and inducibly expressed on other T cells in response to activation. CD25 is also referred to in the literature as the “low affinity” IL2 receptor. hIL2 binds to hCD25 with a K_{d} of approximately 10⁻⁸M. Human CD25 nucleic acid and protein sequences may be found as Genbank accession numbers NM_000417 and NP_0004Q8 respectively. The human CD25 is expressed as a 272 amino acid pre-protein comprising a 21 amino acid signal sequence which is post-translationally removed to render a 251 amino acid mature protein. Amino acids 22-240 of the pre-protein (amino acids 1-219 of the mature protein) correspond to the extracellular domain. Amino acids 241-259 of the pre-protein (amino acids 220-238 of the mature protein) correspond to transmembrane domain. Amino acids 260-272 of the pre-protein (amino acids 239-251 of the mature protein) correspond to intracellular domain. The amino acid sequence of the mature form of hCD25 (without the signal sequence of the pre-protein) is:

(SEQ ID NO. 1)
ELCDDDPPEIPHATFKAMAYKEGTMLNCECKRGFRRIKSGSLYMLC
TGNSSHSSWDNQCQCTSSATRNTTKQVTPQPEEQKERKTTEMQSPM
QPVDQASLPGHCREPPPWENEATERIYHFVVGQMVYYQCVQGYRAL
HRGPAESVCKMTHGKTRWTQPQLICTGEMETSQFPGEEKPQASPEG
RPESETSCLVTTTDFQIQTEMAATMETSIFTTEYQVAVAGCVFLLI
SVLLLSGLTWQRRQRKSRRTI

CD122: As used herein, the terms “CD122”, “interleukin-2 receptor beta”, “IL2Rb”, “IL2Rβ”, “IL15Rβ” and “p70-75” are used interchangeably to refer to the CD122 transmembrane protein. The human CD122 (hCD122) is a single pass type 1 transmembrane receptor and is expressed as a 551 amino acid pre-protein, the first 26 amino acids comprising a signal sequence which is post-translationally cleaved from the mature 525 amino acid protein. Amino acids 27-240 of the pre-protein (amino acids 1-214 of the mature protein) correspond to the extracellular domain, amino acids 241-265 of the pre-protein (amino acids 225-239 of the mature protein) correspond to the transmembrane domain and amino acids 266-551 of the pre-protein (amino acids 240-525 of the mature protein) correspond to the intracellular domain. As used herein, the term hCD122 includes naturally occurring variants of the hCD122 protein including the S57F and D365E (residues numbered in accordance with the mature hCD122 protein). hCD122 is referenced at UniProtKB database as entry P14784. Human CD122 nucleic acid and protein sequences may be found as Genbank accession numbers NM_000878 and NP_000869 respectively. The amino acid sequence of the mature hCD122 protein without the signal sequence is:

(SEQ ID NO: 2)
AVNGTSQFTCFYNSRANISCVWSQDGALQDTSCQVHAWPDRRRWNQ
TCELLPVSQASWACNLILGAPDSQKLTTVDIVTLRVLCREGVRWRV
MAIQDFKPFENLRLMAPISLQVVHVETHRCNISWEISQASHYFERH
LEFEARTLSPGHTWEEAPLLTLKQKQEWICLETLTPDTQYEFQVRV
KPLQGEFTTWSPWSQPLAFRTKPAALGKDTIPWLGHLLVGLSGAFG
FIILVYLLINCRNTGPWLKKVLKCNTPDPSKFFSQLSSEHGGDVQK
WLSSPFPSSSFSPGGLAPEISPLEVLERDKVTQLLLQQDKVPEPAS
LSSNHSLTSCFTNQGYFFFHLPDALEIEACQVYFTYDPYSEEDPDE
GVAGAPTGSSPQPLQPLSGEDDAYCTFPSRDDLLLFSPSLLGGPSP
PSTAPGGSGAGEERMPPSLQERVPRDWDPQPLGPPTPGVPDLVDFQ
PPPELVLREAGEEVPDAGPREGVSFPWSRPPGQGEFRALNARLPLN
TDAYLSLQELQGQDPTHLV

and the amino acid sequence of the extracellular domain (ECD) of hCD122 is:

(SEQ ID NO: 3)
AVNGTSQFTCFYNSRANISCVWSQDGALQDTSCQVHAWPDRRRWNQ
TCELLPVSQASWACNLILGAPDSQKLTTVDIVTLRVLCREGVRWRV
MAIQDFKPFENLRLMAPISLQVVHVETHRCNISWEISQASHYFERH
LEFEARTLSPGHTWEEAPLLTLKQKQEWICLETLTPDTQYEFQVRV
KPLQGEFTTWSPWSQPLAFRTKPAALGKDT

CD132: As used herein, the terms “CD132”, “IL2 receptor gamma”, “IL2Rg” and “IL2Rγ” are used interchangeably to refer to a type 1 cytokine receptor and is shared by the receptor complexes for IL-4, IL-7, IL-9, IL-15, and IL21, hence the reference to the “common” gamma chain. Human CD132 (hCD132) is expressed as a 369 amino acid pre-protein comprising a 22 amino acid N-terminal signal sequence. Amino acids 23-262 of the pre-protein (amino acids 1-240 of the mature protein) correspond to the extracellular domain, amino acids 263-283 of the pre-protein (amino acids 241-262 of the mature protein) correspond to the 21 amino acid transmembrane domain, and amino acids 284-369 of the pre-protein (amino acids 262-347 of the mature protein) correspond to the intracellular domain. hCD132 is referenced at UniProtKB database as entry P31785. Human CD132 nucleic acid and protein sequences may be found as Genbank accession numbers: NM_000206 and NP_000197 respectively. The amino acid sequence of the mature hCD132 protein is:

(SEQ ID NO: 4)
LNTTILTPNGNEDTTADFFLTTMPTDSLSVSTLPLPEVQCFVFNVEY
MNCTWNSSSEPQPTNLTLHYWYKNSDNDKVQKCSHYLFSEEITSGCQ
LQKKEIHLYQTFVVQLQDPREPRRQATQMLKLQNLVIPWAPENLTLH
KLSESQLELNWNNRFLNHCLEHLVQYRTDWDHSWTEQSVDYRHKFSL
PSVDGQKRYTFRVRSRFNPLCGSAQHWSEWSHPIHWGSNTSKENPFL
FALEAVVISVGSMGLIISLLCVYFWLERTMPRIPTLKNLEDLVTEYH
GNFSAWSGVSKGLAESLQPDYSERLCLVSEIPPKGGALGEGPGASPC
NQHSPYWAPPCYTLKPET

CDRs. As used herein, the term “CDR” or “complementarity determining region” refers to the non-contiguous antigen combining sites found within the variable region of both heavy and light chain immunoglobulin polypeptides. CDRs have been described by Kabat et al., J. Biol. Chem. 252:6609-6616 (1977); Kabat et al., U.S. Dept. of Health and Human Services, “Sequences of proteins of immunological interest” (1991) (also referred to herein as Kabat 1991); by Chothia et al., J. Mol. Biol. 196:901-917 (1987) (also referred to herein as Chothia 1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. In the context of the present disclosure, the numbering of the CDR positions is provided according to Kabat numbering conventions.

Circulating Tumor Cell: As used herein the term “circulating tumor cell (CTC)” refers to tumor cells that have been shed from a tumor mass (e.g., neoplasm) into the peripheral circulation.

Comparable: As used herein, the term “comparable” is used to describe the degree of difference in two measurements of an evaluable quantitative or qualitative parameter. For example, where a first measurement of an evaluable quantitative parameter (e.g. a level of IL2 activity as determined by a CTLL-2 proliferation or phospho-STAT5 assay) and a second measurement of the evaluable parameter do not deviate beyond a range that the skilled artisan would recognize as not producing a statistically significant difference in effect between the two results in the circumstances, the two measurements would be considered “comparable.” In some instances, measurements may be considered “comparable” if one measurement deviates from another by less than 35%, alternatively by less than 30%, alternatively by less than 25%, alternatively by less than 20%, alternatively by less than 15%, alternatively by less than 10%, alternatively by less than 7%, alternatively by less than 5%, alternatively by less than 4%, alternatively by less than 3%, alternatively by less than 2%, or by less than 1%. In particular embodiments, one measurement is comparable to a reference standard if it deviates by less than 15%, alternatively by less than 10%, or alternatively by less than 5% from the reference standard.

Derived From: As used herein in the term “derived from”, in the context of an amino acid sequence (e.g., a polypeptide comprising an amino acid sequence “derived from” an IL2 polypeptide or polynucleotide sequence), is meant to indicate that the polypeptide or nucleic acid has a sequence that is based on that of a reference polypeptide or nucleic acid (e.g., a naturally occurring IL2 polypeptide or an IL2-encoding nucleic acid) and is not meant to be limiting as to the source or method in which the protein or nucleic acid is made. By way of example, the term “derived from” includes homologs or variants of reference amino acid or DNA sequences.

Effective Concentration (EC): As used herein, the terms “effective concentration” or its abbreviation “EC” are used interchangeably to refer to the concentration of an agent (e.g., an IL2 ortholog) in an amount sufficient to effect a change in a given parameter in a test system. The abbreviation “E” refers to the magnitude of a given biological effect observed in a test system when that test system is exposed to a test agent. When the magnitude of the response is expressed as a factor of the concentration (“C”) of the test agent, the abbreviation “EC” is used. In the context of biological systems, the term Emax refers to the maximal magnitude of a given biological effect observed in response to a saturating concentration of an activating test agent. When the abbreviation EC is provided with a subscript (e.g., EC₄₀, EC₅₀, etc.) the subscript refers to the percentage of the Emax of the biological observed at that concentration. For example, the concentration of a test agent sufficient to result in the induction of a measurable biological parameter in a test system that is 30% of the maximal level of such measurable biological parameter in response to such test agent, this is referred to as the “EC₃₀” of the test agent with respect to such biological parameter. Similarly, the term “EC₁₀₀” is used to denote the effective concentration of an agent that results the maximal (100%) response of a measurable parameter in response to such agent. Similarly, the term EC₅₀ (which is commonly used in the field of pharmacodynamics) refers to the concentration of an agent sufficient to results in the half-maximal (50%) change in the measurable parameter. The term “saturating concentration” refers to the maximum possible quantity of a test agent that can dissolve in a standard volume of a specific solvent (e.g., water) under standard conditions of temperature and pressure. In pharmacodynamics, a saturating concentration of a drug is typically used to denote the concentration sufficient of the drug such that all available receptors are occupied by the drug, and EC₅₀ is the drug concentration to give the half-maximal effect.

Enriched: As used herein in the term “enriched” refers to a sample that is non-naturally manipulated so that a species (e.g. a molecule or cell) of interest is present in: (a) a greater concentration (e.g., at least 3-fold greater, alternatively at least 5-fold greater, alternatively at least 10-fold greater, alternatively at least 50-fold greater, alternatively at least 100-fold greater, or alternatively at least 1000-fold greater) than the concentration of the species in the starting sample, such as a biological sample (e.g., a sample in which the molecule naturally occurs or in which it is present after administration); or (b) a concentration greater than the environment in which the molecule was made (e.g., as in a recombinantly modified bacterial or mammalian cell). In some embodiments, the term “enriched” is used herein in reference to a population of cells comprising cells that express an orthogonal receptor following contacting the population of cells with cognate ortholog in an amount sufficient to cause a response in those cells that express an orthogonal receptor, the response being proliferation, such that concentration of cells that express the orthogonal receptor in the population is greater (e.g., at least 3-fold greater, alternatively at least 5-fold greater, alternatively at least 10-fold greater, alternatively at least 50-fold greater, alternatively at least 100-fold greater, or alternatively at least 1000-fold greater) after contacting with the population of cells with the cognate ortholog.

Extracellular Domain: As used herein the term “extracellular domain” or its abbreviation “ECD” refers to the portion of a cell surface protein (e.g. a cell surface receptor) which is outside of the plasma membrane of a cell. The ECD may include the entire extra-cytoplasmic portion of a transmembrane protein, a cell surface or membrane associated protein, a secreted protein, a cell surface targeting protein,

hCD122: As used herein the term “hCD122” refers to a naturally occurring human CD122 polypeptide including naturally occurring variants thereof. The amino acid sequence of naturally occurring mature hCD122 is provided as SEQ ID NO 2:

Identity: The term “identity,” as used herein in reference to polypeptide or DNA sequences, refers to the subunit sequence identity between two molecules. When a subunit position in both of the molecules is occupied by the same monomeric subunit (i.e., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof. Algorithms that are suitable for determining percent sequence identity and sequence similarity are the BLAST and BLAST 2.0 algorithms, which are described in Altschul et al. (1990) J Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

IL2: As used herein, the term “interleukin-2” or “IL2” refers to an IL2 polypeptide that possesses IL2 activity. In some embodiments, IL2 refers to mature wild-type human IL2. Mature wild-type human IL2 (hIL2) occurs as a 133 amino acid polypeptide (less the 20 N-terminal amino acids of the signal peptide of the pre-protein), as described in Fujita, et. al., PNAS USA, 80, 7437-7441 (1983). An amino acid sequence of naturally occurring variant of mature wild-type human IL2 (hIL2) is:

APTSSSTKKT QLQLEHLLLD LQMILNGINN YKNPKLTRML TFKFYMPKKA

TELKHLQCLE EELKPLEEVL NLAQSKNFHL RPRDLISNIN VIVLELKGSE

TTFMCEYADE TATIVEFLNR WITFCQSIIS TLT (SEQ ID NO:5)

As used herein, the numbering of residues of IL2 variants is based on the IL2 sequence UniProt ID P60568 excluding the signal peptide which is the same as that of SEQ ID NO: 5

IL2 Activity: The term “IL2 activity” refers to one or more the biological effects on a cell in response to contacting the cell with an effective amount of an IL2 polypeptide. As previously noted, IL2 is a pleitropic cytokine that results one or more biological effects on a variety of cell types. IL2 promotes the proliferation and expansion of activated T lymphocytes, induces proliferation and activation of naïve T cells, potentiates B cell growth, and promotes the proliferation and expansion of NK cells. One example of IL2 activity may be measured in a cell proliferation assay using CTLL-2 mouse cytotoxic T cells, see Gearing, A. J. H. and C. B. Bird (1987) in Lymphokines and Interferons, A Practical Approach. Clemens, M. J. et al. (eds): IRL Press. 295. The specific activity of recombinant human IL2 (rhIL2) is approximately 2.1×10⁴ IU/μg, which is calibrated against recombinant human IL2 WHO International Standard (NIBSC code: 86/500). In some embodiments, for example when the IL2 orthogonal polypeptide ligand of interest exhibits (or is engineered to possess) diminished affinity for CD25, it level of IL2 activity may be assessed in human cells such as YT cells which do not require CD25 to provide signaling through the IL2 receptor but rather are capable of signaling through the intermediate affinity CD122/CD132 receptor. In some embodiments, an orthogonal human IL2 ligand of the present disclosure may exhibit less than about 20%, alternatively less than about 10%, alternatively less than about 8%, alternatively less than about 6%, alternatively less than about 4%, alternatively less than about 2%, alternatively less than about 1%, alternatively less than about 0.5% of the activity of WHO International Standard (NIBSC code: 86/500) wild-type mature human IL2 when evaluated at equivalent concentrations in a comparable assay.

IL2 ortholog: As used herein, the term “IL2 ortholog” refers to a variant of IL2 derived from an IL2 parent polypeptide wherein the IL2 ortholog specifically binds to an orthogonal CD122 ECD and exhibits significantly reduced binding to the extracellular domain of a wild type CD122. In some embodiment the IL2 ortholog exhibits specific binding to a receptor comprising an orthogonal CD122 ECD and (2) the contacting of a cell expressing a membrane spanning receptor comprising the ECD of an orthogonal CD122 polypeptide in an amount sufficient to cause a response results in the a signal characteristic of the signal produced by the intracellular domain (ICD) of said membrane spanning receptor. When the membrane spanning receptor comprises an orthogonal CD122 ECD and CD122 ICD, the binding of an IL2 ortholog to such receptor results in an intracellular signal characteristic of the activation of a Cd25/CD122/CD132 high affinity of CD122/CD132 intermediate affinity IL2 receptor. An IL2 ortholog exhibits significantly reduced binding to wild-type hCD122. The term IL2 orthologs includes IL2 orthogonal variants and modified IL2 orthologs. In some embodiments, the IL2 ortholog is derived from a naturally occurring variant of human IL2 and such human IL2 orthologs may be referred to as “hoCD122” or “hoRb.” Certain modified IL2 polypeptides are provided in Garcia, et al. (United States Patent Application Publication US2018/0228842A1 published Aug. 16, 2018). As used herein, the term IL2 orthologs includes the modified hIL2 polypeptides described in Garcia, et al United State Patent Application Publication US2018/0228842A1 published Aug. 16, 2018. In some embodiments, the affinity of the IL2 ortholog for the extracellular domain of the orthogonal CD122 is comparable to the affinity of wild-type IL2 for ECD of wild-type CD122. In some embodiments, the affinity of the IL2 ortholog for the ECD of the orthogonal CD122 is greater than to the affinity of wild-type IL2 for ECD of wild-type CD122. In some embodiments, the affinity of the IL2 ortholog for the ECD of the orthogonal CD122 is less than to the affinity of wild-type IL2 for the ECD of the wild-type CD122.

In An Amount Sufficient Amount to Effect a Response: As used herein the phrase “in an amount sufficient to cause a response” is used in reference to the amount of a test agent sufficient to provide a detectable change in the level of an indicator measured before (e.g., a baseline level) and after the application of a test agent to a test system. In some embodiments, the test system is a cell, tissue or organism. In some embodiments, the test system is an in vitro test system such as a fluorescent assay. In some embodiments, the test system involves the measurement of a change in the level a parameter of a cell, tissue, or organism reflective of a biological function before and after the application of the test agent to the cell, tissue, or organism. In some embodiments, the indicator (e.g. concentration of phosphorylated STAT5) is reflective of biological function (e.g. activation of the IL2 receptor) of a cell evaluated in a in an assay in response to the administration of a quantity of the test agent (e.g. IL2). In some embodiments, the test system involves the measurement of a change in the level a parameter (e.g. luminescence) of a cell, tissue, or organism (e.g. a mouse injected with luminescent neoplastic cells) reflective of a biological condition (e.g. the presence of a neoplasm) before and after the application of one or more test agents (e.g. a CAR-T cell expressing an orthogonal CD122 in combination with an IL2 ortholog) to the cell, tissue, or organism (e.g. the mouse). In some embodiments, the indicator (e.g. concentration of phosphorylated STAT5) is reflective of biological function (e.g. activation of an IL2 receptor) of a cell (e.g. a T cell) evaluated in a in an assay in response to the administration of a quantity of the test agent (e.g. IL2). “An amount sufficient to effect a response” may be sufficient to be a therapeutically effective amount but “in an amount sufficient to cause a response” may be more or less than a therapeutically effective amount.

In Need of Treatment: The term “in need of treatment” as used herein refers to a judgment made by a physician or other caregiver with respect to a subject that the subject requires or will potentially benefit from treatment. This judgment is made based on a variety of factors that are in the realm of the physician's or caregiver's expertise.

In Need of Prevention: As used herein the term “in need of prevention” refers to a judgment made by a physician or other caregiver with respect to a subject that the subject requires or will potentially benefit from preventative care. This judgment is made based upon a variety of factors that are in the realm of a physician's or caregiver's expertise.

Inhibitor: As used herein the term “inhibitor” refers to a molecule that decreases, blocks, prevents, delays activation of, inactivates, desensitizes, or down-regulates, e.g., a gene, protein, ligand, receptor, or cell. An inhibitor can also be defined as a molecule that reduces, blocks, or inactivates a constitutive activity of a cell or organism.

Isolated: As used herein the term “isolated” is used in reference to a polypeptide of interest that, if naturally occurring, is in an environment different from that in which it can naturally occur. “Isolated” is meant to include polypeptides that are within samples that are substantially enriched for the polypeptide of interest and/or in which the polypeptide of interest is partially or substantially purified. Where the polypeptide is not naturally occurring, “isolated” indicates that the polypeptide has been separated from an environment in which it was made by either synthetic or recombinant means.

Intracellular Domain of the Orthogonal Receptor: As used herein the terms “intracellular domain of the orthogonal receptor” or “ICD-OR” refer to the portion of a transmembrane spanning orthogonal receptor that is inside of the plasma membrane of a cell expressing such transmembrane spanning orthogonal receptor. The ICD-OR may comprise one or more “proliferation signaling domain(s)” or “PSD(s)” which refers to a protein domain which signals the cell to enter mitosis and begin cell growth. Examples include the Janus kinases, including but not limited to, JAK1, JAK2, JAK3, Tyk2, Ptk-2, homologous members of the Janus kinase family from other mammalian or eukaryotic species, the IL2 receptor β and/or γ chains and other subunits from the cytokine receptor superfamily of proteins that may interact with the Janus kinase family of proteins to transduce a signal, or portions, modifications or combinations thereof. Examples of signals include phosphorylation of one or more STAT molecules including but not limited to one or more of STAT1, STAT3, STAT5a, and/or STAT5b.

“In Combination With”: As used herein, the term “in combination with” when used in reference to the administration of multiple agents to a subject refers to the administration of a first agent at least one additional (i.e. second, third, fourth, fifth, etc.) agent to a subject. For purposes of the present invention, one agent (e.g. IL2 ortholog) is considered to be administered in combination with a second agent (e.g. an engineered human immune cell) if the biological effect resulting from the administration of the first agent persists in the subject at the time of administration of the second agent such that the therapeutic effects of the first agent and second agent overlap. For example, an engineered orthogonal cell therapy agent would typically be administered infrequently (typically only a single administration) while the while the IL2 orthologs are administered periodically while the orthogonal cell agent persists in the subject. The engineered orthogonal cell therapy agent provides a therapeutic effect over an extended time (weeks or months) and the administration of the second agent (e.g. an IL2 ortholog) provides its therapeutic effect while the therapeutic effect of the first agent remains ongoing such that the second agent is considered to be administered in combination with the first agent, even though the first agent may have been administered at a point in time significantly distant (e.g. days or weeks) from the time of administration of the second agent. In one embodiment, one agent is considered to be administered in combination with a second agent if the first and second agents are administered simultaneously (within 30 minutes of each other), contemporaneously or sequentially. In some embodiments, a first agent is deemed to be administered “contemporaneously” with a second agent if first and second agents are administered within about 24 hours of each another, preferably within about 12 hours of each other, preferably within about 6 hours of each other, preferably within about 2 hours of each other, or preferably within about 30 minutes of each other. The term “in combination with” shall also understood to apply to the situation where a first agent and a second agent are co-formulated in single pharmaceutically acceptable formulation and the co-formulation is administered to a subject. In certain embodiments, orthogonal cell and IL2 ortholog is further combined with additional supplementary agents. The supplementary agent(s) are administered or applied sequentially, e.g., where one agent is administered prior to one or more other agents. In other embodiments, the IL2 mutein and the supplementary agent(s) are administered simultaneously, e.g., where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

High Affinity IL2 Receptor: As used herein, the term “high affinity IL2 receptor” refers to a trimeric receptor complex comprising the CD25, CD122 and CD132 proteins (also referred to as “IL2Rαβγ”). Wild type hIL2 (SEQ ID NO:5) possesses a Kd of approximately 10⁻¹¹ M with respect to the high IL2 affinity receptor complex.

Intermediate Affinity IL2 Receptor: As used herein, the term “intermediate affinity IL2 receptor” refers to a dimeric IL2 receptor complex comprising CD122 and CD132 (also referred to as “IL2Rβγ”). The association of an IL2 molecule with the intermediate affinity IL2 receptor expressed on the surface of a mammalian immune cell results in IL2 signaling in the cell. Wild type hIL2 (SEQ ID NO:5) possesses a Kd of approximately 10⁻⁹M with respect to the intermediate affinity CD122/CD132 (IL2Rβγ) receptor complex.

Kabat Numbering: The term “Kabat numbering” as used herein is recognized in the art and refers to a system of numbering amino acid residues which are more variable than other amino acid residues (e.g., hypervariable) in the heavy and light chain regions of immunoglobulins (Kabat, et al., (1971) Ann. NY Acad Sci. 190:382-93; Kabat, et al., (1991) Sequences of Proteins of Immunological Interest, Fifth Edition, U.S. Department of Health and Human Services, NIH Publication No. 91-3242). For purposes of the present disclosure, the positioning of CDRs in the variable region of an antibody follows Kabat numbering or simply, “Kabat.”

Ligand: As used herein, the term “ligand” refers to a molecule that specifically binds a receptor and causes a change in the receptor so as to effect a change in the activity of the receptor or a response in cell that expresses that receptor. In one embodiment, the term “ligand” refers to a molecule, or complex thereof, that can act as an agonist or antagonist of a receptor. As used herein, the term “ligand” encompasses natural and synthetic ligands. “Ligand” also encompasses small molecules, peptide mimetics of cytokines and peptide mimetics of antibodies. The complex of a ligand and receptor is termed a “ligand-receptor complex.” A ligand may comprise one domain of a polyprotein or fusion protein (e.g., an antibody-targeted ligand fusion protein).

Metastasis: As used herein the term “metastasis” describes the spread of cancerous cells from the primary tumor to surrounding tissues and to distant organs.

Modified IL2 Ortholog: As used herein the term “modified IL2 orthologs” is used to refer to IL2 orthologs that have been modified by one or more modifications such as pegylation, glycosylation (N- and O-linked), acylation, or polysialylation or by conjugation (either chemical or as fusion proteins) with other polypeptide carrier molecules including but not limited to albumin fusion polypeptides comprising serum albumin (e.g., human serum albumin (HSA) or bovine serum albumin (BSA)), Fc-fusion proteins), targeted IL2 ortholog fusion proteins (such as scFv-IL2 ortholog fusion proteins, VHH-IL2 orthogonal polypeptide fusion proteins) and the like. Modified IL2 orthologs may be prepared to order to enhance one or more properties for example, modulating immunogenicity (conjugation or fusion to immunogens), methods of increasing water solubility, bioavailability, serum half-life, and/or therapeutic half-life; and/or modulating biological activity. Certain modifications can also be useful to, for example, generation of antibodies for use in detection assays (e.g., epitope tags) or to provide for ease of protein purification (e.g., poly-His tags). Modified IL2 orthologs may be prepared to order to enhance one or more properties for example, modulating immunogenicity; methods of increasing water solubility, bioavailability, serum half-life, and/or therapeutic half-life; and/or modulating biological activity. Certain modifications can also be useful to, for example, raise of antibodies for use in detection assays (e.g., epitope tags) and to provide for ease of protein purification. In some embodiments, the modified IL2 ortholog is at least 90%, 91%, 92%, 93%, 94%, 95%, 96%, 97%, 98%, or 99% amino acid sequence identity to SEQ ID NO:5 excluding any modifications as encompassed with the modifications of the Formula 1.

Modulate: As used herein, the terms “modulate”, “modulation” and the like refer to the ability of a test agent to cause a response, either positive or negative or directly or indirectly, in a system, including a biological system or biochemical pathway. The term modulator includes both agonists (including partial agonists, full agonists and superagonists) and antagonists.

Neoplastic Disease: As used herein, the term “neoplastic disease” refers to disorders or conditions in a subject arising from cellular hyper-proliferation or unregulated (or dysregulated) cell replication. The term neoplastic disease refers to disorders arising from the presence of neoplasms in the subject. Neoplasms may be classified as: (1) benign (2) pre-malignant (or “pre-cancerous”); and (3) malignant (or “cancerous”). The term “neoplastic disease” includes neoplastic-related diseases, disorders and conditions referring to conditions that are associated, directly or indirectly, with neoplastic disease, and includes, e.g., angiogenesis and precancerous conditions such as dysplasia or smoldering multiple myeloma.

N-Terminus: As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” or “immediately C-terminal” refers to a position of a first amino acid residue relative to a second amino acid residue where the first and second amino acid residues are covalently bound to provide a contiguous amino acid sequence.

Neoplastic Disease: As used herein, the term “neoplastic disease” refers to disorders or conditions in a subject arising from cellular hyper-proliferation or unregulated (or dysregulated) cell replication. The term neoplastic disease refers to disorders arising from the presence of neoplasms in the subject. Neoplasms may be classified as: (1) benign (2) pre-malignant (or “pre-cancerous”); and (3) malignant (or “cancerous”). The term “neoplastic disease” includes neoplastic-related diseases, disorders and conditions referring to conditions that are associated, directly or indirectly, with neoplastic disease, and includes, e.g., angiogenesis and precancerous conditions such as dysplasia.

Nucleic Acid: The terms “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

Numbered in accordance with IL2: The term “numbered in accordance with IL2” as used herein refers to the identification of a location of particular amino acid with reference to the position at which that amino acid normally occurs in the sequence of the mature wild type IL2. In some embodiments, the IL2 is hIL2 (SEQ ID NO: 5). For example, in reference to hIL2, “R81” refers to the eighty-first (numbered from the N-terminus) amino acid, arginine, that occurs in sequence of the mature wild type hIL2. It should be noted that the amino acid sequences of IL2 molecules of different mammalian species have different numbers and sequences of amino acids. Consequently, when referencing a residue in accordance with this convention it is helpful to identify the IL2 species in question.

Numbered in accordance with CD122: The term “numbered in accordance with CD122” as used herein refers to the identification of a location of particular amino acid with reference to the position at which that amino acid normally occurs in the sequence of the mature wild type CD122 molecules. In one embodiment, the CD122 molecule is human CD122 (SEQ ID NO. 2). For example, in reference to human CD122, H133 refers to the histidine at the one-hundred thirty third (numbered from the N-terminus) amino acid of the sequence of the mature wild type hCD122.

Numbered in accordance with the Extracellular Domain of CD122: The term “numbered in accordance with extracellular domain of CD122” or “numbered in accordance with CD122 ECD” as used herein refers to the identification of a location of particular amino acid with reference to the position at which that amino acid normally occurs in the extracellular domain (ECD) sequence of the mature wild type CD122 molecules. In one embodiment, the CD122 ECD molecule is human the CD122 ECD (SEQ ID NO. 3). For example, in reference to human CD122 ECD, H133 refers to the histidine at the one-hundred thirty third (numbered from the N-terminus) amino acid of the sequence of the mature wild type hCD122 ECD.

Operably Linked: The term “operably linked” is used herein to refer to the relationship between nucleic acid sequences encoding differing functions when combined into a single nucleic acid sequence that, when introduced into a cell, provides a nucleic acid which is capable of effecting the transcription and/or translation of a particular nucleic acid sequence in a cell. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, certain genetic elements such as enhancers need not be contiguous with respect to the sequence to which they provide their effect.

Orthogonal Cell: As used herein, the term “orthogonal cell” refers to a mammalian cell which has been recombinantly modified to express an orthogonal receptor. In some embodiments the orthogonal cell expresses the orthogonal CD122 (oCD122) polypeptide or a receptor comprising an orthogonal CD122 ECD. In some embodiments the orthogonal cell a modified human cell (“human orthogonal cell”). The orthogonal cell may be an immune cell, for example a human immune cell. A “human orthogonal immune cell” is human immune cell recombinantly modified to expression a human orthogonal CD122 (hoCD122) or chimeric receptor comprising the human orthogonal CD122 ECD). In some embodiments, the immune cell for engineering to express the oCD122 (or receptor comprising the oCD122 ECD) selected from the group consisting of myeloid cells, lymphocytes, peripheral blood mononuclear cells (PBMCs), tumor infiltrating lymphocytes (TILs), T cells, CD8+ T cells, CD25+CD8+ T cells, CAR-T cells, NK cells, CD4+ T cells, and Tregs engineered versions thereof including but limited to engineered TILs, engineered Tregs and engineered NK cells. In some embodiments, the orthogonal cell is CAR-T derived from a human immune cell that has been recombinantly modified to express a human orthogonal CD122 (“hoCAR-T” cell). In some embodiments, the orthogonal cell is a TIL isolated from the neoplasm of a human subject that has been recombinantly modified to express a human orthogonal CD122 or receptor comprising the oCD122 ECD (“hoTIL” cell). In some embodiments, the orthogonal cell is NK isolated from a human subject that has been recombinantly modified to express a human orthogonal CD122 or receptor comprising the oCD122 ECD (“hoNK” cell). In some embodiments, the cell is a human hematopoietic stem that has been recombinantly modified to expression a human orthogonal CD122 or receptor comprising the oCD122 ECD (“hoHSC” cell). In some embodiments, the orthogonal cell is TCR engineered cell derived from a human immune cell that has been recombinantly modified to express a non-native T cell receptor further modified to express a human orthogonal CD122 or receptor comprising the oCD122 ECD (“hoTCR cell”). As used herein, the term “orthogonal cell” refers to a mammalian cell which has been recombinantly modified to express an orthogonal CD122 or receptor comprising an oCD122 ECD. The orthogonal cell may incorporate recombinant modifications in addition to the recombinant modifications necessary to express an orthogonal CD122 polypeptide or receptor comprising an oCD122 ECD including recombinant modifications including the introduction of nucleic acid molecules encoding marker proteins operably linked to expression control sequences to facilitate expression in the orthogonal cell including but not limited to nucleic acid molecules encoding marker proteins (proteins conferring antibiotic resistance, fluorescent proteins, or luminescent proteins); biologically active intracellularly proteins including but not limited to DNA or RNA binding proteins, transcription factors including transcriptional repressors or de-repressors, pro-apoptotic proteins, anti-apoptotic proteins and intracellular regulatory proteins; biologically active secreted proteins such as growth factors, peptide hormones, cytokines or chemokines including biologically active therapeutic proteins such as antibodies (the extracellular protein typically comprising a signal peptide or secretion leader sequence to facilitate extracellular transport following expression in the orthogonal cell). Additional recombinant modifications to the orthogonal cells will be apparent to those of skill in the art. In some embodiments, the orthogonal CD122 receptor expressed on the orthogonal cell may comprise a human CD122 intracellular domain (ICD) modified to provide one or more STAT3 binding motifs.

Orthogonal CD122: As used herein the term “orthogonal CD122” or “CD122 orthogonal receptor” are used interchangeably herein to refer to an CD122 polypeptide variant comprising amino acid substitutions that result in specific binding to an IL2 ortholog that is a cognate ligand for such CD122 polypeptide variant but does not specifically bind to a naturally occurring form of IL2.

Orthogonal Receptor: As used herein the term “orthogonal receptor” refers to a variant of a receptor, the orthogonal receptor comprising modifications to the amino acid sequence so that the orthogonal receptor exhibits significantly reduced binding to its cognate ligand but exhibits specific binding for an orthogonal ligand engineered to interact with the orthogonal receptor. In some embodiments, the orthogonal receptor may comprise an extracellular domain that is exhibits significantly reduced binding to its cognate native ligand, while an orthogonal ligand exhibits significantly reduced binding to the ECD of its cognate native receptor(s). In some embodiments, the affinity of the orthogonal ligand for the cognate orthogonal receptor exhibits affinity comparable to the affinity of the native ligand for the native receptor, e.g. having an affinity that is least about 1% of the native cytokine receptor pair affinity, at least about 5%, at least about 10%, at least about 25%, at least about 50%, at least about 75%, at least about 100%, and may be higher, e.g. 2×, 3×, 4×, 5×, 10× or more of the affinity of the native cytokine for the native receptor. An orthogonal receptor may be referred to by the parent molecule from which it was derived (e.g. orthogonal CD122) or by the cognate ligand from which the orthogonal ligand for the orthogonal receptor was derived (e.g. orthogonal IL2 receptor).

Ortholog: As used herein the term “ortholog” or “orthogonal ligand” are used interchangeably herein to refer to the ligand component of an orthogonal ligand/receptor pair and refers to a polypeptide incorporating modifications to its primary structure to provide a polypeptide variant that exhibits: (a) significantly reduced affinity to its native cognate receptor (i.e., the native receptor for the parent polypeptide from which the ortholog is derived); and (b) specific binding a engineered orthogonal receptor which is a variant of the cognate receptor for the ortholog. Upon binding of the ortholog to the orthogonal receptor (which is expressed on surface of cell which has been modified by recombinant DNA technology to incorporate a nucleic acid sequence encoding the orthogonal receptor operably linked to control elements to effect the expression of the orthogonal receptor in the recombinantly modified cell), the activated orthogonal receptor initiates signaling that is transduced through native cellular elements to provide for a biological activity that mimics that native response of the cognate but which is specific to the recombinantly modified cell population expressing the orthogonal receptor. In some embodiments of the invention, orthologs possess significant selectivity for the orthogonal receptor relative to the cognate receptor and optionally possessing significantly reduced potency with respect to the cognate receptor. Selectivity is typically assessed by activity measured in an assay characteristic of the activity induced in response to ligand/receptor binding. In some embodiments, the ortholog possesses at least 5 fold, alternatively at least 10 fold, alternatively at least 20 fold, alternatively at least 30 fold, alternatively at least 40 fold, alternatively at least 50 fold, alternatively at least 100 fold, alternatively at least 200 fold difference in EC50 as measured in the same assay.

Parent Polypeptide: As used herein the terms “parent polypeptide” or “parent protein” are used interchangeably to refer to naturally occurring polypeptide that is subsequently modified to generate a variant polypeptide. A parent polypeptide may be a wild-type (or native) polypeptide. Parent polypeptide may refer to the polypeptide itself or compositions that comprise the parent polypeptide (e.g. glycosylated, pegylated, fusion proteins comprising the parent polypeptide).

Partial Agonist: As used herein, the term “partial agonist” refers to a molecule that specifically binds that bind to and activate a given receptor but possess only partial activation the receptor relative to a full agonist. Partial agonists may display both agonistic and antagonistic effects. For example when both a full agonist and partial agonist are present, the partial agonist acts as a competitive antagonist by competing with the full agonist for the receptor binding resulting in net decrease in receptor activation relative to the contact of the receptor with the full agonist in the absence of the partial agonist. Clinically, partial agonists can be used to activate receptors to give a desired submaximal response when inadequate amounts of the endogenous ligand are present, or they can reduce the overstimulation of receptors when excess amounts of the endogenous ligand are present. The maximum response (Eₘₐₓ) produced by a partial agonist is called its intrinsic activity and may be expressed on a percentage scale where a full agonist produced a 100% response. An IL2 partial agonist of the present disclosure may have greater than 10%, alternatively greater than 20%, alternatively greater than 30%, alternatively greater than 40%, alternatively greater than 50%, alternatively greater than 60%, or alternatively greater than 70% of the activity of WHO International Standard (NIBSC code: 86/500) wild type mature human IL2 when evaluated at similar concentrations in a comparable assay.

PEG-IL2 Ortholog: As used herein the term “PEG-IL2 ortholog” refers to a IL2 ortholog covalently bound to at least one polyethylene glycol (PEG) molecule, the at least one PEG molecule being covalently attached to at least one amino acid residue of an IL2 ortholog. The PEGylated polypeptide may be further referred to as monopegylated, dipegylated, tripegylated (and so forth) to denote PEG-IL2 orthologs comprising one, two, three (or more) PEG moieties attached to the IL2 ortholog, respectively. In some embodiments, the PEG may be covalently attached directly to the IL2 ortholog (e.g., through a lysine side chain, sulfhydryl group of a cysteine or N-terminal amine) or optionally employ a linker between the PEG and the IL2 ortholog. In some embodiments the PEG-IL2 ortholog comprises more than one PEG molecule each of which is attached to a different amino acid residue. In some embodiments, the PEG-IL2 ortholog is derived from (SEQ ID NO: 4, mature human wild-type hIL2). PEGylated forms of IL2 and the methodology of PEGylation of IL2 polypeptides is well known in the art (see, e.g., Katre, et al., U.S. Pat. No. 4,931,544 issued Jun. 5, 1990; Katre, et al., U.S. Pat. No. 5,206,344 issued Apr. 27, 1993; and Bossard, et al., U.S. Pat. No. 9,861,705 issued Jan. 9, 2018). In some embodiments, the IL2 mutein may be modified by the incorporation of non-natural amino acids with non-naturally occurring amino acid side chains to facilitate site specific PEGylation as described in Ptacin, et al. United States Patent Application Publication US20170369871A1 published Dec. 28, 2017. In other embodiments, cysteine residues may be incorporated at various positions within the IL2 molecule to facilitate site-specific PEGylation via the cysteine side chain as described in Greve, et al. PCT International Patent Application Number PCT/US2015/044462 published as WO2016/025385 on Feb. 18, 2016.

Polypeptide: As used herein the terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences; fusion proteins with or without N-terminus methionine residues; fusion proteins with immunologically tagged proteins; fusion proteins of immunologically active proteins (e.g. antigenic diphtheria or tetanus toxin fragments) and the like.

Prevent: As used herein the terms “prevent”, “preventing”, “prevention” and the like refer to a course of action initiated with respect to a subject prior to the onset of a disease, disorder, condition or symptom thereof so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed due to genetic, experiential or environmental factors to having a particular disease, disorder or condition. In certain instances, the terms “prevent”, “preventing”, “prevention” are also used to refer to the slowing of the progression of a disease, disorder or condition from a present its state to a more deleterious state.

Receptor: As used herein, the term “receptor” refers to a polypeptide having a domain that specifically binds a ligand that binding of the ligand results in a change to at least one biological property of the polypeptide. In some embodiments, the receptor is a “soluble” receptor that is not associated with a cell surface. The soluble form of hCD25 is an example of a soluble receptor that specifically binds hIL2. In some embodiments, the receptor is a cell surface receptor that comprises and extracellular domain (ECD) and a membrane associated domain which serves to anchor the ECD to the cell surface. In some embodiments of cell surface receptors, the receptor is a membrane spanning polypeptide comprising an intracellular domain (ICD) and extracellular domain (ECD) linked by a membrane spanning domain typically referred to as a transmembrane domain (TM). The binding of the ligand to the receptor results in a conformational change in the receptor resulting in a measurable biological effect. In some instances, where the receptor is a membrane spanning polypeptide comprising an ECD, TM and ICD, the binding of the ligand to the ECD results in a measurable intracellular biological effect mediated by one or more domains of the ICD in response to the binding of the ligand to the ECD. In some embodiments, a receptor is a component of a multi-component complex to facilitate intracellular signaling. For example, the ligand may bind a cell surface molecule having not associated with any intracellular signaling alone but upon ligand binding facilitates the formation of a heteromultimeric including heterodimeric (e.g. the intermediate affinity CD122/CD132 IL2 receptor), heterotrimeric (e.g. the high affinity CD25/CD122/CD132 hIL2 receptor) or homomultimeric (homodimeric, homotrimeric, homotetrameric) complex that results in the activation of an intracellular signaling cascade (e.g. the Jak/STAT pathway). In some embodiments, the receptor is a membrane spanning single chain polypeptide comprising ECD, TM and ICD domains wherein the ECD, TM and ICD domains are derived from the same or differing naturally occurring receptor variants. In some embodiments, the a receptor may be a hoCD122 receptor. In some embodiments, the receptor is a chimeric antigen receptor (CAR).

Recombinant: As used herein, the term “recombinant” is used as an adjective to refer to the method by a polypeptide, nucleic acid, or cell that was modified using recombinant DNA technology. A recombinant protein is a protein produced using recombinant DNA technology and is frequently abbreviated with a lower case “r” (e.g. rhIL2) to denote the method by which the protein was produced. Similarly a cell is referred to as a “recombinant cell” if the cell has been modified by the incorporation (e.g. transfection, transduction, infection) of exogenous nucleic acids (e.g., ssDNA, dsDNA, ssRNA, dsRNA, mRNA, viral or non-viral vectors, plasmids, cosmids and the like) using recombinant DNA technology. The techniques and protocols for recombinant DNA technology are well known in the art such as those can be found in Sambrook, et al. (1989) Molecular Cloning: A Laboratory Manual (2d ed., Cold Spring Harbor Laboratory Press, Plainview, N.Y.) and other standard molecular biology laboratory manuals.

Response: The term “response,” for example, of a cell, tissue, organ, or organism, encompasses a quantitative or qualitative change in a evaluable biochemical or physiological parameter, (e.g., concentration, density, adhesion, proliferation, activation, phosphorylation, migration, enzymatic activity, level of gene expression, rate of gene expression, rate of energy consumption, level of or state of differentiation, where the change is correlated with activation, stimulation, or treatment, or with internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects. Examples of such standard protocols to assess proliferation of CD3 activated primary human T-cells include bioluminescent assay that generates a luminescent signal that is proportional to the amount of ATP present which is directly proportional to the number of cells present in culture as described in Crouch, et al. (1993) “The use of ATP bioluminescence as a measure of cell proliferation and cytotoxicity” J. Immunol. Methods 160: 81-8 or a standardized commercially available assay system such as the CellTiter-Glo® 2.0 Cell Viability Assay or CellTiter-Glo® 3D Cell Viability kits commercially available from Promega Corporation, 2800 Woods Hollow Road, Madison Wis. 53711 as catalog numbers G9241 and G9681 respectively in substantial accordance with the instructions provided by the manufacturer. In some embodiments, the level of activation of T-cells in response to the administration of a test agent may be determined by flow cytometric methods as described as determined by the level of STAT5 phosphorylation in accordance with methods well known in the art. STAT5 phosphorylation may be measured using flow cytometric techniques as described in Horta, et al. supra., Garcia, et al., supra, or commercially available kits such as the Phospho-STAT5 (Tyr694) kit (commercially available from Perkin-Elmer/cisbio Waltham Mass. as Part Number 64AT5PEG) in substantial accordance with the teaching of the manufacturer. When the abbreviation EC^{ACT} used with a subscript this is provided to indicate the concentration of the test agent sufficient to produce the indicated percentage of maximal STAT5 phosphorylation in a T cell in response to the application of the test agent as measured in accordance with the test protocol. By way of illustration, the abbreviation EC₃₀^{PRO} may be used with respect to a hIL2 ortholog to indicate the concentration associated with 30% of a maximal level of STAT5 phosphorylation in a T cell in in response with respect to such hIL2 ortholog as measured with the Phospho-STAT5 (Tyr694) kit.

In some instances, there are standardized accepted measures of biological activity that have been established for a molecule. For example with respect to hIL2 potency, the standard methodology for the evaluation of hIL2 potency in international units (IU) is measured in the murine cytotoxic T cell line CTLL-2 in accordance with standardized procedures as more fully described in Wadhwa, et al. (2013) “The 2nd International standard for Interleukin-2 (IL2) Report of a collaborative study” Journal of Immunological Methods 397:1-7. It should be noted in the context of the present disclosure that the murine IL2 receptor functions differently than the human IL2 receptor, particularly with respect to need for all components of the trimeric receptor complex to provide intracellular signal transduction signaling (e.g. STAT5 phosphorylation). See, e.g. Horta, et al., (2019) “Human and murine IL2 receptors differentially respond to the human-IL2 component of immunocytokines” Oncoimmunology 8(6):e1238538-1, e1238538-15 and Nemoto, et al. (1995) “Differences in the interleukin-2 (IL2) receptor system in human and mouse: alpha chain is require for formation of the functional mouse IL2 receptor” European J Immunology 25(11) 3001-5. Consequently, when evaluating the activity of a hIL2 variant, particularly with respect to affinity for CD25 or activation of cells with respect to CD25 status the use of human cells or systems that recapitulate the biology of the human low, intermediate and high affinity IL2 receptors and receptor complexes is preferred and a molecule that exhibits selective binding or activation in a murine test system (e.g. an in vitro test system using murine cells or in vivo in mice) may not recapitulate such selective activity in a human system (e.g. an in vitro test system using human cells or in vivo in human subjects).

Selective: As used herein, the term “selective” is used to refer to a property of an agent to preferentially bind to and/or activate a particular cell type. In some embodiments, the presnde disclosure provides IL2 variants (IL2 orthologs) that selectively bind to engineered CD122 ECD polypeptides such that cells expressing receptors comprising such CD122 ECD polypeptides are activated in response to the binding of such IL2 ortholog to receptors comprising such cognate CD122 ECD polypeptides. In some embodiments, the disclosure provides hIL2 orthologs that are selective in that such orthologs display preferential activation of immune cells that expressing the hoCD122 receptors. Selectivity is typically assessed by activity measured in an assay characteristic of the activity induced in response to ligand/receptor binding. In some embodiments, selectivity of IL2 orthologs is measured by comparing the activation of cells expressing CD25 (e.g. YTCD25POS or YTCD25+ cells) versus the activation of that display significantly lower (preferably undetectable) levels of CD25 (e.g. YTCD25NEG or YTCD25− cells). In some embodiments, the selectivity is measured by activation of T cells expressing comparatively high levels of CD25 (e.g. Tregs) versus low comparatively low levels of CD25 (e.g. non stimulated CD8+ T cells).

Significantly Reduced Binding: As used herein, the term “exhibits significantly reduced binding” is used with respect to the affinity of the binding of a variant of a ligand (e.g. an ortholog) to a modified form of a receptor (e.g. an orthogonal CD122) relative to the binding of the variant ligand for the naturally occurring form of a receptor. In some embodiments a ligand (e.g. an ortholog) exhibits significantly reduced binding to the native form of the ligand if the orthogonal ligand binds to the native form of the receptor with and affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the naturally occurring ligand. Similarly and orthogonal receptor exhibits significantly reduced binding with respect to the native form of the ligand if the native form of the ligand binds to the orthogonal form of the receptor with and affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the naturally occurring receptor.

Specifically Binds: As used herein the term “specifically binds” refers to the degree of affinity for which one molecule binds to another. In the context of binding pairs (e.g. a ligand/receptor, antibody/antigen, antibody/ligand, antibody/receptor binding pairs) a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the first molecule of the binding pair when the affinity of the first molecule for the second molecule is at least two-fold greater, alternatively at least five times greater, alternatively at least ten times greater, alternatively at least 20-times greater, or alternatively at least 100-times greater than the affinity of the first molecule for other components present in the sample. In a particular embodiment, where the first molecule of the binding pair is an antibody, the antibody specifically binds to the second molecule of the binding pair (e.g. a protein, antigen, ligand, or receptor) if the equilibrium dissociation constant between antibody and to the second molecule of the binding pair is greater than about 10⁶M, alternatively greater than about 10⁸ M, alternatively greater than about 10¹⁰ M, alternatively greater than about 10¹¹ M, alternatively greater than about 10¹⁰ M, greater than about 10¹² M as determined by, e.g., Scatchard analysis (Munsen, et al. (1980) Analyt. Biochem. 107:220-239). In one embodiment where the ligand is an IL2 ortholog and the receptor comprises an orthogonal CD122 ECD, the IL2 ortholog specifically binds if the equilibrium dissociation constant of the IL2 ortholog/orthogonal CD122 ECD is greater than about 10⁵M, alternatively greater than about 10⁶ M, alternatively greater than about 10⁷M, alternatively greater than about 10⁸M, alternatively greater than about 10⁹M, alternatively greater than about 10¹⁰ M, or alternatively greater than about 10¹¹ M. Specific binding may be assessed using techniques known in the art including but not limited to competition ELISA, radioactive ligand binding assays (e.g., saturation binding, Scatchard plot, nonlinear curve fitting programs and competition binding assays); non-radioactive ligand binding assays (e.g., fluorescence polarization (FP), fluorescence resonance energy transfer (FRET) and surface plasmon resonance assays (see, e.g., Drescher et al., Methods Mol Biol 493:323-343 (2009) with instrumentation commercially available from GE Healthcare Bio-Sciences such as the Biacore 8+, Biacore S200, Biacore T200 (GE Healthcare Bio-Sciences, 100 Results Way, Marlborough Mass. 01752)); liquid phase ligand binding assays (e.g., real-time polymerase chain reaction (RT-qPCR), and immunoprecipitation); and solid phase ligand binding assays (e.g., multiwell plate assays, on-bead ligand binding assays, on-column ligand binding assays, and filter assays).

Subject: The terms “recipient”, “individual”, “subject”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is a human being.

Suffering From: As used herein, the term “suffering from” refers to a determination made by a physician with respect to a subject based on the available information accepted in the field for the identification of a disease, disorder or condition including but not limited to X-ray, CT-scans, conventional laboratory diagnostic tests (e.g. blood count, etc.), genomic data, protein expression data, immunohistochemistry, that the subject requires or will benefit from treatment. The term suffering from is typically used in conjunction with a particular disease state such as “suffering from a neoplastic disease” refers to a subject which has been diagnosed with the presence of a neoplasm.

Substantially Pure: As used herein in the term “substantially pure” indicates that a component (e.g., a polypeptide) makes up greater than about 50% of the total content of the composition, and typically greater than about 60% of the total polypeptide content. More typically, “substantially pure” refers to compositions in which at least 75%, at least 85%, at least 90% or more of the total composition is the component of interest. In some cases, the polypeptide will make up greater than about 90%, or greater than about 95% of the total content of the composition.

T-cell: As used herein the term “T-cell” or “T cell” is used in its conventional sense to refer to a lymphocytes that differentiates in the thymus, possess specific cell-surface antigen receptors, and include some that control the initiation or suppression of cell-mediated and humoral immunity and others that lyse antigen-bearing cells. In some embodiments the T cell includes without limitation naïve CD8⁺ T cells, cytotoxic CD8⁺ T cells, naïve CD4⁺ T cells, helper T cells, e.g. T_{H}1, T_{H}2, T_{H}9, T_{H}11, T_{H}22, T_{FH}; regulatory T cells, e.g. T_{R}1, Tregs, inducible Tregs; memory T cells, e.g. central memory T cells, effector memory T cells, NKT cells, tumor infiltrating lymphocytes (TILs) and engineered variants of such T-cells including but not limited to CAR-T cells, recombinantly modified TILs and TCR engineered cells.

Therapeutically Effective Amount: The phrase “therapeutically effective amount” as used herein in reference to the administration of an agent to a subject, either alone or as part of a pharmaceutical composition or treatment regimen, in a single dose or as part of a series of doses in an amount capable of having any detectable, positive effect on any symptom, aspect, or characteristic of a disease, disorder or condition when administered to the subject. The therapeutically effective amount can be ascertained by measuring relevant physiological effects, and it may be adjusted in connection with a dosing regimen and in response to diagnostic analysis of the subject's condition, and the like. The parameters for evaluation to determine a therapeutically effective amount of an agent are determined by the physician using art accepted diagnostic criteria including but not limited to indicia such as age, weight, sex, general health, ECOG score, observable physiological parameters, blood levels, blood pressure, electrocardiogram, computerized tomography, X-ray, and the like. Alternatively, or in addition, other parameters commonly assessed in the clinical setting may be monitored to determine if a therapeutically effective amount of an agent has been administered to the subject such as body temperature, heart rate, normalization of blood chemistry, normalization of blood pressure, normalization of cholesterol levels, or any symptom, aspect, or characteristic of the disease, disorder or condition, biomarkers (such as inflammatory cytokines, IFN-γ, granzyme, and the like), reduction in serum tumor markers, improvement in Response Evaluation Criteria In Solid Tumors (RECIST), improvement in Immune-Related Response Criteria (irRC), increase in duration of survival, extended duration of progression free survival, extension of the time to progression, increased time to treatment failure, extended duration of event free survival, extension of time to next treatment, improvement objective response rate, improvement in the duration of response, reduction of tumor burden, complete response, partial response, stable disease, and the like that that are relied upon by clinicians in the field for the assessment of an improvement in the condition of the subject in response to administration of an agent. As used herein the terms “Complete Response (CR),” “Partial Response (PR)” “Stable Disease (SD)” and “Progressive Disease (PD)” with respect to target lesions and the terms “Complete Response (CR),” “Incomplete Response/Stable Disease (SD)” and Progressive Disease (PD) with respect to non-target lesions are understood to be as defined in the RECIST criteria. As used herein the terms “immune-related Complete Response (irCR),” “immune-related Partial Response (irPR),” “immune-related Progressive Disease (irPD)” and “immune-related Stable Disease (irSD)” as defined in accordance with the Immune-Related Response Criteria (irRC). As used herein, the term “Immune-Related Response Criteria (irRC)” refers to a system for evaluation of response to immunotherapies as described in Wolchok, et al. (2009) Guidelines for the Evaluation of Immune Therapy Activity in Solid Tumors: Immune-Related Response Criteria, Clinical Cancer Research 15(23): 7412-7420. A therapeutically effective amount may be adjusted over a course of treatment of a subject in connection with the dosing regimen and/or evaluation of the subject's condition and variations in the foregoing factors. In one embodiment, a therapeutically effective amount is an amount of an agent when used alone or in combination with another agent does not result in non-reversible serious adverse events in the course of administration to a mammalian subject.

Transmembrane Domain: The term “transmembrane domain” or “TM.” refers to the domain of a membrane spanning polypeptide (e.g. a membrane spanning polypeptide such as CD122 or CD132 or a CAR) which, when the membrane spanning polypeptide is associated with a cell membrane, is which is embedded. in the cell membrane and is in peptidyl linkage with the extracellular domain (LCD) and the intracellular domain (ICD) of a membrane spanning polypeptide. A transmembrane domain may be homologous (naturally associated with) or heterologous (not naturally associated with) with either or both of the extracellular and/or intracellular domains. In some embodiments the transmembrane domain is the transmembrane domain natively associated with the ECD domain of the cognate receptor from which the orthogonal receptor is derived. In some embodiments the transmembrane domain is the transmembrane domain natively associated with the ICD domain of the cognate receptor from which the orthogonal receptor is derived. In some embodiments the transmembrane domain is the transmembrane domain natively associated with the proliferation signaling domain. In some embodiments the transmembrane domain is the transmembrane domain natively associated with a different protein. Alternatively, the transmembrane domain of the orthogonal receptor may be an artificial amino acid sequence which spans the plasma membrane. in some embodiments, the transmembrane domain of the orthogonal receptor is the transmembrane domain normally associated with the ICD of the cognate receptor from which the orthogonal receptor is derived. In some embodiments, where the receptor is chimeric receptor comprising the intracellular domain derived from a first parental receptor and a second extracellular domains are derived from a second different parental receptor, the transmembrane domain of the chimeric receptor is the transmembrane domain normally associated with either the ICD or the ECD of the parent receptor from which the chimeric receptor is derived.

Treat: The terms “treat”, “treating”, treatment” and the like refer to a course of action (such as administering IL2, a CAR-T cell, or a pharmaceutical composition comprising same) initiated with respect to a subject after a disease, disorder or condition, or a symptom thereof, has been diagnosed, observed, or the like in the subject so as to eliminate, reduce, suppress, mitigate, or ameliorate, either temporarily or permanently, at least one of the underlying causes of such disease, disorder, or condition afflicting a subject, or at least one of the symptoms associated with such disease, disorder, or condition. The treatment includes a course of action taken with respect to a subject suffering from a disease where the course of action results in the inhibition (e.g., arrests the development of the disease, disorder or condition or ameliorates one or more symptoms associated therewith) of the disease in the subject.

Treg Cell or Regulatory T Cell. The terms “regulatory T cell” or “Treg cell” as used herein refers to a type of CD4⁺ T cell that can suppress the responses of other T cells including but not limited to effector T cells (Teff). Treg cells are characterized by expression of CD4, the α-subunit of the IL2 receptor (CD25), and the transcription factor forkhead box P3 (FOXP3) (Sakaguchi, Annu Rev Immunol 22, 531-62 (2004). By “conventional CD4+ T cells” is meant CD4⁺ T cells other than regulatory T cells.

Variant: The terms “protein variant” or “variant protein” or “variant polypeptide” are used interchangeably herein to refer to a polypeptide that differs from a parent polypeptide by virtue of at least one amino acid modification. The parent polypeptide may be a naturally occurring or wild-type (WT) polypeptide or may be a modified version of a WT polypeptide. The term variant polypeptide may refer to the polypeptide itself, a composition comprising the polypeptide, or the nucleic acid sequence that encodes it. In some embodiments, the variant polypeptide comprises from about one to about ten amino acid modifications relative to the parent polypeptide, alternatively from about one to about five amino acid modifications compared to the parent, alternatively from about one to about three amino acid modifications compared to the parent, alternatively from one to two amino acid modifications compared to the parent, alternatively a single amino acid modification compared to the parent. A variant may be at least about 99% identical, alternatively at least about 98% identical, alternatively at least about 97% identical, alternatively at least about 95% identical, or alternatively at least about 90% identical to the parent polypeptide from which the variant is derived.

Wild Type: By “wild type” or “WT” or “native” herein is meant an amino acid sequence or a nucleotide sequence that is found in nature, including allelic variations. A WT protein, polypeptide, antibody, immunoglobulin, IgG, etc. has an amino acid sequence or a nucleotide sequence that has not been modified by the hand of man.

### Description of Certain Embodiments

### Adoptive Cell Therap(y)ies:

“Adoptive Cell Therapy” or simply “Cell Therapy” is used to refer to the administration of exogenously manipulated cells, particularly immune cells. One form of adoptive cell therapy employs exogenously manipulated lymphocytes isolated from tumor tissue (referred to as “tumor infiltrating lymphocytes” or “TILs”). It is believed that such TILs have been exposed to tumor antigens and are therefore capable of attacking the tumor cells but that such TILs are either in such short supply or are “exhausted” such that they are unable to independently eliminate the tumor. In TIL therapy, the isolated TILs cultured ex vivo to expand their numbers, exposed to activating agents and reinfused into the patient from whom the cells were isolated (referred as “autologous cell therapy”). TIL cell therapy is documented as a therapeutic modality having efficacy in the treatment of neoplastic disease in human subjects. See, e.g., Rosenberg (U.S. Pat. No. 5,126,132A issued Jun. 30, 1992 and Spiess, et al (1987) J Natl Cancer Inst 79:1067-1075.

In current practice, human TIL cell therapy consists of ex vivo expansion of TILs obtained from resected tumor material and adoptive transfer into the subject following a applying a lymphodepleting preparative regimen to the subject and subsequent support of the adoptively transferred cells by the administration of interleukin-2 (IL-2). The lymphodepleting preparative regimen depletes Tregs and removes cellular “sinks” and is often characterized as “making room” for the adoptively transferred cells. The systemic administration of IL-2 supports the persistence of the reinfused TILs in vivo. In typical clinical practice, shortly after infusion of the TILS, the patient receives i.v. high-dose IL-2 (720,000 IU/kg every 8 h until maximal tolerance. This subsequent support with IL-2 is thought to further enhance the survival and clinical efficacy of TILs.

Although having demonstrated clinical effect, TIL cell therapy is associated with significant toxicities arise primarily from the lymphodepleting preparative regimens resulting in pancytopenia and febrile neutropenia and the supportive therapy with high dose IL-2 following re-administration of the enriched TIL cell population. The effect of high dose IL2 typically used in supportive regimens of adoptive cell therapy is well documented to result in significant toxicities. The most prevalent side effects seen in arising from the use of IL-2 supportive therapy following adoptive cell transfer (ACT) include chills, high fever, hypotension, oliguria, and edema due to the systemic inflammatory and capillary leak syndrome as well as reports of autoimmune phenomena such as vitiligo or uveitis.

TIL cell therapy also has challenges arising from the ex vivo expansion of the isolated T cells. The ex vivo expansion of TILs is performed in the presence of high dose IL2 for a significant period of time. IL-2 promotes proliferation and expansion of activated T lymphocytes, potentiates B cell growth, and activates monocytes and natural killer cells. However, the during the TIL expansion process, there is an interclonal competition with different T-cell clones increasing or decreasing in frequency. As it is desirable that the final TIL product to be administered be as enriched as possible for the tumor-specific clones, the non-specific nature of hIL2 fails to provide selective support for the tumor-specific, antigen-experienced T cell clones and it is possible that the most efficacious tumor reactive T cell clones will be out-competed and diluted during the ex vivo expansion phase due to the non-specific T-cell proliferative effects of hIL2. As a result, the TIL cell product to be reinfused to the subject may have wherein the population of anti-tumor TILs (tumor antigen experienced cells) represents a suboptimal fraction (e.g. less than 60%, alternatively less than 50%, alternatively less than 40%, alternatively less than 30%) of the total number of cells in the cell product that is reinfused to the subject. Additionally, the degree of T-cell differentiation of the T cells following ex vivo stimulation procedures can affect the survival, proliferative capacity and efficacy of the TILs in vivo following reinfusion. Li, et al. (2010) J Immunol. 2010; 184: 452-465. The high potency of IL2 is and the effects of the exposure of culture TILs to high dose IL2 has been associated with terminal differentiation of the T cells cultured in its presence ex vivo as well as mediation of autoimmunity and transplant rejection in addition to other side effects in vivo.

As Li, et al. state:
A key question emerging from our studies is whether using other methods of performing the REP can yield post-REP T cells with a “younger” phenotype associated with maintenance of CD28 expression and other effector-memory markers that are capable of better persistence in vivo during ACT. In other words, can we have the best of both worlds by generating high numbers of tumor-reactive cytotoxic T cells while maintaining a memory phenotype favorable for continued cell division and long-term survival in vivo?
   Li, et al at page 465. Furthermore, Li, et al suggest the state of terminal differentiation of the TILs resulting from with current ex vivo protocols involving IL2 is an issue for current TIL therapy and suggest that the potential use of other cytokines such as IL-15 or IL-21 to avoid the effects of IL-2 in the ex vivo preparation of TILs. Although, support of TILs with high dose IL2 therapy following ACT is associated with improved therapeutic outcome. However, high dose IL2 therapy is associated with significant toxicity in human subjects and, as previously noted, is one of the major challenges facing TIL therapy. Consequently, desirable to provide a method of supporting the viability and/or proliferation of tumor antigen-experienced T cells ex vivo without driving the desired population of these tumor antigen experienced T cells toward differentiation and/or exhaustion.

### Engineered Cell Therapies

In addition to TIL cell therapy, and in part inspired by its demonstration that the human immune system is capable of eliminating tumors, there have been a wide variety of approaches have been investigated for engineering immune cells to have particularly desirable properties. One such category of engineered immune cells which has demonstrated clinical and is approved for human use is the use of immune cells that have been engineered to express a chimeric antigen receptor (CAR), CAR T cell therapy in early clinical trials involving patients with pre-B cell acute lymphoblastic leukaemia (ALL) or B cell lymphomas was revolutionary and suggested the possibility of curative option for patients who are refractory to standard treatments. These early trials resulted in rapid FDA approvals of anti-CD19 CART cell products for both acute lymphocytic leukemia (ALL) and certain types of B cell lymphoma. The initial clinical responses in the treatment of hematological malignancies disease with CAR T therapies has been very promising with reported initial response rates of 90% or more in human subjects has stimulated significant research into the development of CAR-T and wide variety of CAR-T approaches are in various stages of preclinical and clinical development for the treatment of a wide variety of neoplastic diseases.

The primary targeting and activation component of the CAR-T cells is the CAR, typically a multifunctional polyprotein comprising an tumor antigen specific targeting domain and additional structural (e.g. hinge, transmembrane) and intracellular signaling domains. A wide variety of CARs designs have been proposed in the literature and are frequently categorized first, second, third or fourth generation CARs based primarily on the architecture of the signaling domains. The term first-generation CAR refers to a CAR wherein the intracellular domain transmits the signal from antigen binding through only a single signaling domain, for example a signaling domain derived from the high-affinity receptor for IgE FcεR1g or the CD3ζ chain. The intracellular signaling domain contains one or three immunoreceptor tyrosine-based activating motif(s) [ITAM(s)] for antigen-dependent T-cell activation. The ITAM-based activating signal endows T-cells with the ability to lyse the target tumor cells and secret cytokines in response to antigen binding. Second-generation CARs include a co-stimulatory signal in addition to the CD3 ζ signal. Coincidental delivery of the delivered co-stimulatory signal enhances cytokine secretion and antitumor activity induced by CAR-transduced T-cells. The co-stimulatory domain is usually be membrane proximal relative to the CD3 domain. Third-generation CARs include a tripartite signaling domain, comprising for example a CD28, CD3ζ, OX40 or 4-1BB signaling region. In fourth generation, or “armored car” CAR T-cells are further modified to express or block molecules and/or receptors to enhance immune activity such as the expression of IL-12, IL-18, IL-7, and/or IL-10; 4-1BB ligand, CD-40 ligand.

While the intracellular signaling domains are important to the activation and proliferation of the engineered CAR-T cells, it is the extracellular targeting domain (or ABD) that defines the target of the CAR-T and its corresponding is clinical application. The extracellular targeting antigen binding domain (ABD) typically comprises an antibody or antibody fragment (e.g., scFv or VHH) that specific binds to a cell surface antigen (either peptide or glycan) characteristic of a neoplastic cell to provide selective targeting of the CAR-T cell. In order to minimize potential side effects and toxicity of the CAR-T cell including autoimmune reactions, it is preferable when selecting a tumor antigen for targeting that the antigen be significantly more prevalent on tumor cell types than normal cells of the subject to be treated. Examples of such tumor antigens for which antibody binding molecules have been identified and their clinical therapeutic targets include but are not limited CD19 (e.g., hematological malignancies e.g., ALLs, CLLs, B cell lymphomas), CD20 (e.g., refractory or relapsed CD20⁺ B-cell lymphoma), BCMA (e.g., multiple myeloma, Carpenter, et al. (2013) Clin Cancer Res; 19(8); 2048-60), CD22 (B-cell malignancies including pediatric B cell precursor ALL as described in Pan, et al (2019) Leukemia 33, 2854-2866), CD30 (e.g., CD30+ lymphomas including Hodgkin lymphoma; Grover, (2019) BMC Cancer 19, 203), CD70 (e.g., acute myeloid leukemia (AML; Sauer, et al (2019) Blood 134 (Supplement_1): 1932), Lewis Y (e.g., AML; Ritchie, et al (2013) Molecular Therapy 21(11):2122-9), GD2 (e.g., gliomas; Mount, et. al (2018) Nat Med 24, 572-579), GD3 (e.g., metastatic melanoma and neuroecodermal tumors; Agnes, et al. (2010) DOI: 10.1158/1078-0432.CCR-10-0043, mesothelin (e.g., mesothelioma, lung, pancreas, breast, ovarian and other solid tumors; Beatty, et. Al., (2014) Cancer Immunol Research 2(2)), ROR-1 (e.g., chronic lymphocytic leukemia; Aghebati-Maleki, et al (2017) Biomedicine and Pharmacology 88: 814-822), CD44 (e.g., AML and multiple myeloma; Casuccia, et al (2013) Blood 122 (20): 3461-3472), CD171 (e.g., neuroblastoma; Kunkele, et al (2017) Clin Cancer Research 23(2):466-477); EGP2, EphA2 (e.g., glioblastoma; Yi, et al (2018) Molecular Therapy: Methods & Clinical Development 9:70-80), ErbB2, ErbB3/4, FAP, FAR IL11Ra, PSCA (prostate cancer), PSMA (prostate cancer), NCAM, HER2, NY-ESO-1, MUC1, CD123, FLT3, B7-H3, CD33, IL1RAP, CLL1 (CLEC12A)PSA, CEA, VEGF, VEGF-R2, c-Met, Glycolipid F77, FAP, EGFRvIII, MAGE A3, 5T4, WT1, KG2D ligand, a folate receptor (FRa), and Wnt1 antigens. CD123. Additionally, the ABD may be multivalent in that it has the capacity to bind to more than one antigen, especially more than may have specificity for more than one tumor antigen (e.g. CD19 and CD20 as described in Zah, et al (2016) Cancer Immunol Res; 4(6); 498-508; CD19 and CD22 as described in Tu, et al (2019) Frontiers in Oncology 9:1350).

One particular example of such an antigen the 95 kDa glycoprotein CD19. CD19 is expressed by most B cell lymphonas, acute lymphocytic leukemias (ALLs), chronic lymphocytic leukemias (CLLs), hairy cell leukemias and some acute myelogenous leukemias (AMLs) but is CD19 is not present on most normal tissues, other than normal B cells. Although there are multiple CAR-T product candidates in various stages of clinical development the anti-CD19 CAR cell products axicabtagene ciloleucel (marketed as Yescarta® commercially available from Gilead Pharmaceuticals) and tisagenlecleucel (marketed as Kymriah® commercially available from Novartis) are currently the only CAR-T cell therapies approved by major regulatory agencies for use in human beings, a wide variety of CAR-T therapies are in various stages of preclinical and clinical development for the treatment of a wide variety of neoplastic diseases.

The initial clinical responses in the treatment of neoplastic disease with CAR T therapies has been very promising with reported initial response rates of 90% or more in human subjects, the growing body of literature relating to the clinical experience with the approved anti-CD19 CAR agents has revealed that a substantial fraction of the subjects treated with such agents suffer recurrence of the disease state. The lack of durable response in such patients is attributed to primarily to poor CAR-T cell persistence following administration of the CAR-T cell product and/or cancer cell resistance resulting from antigen loss or modulation. The administration of IL2 at doses that can be tolerated by the patient fail to provide long term selective maintenance of an activated population of the adoptively transferred cells leading to relapse and recurrence of the neoplastic disease.

The present disclosure provides methods and compositions that overcome these issues and opens up new opportunities for the use of adoptive engineered cell therapies including CART therapies, particularly in the treatment of solid tumors where persistence of the engineered cells is of particular note.

It is well established that adoptively transferred human immune cells lose their activity relatively rapidly following administration. Consequently, the typical means to address this rapid loss of function are: (a) administration excessively high doses of the cell therapy agent to maximize the exposure of the cell therapy agent to the tumor before the cells lose effectiveness, and/or (b) systemic administration of HD-hIL2 therapy to attempt to support the efficacy of the adoptively transferred cell. Both of these approaches present significant toxicity. The toxicities associate with HD-hIL2 therapy have already been discussed above. High doses of engineered cell therapy agents are associated with life threating cytokine release syndrome (CRS). Currently available products have shown CRS of all grades in the majority of subjects treated and Grade 3 or greater CRS in a significant fraction of patients. Significant neurotoxicity is also observed in a majority of patients. However, lower doses of the cell therapy agents have been associated with a significant decrease in clinical outcome. Additionally, due primarily to lack of persistence of the cell therapy product, many patients who at first appear be responding well to the cell therapy relapse. Currently, it is reported that approximately 60% of patients treated with existing CD-19 cell therapy agents relapse. Byrne M, et al (2019) Biology of Blood and Marrow Transplantation 25(11):344-251.

As the results of the experimentation described in more detail below demonstrates, the administration of an orthogonal cell in combination with an orthogonal ligand address many of the issues of current cell therapies and provide improved methods of treatment of diseases amenable to cell therapy including but not limited to:
compositions and methods that enable the selective expansion a population of adoptively transferred human immune cells in vivo without significant off-target systemic activation of other immune cells;
compositions and methods that support the persistence of activated orthogonal cell adoptively transferred human immune cells the without significant toxicity associated with the supportive agent;
compositions and methods that achieve in vivo therapeutic effectiveness of a cell therapy product in the treatment of neoplastic disease in a mammalian subject using an initial dose of the cell therapy agent at doses that have previously been reported as non-efficacious and significantly (10-1000 fold) below current dosages of similar cell therapy products;
compositions and methods that enable the maintenance of a therapeutic level of an orthogonal immune cell at a therapeutically effective level for extended periods of time by periodic administration of an orthogonal ligand;
compositions and methods that enable the treatment of relapse of a neoplastic condition by the administration of an orthogonal ligand to revive the effectiveness of the of previously administered orthogonal cells without the need to administer additional engineered orthogonal cells;
compositions and methods that provide selective modulation of the activity and proliferation of orthogonal cells enabling the temporary or permanent cessation of exposure of the subject to the activated form of the orthogonal cell therapy by removal of the activating orthogonal ligand and without the need to further engineer the cell to include a “kill switch” or employ immunodepletive or immunosuppressive treatment regimens; a cell for response to a without hancing the proliferation of adoptively transferred cells in a mammalian subject following administration of the cell product without significant toxicity;
compositions and methods that avoid the need for prior immunodepletion of the the subject prior to adoptive cell therapy;
pharmaceutical formulations of orthogonal ligands, particularly orthogonal ligands having extended duration of action enabling less frequent dosing of the supportive orthogonal ligand;
compositions and methods that provide demonstrable therapeutic anti-tumor efficacy superior to existing cell therapy agents for similar indiations; and
compositions and methods that when combined with supplementary therapeutic agents provide enhanced anti-tumor efficacy.

A series of experiments were performed to demonstrate the utility and functionality of the compositions and methods of the present disclosure, in particular in the treatment of a mammalian subject suffering from a neoplastic disease condition in a mammalian subject suffer orthogonal system in the context of adoptive cell therapy. The utility of the compositions and methods of the disclosure were evaluated in a series of experiments as more fully detailed in the attached examples and the data provided in the attached figures.

Briefly, a series of in vitro and in vivo experiments were conducted using a representative human IL2 ortholog of Formula 1, an hIL2 variant containing the set of amino acid substitutions: [desAla1-E15S-H16Q-L19V-D20L-Q22K-C125A], numbered in accordance with wt hIL2 and having the amino acid sequence:

(SEQ ID NO: 23)
PTSSSTKKTQLQLSQLLVLLKAILNGINNYKNPKLTR
MLTFKFYMPKKATELKHLQCLEEELKPLEEVLNLAQS
KNFHLRPRDLISNINVIVLELKGSETTFMCEYADETA
TIVEFLNRWITFCQSIISTLT

also sometimes referred to herein as “orthoIL2” and “hoIL2”.

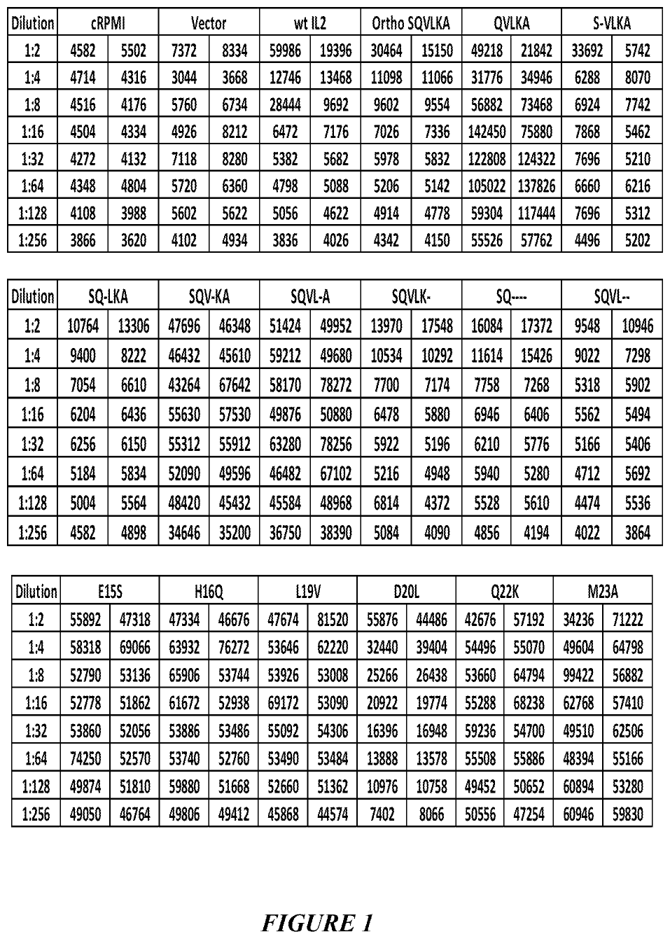

To confirm the selective binding properties of STK-007, a Biacore surface plasmon resonance study was performed to confirm that STK-007 retains substantial binding to the CD25 (IL2Rα) and CD132 (IL2Rg) components of the IL2 receptor and exhibits specific binding to hoRb while wt hIL2 does not significantly bind to hoRb. Briefly, C-terminal HIS-tagged versions of wtCD25, wtCD122, hoCD122 and wtCD132 were prepared and immobilized on an anti-HIS capture chip and evaluated for binding using a Biacore by flowing a solution of the STK-007 and wt hIL2 molecule (Shenandoah Biotechnology, Inc.) over the immobilized receptor subunits and evaluated for binding. The results of this experiment are shown in FIG. 1 of the attached drawings. Panels A, C, E, and G represent the binding of STK-007 to wtCD25, wtCD122, wtCD132 and hoCD122 respectively. Panels B, D, F, and H represent the binding of wtIL2 to wtCD25, wtCD122, wtCD132 and hoCD122 respectively. The data presented indicate that STK-007 retains binding to wtCD25 and wtCD132 similar to wt hIL2 but very low affinity for wt CD122. However, STK-007 does bind with affinity to hoCD122 (Panel G) similar to the binding of wtIL2 to wtCD122. Similarly, wt hIL2 demonstrates binding to wtCD25, wt CD122 and wtCD132 similar to very low affinity for hoCD122.

For in vivo studies, the STK-007 molecule was modified by the addition of an N-terminal 40 kDa branched PEG molecule of the structure:

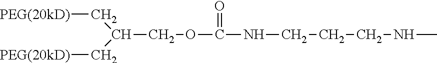

which resulted in the hIL2 ortholog of the Formula 2 referred to hereinafter as STK-009 having the structure of

40 kD-PEG-linker-desAla1-hIL2[E15S-H16Q-L19V-D20L-Q22K-M23A]-COOH. [2] (also sometimes referred to herein “PEGortho,” “PEGorthoIL2” or “PEGhoIL2.” A representative orthogonal cell used for these studies was a CD19 orthogonal CAR-T cell modified to express an orthogonal CD122 (IL2Rb) receptor with the amino acid sequence:

(SEQ ID NO: 29)
AVNGTSQFTCFYNSRANISCVWSQDGALQDTSCQVHAWPDRRRWNQ
TCELLPVSQASWACNLILGAPDSQKLTTVDIVTLRVLCREGVRWRV
MAIQDFKPFENLRLMAPISLQVVHVETHRCNISWEISQASDFFERH
LEFEARTLSPGHTWEEAPLLTLKQKQEWICLETLTPDTQYEFQVRV
KPLQGEFTTWSPWSQPLAFRTKPAALGKDTIPWLGHLLVGLSGAFG
FIILVYLLINCRNTGPWLKKVLKCNTPDPSKFFSQLSSEHGGDVQK
WLSSPFPSSSFSPGGLAPEISPLEVLERDKVTQLLLQQDKVPEPAS
LSSNHSLTSCFTNQGYFFFHLPDALEIEACQVYFTYDPYSEEDPDE
GVAGAPTGSSPQPLQPLSGEDDAYCTFPSRDDLLLFSPSLLGGPSP
PSTAPGGSGAGEERMPPSLQERVPRDWDPQPLGPPTPGVPDLVDFQ
PPPELVLREAGEEVPDAGPREGVSFPWSRPPGQGEFRALNARLPLN
TDAYLSLQELQGQDPTHLV

also sometimes referred to herein as “hoCD122” or “hoRb” (human ortho receptor beta). The extracellular domain of the hoRb receptor comprises two amino acid substitutions H133D and Y134F (numbered in accordance with wt hCD122).

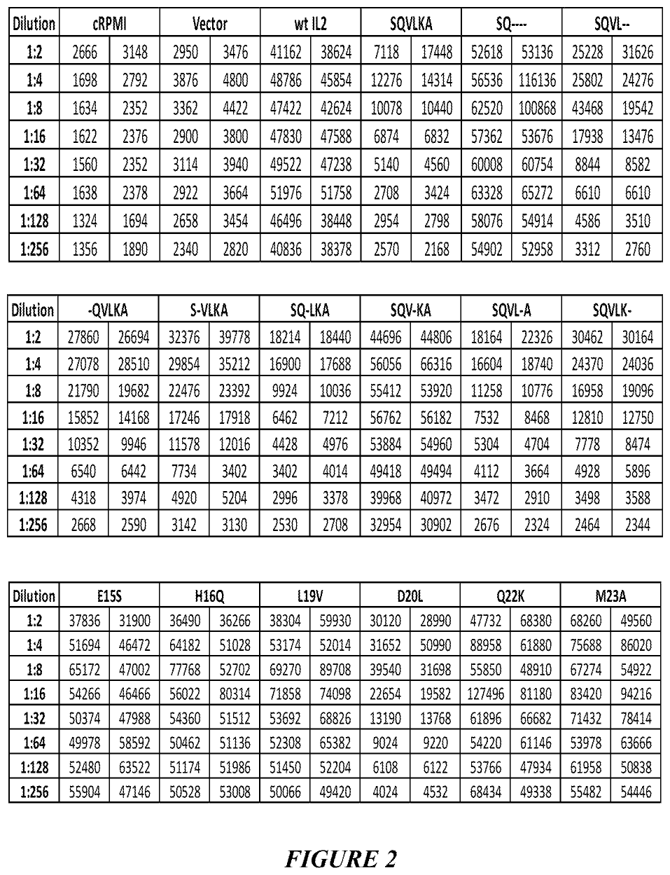

The exemplary CAR used in these studies is an anti-CD19 chimeric antigen receptor comprising the FMC63 anti-CD19 scFv as the antigen binding domain, the CD28 transmembrane and co-stimulatory domain and the CD3z domain (FIG. 2, Panel A) and having the amino acid sequence:

(SEQ ID NO: 30)
MLLLVTSLLLCELPHPAFLLIPDIQMTQTTSSLSASLGDRVTISCRA
SQDISKYLNWYQQKPDGTVKLLIYHTSRLHSGVPSRFSGSGSGTDYS
LTISNLEQEDIATYFCQQGNTLPYTFGGGTKLEITGSTSGSGKPGSG
EGSTKGEVKLQESGPGLVAPSQSLSVTCTVSGVSLPDYGVSWIRQPP
RKGLEWLGVIWGSETTYYNSALKSRLTIIKDNSKSQVFLKMNSLQTD
DTAIYYCAKHYYYGGSYAMDYWGQGTSVTVSSAAAIEVMYPPPYLDN
EKSNGTIIHVKGKHLCPSPLFPGPSKPFWVLVVVGGVLACYSLLVTV
AFIIFWVRSKRSRLLHSDYMNMTPRRPGPTRKHYQPYAPPRDFAAYR
SRVKFSRSADAPAYQQGQNQLYNELNLGRREEYDVLDKRRGRDPEMG
GKPRRKNPQEGLYNELQKDKMAEAYSEIGMKGERRRGKGHDGLYQGL
STATKDTYDALHMQALPPR

A nucleic acid sequence was synthesized encoding: the CD19_28 z CAR, a T2A peptide and the hoRb sequence

(SEQ ID NO: 31)
MLLLVTSLLLCELPHPAFLLIPDIQMTQTTSSLSASLGDRVTISCRA
SQDISKYLNWYQQKPDGTVKLLIYHTSRLHSGVPSRFSGSGSGTDYS
LTISNLEQEDIATYFCQQGNTLPYTFGGGTKLEITGSTSGSGKPGSG
EGSTKGEVKLQESGPGLVAPSQSLSVTCTVSGVSLPDYGVSWIRQPP
RKGLEWLGVIWGSETTYYNSALKSRLTIIKDNSKSQVFLKMNSLQTD
DTAIYYCAKHYYYGGSYAMDYWGQGTSVTVSSAAAIEVMYPPPYLDN
EKSNGTIIHVKGKHLCPSPLFPGPSKPFWVLVVVGGVLACYSLLVTV
AFIIFWVRSKRSRLLHSDYMNMTPRRPGPTRKHYQPYAPPRDFAAYR
SRVKFSRSADAPAYQQGQNQLYNELNLGRREEYDVLDKRRGRDPEMG
GKPRRKNPQEGLYNELQKDKMAEAYSEIGMKGERRRGKGHDGLYQGL
STATKDTYDALHMQALPPGSGEGRGSLLTCGDVEENPGPMAAPALSW
RLPLLILLLPLATSWASAAVNGTSQFTCFYNSRANISCVWSQDGALQ
DTSCQVHAWPDRRRWNQTCELLPVSQASWACNLILGAPDSQKLTTVD
IVTLRVLCREGVRWRVMAIQDFKPFENLRLMAPISLQVVHVETHRCN
ISWEISQASDFFERHLEFEARTLSPGHTWEEAPLLTLKQKQEWICLE
TLTPDTQYEFQVRVKPLQGEFTTWSPWSQPLAFRTKPAALGKDTIPW
LGHLLVGLSGAFGFIILVYLLINCRNTGPWLKKVLKCNTPDPSKFFS
QLSSEHGGDVQKWLSSPFPSSSFSPGGLAPEISPLEVLERDKVTQLL
LQQDKVPEPASLSSNHSLTSCFTNQGYFFFHLPDALEIEACQVYFTY
DPYSEEDPDEGVAGAPTGSSPQPLQPLSGEDDAYCTFPSRDDLLLFS
PSLLGGPSPPSTAPGGSGAGEERMPPSLQERVPRDWDPQPLGPPTP
GVPDLVDFQPPPELVLREAGEEVPDAGPREGVSFPWSRPPGQGEFR
ALNARLPLNTDAYLSLQELQGQDPTHLV

as illustrated in FIG. 1A. These constructs were synthesized and cloned downstream of the EF1a-promoter of a 3^{rd} generation p14Syn lentiviral backbone (Alstem Bio). Isolated and stimulated to generate “CD19_28 z orthoCAR” T cells also referred to SYNCAR-001.

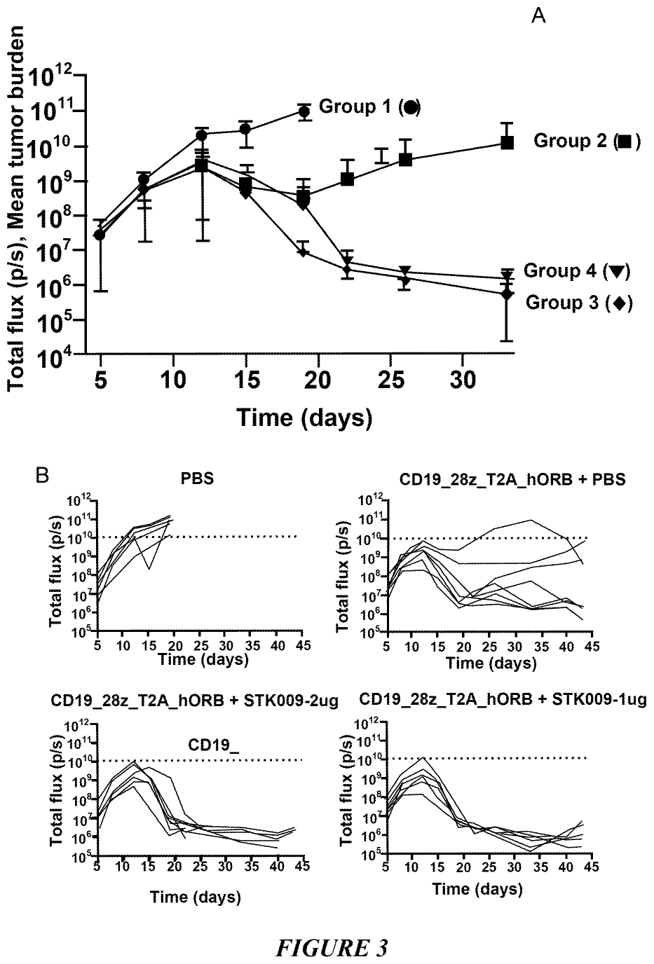

In the disseminated model as demonstrated by the data provided in FIG. 3, the administration of PBS failed to control the tumor burden FIG. 3, Panel A, Group 1 and FIG. 3, Panel B, upper left) resulting the animals needing to be sacrificed due to toxicity on approximately day 21 of the study. In contrast, the administration of CD19_28 z orthoCAR T cells led to an antitumor response in 4/8 mice (FIG. 3, Panel A, Group 2 and FIG. 3, Panel B, upper right). The combined treatment of both CD19_28z orthoCAR T cells with STK-009 at both dose levels (FIG. 3, Panel A, 1 μg (Group 3, and FIG. 3, Panel B lower right) and 2 μg (Group 4 and FIG. 3, Panel B lower left) provided additional anti-tumor function compared to CD19_28z orthoCAR T cell treatment alone.

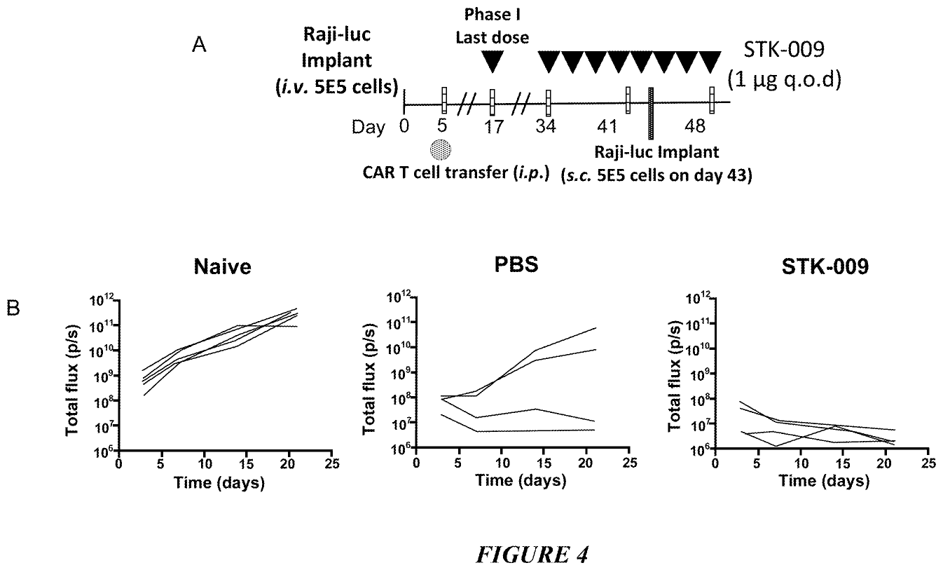

Additionally, the data provided in FIG. 4 resulting from the rechallenge model described in Example 8 demonstrates that These data demonstrate that STK009 redosing is capable of restoring the anti-tumor activity of CAR T cells even a prolonged period of no antigen or tumor ligand exposure.

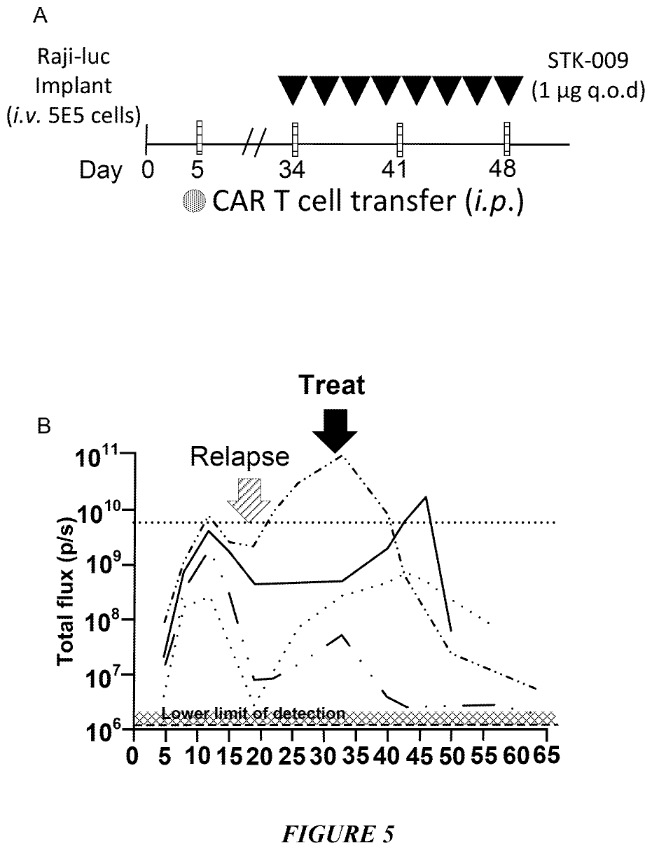

The relapse model described in Example 9 with data provided in FIG. 5 demonstrates that the administration of STK-009 alone is capable of effectuating anti-tumor activity of CAR-T cells in animal that have relapsed from a prior course of therapy.

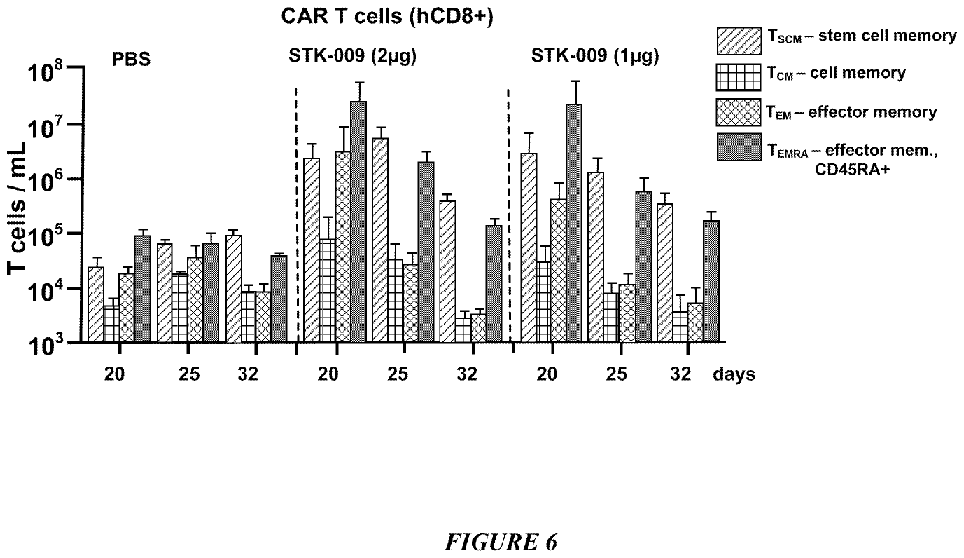

Additionally as discussed in Example 11 and provided FIG. 6, the orthogonal CAR-T cells contacted with the orthogonal ligand (STK-009) retained the SCM phenotype in vivo demonstrating an enhanced persistence of the CAR T cells providing a more durable antitumor effect.

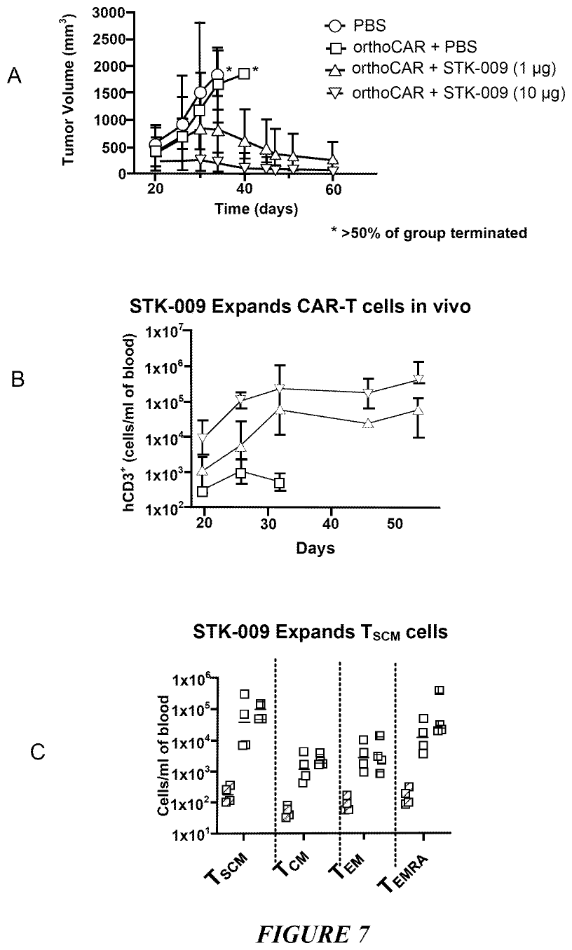

It is particularly noteworthy that the data obtained from the solid tumor model in the solid tumor model discussed in Example 12 with the data provided in FIG. 7-10. This data demonstrates that the orthogonal CAR-T cell in combination with an orthogonal cognate ligand is capable of inducing a response in solid tumors and increasing the infiltration of the CAR-T cells into the solid tumor demonstrating that this system is useful in the treatment of solid tumors not previously treatable using adoptive cell transfer protocols such as CARTs.

The data demonstrate the improved persistence of the therapeutic engineered cells that express the orthogonal receptor (e.g. hoCART, hoTIL), the ability to selectively and potently activate in a dose dependent manner, the engineered orthogonal immune cells in response to the contacting with a cognate orthogonal ligand, the specificity of the ligand for the cells that express the orthogonal ligand demonstrating a significant reduction in toxicity typically associated with the does not provide non-specific off-target toxicity such as that observed with the administration of non specific T cell proliferative agents such as hIL2 which is a well-documented source of toxicity in adoptive cell therapy protocols.

The selectivity of the orthogonal ligand for the orthogonal receptor results in a molecule with low toxicity in vivo. This was demonstrated in a non-human primate model toxicology study where the primates were exposed to high doses of a long acting PEGylated orthogonal hIL2 molecule as described herein. Despite the persistence of the orthogonal IL2 compound at high doses as observed in this study, there were no significant toxicities observed at a dose significantly greater (likely at least 10-fold to 100-fold greater) that a therapeutically effective dose. The selectivity and efficacy of the orthogonal ligand to selectively activate the therapeutic cells enables the use of adoptive cell therapy on a preventative basis for the prevention of progression of disease such as the treatment of slowly progressing pre-cancerous conditions such as smoldering multiple myeloma in conjunction with a BCMA CAR T cell.

The orthogonal ligands were successful in re-enervating the CAR T cells enabling the ability to reestablish the therapeutic effect of the engineered cell without readmission of the cell demonstrating the utility of the technology in preventing relapse, recurrence and metastasis of neoplastic disease.

In some embodiments, the present disclosure provides a method of treating a disease disorder or condition in a human subject comprising the steps of: (a) administering to a subject a therapeutically effective amount of an orthogonal ligand; (b) administering to a subject a mammalian immune cell comprising a nucleic acid sequence encoding an orthogonal hCD122 receptor operably linked to one or more expression control elements such that the mammalian immune cell expresses the orthogonal hCD122 receptor.

The present disclosure provides methods and compositions for treating a subject suffering from a neoplastic disease by the administration of a plurality of engineered T cells expressing an orthogonal CD122 receptor and a chimeric antigen receptor the extracellular domain of which specifically binds a tumor antigen and the contemporaneous administration of orthogonal IL2 ligand the prevention of relapse of said neoplastic disease by the administration to said subject of a maintenance therapy comprising the periodic administration of an orthogonal IL2 ligand of Formula 1, wherein the orthogonal ligand used in the treatment phase is the same or different than the orthogonal ligand used in the maintenance phase. In some embodiments, the orthogonal ligand is modified to extend half-life. In one embodiment the orthogonal ligand is pegylated, fusion protein, etc. In one embodiment the orthogonal ligand is comprises a 40 kD N terminally PEG moiety. In some embodiments of the method, the orthogonal ligand is dosed in a first therapeutic phase ligand above the concentration sufficient to provide expansion (e.g >EC₁₀^{PRO} but below the concentration sufficient to induce significant differentiation (e.g. <EC₉₀^{ACT}) for a period of at least 24 hours, optionally for period of at 30, 60, 90 or longer days. In some embodiments, the maintenance phase optionally including the administration of an orthogonal ligand of Formula 1 sufficient to induce ortho CART activation maintaining a serum level in excess of the concentration for activation (e.g. >EC₅₀^{ACT}) for a period of at least 24 hours. In some embodiments of the method the orthogonal ligand is provided to the subject by the administration of a recombinant viral or non viral vector comprising the nucleic acid of encoding an orthogonal IL2 ligand of Formula 1. In some embodiments of the method the neoplastic disease is selected from solid tumors and hematologic malignancies. In some embodiments of the method. In some embodiments of the method the method further comprising one or more supplementary anti-neoplastic agents during the treatment phase and/or maintenance phase. In some embodiments of the method supplementary anti-neoplastic agents during the treatment phase and/or maintenance phase can be same or different. In some embodiments of the method, the supplementary neoplastic agent is selected from the group consisting of chemotherapeutic agents, small molecules, supplementary biologics including but not limited to checkpoint inhibitors (anti-PD1, Keytruda, Opdivo), anti-tumor antigen antibodies (Herceptin), and/or physical methods (surgery, radiation, etc). In some embodiments, the orthogonal receptor is an orthogonal CD122 comprising one or more STAT3 binding motifs.

### Prevention of Metastasis

The present disclosure provides methods and compositions for treating a subject suffering from a neoplastic disease by the administration of a plurality of engineered T cells expressing an orthogonal CD122 receptor and a chimeric antigen receptor the extracellular domain of which specifically binds a tumor antigen and the contemporaneous combination administration of orthogonal IL2 ligand Formula 1 and the prevention of metastasis of said neoplastic disease by the administration to said subject of a maintenance therapy comprising the periodic administration of an orthogonal IL2 ligand of Formula 1 wherein the orthogonal ligand used in the treatment phase is the same or different than the orthogonal ligand used in the maintenance phase. In some embodiments, the orthogonal ligand is modified to extend half-life. In one embodiment the orthogonal ligand is pegylated, fusion protein, etc. In one embodiment the orthogonal ligand is comprises a 40 kD N terminally PEG moiety. In some embodiments of the method, the orthogonal ligand is dosed in a first therapeutic phase ligand above the concentration sufficient to provide expansion (e.g >EC₁₀^{PRO} but below the concentration sufficient to induce significant differentiation (e.g. <EC₉₀^{ACT}) for a period of at least 24 hours, optionally for period of at 30, 60, 90 or longer days. In some embodiments, the maintenance phase optionally including the administration of an orthogonal ligand of Formula 1 sufficient to induce ortho CAR T activation maintaining a serum level in excess of the concentration for activation (e.g. >EC₅₀^{ACT}) for a period of at least 24 hours. In some embodiments of the method the orthogonal ligand is provided to the subject by the administration of a recombinant viral or non-viral vector comprising the nucleic acid of encoding an orthogonal IL2 ligand of Formula 1. In some embodiments of the method the neoplastic disease is selected from solid tumors and hematologic malignancies. In some embodiments of the method. In some embodiments of the method the method further comprising one or more supplementary anti-neoplastic agents during the treatment phase and/or maintenance phase. In some embodiments of the method supplementary anti-neoplastic agents during the treatment phase and/or maintenance phase can be same or different. In some embodiments of the method, the supplementary neoplastic agent is selected from the group consisting of chemotherapeutic agents, small molecules, supplementary biologics including but not limited to checkpoint inhibitors (anti-PD1, Keytruda, Opdivo), anti-tumor antigen antibodies (Herceptin), and/or physical methods (surgery, radiation, etc). In some embodiments, the orthogonal receptor is an orthogonal CD122 comprising one or more STAT3 binding motifs.

### CD19 CAR For Hematologic Malignancies:

In one embodiment, the present disclosure provides a method of treating or preventing hematological malignancies in a subject in need of treatment or prevention by the administration of therapeutically effective amount of an orthogonal CD19 CAR in combination with the administration of a therapeutically effective amount of an orthogonal IL2 ligand of Formula 1.

In one embodiment, the present disclosure provides an orthogonal CD19 CAR for use in a method of the prevention of relapse and/or metastasis in a subject suffering from a hematological neoplastic disease comprising the step of administration of a plurality of engineered T cells expressing an orthogonal CD122 polypeptide and a chimeric antigen receptor the extracellular domain of which specifically binds to CD19 in combination with administration of orthogonal IL2 ligand of Formula 1. In some embodiments, the CARs is SYNCAR-001 as described herein.

In some embodiments, the present invention provides a method of preventing relapse in a subject previously treated with an orthogonal CD19 CART cell in combination with a orthogonal IL2 ligand following partial response or complete response to the initial orthogonal CD19 CAR-T/orthogonal IL2 ligand treatment phase, the method comprising the administration of a course of a maintenance therapy comprising the periodic administration of an orthogonal IL2 ligand of Formula 1 at a concentration lower than that administered during the treatment phase. The orthogonal ligand administered during the treatment phase may be the same or different than the orthogonal ligand administered during the maintenance phase.

In some embodiments, the hematological malignancy is a relapsed or refractory hematological malignancy, including but not limited to relapsed or refractory non-Hodgkins's lymphoma, relapsed or refractory myeloma, relapsed or refractory large B cell lymphoma, relapsed or refractory mantle cell lymphoma. Relapsed hematological malignancies (e.g., relapsed myeloma) involve the situation where the patient had an initially successful course of therapy but the disease reappears. A refractory hematological malignancy refers to a disease that progresses despite active treatment. Patients suffering from refractory myeloma are referred to has having primary refractory myeloma if the disease has not demonstrated a response and continue to progress on chemotherapy and secondary refractory patients who had an initial response at the initiation of treatment but the treatment is no longer having an effect.

In some embodiments, the orthogonal ligand is modified to extend half-life. In one embodiment the orthogonal ligand is pegylated, an Fc fusion protein, albumin fusion and the like. In one embodiment the orthogonal ligand is comprises a 40 kD N terminally PEG moiety.

In some embodiments of the method, the orthogonal ligand is dosed in a first therapeutic phase ligand above the concentration sufficient to provide expansion (e.g)>EC₁₀^{PRO} but below the concentration sufficient to induce significant differentiation (e.g. <EC₉₀^{ACT}) for a period of at least 24 hours, alternatively for a period of one week, alternatively for a period of two weeks, alternatively for a period of a period of at least 30 days, alternatively for a period of at least 60 days, or alternatively for a period of at least 90 or longer days. In some embodiments, the maintenance phase optionally including the administration of an orthogonal ligand of Formula 1 sufficient to induce ortho CAR T activation maintaining a serum level in excess of the concentration for activation (e.g. >EC₅₀^{ACT}) for a period of at least 24 hours.

In some embodiments of the method the orthogonal ligand is provided to the subject by the administration of a recombinant viral or non-viral vector comprising the nucleic acid of encoding an orthogonal IL2 ligand of Formula 1. In some embodiments of the method the neoplastic disease is selected from solid tumors and hematologic malignancies.

In some embodiments of the method the method further comprising one or more supplementary anti-neoplastic agents during the treatment phase and/or maintenance phase. In some embodiments of the method the anti-neoplastic agent(s) during the treatment phase and/or maintenance phase can be same or different. In some embodiments of the method, the supplementary neoplastic agent is selected from the group consisting of chemotherapeutic agents, small molecules, supplementary biologics including but not limited to checkpoint inhibitors (anti-PD1, Keytruda, Opdivo), anti-tumor antigen antibodies (Herceptin), and/or physical methods (surgery, radiation, etc). In some embodiments, the orthogonal receptor is an orthogonal CD122 comprising one or more STAT3 binding motifs. In some embodiments, the extracellular domain of the CAR comprises an antibody that specifically binds to CD19 comprising the CDRs or one or more of antibodies selected from the group consisting of FMC63.

In some embodiments the hematologic neoplastic disease is selected from acute lymphoblastic leukemia (ALL) including Philadelphia Chromosome Positive ALL, chronic lymphocytic leukemia (CLL), and B-cell lymphomas. In some embodiments the method further comprises co-administration one or more chemotherapeutic agents.

When the hematologic malignancy is ALL, the supplementary agent may be vincristine or liposomal vincristine (Marqibo), daunorubicin (daunomycin) or doxorubicin (Adriamycin), cytarabine (cytosine arabinoside, ara-C), L-asparaginase or PEG-L-asparaginase (pegaspargase or Oncaspar), 6-mercaptopurine (6-MP), methotrexate, cyclophosphamide, prednisone, dexamethasone, delarabine (Arranon). When the hematologic malignancy is Philadelphia Chromosome Positive ALL, the supplementary agent may be Imatinib (Gleevec®), dasatinib (Sprycel®) nilotinib (Tasigna®), ponatinib (Iclusig®), and bosutinib (Bosulif®).

When the hematologic malignancy is CLL, the supplementary agent may be fludarabine-containing regimens including but not limited to “FCR” (fludarabine, cyclophosphamide, and rituximab) and FR (fludarabine and rituximab), pentostatin-based therapeutic regimens including but not limited to PCR (pentostatin, cyclophosphamide, and rituximab), alemtuzumab (Campath®), chlorambucil, chlorambucil in combination with obinutuzumab (Gazyva® anti-CD20 Mab), tyrosine kinase inhibitors including but not limited to ibrutinib.

When the hematologic malignancy is refractory or relapsed CLL the supplementary agent is selected from one or more of lenalidomide, ofatumumab, phosphoinositide 3-kinase (PI3K) inhibitors such as duvelisib and idelalisib; venetoclax alone or in combination with obinutuzumab or rituximab. When the hematologic malignancy is B cell lymphomas the supplementary agent is selected from one or more of Rituximab (Rituxan®) optionally in combination with cyclophosphamide; bendamustine in combination with obinutuzum or rituximab; CHOP (cyclophosphamide, doxorubicin or hydroxydaunorubicin, vincristine (Oncovin®), and prednisone) optionally in combination with obinutuzumab or rituximab; CVP (cyclophosphamide, vincristine, prednisone) optionally in combination with obinutuzumab or rituximab; lenalidomide and rituximab; cyclophosphamide; chlorambucil; and ibritumomab tiuxetang (Zevalin®);

When the hematologic malignancy is Burkitts Lymphoma the supplementary agent is selected from one or more of: CODOX-M (cyclophosphamide, doxorubicin, vincristine with intrathecal methotrexate and cytarabine followed by high-dose systemic methotrexate) optionally in combination with rituximab; dose-adjusted EPOCH (etoposide, prednisone, vincristine, cyclophosphamide, doxorubicin) optionally in combination with rituximab; hyperCVAD (cyclophosphamide, vincristine, doxorubicin, and dexamethasone) optionally in combination with high-dose methotrexate and cytarabine optionally in combination with rituximab; RICE (rituximab, ifosfamide, carboplatin, etoposide) optionally in combination with intrathecal methotrexate; RIVAC (rituximab, ifosfamide, cytarabine, etoposide) optionally in combination with intrathecal methotrexate and RGDP (rituximab, gemcitabine, dexamethasone, cisplatin); and cytarabine optionally in combination with rituximab.

In some embodiments, the orthogonal receptor is an orthogonal CD122 comprising one or more STAT3 binding motifs.

Examples of anti-CD19 CARs useful in the practice of the methods of the present disclosure include the following constructs:

CD19_28 z: a construct comprising a GMCSF receptor signal peptide, FMC63 scFv, AAA spacer, CD28 hinge and costimulatory domain and CD3 zeta:

(SEQ ID NO: 32)
MLLLVTSLLLCELPHPAFLLIPDIQMTQTTSSLSASLGDRVTISCR
ASQDISKYLNWYQQKPDGTVKLLIYHTSRLHSGVPSRFSGSGSGTD
YSLTISNLEQEDIATYFCQQGNTLPYTFGGGTKLEITGSTSGSGKP
GSGEGSTKGEVKLQESGPGLVAPSQSLSVTCTVSGVSLPDYGVSWI
RQPPRKGLEWLGVIWGSETTYYNSALKSRLTIIKDNSKSQVFLKMN
SLQTDDTAIYYCAKHYYYGGSYAMDYWGQGTSVTVSSAAAIEVMYP
PPYLDNEKSNGTIIHVKGKHLCPSPLFPGPSKPFWVLVVVGGVLAC
YSLLVTVAFIIFWVRSKRSRLLHSDYMNMTPRRPGPTRKHYQPYAP
PRDFAAYRSRVKFSRSADAPAYQQGQNQLYNELNLGRREEYDVLDK
RRGRDPEMGGKPRRKNPQEGLYNELQKDKMAEAYSEIGMKGERRRG
KGHDGLYQGLSTATKDTYDALHMQALPP

CD19_4-1bbz: a construct comprising a CD8a receptor signal peptide, FMC63 scFv, CD8 hinge and transmembrane domain, a 4-1BB hinge and costimulatory domain and CD3 zeta:

(SEQ ID NO: 33)
MALPVTALLLPLALLLHAARPDIQMTQTTSSLSASLGDRVTISCRA
SQDISKYLNWYQQKPDGTVKLLIYHTSRLHSGVPSRFSGSGSGTDY
SLTISNLEQEDIATYFCQQGNTLPYTFGGGTKLEITGGGGSGGGGS
GGGGSEVKLQESGPGLVAPSQSLSVTCTVSGVSLPDYGVSWIRQPP
RKGLEWLGVIWGSETTYYNSALKSRLTIIKDNSKSQVFLKMNSLQT
DDTAIYYCAKHYYYGGSYAMDYWGQGTSVTVSSTTTPAPRPPTPAP
TIASQPLSLRPEACRPAAGGAVHTRGLDFACDIYIWAPLAGTCGVL
LLSLVITLYCKRGRKKLLYIFKQPFMRPVQTTQEEDGCSCRFPEEE
EGGCELRVKFSRSADAPAYKQGQNQLYNELNLGRREEYDVLDKRRG
RDPEMGGKPRRKNPQEGLYNELQKDKMAEAYSEIGMKGERRRGKGH
DGLYQGLSTATKDTYDALHMQALPPR

In an alternative embodiment, the CD19 CAR comprises the amino acid sequence:

(SEQ ID NO: 33)
MALPVTALLLPLALLLHAARPDIQMTQTTSSLSASLGDRVTISCRA
SQDISKYLNWYQQKPDGTVKLLIYHTSRLHSGVPSRFSGSGSGTDY
SLTISNLEQEDIATYFCQQGNTLPYTFGGGTKLEITGGGGSGGGGS
GGGGSEVKLQESGPGLVAPSQSLSVTCTVSGVSLPDYGVSWIRQPP
RKGLEWLGVIWGSETTYYNSALKSRLTIIKDNSKSQVFLKMNSLQT
DDTAIYYCAKHYYYGGSYAMDYWGQGTSVTVSSTTTPAPRPPTPAP
TIASQPLSLRPEACRPAAGGAVHTRGLDFACDIYIWAPLAGTCGVL
LLSLVITLYCKRGRKKLLYIFKQPFMRPVQTTQEEDGCSCRFPEEE
EGGCELRVKFSRSADAPAYKQGQNQLYNELNLGRREEYDVLDKRRG
RDPEMGGKPRRKNPQEGLYNELQKDKMAEAYSEIGMKGERRRGKGH
DGLYQGLSTATKDTYDALHMQALPPR

In an alternative embodiment, the CD19 CAR comprises the amino acid sequence:

(SEQ ID NO: 30)
MLLLVTSLLLCELPHPAFLLIPDIQMTQTTSSLSASLGDRVTISC
RASQDISKYLNWYQQKPDGTVKLLIYHTSRLHSGVPSRFSGSGSG
TDYSLTISNLEQEDIATYFCQQGNTLPYTFGGGTKLEITGSTSGS
GKPGSGEGSTKGEVKLQESGPGLVAPSQSLSVTCTVSGVSLPDYG
VSWIRQPPRKGLEWLGVIWGSETTYYNSALKSRLTIIKDNSKSQV
FLKMNSLQTDDTAIYYCAKHYYYGGSYAMDYWGQGTSVTVSSAAA
IEVMYPPPYLDNEKSNGTIIHVKGKHLCPSPLFPGPSKPFWVLVV
VGGVLACYSLLVTVAFIIFWVRSKRSRLLHSDYMNMTPRRPGPTR
KHYQPYAPPRDFAAYRSRVKFSRSADAPAYQQGQNQLYNELNLGR
REEYDVLDKRRGRDPEMGGKPRRKNPQEGLYNELQKDKMAEAYSE
IGMKGERRRGKGHDGLYQGLSTATKDTYDALHMQALPPR

### BCMA CARs:

The present disclosure provides methods and compositions for treating a subject suffering from multiple myeloma by the administration of a plurality of engineered T cells expressing an orthogonal CD122 polypeptide and a chimeric antigen receptor the extracellular domain of which specifically binds to BCMA in combination administration of orthogonal IL2 ligand of Formula 1 for a first period of time until the subject has achieve a partial or complete response (treatment phase) and the prevention of metastasis of said neoplastic disease by the administration to said subject of a maintenance therapy comprising the periodic administration of an orthogonal IL2 ligand of Formula 1 over a period of at least 30, optionally at least 90, optionally at least 180 days, or longer if it is deemed necessary by the physician.

The present disclosure provides methods and compositions achieving a stringent complete response in the treatment of a subject suffering from multiple myeloma by the administration of a plurality of engineered T cells expressing an orthogonal CD122 polypeptide and a chimeric antigen receptor the extracellular domain of which specifically binds to BCMA and the contemporaneous combination administration of orthogonal IL2 ligand of Formula 1 and the subsequent administration to said subject of a maintenance therapy comprising the periodic administration of an orthogonal IL2 ligand of Formula 1.

In the foregoing methods, the orthogonal ligand used in the treatment phase may be the same or different than the orthogonal ligand used in the maintenance phase. In some embodiments, the orthogonal ligand is modified to extend half-life. In one embodiment the orthogonal ligand is pegylated, fusion protein, etc. In one embodiment the orthogonal ligand is comprises a 40 kD N terminally PEG moiety.

In some embodiments of the method, the orthogonal ligand is dosed in a first therapeutic phase ligand above the concentration sufficient to provide expansion (e.g >EC₁₀^{PRO} but below the concentration sufficient to induce significant differentiation (e.g. <EC₉₀^{ACT}) for a period of at least 24 hours, optionally for period of at 30, 60, 90 or longer days. In some embodiments, the maintenance phase optionally including the administration of an orthogonal ligand of Formula 1 sufficient to induce ortho CAR T activation maintaining a serum level in excess of the concentration for activation (e.g. >EC₅₀^{ACT}) for a period of at least 24 hours.

In some embodiments of the method the orthogonal ligand is provided to the subject by the administration of a recombinant viral or non-viral vector comprising the nucleic acid of encoding an orthogonal IL2 ligand of Formula 1. In some embodiments of the method the neoplastic disease is selected from solid tumors and hematologic malignancies. In some embodiments of the method. In some embodiments of the method the method further comprises the administration of one or more supplementary anti-neoplastic agents during the treatment phase and/or maintenance phase. In some embodiments of the method supplementary anti-neoplastic agents during the treatment phase and/or maintenance phase can be same or different. In some embodiments of the method, the supplementary neoplastic agent is selected from the group consisting of chemotherapeutic agents, small molecules, supplementary biologics including but not limited to checkpoint inhibitors (anti-PD1, Keytruda, Opdivo), anti-tumor antigen antibodies (Herceptin), and/or physical methods (surgery, radiation, etc). In some embodiments, the orthogonal receptor is an orthogonal CD122 comprising one or more STAT3 binding motifs.

In some embodiments, supplementary agents useful in the treatment of multiple myeloma include one or more agents selected from the group consisting of thalidomide, lenalidomide, dexamethasone, bortezomib, vincristine, doxorubicin, dexamethasone, melphalan, carfilzomib, cyclophosphamide, cisplatin, etoposide, bortezomib, prednisone, daratumumab, carfilzomib, and ixazomib. In some embodiments, supplementary agents useful in the treatment of multiple myeloma include combination treatment regimens of chemotherapeutic agents for use in the treatment of multiple myeloma that include theortezomib/lenalidomide/dexamethasone; bortezomib/cyclophosphamide/dexamethasone; bortezomib/doxorubicin/dexamethasone; carfilzomib/lenalidomide/dexamethasone; ixazomib/lenalidomide/dexamethasone; bortezomib/dexamethasone; bortezomib/thalidomide/dexamethasone; lenalidomide/dexamethasone; dexamethasone/thalidomide/cisplatin/doxorubicin/cyclophosph amide/etoposide/bortezomib (VTD-PACE); lenalidomide/low-dose dexamethasone; daratumumab/bortezomib/melphalan/prednisone; carfilzomib/lenalidomide/dexamethasone; carfilzomib/cyclophosphamide/dexamethasone; and ixazomib/lenalidomide/dexamethasone

The present disclosure provides methods and compositions for treating a subject from smoldering multiple myeloma by the administration of a plurality of engineered T cells expressing an orthogonal CD122 polypeptide and a chimeric antigen receptor (CAR) the extracellular domain of said CAR specifically binds to BCMA in combination with a orthogonal IL2 ligand of Formula 1 and the subsequent administration to said subject of a maintenance therapy comprising the periodic administration of an orthogonal IL2 ligand of Formula 1. The present disclosure provides methods and compositions for preventing the progression of smoldering multiple myeloma to multiple myeloma in a subject suffering from smoldering multiple myeloma by the administration of a plurality of engineered T cells expressing an orthogonal CD122 polypeptide and a chimeric antigen receptor (CAR) the extracellular domain of said CAR specifically binds to BCMA and the contemporaneous combination administration of orthogonal IL2 ligand of Formula 1 and the prevention of progression of the smoldering multiple myeloma to multiple myeloma of said neoplastic disease by the administration to said subject of a maintenance therapy comprising the periodic administration of an orthogonal IL2 ligand of Formula 1. In some embodiments, the ABD of the CAR binds specifically to BCMA. The method further comprising one or more supplementary anti-neoplastic agents administered to the subject during the treatment phase and/or maintenance phase of supplementary agents useful in the treatment of multiple myeloma that include one or more of selected from the group consisting of thalidomide, lenalidomide, dexamethasone, bortezomib, vincristine, doxorubicin, dexamethasone, melphalan, carfilzomib, cyclophosphamide, cisplatin, etoposide, bortezomib, prednisone, daratumumab, carfilzomib, and ixazomib or combination treatment regimens of chemotherapeutic agents for use in the treatment of multiple myeloma that include the bortezomib/lenalidomide/dexamethasone; bortezomib/cyclophosphamide/dexamethasone; bortezomib/doxorubicin/dexamethasone; carfilzomib/lenalidomide/dexamethasone; ixazomib/lenalidomide/dexamethasone; bortezomib/dexamethasone; bortezomib/thalidomide/dexamethasone; lenalidomide/dexamethasone; dexamethasone/thalidomide/cisplatin/doxorubicin/cyclophosphamide/etoposide/bortezomib (VTD-PACE); lenalidomide/low-dose dexamethasone; daratumumab/bortezomib/melphalan/prednisone; carfilzomib/lenalidomide/dexamethasone; carfilzomib/cyclophosphamide/dexamethasone; and ixazomib/lenalidomide/dexamethasone

Examples of anti-BCMA CARs useful in the practice of the present invention include the following constructs:

BCMA4_41bbz CAR: a construct comprising a CD8a receptor signal peptide, BCMA4 scFv, CD8 hinge and transmembrane domain, a 4-1BB hinge and costimulatory domain and CD3 zeta,

(SEQ ID NO: 34)
MALPVTALLLPLALLLHAARPQVQLVESGGGLVQPGRSLRLSCAAS
GFTFSNYAMSWVRQAPGKGLGWVSGISRSGENTYYADSVKGRFTIS
RDNSKNTLYLQMNSLRDEDTAVYYCARSPAHYYGGMDVWGQGTTVT
VSSASGGGGSGGRASGGGGSDIVLTQSPGTLSLSPGERATLSCRAS
QSISSSFLAWYQQKPGQAPRLLIYGASRRATGIPDRFSGSGSGTDF
TLTISRLEPEDSAVYYCQQYHSSPSWTFGQGTKLEIKTTTPAPRPP
TPAPTIASQPLSLRPEACRPAAGGAVHTRGLDFACDIYIWAPLAGT
CGVLLLSLVITLYCKRGRKKLLYIFKQPFMRPVQTTQEEDGCSCRF
PEEEEGGCELRVKFSRSADAPAYKQGQNQLYNELNLGRREEYDVLD
KRRGRDPEMGGKPRRKNPQEGLYNELQKDKMAEAYSEIGMKGERRR
GKGHDGLYQGLSTATKDTYDALHMQALPPR

which can be co-expressed with the ortho CD122 receptor using a T2a linker with an amino acid sequence of:

(SEQ ID NO: 35)
MALPVTALLLPLALLLHAARPQVQLVESGGGLVQPGRSLRLSCAAS
GFTFSNYAMSWVRQAPGKGLGWVSGISRSGENTYYADSVKGRFTIS
RDNSKNTLYLQMNSLRDEDTAVYYCARSPAHYYGGMDVWGQGTTVT
VSSASGGGGSGGRASGGGGSDIVLTQSPGTLSLSPGERATLSCRAS
QSISSSFLAWYQQKPGQAPRLLIYGASRRATGIPDRFSGSGSGTDF
TLTISRLEPEDSAVYYCQQYHSSPSWTFGQGTKLEIKTTTPAPRPP
TPAPTIASQPLSLRPEACRPAAGGAVHTRGLDFACDIYIWAPLAGT
CGVLLLSLVITLYCKRGRKKLLYIFKQPFMRPVQTTQEEDGCSCRF
PEEEEGGCELRVKFSRSADAPAYKQGQNQLYNELNLGRREEYDVLD
KRRGRDPEMGGKPRRKNPQEGLYNELQKDKMAEAYSEIGMKGERRR
GKGHDGLYQGLSTATKDTYDALHMQALPPRGSGEGRGSLLTCGDVE
ENPGPMAAPALSWRLPLLILLLPLATSWASAAVNGTSQFTCFYNSR
ANISCVWSQDGALQDTSCQVHAWPDRRRWNQTCELLPVSQASWACN
LILGAPDSQKLTTVDIVTLRVLCREGVRWRVMAIQDFKPFENLRLM
APISLQVVHVETHRCNISWEISQASDFFERHLEFEARTLSPGHTWE
EAPLLTLKQKQEWICLETLTPDTQYEFQVRVKPLQGEFTTWSPWSQ
PLAFRTKPAALGKDTIPWLGHLLVGLSGAFGFIILVYLLINCRNTG
PWLKKVLKCNTPDPSKFFSQLSSEHGGDVQKWLSSPFPSSSFSPGG
LAPEISPLEVLERDKVTQLLLQQDKVPEPASLSSNHSLTSCFTNQG
YFFFHLPDALEIEACQVYFTYDPYSEEDPDEGVAGAPTGSSPQPLQ
PLSGEDDAYCTFPSRDDLLLFSPSLLGGPSPPSTAPGGSGAGEERM
PPSLQERVPRDWDPQPLGPPTPGVPDLVDFQPPPELVLREAGEEVP
DAGPREGVSFPWSRPPGQGEFRALNARLPLNTDAYLSLQELQGQDP
THLV

GSI5021_41 bbz CAR:

(SEQ ID NO: 36)
MALPVTALLLPLALLLHAARPQVKLEESGGGLVQAGRSLRLSCAASE
HTFSSHVMGWFRQAPGKERESVAVIGWRDISTSYADSVKGRFTISRD
NAKKTLYLQMNSLKPEDTAVYYCAARRIDAADFDSWGQGTQVTVSSG
GGGSEVQLVESGGGLVQAGGSLRLSCAASGRTFTMGWFRQAPGKERE
FVAAISLSPTLAYYAESVKGRFTISRDNAKNTVVLQMNSLKPEDTAL
YYCAADRKSVMSIRPDYWGQGTQVTVSSTSTTTPAPRPPTPAPTIAS
QPLSLRPEACRPAAGGAVHTRGLDFACDIYIWAPLAGTCGVLLLSLV
ITLYCKRGRKKLLYIFKQPFMRPVQTTQEEDGCSCRFPEEEEGGCEL
RVKFSRSADAPAYQQGQNQLYNELNLGRREEYDVLDKRRGRDPEMGG
KPRRKNPQEGLYNELQKDKMAEAYSEIGMKGERRRGKGHDGLYQGLS
TATKDTYDALHMQALPPR

which can be co-expressed with the ortho CD122 receptor using a T2a linker with an amino acid sequence of:

(SEQ ID NO: 37)
MALPVTALLLPLALLLHAARPQVKLEESGGGLVQAGRSLRLSCAASE
HTFSSHVMGWFRQAPGKERESVAVIGWRDISTSYADSVKGRFTISRD
NAKKTLYLQMNSLKPEDTAVYYCAARRIDAADFDSWGQGTQVTVSSG
GGGSEVQLVESGGGLVQAGGSLRLSCAASGRTFTMGWFRQAPGKERE
FVAAISLSPTLAYYAESVKGRFTISRDNAKNTVVLQMNSLKPEDTAL
YYCAADRKSVMSIRPDYWGQGTQVTVSSTSTTTPAPRPPTPAPTIAS
QPLSLRPEACRPAAGGAVHTRGLDFACDIYIWAPLAGTCGVLLLSLV
ITLYCKRGRKKLLYIFKQPFMRPVQTTQEEDGCSCRFPEEEEGGCEL
RVKFSRSADAPAYQQGQNQLYNELNLGRREEYDVLDKRRGRDPEMGG
KPRRKNPQEGLYNELQKDKMAEAYSEIGMKGERRRGKGHDGLYQGLS
TATKDTYDALHMQALPPRGSGEGRGSLLTCGDVEENPGPMAAPALSW
RLPLLILLLPLATSWASAAVNGTSQFTCFYNSRANISCVWSQDGALQ
DTSCQVHAWPDRRRWNQTCELLPVSQASWACNLILGAPDSQKLTTVD
IVTLRVLCREGVRWRVMAIQDFKPFENLRLMAPISLQVVHVETHRCN
ISWEISQASDFFERHLEFEARTLSPGHTWEEAPLLTLKQKQEWICLE
TLTPDTQYEFQVRVKPLQGEFTTWSPWSQPLAFRTKPAALGKDTIPW
LGHLLVGLSGAFGFIILVYLLINCRNTGPWLKKVLKCNTPDPSKFFS
QLSSEHGGDVQKWLSSPFPSSSFSPGGLAPEISPLEVLERDKVTQLL
LQQDKVPEPASLSSNHSLTSCFTNQGYFFFHLPDALEIEACQVYFTY
DPYSEEDPDEGVAGAPTGSSPQPLQPLSGEDDAYCTFPSRDDLLLFS
PSLLGGPSPPSTAPGGSGAGEERMPPSLQERVPRDWDPQPLGPPTPG
VPDLVDFQPPPELVLREAGEEVPDAGPREGVSFPWSRPPGQGEFRAL
NARLPLNTDAYLSLQELQGQDPTHLV

B2121 BCMA_41 bbz) CAR

(SEQ ID NO: 38)
MALPVTALLLPLALLLHAARPDIVLTQSPPSLAMSLGKRATISCRAS
ESVTILGSHLIHWYQQKPGQPPTLLIQLASNVQTGVPARFSGSGSRT
DFTLTIDPVEEDDVAVYYCLQSRTIPRTFGGGTKLEIKGSTSGSGKP
GSGEGSTKGQIQLVQSGPELKKPGETVKISCKASGYTFTDYSINWVK
RAPGKGLKWMGWINTETREPAYAYDFRGRFAFSLETSASTAYLQINN
LKYEDTATYFCALDYSYAMDYWGQGTSVTVSSAAATTTPAPRPPTPA
PTIASQPLSLRPEACRPAAGGAVHTRGLDFACDIYIWAPLAGTCGVL
LLSLVITLYCKRGRKKLLYIFKQPFMRPVQTTQEEDGCSCRFPEEEE
GGCELRVKFSRSADAPAYQQGQNQLYNELNLGRREEYDVLDKRRGRD
PEMGGKPRRKNPQEGLYNELQKDKMAEAYSEIGMKGERRRGKGHDGL
YQGLSTATKDTYDALHMQALPPR

which can be co-expressed with the ortho CD122 receptor using a T2a linker with an amino acid sequence of:

(SEQ ID NO: 37)
MALPVTALLLPLALLLHAARPQVKLEESGGGLVQAGRSLRLSCAAS
EHTFSSHVMGWFRQAPGKERESVAVIGWRDISTSYADSVKGRFTIS
RDNAKKTLYLQMNSLKPEDTAVYYCAARRIDAADFDSWGQGTQVTV
SSGGGGSEVQLVESGGGLVQAGGSLRLSCAASGRTFTMGWFRQAPG
KEREFVAAISLSPTLAYYAESVKGRFTISRDNAKNTVVLQMNSLKP
EDTALYYCAADRKSVMSIRPDYWGQGTQVTVSSTSTTTPAPRPPTP
APTIASQPLSLRPEACRPAAGGAVHTRGLDFACDIYIWAPLAGTCG
VLLLSLVITLYCKRGRKKLLYIFKQPFMRPVQTTQEEDGCSCRFPE
EEEGGCELRVKFSRSADAPAYQQGQNQLYNELNLGRREEYDVLDKR
RGRDPEMGGKPRRKNPQEGLYNELQKDKMAEAYSEIGMKGERRRGK
GHDGLYQGLSTATKDTYDALHMQALPPRGSGEGRGSLLTCGDVEEN
PGPMAAPALSWRLPLLILLLPLATSWASAAVNGTSQFTCFYNSRAN
ISCVWSQDGALQDTSCQVHAWPDRRRWNQTCELLPVSQASWACNLI
LGAPDSQKLTTVDIVTLRVLCREGVRWRVMAIQDFKPFENLRLMAP
ISLQVVHVETHRCNISWEISQASDFFERHLEFEARTLSPGHTWEEA
PLLTLKQKQEWICLETLTPDTQYEFQVRVKPLQGEFTTWSPWSQPL
AFRTKPAALGKDTIPWLGHLLVGLSGAFGFIILVYLLINCRNTGPW
LKKVLKCNTPDPSKFFSQLSSEHGGDVQKWLSSPFPSSSFSPGGLA
PEISPLEVLERDKVTQLLLQQDKVPEPASLSSNHSLTSCFTNQGYF
FFHLPDALEIEACQVYFTYDPYSEEDPDEGVAGAPTGSSPQPLQPL
SGEDDAYCTFPSRDDLLLFSPSLLGGPSPPSTAPGGSGAGEERMPP
SLQERVPRDWDPQPLGPPTPGVPDLVDFQPPPELVLREAGEEVPDA
GPREGVSFPWSRPPGQGEFRALNARLPLNTDAYLSLQELQGQDPTH
LV

BMCA10_41bbz CAR

(SEQ ID NO: 39)
MALPVTALLLPLALLLHAARPEVQLVESGGGLVQPGGSLRLSCAVS
GFALSNHGMSWVRRAPGKGLEWVSGIVYSGSTYYAASVKGRFTISR
DNSRNTLYLQMNSLRPEDTAIYYCSAHGGESDVWGQGTTVTVSSAS
GGGGSGGRASGGGGSDIQLTQSPSSLSASVGDRVTITCRASQSISS
YLNWYQQKPGKAPKLLIYAASSLQSGVPSRFSGSGSGTDFTLTISS
LQPEDFATYYCQQSYSTPYTFGQGTKVEIKTTTPAPRPPTPAPTIA
SQPLSLRPEACRPAAGGAVHTRGLDFACDIYIWAPLAGTCGVLLLS
LVITLYCKRGRKKLLYIFKQPFMRPVQTTQEEDGCSCRFPEEEEGG
CELRVKFSRSADAPAYKQGQNQLYNELNLGRREEYDVLDKRRGRDP
EMGGKPRRKNPQEGLYNELQKDKMAEAYSEIGMKGERRRGKGHDGL
YQGLSTATKDTYDALHMQALPPR

which can be co-expressed with the ortho CD122 receptor using a T2a linker with an amino acid sequence of:

(SEQ ID NO: 40)
MALPVTALLLPLALLLHAARPEVQLVESGGGLVQPGGSLRLSCAVS
GFALSNHGMSWVRRAPGKGLEWVSGIVYSGSTYYAASVKGRFTISR
DNSRNTLYLQMNSLRPEDTAIYYCSAHGGESDVWGQGTTVTVSSAS
GGGGSGGRASGGGGSDIQLTQSPSSLSASVGDRVTITCRASQSISS
YLNWYQQKPGKAPKLLIYAASSLQSGVPSRFSGSGSGTDFTLTISS
LQPEDFATYYCQQSYSTPYTFGQGTKVEIKTTTPAPRPPTPAPTIA
SQPLSLRPEACRPAAGGAVHTRGLDFACDIYIWAPLAGTCGVLLLS
LVITLYCKRGRKKLLYIFKQPFMRPVQTTQEEDGCSCRFPEEEEGG
CELRVKFSRSADAPAYKQGQNQLYNELNLGRREEYDVLDKRRGRDP
EMGGKPRRKNPQEGLYNELQKDKMAEAYSEIGMKGERRRGKGHDGL
YQGLSTATKDTYDALHMQALPPRGSGEGRGSLLTCGDVEENPGPMA
APALSWRLPLLILLLPLATSWASAAVNGTSQFTCFYNSRANISCVW
SQDGALQDTSCQVHAWPDRRRWNQTCELLPVSQASWACNLILGAPD
SQKLTTVDIVTLRVLCREGVRWRVMAIQDFKPFENLRLMAPISLQV
VHVETHRCNISWEISQASDFFERHLEFEARTLSPGHTWEEAPLLTL
KQKQEWICLETLTPDTQYEFQVRVKPLQGEFTTWSPWSQPLAFRTK
PAALGKDTIPWLGHLLVGLSGAFGFIILVYLLINCRNTGPWLKKVL
KCNTPDPSKFFSQLSSEHGGDVQKWLSSPFPSSSFSPGGLAPEISP
LEVLERDKVTQLLLQQDKVPEPASLSSNHSLTSCFTNQGYFFFHLP
DALEIEACQVYFTYDPYSEEDPDEGVAGAPTGSSPQPLQPLSGEDD
AYCTFPSRDDLLLFSPSLLGGPSPPSTAPGGSGAGEERMPPSLQER
VPRDWDPQPLGPPTPGVPDLVDFQPPPELVLREAGEEVPDAGPREG
VSFPWSRPPGQGEFRALNARLPLNTDAYLSLQELQGQDPTHLV

### GD2 CARs

The present disclosure provides methods and compositions for treating a subject suffering from a neoplastic disease of neuroectodermal origin (including human neuroblastoma and melanoma) or high risk osteosarcoma by the administration of a plurality of engineered T cells expressing an orthogonal CD122 polypeptide and a chimeric antigen receptor (CAR) the extracellular domain of said CAR specifically binds to GD2 and the contemporaneous combination administration of orthogonal IL2 ligand of Formula 1 and the subsequent administration to said subject of a maintenance therapy comprising the periodic administration of an orthogonal IL2 ligand of Formula 1. The present disclosure provides methods and compositions for preventing the metastasis or relapse of a neoplastic disease of neuroectodermal origin (including human neuroblastoma and melanoma) by the administration of a plurality of engineered T cells expressing an orthogonal CD122 polypeptide and a chimeric antigen receptor (CAR) the extracellular domain of said CAR specifically binds to BCMA and the contemporaneous combination administration of orthogonal IL2 ligand of Formula 1 and the prevention of progression of the smoldering multiple myeloma to multiple myeloma of said neoplastic disease by the administration to said subject of a maintenance therapy comprising the periodic administration of an orthogonal IL2 ligand of Formula 1. In some embodiments, the extracellular domain of the CAR specifically binds to GD2. In some embodiments the ABD of the CAR comprises an antibody that specifically binds to GD2 comprising the CDRs or one or more of antibodies 3F8, hu14.18, 14G2a, optionally coadministering chemotherapeutic agents include cisplatin, doxorubicin, cyclophosphamide and epipodophyllotoxins such as teniposide and etoposide.

### PSMA CARs for Prostate Cancer

Prostate cancer is the second most frequent malignancy among men worldwide, with an estimated 1.1 million new cases per year. Prostate cancer is implicated in 307,000 deaths making it the fifth leading cause of cancer mortality. Although localized primary tumors can be successfully treated by surgery or local radiation therapy, these methodologies do not provide satisfactory results in for the advanced states of the disease.

Prostate-specific membrane antigen (PSMA) is considered an ideal target for antigen-redirected immunotherapy because it is expressed at the surface of prostate cancer cells at all tumor stages, and in particular shows an increased expression in the more severe androgen-independent and metastatic stages of the disease. A variety of antibodies targeting PSM are described in the literature which may be modified for use in the context of CAR including but not limited to J591, 3D8, D2B, and 3/F11.

A number of CAR T cells targeting PSMA are in clinical development (see e.g. NCT01140373, NCT01929239, and NCT03089203). However, the oncolytic potency of these PSMA CART cells is still uncertain. In particular, engineered T cells expressing first-generation CARs using 3D8 or J591 scFvs ABDs showed low potency due to lack of persistence of the PSMA CAR T cells. Although second and third generation CARs have been tried in prostate cancer treatments, their success remains low and required high doses of CAR T or multiple CAR T infusions. Alzubi, et al (2020) Molecular Therapy Oncolytics 18:226-235.

As previously discussed, the compositions and methods of the present disclosure provide the ability of overcoming the lack of persistence observed in conventional CAR therapy and attributed to the lack of efficacy of PSMA CARs in clinical practice. The methods and compositions of the present disclosure enable the clinician to maintaining the CARs in an active state for a prolonged period of time enabling much greater exposure (“Area Under the Curve” or AUC) than conventional CAR T cell therapies and thus providing a means to treat cancers that have previously been resistant to CAR T therapy, particularly solid tumors that require prolonged exposure to the CAR for effective treatment and to achieve as extravasation and penetration into the solid tumor environment. Conventional non-orthogonal CAR T cells are unable to provide the duration of exposure required without excessive toxicity or multiple dosing of the CART cell.

The present disclosure provides a method of treating prostate cancer, an optionally preventing relapse or recurrence, the method comprising the administration of a therapeutically effective amount of an orthogonal PSMA CAR T cell in combination with an orthogonal ligand of Formula 1.

In some embodiments, the present disclosure provides an orthogonal PSMA CAR T cell. comprising a PSMA CAR is PSMA_28z: An anti-PSMA CAR comprising a CD8a signal peptide, a deimmunized J591 scFv, a AAA spacer, CD28 hinge/transmembrane/co-stimulatory domain and CD3zeta:

(SEQ ID NO: 41)
MALPVTALLLPLALLLHAARPDIQMTQSPSSLSTSVGDRVTLTCKA
SQDVGTAVDWYQQKPGPSPKLLIYWASTRHTGIPSRFSGSGSGTDF
TLTISSLQPEDFADYYCQQYNSYPLTFGPGTKVDIKGSTSGGGSGG
GSGGGGSSEVQLVQSGPEVKKPGATVKISCKTSGYTFTEYTIHWVK
QAPGKGLEWIGNINPNNGGTTYNQKFEDKATLTVDKSTDTAYMELS
SLRSEDTAVYYCAAGWNFDYWGQGTLLTVSSAAAIEVMYPPPYLDN
EKSNGTIIHVKGKHLCPSPLFPGPSKPFWVLVVVGGVLACYSLLVT
VAFIIFWVRSKRSRLLHSDYMNMTPRRPGPTRKHYQPYAPPRDFAA
YRSRVKFSRSADAPAYQQGQNQLYNELNLGRREEYDVLDKRRGRDP
EMGGKPRRKNPQEGLYNELQKDKMAEAYSEIGMKGERRRGKGHDGL
YQGLSTATKDTYDALHMQALPPR

which can be co-expressed with the ortho CD122 receptor using a T2a linker with an amino acid sequence of:

(SEQ ID NO: 42)
MALPVTALLLPLALLLHAARPDIQMTQSPSSLSTSVGDRVTLTCK
ASQDVGTAVDWYQQKPGPSPKLLIYWASTRHTGIPSRFSGSGSGT
DFTLTISSLQPEDFADYYCQQYNSYPLTFGPGTKVDIKGSTSGGG
SGGGSGGGGSSEVQLVQSGPEVKKPGATVKISCKTSGYTFTEYTI
HWVKQAPGKGLEWIGNINPNNGGTTYNQKFEDKATLTVDKSTDTA
YMELSSLRSEDTAVYYCAAGWNFDYWGQGTLLTVSSAAAIEVMYP
PPYLDNEKSNGTIIHVKGKHLCPSPLFPGPSKPFWVLVVVGGVLA
CYSLLVTVAFIIFWVRSKRSRLLHSDYMNMTPRRPGPTRKHYQPY
APPRDFAAYRSRVKFSRSADAPAYQQGQNQLYNELNLGRREEYDV
LDKRRGRDPEMGGKPRRKNPQEGLYNELQKDKMAEAYSEIGMKGE
RRRGKGHDGLYQGLSTATKDTYDALHMQALPPRGSGEGRGSLLTC
GDVEENPGPMAAPALSWRLPLLILLLPLATSWASAAVNGTSQFTC
FYNSRANISCVWSQDGALQDTSCQVHAWPDRRRWNQTCELLPVSQ
ASWACNLILGAPDSQKLTTVDIVTLRVLCREGVRWRVMAIQDFKP
FENLRLMAPISLQVVHVETHRCNISWEISQASDFFERHLEFEART
LSPGHTWEEAPLLTLKQKQEWICLETLTPDTQYEFQVRVKPLQGE
FTTWSPWSQPLAFRTKPAALGKDTIPWLGHLLVGLSGAFGFIILV
YLLINCRNTGPWLKKVLKCNTPDPSKFFSQLSSEHGGDVQKWLSS
PFPSSSFSPGGLAPEISPLEVLERDKVTQLLLQQDKVPEPASLSS
NHSLTSCFTNQGYFFFHLPDALEIEACQVYFTYDPYSEEDPDEGV
AGAPTGSSPQPLQPLSGEDDAYCTFPSRDDLLLFSPSLLGGPSPP
STAPGGSGAGEERMPPSLQERVPRDWDPQPLGPPTPGVPDLVDFQ
PPPELVLREAGEEVPDAGPREGVSFPWSRPPGQGEFRALNARLPL
NTDAYLSLQELQGQDPTHLV

in some embodiments, the PSMA CAR is PSMA 4-1BBz: a deimmunized J591 scfv; CD8a signal peptide, CD8 hinge and transmembrane domain and 4-1bb costimulatory domain and CD3z:

(SEQ ID NO: 43)
MALPVTALLLPLALLLHAARPDIQMTQSPSSLSTSVGDRVTLTCK
ASQDVGTAVDWYQQKPGPSPKLLIYWASTRHTGIPSRFSGSGSGT
DFTLTISSLQPEDFADYYCQQYNSYPLTFGPGTKVDIKGSTSGGG
SGGGSGGGGSSEVQLVQSGPEVKKPGATVKISCKTSGYTFTEYTI
HWVKQAPGKGLEWIGNINPNNGGTTYNQKFEDKATLTVDKSTDTA
YMELSSLRSEDTAVYYCAAGWNFDYWGQGTLLTVSSTTTPAPRPP
TPAPTIASQPLSLRPEACRPAAGGAVHTRGLDFACDIYIWAPLAG
TCGVLLLSLVITKRGRKKLLYIFKQPFMRPVQTTQEEDGCSCRFP
EEEEGGCELRVKFSRSADAPAYQQGQNQLYNELNLGRREEYDVLD
KRRGRDPEMGGKPQRRKNPQEGLYNELQKDKMAEAYSEIGMKGER
RRGKGHDGLYQGLSTATKDTYDALHMQALPPR

which can be co-expressed with the ortho CD122 receptor using a T2a linker with an amino acid sequence of:

(SEQ ID NO: 44)
MALPVTALLLPLALLLHAARPDIQMTQSPSSLSTSVGDRVTLTC
KASQDVGTAVDWYQQKPGPSPKLLIYWASTRHTGIPSRFSGSGS
GTDFTLTISSLQPEDFADYYCQQYNSYPLTFGPGTKVDIKGSTS
GGGSGGGSGGGGSSEVQLVQSGPEVKKPGATVKISCKTSGYTFT
EYTIHWVKQAPGKGLEWIGNINPNNGGTTYNQKFEDKATLTVDK
STDTAYMELSSLRSEDTAVYYCAAGWNFDYWGQGTLLTVSSTTT
PAPRPPTPAPTIASQPLSLRPEACRPAAGGAVHTRGLDFACDIY
IWAPLAGTCGVLLLSLVITKRGRKKLLYIFKQPFMRPVQTTQEE
DGCSCRFPEEEEGGCELRVKFSRSADAPAYQQGQNQLYNELNLG
RREEYDVLDKRRGRDPEMGGKPQRRKNPQEGLYNELQKDKMAEA
YSEIGMKGERRRGKGHDGLYQGLSTATKDTYDALHMQALPPRGS
GEGRGSLLTCGDVEENPGPMAAPALSWRLPLLILLLPLATSWAS
AAVNGTSQFTCFYNSRANISCVWSQDGALQDTSCQVHAWPDRRR
WNQTCELLPVSQASWACNLILGAPDSQKLTTVDIVTLRVLCREG
VRWRVMAIQDFKPFENLRLMAPISLQVVHVETHRCNISWEISQA
SDFFERHLEFEARTLSPGHTWEEAPLLTLKQKQEWICLETLTPD
TQYEFQVRVKPLQGEFTTWSPWSQPLAFRTKPAALGKDTIPWLG
HLLVGLSGAFGFIILVYLLINCRNTGPWLKKVLKCNTPDPSKFF
SQLSSEHGGDVQKWLSSPFPSSSFSPGGLAPEISPLEVLERDKV
TQLLLQQDKVPEPASLSSNHSLTSCFTNQGYFFFHLPDALEIEA
CQVYFTYDPYSEEDPDEGVAGAPTGSSPQPLQPLSGEDDAYCTF
PSRDDLLLFSPSLLGGPSPPSTAPGGSGAGEERMPPSLQERVPR
DWDPQPLGPPTPGVPDLVDFQPPPELVLREAGEEVPDAGPREGV
SFPWSRPPGQGEFRALNARLPLNTDAYLSLQELQGQDPTHLV

### GPC3 CARs

In some embodiments, the present disclosure provides an orthogonal GPC3 CAR T cell. In some embodiments, the methods and compositions of the present disclosure are useful in the treatment of GPC3 expressing cancers including but not limited to liver cancer. Hepatocellular carcinoma (HCC) is the second leading cause of cancer deaths in the world. Glypican-3, a cell-surface glycoprotein, is overexpressed in HCC tissues but not in the healthy adult liver and as such provides a useful targeting domain for the ABD of the CAR. A variety of GPC targeting domains may be employed in the construction of a CAR which may be incorporated into an orthogonal cell for use in combination with a IL2 ortholog of Formula 1 for use in the treatment of liver cancer. Examples GPC3 CARs which may be used in the preparation of an orthogonal GPC3 CAR T cell include but are not limited to a CAR GPC3_28z having the amino acid sequence:

(SEQ ID NO: 45)
DVVMTQSPLSLPVTPGEPASISCRSSQSLVHSNANTYLHWYLQKP
GQSPQLLIYKVSNRFSGVPDRFSGSGSGTDFTLKISRVEAEDVGV
YYCSQNTHVPPTFGQGTKLEIKRGGGGSGGGGSGGGGSQVQLVQS
GAEVKKPGASVKVSCKASGYTFTDYEMHWVRQAPGQGLEWMGALD
PKTGDTAYSQKFKGRVTLTADESTSTAYMELSSLRSEDTAVYYCT
RFYSYTYWGQGTLVTVSSTTTPAPRPPTPAPTIASQPLSLRPEAC
RPAAGGAVHTRGLDFACDFWVLVVVGGVLACYSLLVTVAFIIFWV
RSKRSRLLHSDYMNMTPRRPGPTRKHYQPYAPPRDFAAYRSRVKF
SRSADAPAYQQGQNQLYNELNLGRREEYDVLDKRRGRDPEMGGKP
QRRKNPQEGLYNELQKDKMAEAYSEIGMKGERRRGKGHDGLYQGL
STATKDTYDALHMQALPPR

and GPC3_4-1BB having the amino acid sequence:

(SEQ ID NO: 46)
DVVMTQSPLSLPVTPGEPASISCRSSQSLVHSNANTYLHWYLQK
PGQSPQLLIYKVSNRFSGVPDRFSGSGSGTDFTLKISRVEAEDV
GVYYCSQNTHVPPTFGQGTKLEIKRGGGGSGGGGSGGGGSQVQL
VQSGAEVKKPGASVKVSCKASGYTFTDYEMHWVRQAPGQGLEWM
GALDPKTGDTAYSQKFKGRVTLTADESTSTAYMELSSLRSEDTA
VYYCTRFYSYTYWGQGTLVTVSSTTTPAPRPPTPAPTIASQPLS
LRPEACRPAAGGAVHTRGLDFACDIYIWAPLAGTCGVLLLSLVI
TKRGRKKLLYIFKQPFMRPVQTTQEEDGCSCRFPEEEEGGCELR
VKFSRSADAPAYQQGQNQLYNELNLGRREEYDVLDKRRGRDPEM
GGKPQRRKNPQEGLYNELQKDKMAEAYSEIGMKGERRRGKGHDG
LYQGLSTATKDTYDALHMQALPPR

in some embodiments, the present disclosure provides an orthogonal HPV-16 E6 TCR cell for use in the treatment of HPV related tumors. Examples of HPV-16 E6 CARs which may be incorporated into the an orthogonal cell of the present disclosure include but are not limited to a CAR having the sequence:

(SEQ ID NO: 47)
MALEHLLIILWMQLTWVSGQQLNQSPQSMFIQEGEDVSMNCTSS
SIFNTWLWYKQDPGEGPVLLIALYKAGELTSNGRLTAQFGITRK
DSFLNISASIPSDVGIYFCAGHPSSNSGYALNFGKGTSLLVTPD
IQNPEPAVYQLKDPRSQDSTLCLFTDFDSQINVPKTMESGTFIT
DKTVLDMKAMDSKSNGAIAWSNQTSFTCQDIFKETNATYPSSDV
PCDATLTEKSFETDMNLNFQNLSVMGLRILLLKVAGFNLLMTLR
LWSSRAKRSGSGATNFSLLKQAGDVEENPGPMGTSLLCWVVLGF
LGTDHTGAGVSQSPRYKVTKRGQDVALRCDPISGHVSLYWYRQA
LGQGPEFLTYFNYEAQQDKSGLPNDRFSAERPEGSISTLTIQRT
EQRDSAMYRCASSSQTGARTDTQYFGPGTRLTVLEDLRNVTPPK
VSLFEPSKAEIANKQKATLVCLARGFFPDHVELSWWVNGKEVHS
GVSTDPQAYKESNYSYCLSSRLRVSATFWHNPRNHFRCQVQFHG
LSEEDKWPEGSPKPVTQNISAEAWGRADCGITSASYQQGVLSAT
ILYEILLGKATLYAVLVSTLVVMAMVKRKNS

Or:

(SEQ ID NO: 48)
MAPGLLCWALLCLLGAGLVDAGVTQSPTHLIKTRGQQVTLRCSP
KSGHDTVSWYQQALGQGPQFIFQYYEEEERQRGNFPDRFSGHQF
PNYSSELNVNALLLGDSALYLCASSLGWRGGRYNEQFFGPGTRL
TVLEDLRNVTPPKVSLFEPSKAEIANKQKATLVCLARGFFPDHV
ELSWWVNGKEVHSGVCTDPQAYKESNYSYCLSSRLRVSATFWHN
PRNHFRCQVQFHGLSEEDKWPEGSPKPVTQNISAEAWGRADCGI
TSASYQQGVLSATILYEILLGKATLYAVLVSTLVVMAMVKRKNS
RAKRSGSGATNFSLLKQAGDVEENPGPMWGVFLLYVSMKMGGTT
GQNIDQPTEMTATEGAIVQINCTYQTSGFNGLFWYQQHAGEAPT
FLSYNVLDGLEEKGRFSSFLSRSKGYSYLLLKELQMKDSASYLC
ASVDGNNRLAFGKGNQVVVIPNIQNPEPAVYQLKDPRSQDSTLC
LFTDFDSQINVPKTMESGTFITDKCVLDMKAMDSKSNGAIAWSN
QTSFTCQDIFKETNATYPSSDVPCDATLTEKSFETDMNLNFQNL
LVIVLRILLLKVAGFNLLMTLRLWSS

### GD2 CARs for Neuroblastoma

GD2 is highly expressed by almost all neuroblastomas, by most melanomas and retinoblastomas, and by many Ewing sarcomas and, to a more variable degree, by small cell lung cancer, gliomas, osteosarcomas, and soft tissue sarcomas. Consequently, GD2 was identified as a target for CAR T cell therapy and a variety of GD2 CARs are known in the art. However, persistence remains an issue in the treatment of GD2 expressing tumors. Consequently, the ability to extend the duration of action of orthogonal immune cells by the administration of an ortho ligand would be of significant benefit in the development of GD2 cell therapeutics. In some embodiments, the present invention provides an orthoGD2 targeted immune cell. In one embodiments, targeting domain of the orthogonal GD2 immune cell, e.g. orthogonal GD2 CAR-T cells, incorporates the 14g2a scFv which has the amino acid sequences:

(SEQ ID NO: 49)
DVVMTQTPLSLPVSLGDQASISCRSSQSLVHRNGNTYLHWYLQK
PGQSPKLLIHKVSNRFSGVPDRFSGSGSGTDFTLKISRVEAEDL
GVYFCSQSTHVPPLTFGAGTKLELGGGGSGGGGSGGGGSGGGGS
EVQLLQSGPELEKPGASVMISCKASGSSFTGYNMNWVRQNIGKS
LEWIGAIDPYYGGTSYNQKFKGRATLTVDKSSSTAYMHLKSLTS
EDSAVYYCVSGMEYWGQGTSVTVSS

### Making Cell a More Homogenous Cell Product:

The present disclosure provide methods of preparing a cell product comprising an engineered immune cell species, wherein the engineered cell immune species is an immune cell that that has been recombinantly modified to express a receptor comprising the extracellular domain of an orthogonal CD122 polypeptide, and wherein the cell product comprises at least 40%, alternatively at least 50%. alternatively at least 60%, alternatively at least 70%, alternatively at least 80%, alternatively at least 90%, of the engineered immune cells, the method comprising culturing obtaining a population of immune cells, trans selective expanding engineered immune cells expressing an orthogonal CD122 receptor in a mixed cell population of the engineered cell product. Currently the most significant barrier to barrier to the success of adoptive cell therapy and CAR-T therapy in particular is the high rate of disease relapse due to the poor persistence of engineered cell CAR T cells.

The selectivity of the molecule enables the use of the orthogonal ligand ex vivo to selectively proliferate the engineered cells in a mixed cell population. Consequently the technology as described herein is useful in the preparation of cell therapy products that are significantly enriched for therapeutic cells. Conventional non-specific ex vivo stimulation with IL2 in the absence of sorting leads to a cell population for administration to the patient that provides only a comparatively small fraction (10%-20%) of the desirable TIL or CAR cells. The orthogonal IL2 can be used to selectively activate during the preparation phase of the CARs enabling the generation of a cell product with a significantly higher percentage of the therapeutic hoCARs or hoTILs ex vivo. These more homogenous cell products may be used in the treatment of neoplastic disease resulting greater efficacy and less toxicity.

In some embodiments the present disclosure provides a method of treating a disease, disorder or condition in a subject suffering therefrom by administering to the subject:
a. an engineered mammalian cell comprising a nucleic acid sequence encoding a transmembrane receptor molecule comprising an extracellular domain (ECD) of an orthogonal hCD122 operably linked to one or more expression control elements capable of effecting the expression and surface presentation of the ECD of the transmembrane receptor molecule; and
b. administering to said subject a therapeutically effective dose of hIL2 ortholog of Formula #1

In some embodiments the present disclosure provides a preparing an engineered T cell product said T cell product comprising at least 20% of an hoCD122 T cell, the method comprising the steps of:
a. isolated a population a population of T cells from a mammalian subject;
b. contacting the isolated a population of T cells ex vivo with recombinant vector comprising a nucleic acid sequence encoding a hoCD122 operably linked to one or more expression control sequences so as to facilitate expression in a mammalian T cell under conditions that permit uptake of the recombinant vector by a T cell;
c. contacting the isolated a population of T cells an effective amount of an hIL2 ortholog of claim 1.

In some embodiments the present disclosure provides a cell population at least 20% engineered hoCD122 T cells.

The present disclosure further provides methods of making the hIL2 orthologs of the present invention. In particular, the present disclosure provides recombinant expression vectors comprising a nucleic acid sequence encoding the hIL2 orthologs operably linked to control elements to provide for expression of the nucleic acid sequence encoding the hIL2 ortholog in a host cell.

The present disclosure further provides a composition comprising a mixed cell population comprising at least 10%, alternatively at least 20%, alternatively at least 30%, alternatively at least 40%, alternatively at least 50%, alternatively at least 60%, alternatively at least 70%, of a T cell (e.g., T cell, CD8+ T cell, Treg, TIL, NK cell, TCR modified cell, CAR-T cell, etc.), wherein the T cell has been recombinantly modified to express an orthogonal hCD122 receptor polypeptide. The present disclosure further provides a method of generating a pharmaceutically acceptable dosage form of an engineered cell therapy product the dosage form comprising a population of T cells wherein the population of T cells is substantially enriched for one or more species of engineered T cells, the engineered T cells expressing a receptor comprising the extracellular domain of an hCD122 orthogonal polypeptide, the method comprising the steps culturing the population of T cells comprising engineered T cells expressing a receptor comprising the extracellular domain of an hCD122 orthogonal polypeptide ex vivo in the presence of an hIL2 ortholog of the present invention for a period of time sufficient to enrich the cell population in of one or more such engineered T cells.

In some embodiments, the present disclosure provides a recombinant vector comprising a nucleic acid sequence encoding the hIL2 ortholog described herein operably linked to control elements to facilitate expression and secretion of the hIL2 ortholog from a mammalian cell is administered to the subject to provide for in situ expression of the hIL2 ortholog. In some embodiments, the recombinant vector is administered intratumorally to a subject suffering from cancer. In some embodiments, the recombinant vector is a recombinant viral vector. In some embodiments the recombinant viral vector is a recombinant adeno-associated virus (rAAV) or recombinant adenovirus (rAd), for example in some embodiments, a replication deficient adenovirus derived from human adenovirus serotypes 3 and/or 5. In some embodiments, the replication deficient adenovirus has one or more modifications to the E1 region which interfere with the ability of the virus to initiate the cell cycle and/or apoptotic pathways. The replication deficient adenoviral vector may optionally comprise deletions in the E3 domain. In some embodiments the adenovirus is a replication competent adenovirus. In some embodiments the adenovirus is a replication competent recombinant virus engineered to selectively replicate in neoplastic cells.

### IL2 Orthologs

### Nomenclature:

The present disclosure provides a variety of polypeptide ligands of IL2 receptor polypeptide variants. The following nomenclature is used herein to refer to substitutions, deletions or insertions. Residues may be designated herein by the one-letter or three-letter amino acid code of the naturally occurring amino acid found in the wild-type molecule but followed by the IL2 amino acid position of the mature IL2 molecule, e.g., “Cys125” or “C125” refers to the cysteine residue at position 125 of the wild-type hIL2 molecule. In reference to the IL2 orthologs, substitutions are designated herein by the one letter amino acid code followed by the IL2 amino acid position followed by the one letter amino acid code which is substituted. For example, an IL2 ortholog having the modification “K35A” refers to a substitution of the lysine (K) residue at position 35 of the wild-type IL2 sequence with an alanine (A) residue at this position. A deletion of an amino acid reside is referred to as “des” followed by the amino acid residue and its position in SEQ ID NO:4. For example the term “des-Ala1” or “desA1” refers to the deletion of the alanine at position 1 of the polypeptide of wild-type IL2 sequence. Similarly, in reference to amino acid substitutions in the orthogonal CD122, amino acid substitutions are designated herein by the one letter amino acid code of the naturally occurring amino acid followed by the number of its position in the wild-type IL2 sequence followed by the one letter amino acid code of the amino acid which is substituted at that position. For example, the hCD122 ortholog having a substitution of the tyrosine residue at position 134 with a phenylalanine residue, the substitution is abbreviated “Y134F”

Orthologs (Cognate Ligands for Receptors Comprising and Orthogonal CD122 ECD): In some embodiments, the present disclosure provides methods of use of IL2 orthologs that are cognate ligands of receptors comprising an orthogonal CD122 ECD. In some embodiments, the IL2 orthologs are ligands for an orthogonal CD122 the ICD of which comprises one or more STAT3 binding motifs. In some embodiments, the term IL2 orthologs refers to hIL2 variants that ligands for a receptor comprising the extracellular domain of human CD122 comprising amino acid substitutions at positions H133 and/or Y134 the ICD of which optionally comprises one or more STAT3 binding motifs. In some embodiments, the IL2 orthologs are cognate ligands for a receptor comprising the extracellular domain of human CD122 comprising amino acid substitutions at positions H133 and/or Y134. In some embodiments, the IL2 orthologs are ligands for a receptor comprising the extracellular domain of human CD122 comprising amino acid substitutions at positions H133 and/or Y134 the ICD of which comprises one or more STAT3 binding motifs. In some embodiments, the IL2 orthologs are ligands for an orthogonal human CD122 comprising amino acid substitutions at positions H133 and Y134. In some embodiments, the IL2 orthologs are ligands for an orthogonal CD122 comprising the amino acid substitutions H133D and Y134F. In some embodiments, the orthologs of the present disclosure are hIL2 orthologs that are cognate ligands of a receptor comprising the extracellular domain of a hCD122 molecule comprising amino acid substitutions at position H133 and Y134 the ICD of which comprises one or more STAT3 binding motifs.

In some embodiments, the orthologs of the present disclosure are hIL2 orthologs that are cognate ligands of a receptor comprising the extracellular domain of a hCD122 molecule comprising and amino acid substitutions at position H133. In some embodiments, the orthologs of the present disclosure are hIL2 orthologs that are cognate ligands of a receptor comprising the extracellular domain of a hCD122 molecule comprising amino acid substitutions at position H133 the ICD of which comprises one or more STAT3 binding motifs. In some embodiments, the orthologs of the present disclosure are hIL2 orthologs that are cognate ligands of a receptor comprising the extracellular domain of a hCD122 molecule comprising amino acid substitutions at position H133D. In some embodiments, the orthologs of the present disclosure are hIL2 orthologs that are cognate ligands of a receptor comprising the extracellular domain of a hCD122 molecule comprising amino acid substitutions at position H133D the ICD of which comprises one or more STAT3 binding motifs. In some embodiments, the orthologs of the present disclosure are hIL2 orthologs that are cognate ligands of a receptor comprising the extracellular domain of a hCD122 molecule comprising and amino acid substitutions at position Y134. In some embodiments, the orthologs of the present disclosure are hIL2 orthologs that are cognate ligands of a receptor comprising the extracellular domain of a hCD122 molecule comprising amino acid substitutions at position Y134 the ICD of which comprises one or more STAT3 binding motifs. In some embodiments, the orthologs of the present disclosure are hIL2 orthologs that are cognate ligands of a receptor comprising the extracellular domain of a hCD122 molecule comprising amino acid substitutions at position Y134F. In some embodiments, the orthologs of the present disclosure are hIL2 orthologs that are cognate ligands of a receptor comprising the extracellular domain of a hCD122 molecule comprising amino acid substitutions at position Y134F the ICD of which comprises one or more STAT3 binding motifs.

In some embodiments, the orthologs of the present disclosure are hIL2 orthologs that are cognate ligands of a receptor comprising the extracellular domain of a hCD122 molecule comprising amino acid substitutions at position Y134 the ICD of which comprises one or more STAT3 binding motifs. In some embodiments, the orthologs of the present disclosure are hIL2 orthologs that are cognate ligands of receptor comprising the extracellular domain of orthogonal human CD122 comprising the amino acid substitutions H133D and Y134F. In some embodiments, the orthologs of the present disclosure are hIL2 orthologs that are cognate ligands of and orthogonal human CD122 comprising the amino acid substitutions H133D and Y134F.

IL2 Orthologs (FORMULA #1): In one embodiment, the present disclosure provides an hIL2 ortholog, the amino acid sequence of which has at least 80%, 90%, 95%, 98%, 99% or 100% identity to polypeptide of the Formula #1 (SEQ ID NO: 50):

$$[ ( AA 1 ) - ( AA 2 ) ‐ ( AA 3 ) ‐ ( AA 4 ) ‐ ( AA 5 ) ‐ ( AA 6 ) ‐ ( AA 7 ) ‐ ( AA 8 ) ‐ {( AA 9 )}_{i} ‐ T 10 ‐ Q 11 ‐ L 12 ‐ ( AA 13 ) ‐ ( AA 14 ) ‐ ( AA 15 ) ‐ ( AA 16 ) ‐ L 17 ‐ ( AA 18 ) ‐ ( AA 19 ) ‐ ( AA 20 ) ‐ L 21 ‐ ( AA 22 ) ‐ ( AA 23 ) ‐ I 24 ‐ L 25 ‐ N 26 ‐ ( AA 27 ) ‐ I 28 ‐ N 29 ‐ N 30 ‐ Y 31 ‐ K 32 ‐ N 33 ‐ P 34 ‐ K 35 ‐ L 36 ‐ T 37 ‐ ( AA 38 ) ‐ ( AA 39 ) ‐ L 40 ‐ T 41 ‐ ( AA 42 ) ‐ K 43 ‐ F 44 ‐ Y 45 ‐ M 46 ‐ P 47 ‐ K 48 ‐ K 49 ‐ A 50 ‐ ( AA 51 ) ‐ E 52 ‐ L 53 ‐ K 54 ‐ ( AA 55 ) ‐ L 56 ‐ Q 57 ‐ C 58 ‐ L 59 ‐ E 60 ‐ E 61 ‐ E 62 ‐ L 63 ‐ K 64 ‐ P 65 ‐ L 66 ‐ E 67 ‐ E 68 ‐ V 69 ‐ L 70 ‐ N 71 ‐ L 72 ‐ A 73 ‐ ( AA 74 ) ‐ S 75 ‐ K 76 ‐ N 77 ‐ F 78 ‐ H 79 ‐ ( AA 80 ‐ ( AA 81 ) ‐ P 82 ‐ R 83 ‐ D 84 ‐ ( AA 85 ) ‐ ( AA 86 ) ‐ S 87 ‐ N 88 ‐ ( AA 89 ) ‐ N 90 ‐ ( AA 91 ) ‐ ( AA 92 ) ‐ V 93 ‐ L 94 ‐ E 95 ‐ L 96 ‐ ( AA 97 ) ‐ G 98 ‐ S 99 ‐ E 100 ‐ T 101 ‐ T 102 ‐ F 103 ‐ ( AA 104 ) ‐ C 105 ‐ E 106 ‐ Y 107 ‐ A 108 ‐ ( AA 109 ) ‐ E 110 ‐ T 111 ‐ A 112 ‐ ( AA 113 ) ‐ I 114 ‐ V 115 ‐ E 116 ‐ F 117 ‐ L 118 ‐ N 119 ‐ R 120 ‐ W 121 ‐ I 122 ‐ T 123 ‐ F 124 ‐ ( AA 125 ) ‐ ( AA 126 ) ‐ S 127 ‐ I 128 ‐ I 129 ‐ ( AA 130 ) ‐ T 131 ‐ L 132 ‐ T 133 ] \qquad [ 1 ]$$

wherein:
AA1 is A (wild type) or deleted;
AA2 is P (wild type) or deleted;
AA3 is T (wild type), C, A, G, Q, E, N, D, R, K, P, or deleted;
AA4 is S (wild type) or deleted;
AA5 is S (wild type) or deleted;
AA6 is S (wild type) or deleted;
AA7 is T (wild type) or deleted;
AA8 is K (wild type) or deleted;
AA9 is K (wild type) or deleted;
AA13 is Q (wild type), W or deleted;
AA14 is L (wild type), M, W or deleted;
AA15 is E (wildtype), K, D, T, A, S, Q, H or deleted;
AA16 is H (wildtype), N or Q or deleted;
AA18 is L (wild type) or R, L, G, M, F, E, H, W, K, Q, S, V, I, Y, H, D or T;
AA19 is L (wildtype), A, V, I or deleted;
AA20 is D (wildtype), T, S M L, or deleted;
AA22 is Q (wild type) or F, E, G, A, L, M, F, W, K, S, V, I, Y, H, R, N, D, T, F or deleted;
AA23 is M (wild type), A, W, H, Y, F, Q, S, V, L, T, or deleted;
AA27 IS G (wildtype), K, S or deleted;
AA38 is R (wild type), W or G;
AA39 is M (wildtype), L or V;
AA42 is F (wildtype) or K;
AA51 is T (wildtype), I or deleted
AA55 is H (wildtype) or Y;
AA74 is Q (wild type), N, H, S;
AA80 is L (wild type), F or V;
AA81 is R (wild type), I, D, Y, T or deleted
AA85 is L (wild type) or V;
AA86 is I (wild type) or V;
AA89 is I (wild type) or V;
AA91 is V (wild type), R or K;
AA92 is I (wild type) or F;
AA97 is K (wild type) or Q;
AA104 is M (wild type) or A;
AA109 is D (wildtype), C or a non-natural amino acid with an activated side chain;
AA113 is T (wild type) or N;
AA125 is C (wild type), A or S;
AA126 is Q (wild type) or H, M, K, C, D, E, G, I, R, S, or T; and/or
AA130 is S (wild type), T or R.

In some embodiments, the present disclosure provides hIL2 orthologs which are hIL2 polypeptides comprising the following sets of amino acid modifications:
[E15S-H16Q-L19V-D20L-Q22K]
[H16N, L19V, D20N, Q22T, M23H, G27K];
[E15D, H16N, L19V, D20L, Q22T, M23H];
[E15D, H16N, L19V, D20L, Q22T, M23A],
[E15D, H16N, L19V, D20L, Q22K, M23A];
[E15S; H16Q; L19V, D20T; Q22K, M23L];
[E15S; H16Q; L19V, D20T; Q22K, M23S];
[E15S; H16Q; L19V, D20S; Q22K, M23S];
[E15S; H16Q; L191, D20S; Q22K; M23L];
[E15S; L19V; D20M; Q22K; M23S];
[E15T; H16Q; L19V; D20S; M23S];
[E15Q; L19V; D20M; Q22K; M23S];
[E15Q; H16Q; L19V; D20T; Q22K; M23V];
[E15H; H16Q; L191; D20S; Q22K; M23L];
[E15H; H16Q; L191; D20L; Q22K; M23T]; or
[L19V; D20M; Q22N; M23S].

Cys125: In some embodiments, the present disclosure provides hIL2 orthologs to facilitate recombinant expression in bacterial cells by eliminating the unpaired cysteine residue at position 125 and/or elimination of the N-terminal Met of the directly expressed IL2 polypeptide as well as the alanine at position 1 by post-translational processing by endogenous bacterial proteases. When an amino acid is missing, it is referred to as “des”. In some embodiments, the cysteine at position 125 is substituted with alanine or serine (C125A or C125S). Such mutations are typically used to avoid misfolding of the protein when expressed recombinantly in bacteria and isolated from inclusion bodies.

In some embodiments, the IL2 orthologs or the present invention comprise one of the following sets of amino acid modifications:
[E15S-H16Q-L19V-D20L-Q22K-M23A-C125S];
[E15S-H16Q-L19V-D20L-Q22K-C125S];
[E15S-H16Q-L19V-D20L-M23A-C12S];
[E15S-H16Q-L19V-D20L-C12S];
[E15S-H16Q-L19V-D20L-Q22K-M23A-C125A];
[E15S-H16Q-L19V-D20L-M23A-C125A];
[E15S-H16Q-L19V-D20L-Q22K-C125A];
[E15S-H16Q-L19V-D20L-C125A];
[desAla1-E15S-H16Q-L19V-D20L-Q22K-M23A-C125S];
[desAla1-E15S-H16Q-L19V-D20L-Q22K-C125S];
[desAla1-E15S-H16Q-L19V-D20L-C125S];
[desAla1-E15S-H16Q-L19V-D20L-Q22K-M23A-C125A];
[desAla1-E15S-H16Q-L19V-D20L-Q22K-C125A];
[desAla1-E15S-H16Q-L19V-D20L-C125A];
[desAla1-E15S-H16Q-L19V-D20L-Q22K-M23A];
[desAla1-E15S-H16Q-L19V-D20L-M23A];
[desAla1-E15S-H16Q-L19V-D20L-Q22K]; or
[desAla1-E15S-H16Q-L19V-D20L].

### Mutations to Increase CD122 Affinity

In some embodiments, hIL2 orthologs contain one or more mutations in positions of the hIL2 sequence that either contact hCD122 or alter the orientation of other positions contacting CD122, resulting in an IL2 ortholog having increased affinity for CD122. IL2 residues that have been identified as being involved in the binding of IL2 to CD122 include L12, Q13, H16, L19, D20, M23, Q74, L80, R81, D84, L85, 186, S87, N88, I89 V91, I92, and E95. In some embodiments, the IL2 ortholog comprises one or more of the amino acid substitutions: Q74N, Q74H, Q74S, L80F, L80V, R81D, R81T, L85V, I86V, I89V, and/or I92F or combinations thereof. In some embodiments, the IL2 ortholog comprises one or more of the amino acid substitutions: L80F, R81D, L85V, I86V and I92F. In some embodiments, the IL2 ortholog comprises one or more of the amino acid substitutions: N74Q, L80F, R81D, L85V, I86V, I89V, and I92F. In some embodiments, the IL2 ortholog comprises one or more of the amino acid substitutions: Q74N, L80V, R81T, L85V, I86V, and I92F. In some embodiments, the IL2 ortholog comprises one or more of the amino acid substitutions: Q74H, L80F, R81D, L85V, I86V and I92F. In some embodiments, the IL2 ortholog comprises one or more of the amino acid substitutions: Q74S, L80F, R81D, L85V, I86V and I92F. In some embodiments, the IL2 ortholog comprises one or more of the amino acid substitutions: Q74N, L80F, R81D, L85V, I86V and I92F. In some embodiments, the IL2 ortholog comprises one or more of the amino acid substitutions: Q74S, R81T, L85V, and I92F. In some embodiments, the IL2 ortholog comprises [L80F-R81D-L85V-I86V-I92F]. In some embodiments, the present disclosure provides hIL2 orthologs which comprise one of the following sets of amino acid modifications:
[E15S-H16Q-L19V-D20L-M23A-L80E-R81D-L85V-I86V-I92F];
[E15S-H16Q-L19V-D20L-Q22K-L80E-R81D-L85V-I86V-I92F];
[E15S-H16Q-L19V-D20L-Q22K-M23A L80E-R81D-L85V-I86V-I92F];
[E15S-H16Q-L19V-D20L-M23A-L80E-R81D-L85V-I86V-I92F-Q126H];
[E15S-H16Q-L19V-D20L-Q22K-L80E-R81D-L85V-I86V-I92F-Q126H];
[E15S-H16Q-L19V-D20L-Q22K-M23A-L80E-R81D-L85V-I86V-I92F-Q126H];
[E15S-H16Q-L19V-D20L-M23A-L80E-R81D-L85V-I86V-I92F-Q126M];
[E15S-H16Q-L19V-D20L-Q22K-L80E-R81D-L85V-I86V-I92F-Q126M]; or
[E15S-H16Q-L19V-D20L-Q22K-M23A-L80E-R81D-L85V-I86V-I92F-Q126M].

In some embodiments, the orthologs comprise the substitution L85V that has been identified as increasing affinity of IL2 to CD122. In some embodiments, the present disclosure provides hIL2 orthologs which are hIL2 polypeptides comprising one of the following sets of amino acid modifications:
[E15S-H16Q-L19V-D20L-M23A-L85V];
[E15S-H16Q-L19V-D20L-Q22K-M23A-L85V];
[E15S-H16Q-L19V-D20L-M23A-L85V];
[E15S-H16Q-L19V-D20L-Q22K-M23A-L85V];
[E15S-H16Q-L19V-D20L-M23A-L85V-Q126H];
[E15S-H16Q-L19V-D20L-Q22K-M23A-L85V-Q126H];
[E15S-H16Q-L19V-D20L-M23A-L85V-Q126M]; or
[E15S-H16Q-L19V-D20L-Q22K-M23A-L85V-Q126M].

### Modifications to Modulate CD25 Affinity

In some embodiments, the IL2 orthologs contain one or more mutations in positions of the IL2 sequence that either contact CD25 or alter the orientation of other positions contacting CD25 resulting in a decreased affinity for CD25. The mutations may be in or near areas known to be in close proximity to CD25 based on published crystal structures (Wang, et al Science 310:1159 2005). IL2 residues believed to contact CD25 include K35, R38, T41, F42, K43, F44, Y45, E61, E62, K64, P65, E68, V69, L72, and Y107. In some embodiments, the IL2 orthologs of the present disclosure comprise one or more of the point mutations of R38A, F41A and F42A (Suave, et al (1991) PNAS (USA) 88:4636-4640); P65L (Chen et al. Cell Death and Disease (2018) 9:989); F42A/G/S/T/Q/E/N/R/K, Y45A/G/S/T/Q/E/N/D/R/K/and/or L72G/A/S/T/Q/E/N/D/R/K (Ast, et al United States Patent Application Publication 2012/0244112A1 published Sep. 27, 2012; U.S. Pat. No. 9,266,938B2 issued Feb. 23, 2016). Particular combinations of substitutions have been identified as reducing binding to CD25. In some embodiments, the IL2 orthologs of the present disclosure comprise one or more of the of the sets of substitutions [R38A-F42A-Y45A-E62A] as described in Carmenate, et al (2013) J Immunol 190:6230-6238; [F42A-Y45A-L72G] (Roche RG7461 (R06874281); and/or [T41P-T51P] (Chang, et al (1995) Molecular Pharmacology 47:206-211). In some embodiments, the present disclosure provides hIL2 orthologs which are hIL2 polypeptides comprising one of the following sets of amino acid modifications:
[E15S-H16Q-L19V-D20L-M23A-R38A-F42A-Y45A-E62A];
[E15S-H16Q-L19V-D20L-M23A-R38A-F42A-Y45A-E62A];
[E15S-H16Q-L19V-D20L-Q22K-M23A-R38A-F42A-Y45A-E62A];
[E15S-H16Q-L19V-D20L-M23A-R38A-F42A-Y45A-E62A-Q126H];
[E15S-H16Q-L19V-D20L-M23A-R38A-F42A-Y45A-E62A-Q126H];
[E15S-H16Q-L19V-D20L-Q22K-M23A-R38A-F42A-Y45A-E62A-Q126H];
[E15S-H16Q-L19V-D20L-M23A-R38A-F42A-Y45A-E62A-Q126M]; or
[E15S-H16Q-L19V-D20L-M23A-R38A-F42A-Y45A-E62A-Q126M].

### Modifications to Modulate CD132 Affinity

In some embodiments of the invention, the IL2 orthologs contain one or more mutations in positions of the IL2 sequence that either contact CD132 or alter the orientation of other positions contacting CD132 resulting in an altered binding to CD132. Exemplary IL2 orthologs contain one or more mutations in positions of the IL2 sequence that either contact CD132 or alter the orientation of other positions contacting CD122, resulting in an altered binding to CD132. IL2 residues believed to contact CD132 include Q11, L18, Q22, E110, N119, T123, Q126, S127, 1129, S130, and T133. In some embodiments, the IL2 comprises modifications at L18 AA18 is L (wild type) or R, L, G, M, F, E, H, W, K, Q, S, V, I, Y, H, D or T; AA126 is Q (wild type) or H, M, K, C, D, E, G, I, R, S, or T; and/or AA22 is Q (wild type) or F, E, G, A, L, M, F, W, K, S, V, I, Y, H, R, N, D, T, or F.

In some embodiments, the present disclosure provides hIL2 orthologs which are hIL2 polypeptides comprising one the following sets of amino acid modifications:
[E15S-H16Q-L19V-D20L-M23A-Q126H];
[E15S-H16Q-L19V-D20L-Q22K-M23A-Q126H];
[E15S-H16Q-L19V-D20L-Q22K-Q126H];
[E15S-H16Q-L19V-D20L-M23A-Q126M];
[E15S-H16Q-L19V-D20L-Q22K-M23A-Q126M];
[E15S-H16Q-L19V-D20L-Q22K-Q126M];
[desAla1-E15S-H16Q-L19V-D20L-Q126M];
[desAla1-E15S-H16Q-L19V-D20L-Q22K-Q126M];
[desAla1-E15S-H16Q-L19V-D20L-M23A-Q126M];
[desAla1-E15S-H16Q-L19V-D20L-Q22K-M23A-Q126M];
[desAla1-E15S-H16Q-L19V-D20L-Q22K-M23A-Q126M];
[E15S-H16Q-L19V-D20L-M23A-L80E-R81D-I86V-I92F-Q126H];
[E15S-H16Q-L19V-D20L-Q22K-L80E-R81D-I86V-I92F-Q126H];
[E15S-H16Q-L19V-D20L-Q22K-M23A-L80E-R81D-I86V-I92F-Q126H];
[E15S-H16Q-L19V-D20L-M23A-L80E-R81D-I86V-I92F-Q126M];
[E15S-H16Q-L19V-D20L-Q22K-M23A-L80E-R81D-I86V-I92F-Q126M];
[E15S-H16Q-L19V-D20L-M23A-L85V-Q126H];
[E15S-H16Q-L19V-D20L-Q22K-L85V-Q126H];
[E15S-H16Q-L19V-D20L-Q22K-M23A-L85V-Q126H1;
[E15S-H16Q-L19V-D20L-M23A-L85V-Q126M];
[E15S-H16Q-L19V-D20L-Q22K-L85V-Q126H]; or
[E15S-H16Q-L19V-D20L-Q22K-M23A-L85V-Q126M].

When produced recombinantly in bacterial expression systems directly in the absence of a leader sequence, endogenous proteases result in the deletion of the N-terminal Met-Ala1 residues to provide “desAla1” IL2 orthologs. In some embodiments, the present disclosure provides hIL2 orthologs which are hIL2 polypeptides comprising one of the following sets of amino acid modifications:
[desAla1-E15S-H16Q-L19V-D20L];
[desAla1-E15S-H16Q-L19V-D20L-Q22K];
[desAla1-E15S-H16Q-L19V-D20L-Q22K-M23A];
[desAla1-E15S-H16Q-L19V-D20L-Q126H];
[desAla1-E15S-H16Q-L19V-D20L-Q22K-Q126H];
[desAla1-E15S-H16Q-L19V-D20L-Q22K-M23A-Q126H];
[desAla1-E15S-H16Q-L19V-D20L-Q126M];
[desAla1-E15S-H16Q-L19V-D20L-Q22K-Q126H];
[desAla1-E15S-H16Q-L19V-D20L-Q22K-M23A-Q126H];
[desAla1-E15S-H16Q-L19V-D20L-C125A];
[desAla1-E15S-H16Q-L19V-D20L-Q22K-C125A];
[desAla1-E15S-H16Q-L19V-D20L-Q22K-M23A-C125A];
[desAla1-E15S-H16Q-L19V-D20L-C125A-Q126H];
[desAla1-E15S-H16Q-L19V-D20L-Q22K-C125A-Q126H];
[desAla1-E15S-H16Q-L19V-D20L-Q22K-M23A-C125A-Q126H];
[desAla1-E15S-H16Q-L19V-D20L-C125A-Q126M];
[desAla1-E15S-H16Q-L19V-D20L-Q22K-C125A-Q126H];
[desAla1-E15S-H16Q-L19V-D20L-Q22K-M23A-C125A-Q126H];
[desAla1-E15S-H16Q-L19V-D20L-C125S];
[desAla1-E15S-H16Q-L19V-D20L-Q22K-C125 S];
[desAla1-E15S-H16Q-L19V-D20L-Q22K-M23A-C125S];
[desAla1-E15S-H16Q-L19V-D20L-C125S-Q126H];
[desAla1-E15S-H16Q-L19V-D20L-Q22K-C125S-Q126H];
[desAla1-E15S-H16Q-L19V-D20L-Q22K-M23A-C125S-Q126H];
[desAla1-E15S-H16Q-L19V-D20L-C125 S-Q126M];
[desAla1-E15S-H16Q-L19V-D20L-Q22K-C125S-Q126M]; or
[desAla1-E15S-H16Q-L19V-D20L-Q22K-M23A-C125S-Q126M].

### Conservative Amino Acid Substitutions

In addition to the foregoing modifications that contribute to the activity and selectivity of the IL2 ortholog for the CD122 orthogonal receptor, the IL2 ortholog may comprise one or more modifications to its primary structure that provide minimal effects on the activity IL2. In some embodiments, the IL2 orthologs of the present disclosure may further comprise one more conservative amino acid substitution within the wild type IL-2 amino acid sequence. Such conservative substitutions include those described by Dayhoff in The Atlas of Protein Sequence and Structure 5 (1978), and by Argos in EMBO J., 8:779-785 (1989). Conservative substitutions are generally made in accordance with the following chart depicted as Table XXX

TABLE X
Exemplary Conservative Amino Acid Substitutions

| Wild type Residue | Substitution(s) |
|---|---|
| Ala | Ser |
| Arg | Lys |
| Asn | Gln, His |
| Asp | Glu |
| Cys | Ser, Ala |
| Gln | Asn |
| Glu | Asp |
| Gly | Pro |
| His | Asn, Gln |
| Ile | Leu, Val |
| Leu | Ile, Val |
| Lys | Arg, Gln, Glu, Met, Leu, Ile |
| Phe | Met, Leu, Tyr, Trp |
| Ser | Thr |
| Thr | Ser |
| Trp | Tyr, Phe |
| Tyr | Trp, Phe |
| Val | Ile, Leu |

Substantial changes in function or immunological identity may be made by selecting amino acid substitutions that are less conservative than those indicated in Table 3. For example substitutions may be made which more significantly affect the structure of the polypeptide backbone or disrupt secondary or tertiary elements including the substitution of an amino acid with a small uncharged side chain (e.g. glycine) with a large charge bulky side chain (asparagine). In particular, substitution of those IL2 residues which are involved in the amino acids that interact with one or more of CD25, CD122 and/or CD123 as may be discerned from the crystal structure of IL2 in association with its receptors as described in

In addition to the foregoing modifications that contribute to the activity and selectivity of the IL2 ortholog for the CD122 orthogonal receptor, the IL2 ortholog may comprise one or more modifications to its primary structure. Modifications to the primary structure as provided above may optionally further comprise modifications do not substantially diminish IL2 activity of the IL2 ortholog including but not limited to the substitutions: N30E; K32E; N33D; P34G; T37I, M39Q, F42Y, F44Y, P47G, T51I, E52K, L53N, Q57E, M104A (see U.S. Pat. No. 5,206,344).

### Removal of Glycosylation Site

The IL2 orthologs of the present disclosure may comprises comprise modifications to eliminate the O-glycosylation site at position Thr3 to facilitate the production of an aglycosylated IL2 ortholog when the IL2 ortholog expressed in mammalian cells such as CHO or HEK cells. Thus, in certain embodiments the IL2 ortholog comprise a modification which eliminates the O-glycosylation site of IL-2 at a position corresponding to residue 3 of human IL-2. In one embodiment said modification which eliminates the O-glycosylation site of IL-2 at a position corresponding to residue 3 of human IL-2 is an amino acid substitution. Exemplary amino acid substitutions include T3A, T3G, T3Q, T3E, T3N, T3D, T3R, T3K, and T3P which removes the glycosylation site at position 3 without eliminating biological activity (see U.S. Pat. No. 5,116,943; Weiger et al., (1989) Eur. J. Biochem., 180:295-300). In a specific embodiment, said modification is the amino acid substitution T3A. In some embodiments, the present disclosure provides hIL2 orthologs which are hIL2 polypeptides comprising one of the following sets of amino acid modifications:
[T3A-E15S-H16Q-L19V-D20L-Q22K-M23A-C125S];
[T3A-E15S-H16Q-L19V-D20L-Q22K-C125S];
[T3A-E15S-H16Q-L19V-D20L-M23A-C125S];
[T3A-E15S-H16Q-L19V-D20L-C125S];
[T3A-E15S-H16Q-L19V-D20L-Q22K-M23A-C125A];
[T3A-E15S-H16Q-L19V-D20L-M23A-C125A];
[T3A-E15S-H16Q-L19V-D20L-Q22K-C125A];
[T3A-E15S-H16Q-L19V-D20L-C125A];
[T3A-E15S-H16Q-L19V-D20L-Q22K-M23A];
[T3A-E15S-H16Q-L19V-D20L-M23A];
[T3A-E15S-H16Q-L19V-D20L-Q22K];
[T3A-E15S-H16Q-L19V-D20L];
[desAla1-T3A-E15S-H16Q-L19V-D20L-Q22K-M23A-C125S];
[desAla1-T3A-E15S-H16Q-L19V-D20L-M23A-C125S];
[desAla1-T3A-E15S-H16Q-L19V-D20L-Q22K-C125S];
[desAla1-T3A-E15S-H16Q-L19V-D20L-C125S];
[desAla1-T3A-E15S-H16Q-L19V-D20L-Q22K-M23A-C125A];
[desAla1-T3A-E15S-H16Q-L19V-D20L-M23A-C125A];
[desAla1-T3A-E15S-H16Q-L19V-D20L-Q22K-C125A];
[desAla1-T3A-E15S-H16Q-L19V-D20L-C125A];
[desAla1-T3A-E15S-H16Q-L19V-D20L-Q22K-M23A];
[desAla1-T3A-E15S-H16Q-L19V-D20L-M23A];
[desAla1-T3A-E15S-H16Q-L19V-D20L-Q22K]; or
[desAla1-T3A-E15S-H16Q-L19V-D20L].

IL2 orthologs may comprise deletion of the first two amino acids (desAla1-desPro2) as well as substitution of the Thr3 glycosylation with a cysteine residue to facilitate for selective N-terminal modification, especially PEGylation of the sulfhydryl group of the cysteine (See, e.g. Katre, et al. U.S. Pat. No. 5,206,344 issued Apr. 27, 1993). In some embodiments, the present disclosure provides hIL2 orthologs which are hIL2 polypeptides comprising one of the following sets of amino acid modifications:
[desAla1-desPro2-T3C-E15S-H16Q-L19V-D20L-Q22K-M23A-C125S];
[desAla1-desPro2-T3C-E15S-H16Q-L19V-D20L-Q22K-C125S];
[desAla1-desPro2-T3C E15S-H16Q-L19V-D20L-M23A-C125S];
[desAla1-desPro2-T3C-E15S-H16Q-L19V-D20L-C125S];
[desAla1-desPro2-T3C-E15S-H16Q-L19V-D20L-Q22K-M23A-C125A];
[desAla1-desPro2-T3C-E15S-H16Q-L19V-D20L-Q22K-C125A];
[desAla1-desPro2-T3C-E15S-H16Q-L19V-D20L-M23A-C125A];
[desAla1-desPro2-T3C-E15S-H16Q-L19V-D20L-C125A];
[desAla1-desPro2-T3C-E15S-H16Q-L19V-D20L-Q22K-M23A];
[desAla1-desPro2-T3C-E15S-H16Q-L19V-D20L-Q22K];
[desAla1-desPro2-T3C-E15S-H16Q-L19V-D20L-M23A]; or
[desAla1-desPro2-T3C-E15S-H16Q-L19V-D20L].

### Oxidation Stabilized M104A:

The IL2 orthologs may optionally further comprise a modification at position M104, in one embodiment the substitution of methionine 104 with an alanine residue (M104A) to provide a more oxidation-resistant ortholog (See Koths, et al. U.S. Pat. No. 4,752,585 issued Jun. 21, 1988).

### N Terminal Deletions:

When produced recombinantly in bacterial expression systems directly in the absence of a leader sequence, endogenous proteases result in the deletion of the N-terminal Met-Ala1 residues to provide “desAla1” IL2 orthologs. IL2 orthologs may comprise deletion of the first two amino acids (desAla1-desPro2) as well as substitution of the Thr3 glycosylation with a cysteine residue (T3C) to facilitate for N-terminal modification, especially PEGylation of the sulfhydryl group of the cysteine (See, e.g. Katre, et al. U.S. Pat. No. 5,206,344 issued Apr. 27, 1993).

The IL2 orthologs may further comprise elimination of N-terminal amino acids at one or more of positions 1-9, alternatively positions 1-8, alternatively positions 1-7 alternatively positions 1-6, alternatively positions 1-5, alternatively positions 1-4, alternatively positions 1-3, alternatively positions 1-2. In some embodiments, the present disclosure provides hIL2 orthologs which are hIL2 polypeptides comprising one of the following sets of amino acid modifications:
[desAla1-desPro2-E15S-H16Q-L19V-D20L-Q22K-M23A];
[desAla1-desPro2-E15S-H16Q-L19V-D20L-Q22K];
[desAla1-desPro2-E15S-H16Q-L19V-D20L-M23A];
[desAla1-desPro2-E15S-H16Q-L19V-D20L];
[desAla1-desPro2-desThr3-E15S-H16Q-L19V-D20L-Q22K-M23A];
[desAla1-desPro2-desThr3-E15S-H16Q-L19V-D20L-Q22K];
[desAla1-desPro2-desThr3-E15S-H16Q-L19V-D20L-M23A];
[desAla1-desPro2-desThr3-E15S-H16Q-L19V-D20L];
[desAla1-desPro2-desThr3-desSer4-E15S-H16Q-L19V-D20L-Q22K-M23A];
[desAla1-desPro2-desThr3-desSer4-E15S-H16Q-L19V-D20L-Q22K];
[desAla1-desPro2-desThr3-desSer4-E15S-H16Q-L19V-D20L-M23A];
[desAla1-desPro2-desThr3-desSer4-E15S-H16Q-L19V-D20L];
[desAla1-desPro2-desThr3-desSer4-desSer5-E15S-H16Q-L19V-D20L-Q22K-M23A];
[desAla1-desPro2-desThr3-desSer4-desSer5-E15S-H16Q-L19V-D20L-Q22K];
[desAla1-desPro2-desThr3-desSer4-desSer5-E15S-H16Q-L19V-D20L-M23A];
[desAla1-desPro2-desThr3-desSer4-desSer5-E15S-H16Q-L19V-D20L];
[desAla1-desPro2-desThr3-desSer4-desSer5-desSer6-E15S-H16Q-L19V-D20L-Q22K-M23A];
[desAla1-desPro2-desThr3-desSer4-desSer5-desSer6-E15S-H16Q-L19V-D20L-Q22K];
[desAla1-desPro2-desThr3-desSer4-desSer5-desSer6-E15S-H16Q-L19V-D20L-M23A]; or
[desAla1-desPro2-desThr3-desSer4-desSer5-desSer6-E15S-H16Q-L19V-D20L].

### Modifications to Minimize Vascular Leak Syndrome

In some embodiments of the disclosure, the IL2 ortholog comprises amino acid substitutions to avoid vascular leak syndrome, a substantial negative and dose limiting side effect of the use of IL2 therapy in human beings without out substantial loss of efficacy. See, Epstein, et al., U.S. Pat. No. 7,514,073B2 issued Apr. 7, 2009. Examples of such modifications which are included in the IL2 orthologs of the present disclosure include one or more of R38W, R38G, R39L, R39V, F42K, and H55Y.

### Affinity Maturation:

In some embodiments, IL2 orthologs may be affinity matured to enhance their activity with respect to the orthogonal CD122. An “affinity matured” polypeptide is one having one or more alteration(s) in one or more residues which results in an improvement in the affinity of the orthogonal polypeptide for the cognate orthogonal receptor, or vice versa, compared to a parent polypeptide which does not possess those alteration(s). Affinity maturation can be done to increase the binding affinity of the IL2 ortholog by at least about 10%, alternatively at least about 50%, alternatively at least about 100% alternatively at least about 150%, or from 1 to 5-fold as compared to the “parent” polypeptide. An engineered IL2 ortholog of the present invention activates its cognate orthogonal receptor, as discussed above, but has significantly reduced binding and activation of the wild-type IL2 receptor when assessed by ELISA and/or FACS analysis using sufficient amounts of the molecules under suitable assay conditions.

### Modifications to Extend Duration of Action In Vivo

As discussed above, the compositions of the present disclosure include IL2 orthologs that have been modified to provide for an extended lifetime in vivo and/or extended duration of action in a subject. Such modifications to provided extended lifetime and/or duration of action include modifications to the primary sequence of the IL2 ortholog, conjugation to carrier molecules, (e.g. albumin, acylation, PEGylation), and Fc fusions.

### Sequence Modifications to Extend Duration of Action In Vivo

As discussed above, the term IL2 ortholog includes modifications of the IL2 ortholog to provide for an extended lifetime in vivo and/or extended duration of action in a subject.

In some embodiments, the IL2 ortholog may comprise certain amino acid substitutions that result in prolonged in vivo lifetime. For example, Dakshinamurthi, et al. (International Journal of Bioinformatics Research (2009) 1(2):4-13) state that one or more of the substitutions in the IL2 polypeptide V91R, K97E and T113N will result in an IL2 variant possessing enhanced stability and activity. In some embodiments, the IL2 orthologs of the present disclosure comprise one, two or all three of the V91R, K97E and T113N modifications.

### Conjugates and Carrier Molecules

In some embodiments the IL2 ortholog is modified to provide certain properties to the IL2 ortholog (e.g. extended duration of action in a subject) which may be achieve through conjugation to carrier molecules to provide desired pharmacological properties such as extended half-life. In some embodiments, the IL2 ortholog can be covalently linked to the Fc domain of IgG, albumin, or other molecules to extend its half-life, e.g. by PEGylation, glycosylation, fatty acid acylation, and the like as known in the art.

### Albumin Fusions

In some embodiments, the IL2 ortholog is expressed as a fusion protein with an albumin molecule (e.g. human serum albumin) which is known in the art to facilitate extended exposure in vivo.

In one embodiment of the invention, the hIL2 ortholog is conjugated to albumin referred to herein as an “IL2 ortholog albumin fusion.” The term “albumin” as used in the context hIL2 ortholog albumin fusions include albumins such as human serum albumin (HSA), cyno serum albumin, and bovine serum albumin (BSA). In some embodiments, the HSA the HSA comprises a C34S or K573P amino acid substitution relative to the wild type HSA sequence According to the present disclosure, albumin can be conjugated to a hIL2 ortholog at the carboxyl terminus, the amino terminus, both the carboxyl and amino termini, and internally (see, e.g., U.S. Pat. Nos. 5,876,969 and 7,056,701). In the HSA-hIL2 ortholog polypeptide conjugates contemplated by the present disclosure, various forms of albumin can be used, such as albumin secretion pre-sequences and variants thereof, fragments and variants thereof, and HSA variants. Such forms generally possess one or more desired albumin activities. In additional embodiments, the present disclosure involves fusion proteins comprising a hIL2 ortholog polypeptide fused directly or indirectly to albumin, an albumin fragment, and albumin variant, etc., wherein the fusion protein has a higher plasma stability than the unfused drug molecule and/or the fusion protein retains the therapeutic activity of the unfused drug molecule. In some embodiments, the indirect fusion is effected by a linker such as a peptide linker or modified version thereof as more fully discussed below.

Alternatively, the hIL2 ortholog albumin fusion comprises IL2 orthologs that are fusion proteins which comprise an albumin binding domain (ABD) polypeptide sequence and an IL2 ortholog polypeptide. As alluded to above, fusion proteins which comprise an albumin binding domain (ABD) polypeptide sequence and an hIL2 ortholog polypeptide can, for example, be achieved by genetic manipulation, such that the nucleic acid coding for HSA, or a fragment thereof, is joined to the nucleic acid coding for the one or more IL2 ortholog sequences. In some embodiments, the albumin-binding peptide comprises the amino acid sequence DICLPRWGCLW (SEQ ID NO: 22).

The IL2 ortholog polypeptide can also be conjugated to large, slowly metabolized macromolecules such as proteins; polysaccharides, such as sepharose, agarose, cellulose, or cellulose beads; polymeric amino acids such as polyglutamic acid, or polylysine; amino acid copolymers; inactivated virus particles; inactivated bacterial toxins such as toxoid from diphtheria, tetanus, cholera, or leukotoxin molecules; inactivated bacteria, dendritic cells, thyroglobulin; tetanus toxoid; Diphtheria toxoid; polyamino acids such as poly(D-lysine:D-glutamic acid); VP6 polypeptides of rotaviruses; influenza virus hemaglutinin, influenza virus nucleoprotein; Keyhole Limpet Hemocyanin (KLH); and hepatitis B virus core protein and surface antigen Such conjugated forms, if desired, can be used to produce antibodies against a polypeptide of the present disclosure.

In some embodiments, the IL2 ortholog is conjugated (either chemically or as a fusion protein) with an XTEN which provides extended duration of akin to PEGylation and may be produced as a recombinant fusion protein in E. coli. XTEN polymers suitable for use in conjunction with the IL2 orthologs of the present disclosure are provided in Podust, et al. (2016) “Extension of in vivo half-life of biologically active molecules by XTEN protein polymers”, J Controlled Release 240:52-66 and Haeckel et al. (2016) “XTEN as Biological Alternative to PEGylation Allows Complete Expression of a Protease-Activatable Killin-Based Cytostatic” PLOS ONE DOI:10.1371/journal.pone.0157193 Jun. 13, 2016. The XTEN polymer may fusion protein may incorporate a protease sensitive cleavage site between the XTEN polypeptide and the IL2 ortholog such as an MMP-2 cleavage site.

Additional candidate components and molecules for conjugation include those suitable for isolation or purification. Particular non-limiting examples include binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes.

In some embodiments, the IL-2 mutein also may be linked to additional therapeutic agents including therapeutic compounds such as anti-inflammatory compounds or antineoplastic agents, therapeutic antibodies (e.g. Herceptin), immune checkpoint modulators, immune checkpoint inhibitors (e.g. anti-PD1 antibodies), cancer vaccines as described elsewhere in this disclosure. Anti-microbial agents include aminoglycosides including gentamicin, antiviral compounds such as rifampicin, 3′-azido-3′-deoxythymidine (AZT) and acylovir, antifungal agents such as azoles including fluconazole, plyre macrolides such as amphotericin B, and candicidin, anti-parasitic compounds such as antimonials, and the like. The IL2 ortholog may be conjugated to additional cytokines as CSF, GSF, GMCSF, TNF, erythropoietin, immunomodulators or cytokines such as the interferons or interleukins, a neuropeptide, reproductive hormones such as HGH, FSH, or LH, thyroid hormone, neurotransmitters such as acetylcholine, hormone receptors such as the estrogen receptor. Also included are non-steroidal anti-inflammatories such as indomethacin, salicylic acid acetate, ibuprofen, sulindac, piroxicam, and naproxen, and anesthetics or analgesics. Also included are radioisotopes such as those useful for imaging as well as for therapy.

The IL2 orthologs of the present disclosure may be chemically conjugated to such carrier molecules using well known chemical conjugation methods. Bi-functional cross-linking reagents such as homofunctional and heterofunctional cross-linking reagents well known in the art can be used for this purpose. The type of cross-linking reagent to use depends on the nature of the molecule to be coupled to IL-2 mutein and can readily be identified by those skilled in the art. Alternatively, or in addition, the IL2 ortholog and/or the molecule to which it is intended to be conjugated may be chemically derivatized such that the two can be conjugated in a separate reaction as is also well known in the art.

### PEGylation:

In some embodiments, the IL2 ortholog is conjugated to one or more water-soluble polymers. Examples of water soluble polymers useful in the practice of the present invention include polyethylene glycol (PEG), poly-propylene glycol (PPG), polysaccharides (polyvinylpyrrolidone, copolymers of ethylene glycol and propylene glycol, poly(oxyethylated polyol), polyolefinic alcohol, polysaccharides, poly-alpha-hydroxy acid, polyvinyl alcohol (PVA), polyphosphazene, polyoxazolines (POZ), poly(N-acryloylmorpholine), or a combination thereof.

In some embodiments the IL2 ortholog is conjugated to one or more polyethylene glycol molecules or “PEGylated.” Although the method or site of PEG attachment to IL2 ortholog may vary, in certain embodiments the PEGylation does not alter, or only minimally alters, the activity of the IL2 ortholog.

In some embodiments, a cysteine may be substituted for the threonine at position 3 (3TC) to facilitate N-terminal PEGylation using particular chemistries.

In some embodiments, selective PEGylation of the IL2 ortholog (for example by the incorporation of non-natural amino acids having side chains to facilitate selective PEG conjugation chemistries as described Ptacin, et al., (PCT International Application No. PCT/US2018/045257 filed Aug. 3, 2018 and published Feb. 7, 2019 as International Publication Number WO 2019/028419A1 may be employed to generate an IL2 ortholog with having reduced affinity for one or more subunits (e.g. CD25, CD132) of an IL2 receptor complex. For example, an hIL2 ortholog incorporating non-natural amino acids having a PEGylatable specific moiety at those sequences or residues of IL2 identified as interacting with CD25 including amino acids 34-45, 61-72 and 105-109 typically provides an IL2 ortholog having diminished binding to CD25. Similarly, an hIL2 ortholog incorporating non-natural amino acids having a PEGylatable specific moiety at those sequences or residues of IL2 identified as interacting with hCD132 including amino acids 18, 22, 109, 126, or from 119-133 provides an IL2 ortholog having diminished binding to hCD132.

In certain embodiments, the increase in half-life is greater than any decrease in biological activity. PEGs suitable for conjugation to a polypeptide sequence are generally soluble in water at room temperature, and have the general formula R(O-CH2-CH*0-R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure.

A molecular weight of the PEG used in the present disclosure is not restricted to any particular range. The PEG component of the PEG-IL2 ortholog can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa or from about 10 kDa to about 30 kDa. Linear or branched PEG molecules having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, alternatively about 30,000 to about 40,000 daltons. In one embodiment of the invention, the PEG is a 40 kD branched PEG comprising two 20 kD arms.

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods known in the art. Chromatography may be used to resolve conjugate fractions, and a fraction is then identified which contains the conjugate having, for example, the desired number of PEGs attached, purified free from unmodified protein sequences and from conjugates having other numbers of PEGs attached.

PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons.

Two widely used first generation activated monomethoxy PEGs (mPEGs) are succinimdyl carbonate PEG (SC-PEG; see, e.g., Zalipsky, et al. (1992) Biotehnol. Appl. Biochem 15:100-114) and benzotriazole carbonate PEG (BTC-PEG; see, e.g., Dolence, et al. U.S. Pat. No. 5,650,234), which react preferentially with lysine residues to form a carbamate linkage but are also known to react with histidine and tyrosine residues. Use of a PEG-aldehyde linker targets a single site on the N-terminus of a polypeptide through reductive amination.

Pegylation most frequently occurs at the α-amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General pegylation strategies known in the art can be applied herein.

The PEG can be bound to an IL2 ortholog of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide.

In some embodiments, the PEGylation of IL2 orthologs is facilitated by the incorporation of non-natural amino acids bearing unique side chains to facilitate site specific PEGylation. The incorporation of non-natural amino acids into polypeptides to provide functional moieties to achieve site specific pegylation of such polypeptides is known in the art. See e.g. Ptacin, et al., (PCT International Application No. PCT/US2018/045257 filed Aug. 3, 2018 and published Feb. 7, 2019 as International Publication Number WO 2019/028419A1. In one embodiment, the IL2 orthologs of the present invention incorporate a non-natural amino acid at position D109 of the IL2 ortholog. In one embodiment of the invention the IL2 ortholog is a PEGylated at position 109 of the IL2 ortholog to a PEG molecule having a molecular weight of about 20 kD, alternatively about 30 kD, alternatively about 40 kD.

The PEG conjugated to the polypeptide sequence can be linear or branched. Branched PEG derivatives, “star-PEGs” and multi-armed PEGs are contemplated by the present disclosure. Specific embodiments PEGs useful in the practice of the present invention include a 10 kDa linear PEG-aldehyde (e.g., Sunbright® ME-100AL, NOF America Corporation, One North Broadway, White Plains, N.Y. 10601 USA), 10 kDa linear PEG-NHS ester (e.g., Sunbright® ME-100CS, Sunbright® ME-100AS, Sunbright® ME-100GS, Sunbright® ME-100HS, NOF), a 20 kDa linear PEG-aldehyde (e.g. Sunbright® ME-200AL, NOF, a 20 kDa linear PEG-NHS ester (e.g., Sunbright® ME-200CS, Sunbright® ME-200AS, Sunbright® ME-200GS, Sunbright® ME-200HS, NOF), a 20 kDa 2-arm branched PEG-aldehyde the 20 kDA PEG-aldehyde comprising two 10kDA linear PEG molecules (e.g., Sunbright® GL2-200AL3, NOF), a 20 kDa 2-arm branched PEG-NHS ester the 20 kDA PEG-NHS ester comprising two 10kDA linear PEG molecules (e.g., Sunbright® GL2-200TS, Sunbright® GL200GS2, NOF), a 40 kDa 2-arm branched PEG-aldehyde the 40 kDA PEG-aldehyde comprising two 20kDA linear PEG molecules (e.g., Sunbright® GL2-400AL3), a 40 kDa 2-arm branched PEG-NHS ester the 40 kDA PEG-NHS ester comprising two 20kDA linear PEG molecules (e.g., Sunbright® GL2-400AL3, Sunbright® GL2-400GS2, NOF), a linear 30 kDa PEG-aldehyde (e.g., Sunbright® ME-300AL) and a linear 30 kDa PEG-NHS ester.

As previously noted, the PEG may be attached directly to the IL2 ortholog or via a linker molecule. Suitable linkers include “flexible linkers” which are generally of sufficient length to permit some movement between the modified polypeptide sequences and the linked components and molecules. The linker molecules are generally about 6-50 atoms long. The linker molecules can also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids. Examples of flexible linkers include glycine polymers (G)n, glycine-serine polymers, glycine-alanine polymers, alanine-serine polymers, and other flexible linkers. Glycine and glycine-serine polymers are relatively unstructured, and therefore can serve as a neutral tether between components. Further examples of flexible linkers include glycine polymers (G)n, glycine-alanine polymers, alanine-serine polymers, glycine-serine polymers. Glycine and glycine-serine polymers are relatively unstructured, and therefore may serve as a neutral tether between components. A multimer (e.g., 1, 2, 3, 4, 5, 6, 7, 8, 9, 10, 10-20, 20-30, or 30-50) of these linker sequences may be linked together to provide flexible linkers that may be used to conjugate a heterologous amino acid sequence to the polypeptides disclosed herein.

Further, such linkers may be used to link the IL2 ortholog to additional heterologous polypeptide components as described herein, the heterologous amino acid sequence may be a signal sequence and/or a fusion partner, such as, albumin, Fc sequence, and the like.

In one embodiment of the disclosure, the IL2 ortholog is a human IL2 ortholog of the structure:

[PEG]-[linker]ₙ-[hoIL2]

wherein n=0 or 1, or

[PEG]-[linker]ₙ-[desAla1-hIL2[E15S-H16Q-L19V-D20L-Q22K-M23A]

wherein n=0 or 1, or

In another embodiment of the invention, the IL2 ortholog is a human IL2 ortholog of the structure

(SEQ ID NO: 23)
40 kD-PEG-(linker)n-PTSSSIKKTQLQLSQLLVLLKAIL
NGINNYKNPKLTRMLTFKFYMPKKATELKHLQCLEEELKPLEEV
LNLAQSKNFHLRPRDLISNINVIVLELKGSETTFMCEYADETAT
IVEFLNRWITFCQSIISTLT

wherein n=0 or 1.

### Acylation

In some embodiments, the IL2 ortholog of the present disclosure may be acylated by conjugation to a fatty acid molecule as described in Resh (2016) Progress in Lipid Research 63: 120-131. Examples of fatty acids that may be conjugated include myristate, palmitate and palmitoleic acid. Myristoylate is typically linked to an N-terminal glycine but lysines may also be myristoylated. Palmitoylation is typically achieved by enzymatic modification of free cysteine —SH groups such as DHHC proteins catalyze S-palmitoylation. Palmitoleylation of serine and threonine residues is typically achieved enzymatically using PORCN enzymes.

### Acetylation

In some embodiments, the IL-2 mutein is acetylated at the N-terminus by enzymatic reaction with N-terminal acetyltransferase and, for example, acetyl CoA. Alternatively, or in addition to N-terminal acetylation, the IL-2 mutein is acetylated at one or more lysine residues, e.g. by enzymatic reaction with a lysine acetyltransferase. See, for example Choudhary et al. (2009) Science 325 (5942):834L2 ortho840.

### Fc Fusions

In some embodiments, the IL2 fusion protein may incorporate an Fc region derived from the IgG subclass of antibodies that lacks the IgG heavy chain variable region. The “Fc region” can be a naturally occurring or synthetic polypeptide that is homologous to the IgG C-terminal domain produced by digestion of IgG with papain. IgG Fc has a molecular weight of approximately 50 kDa. The mutant IL-2 polypeptides can include the entire Fc region, or a smaller portion that retains the ability to extend the circulating half-life of a chimeric polypeptide of which it is a part. In addition, full-length or fragmented Fc regions can be variants of the wild type molecule. That is, they can contain mutations that may or may not affect the function of the polypeptides; as described further below, native activity is not necessary or desired in all cases. In certain embodiments, the IL-2 mutein fusion protein (e.g., an IL-2 partial agonist or antagonist as described herein) includes an IgG1, IgG2, IgG3, or IgG4 Fc region. Exemplary Fc regions can include a mutation that inhibits complement fixation and Fc receptor binding, or it may be lytic, i.e., able to bind complement or to lyse cells via another mechanism such as antibody-dependent complement lysis (ADCC).

In some embodiments, the IL2 ortholog comprises a functional domain of an Fc-fusion chimeric polypeptide molecule. Fc fusion conjugates have been shown to increase the systemic half-life of biopharmaceuticals, and thus the biopharmaceutical product can require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates. The “Fc region” useful in the preparation of Fc fusions can be a naturally occurring or synthetic polypeptide that is homologous to an IgG C-terminal domain produced by digestion of IgG with papain. IgG Fc has a molecular weight of approximately 50 kDa. The IL2 orthologs may provide the entire Fc region, or a smaller portion that retains the ability to extend the circulating half-life of a chimeric polypeptide of which it is a part. In addition, full-length or fragmented Fc regions can be variants of the wild type molecule. In a typical presentation, each monomer of the dimeric Fc carries a heterologous polypeptide, the heterologous polypeptides being the same or different.

In some embodiments, when the IL2 ortholog is to be administered in the format of an Fc fusion, particularly in those situations when the polypeptide chains conjugated to each subunit of the Fc dimer are different, the Fc fusion may be engineered to possess a “knob-into-hole modification.” The knob-into-hole modification is more fully described in Ridgway, et al. (1996) Protein Engineering 9(7):617-621 and U.S. Pat. No. 5,731,168, issued Mar. 24, 1998. The knob-into-hole modification refers to a modification at the interface between two immunoglobulin heavy chains in the CH3 domain, wherein: i) in a CH3 domain of a first heavy chain, an amino acid residue is replaced with an amino acid residue having a larger side chain (e.g. tyrosine or tryptophan) creating a projection from the surface (“knob”) and ii) in the CH3 domain of a second heavy chain, an amino acid residue is replaced with an amino acid residue having a smaller side chain (e.g. alanine or threonine), thereby generating a cavity (“hole”) within at interface in the second CH3 domain within which the protruding side chain of the first CH3 domain (“knob”) is received by the cavity in the second CH3 domain. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. Furthermore, the Fc domains may be modified by the introduction of cysteine residues at positions S354 and Y349 which results in a stabilizing disulfide bridge between the two antibody heavy chains in the Fe region (Carter, et al. (2001) Immunol Methods 248, 7-15). The knob-into-hole format is used to facilitate the expression of a first polypeptide (e.g. an IL2 ortholog) on a first Fc monomer with a “knob” modification and a second polypeptide on the second Fc monomer possessing a “hole” modification to facilitate the expression of heterodimeric polypeptide conjugates.

The Fc region can be “lytic” or “non-lytic,” but is typically non-lytic. A non-lytic Fc region typically lacks a high affinity Fc receptor binding site and a Clq binding site. The high affinity Fc receptor binding site of murine IgG Fc includes the Leu residue at position 235 of IgG Fc. Thus, the Fc receptor binding site can be inhibited by mutating or deleting Leu 235. For example, substitution of Glu for Leu 235 inhibits the ability of the Fc region to bind the high affinity Fc receptor. The murine Clq binding site can be functionally destroyed by mutating or deleting the Glu 318, Lys 320, and Lys 322 residues of IgG. For example, substitution of Ala residues for Glu 318, Lys 320, and Lys 322 renders IgG1 Fc unable to direct antibody-dependent complement lysis. In contrast, a lytic IgG Fc region has a high affinity Fc receptor binding site and a Clq binding site. The high affinity Fc receptor binding site includes the Leu residue at position 235 of IgG Fc, and the Clq binding site includes the Glu 318, Lys 320, and Lys 322 residues of IgG 1. Lytic IgG Fc has wild type residues or conservative amino acid substitutions at these sites. Lytic IgG Fc can target cells for antibody dependent cellular cytotoxicity or complement directed cytolysis (CDC). Appropriate mutations for human IgG are also known (see, e.g., Morrison et al., The Immunologist 2:119-124, 1994; and Brekke et al., The Immunologist 2: 125, 1994).

In certain embodiments, the amino- or carboxyl-terminus of an IL2 ortholog of the present disclosure can be fused with an immunoglobulin Fc region (e.g., human Fc) to form a fusion conjugate (or fusion molecule). Fc fusion conjugates have been shown to increase the systemic half-life of biopharmaceuticals, and thus the biopharmaceutical product can require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates.

In some embodiments, the Fc domain monomer comprises at least one mutation relative to a wild-type human IgG1, IgG2, or IgG4 Fc region as described in U.S. Pat. No. 10,259,859B2, the entire teaching of which is herein incorporated by reference. As disclosed therein, the Fc domain monomer comprises:
(a) one of the following amino acid substitutions relative to wild type human IgG1: T366W, T366S, L368A, Y407V, T366Y, T394W, F405W, Y349T, Y349E, Y349V, L351T, L351H, L351N, L351K, P353S, S354D, D356K, D356R, D356S, E357K, E357R, E357Q, S364A, T366E, L368T, L368Y, L368E, K370E, K370D, K370Q, K392E, K392D, T394N, P395N, P396T, V397T, V397Q, L398T, D399K, D399R, D399N, F405T, F405H, F405R, Y407T, Y407H, Y4071, K409E, K409D, K409T, or K4091;
   or
(b) (i) a N297A mutation relative to a human IgG1 Fc region;
   (ii) a L234A, L235A, and G237A mutation relative to a human IgG1 Fc region;
   (iii) a L234A, L235A, G237A, and N297A mutation relative to a human IgG1 Fc region;
   (iv) a N297A mutation relative to a human IgG2 Fc region;
   (v) a A330S and P331S mutation relative to a human IgG2 Fc region;
   (vi) a A330S, P331S, and N297A mutation relative to a human IgG2 Fc region;
   (vii) a S228P, E233P, F234V, L235A, and delG236 mutation relative to a human IgG4 Fc region; or
   (viii) a S228P, E233P, F234V, L235A, delG236, and N297A mutation relative to a human IgG4 Fc region.

In some embodiments, the Fc domain monomer comprises:
(a) one of the following amino acid substitutions relative to wild type human IgG1: T366W, T366S, L368A, Y407V, T366Y, T394W, F405W, Y349T, Y349E, Y349V, L35 IT, L351H, L351N, L351K, P353S, S354D, D356K, D356R, D356S, E357K, E357R, E357Q, S364A, T366E, L368T, L368Y, L368E, K370E, K370D, K370Q. K392E, K392D, T394N, P395N, P396T, V397T, V397Q, L398T, D399K, D399R, D399N, F405T, F405H, F405R, Y407T, Y407H, Y4071, K409E, K409D, K409T, or K4091;
   and
(b) the Fc domain monomer further comprises:
   (i) a N297A mutation relative to a human IgG1 Fc region;
   (ii) a L234A, L235A, and G237A mutation relative to a human IgG1 Fc region;
   (iii) a L234A, L235A, G237A, and N297A mutation relative to a human IgG1 Fc region;
   (iv) a N297A mutation relative to a human IgG2 Fc region;
   (v) a A330S and P331S mutation relative to a human IgG2 Fc region;
   (vi) a A330S, P331S, and N297A mutation relative to a human IgG2 Fc region;
   (vii) a S228P, E233P, F234V, L235A, and delG236 mutation relative to a human IgG4 Fc region; or
   (viii) a S228P, E233P, F234V, L235A, delG236, and N297A mutation relative to a human IgG4 Fc region.

In some embodiments, the polypeptide exhibits a reduction of phagocytosis in a phagocytosis assay compared to a polypeptide with a wild-type human IgG Fc region. In some embodiments, the Fc domain monomer is linked to a second polypeptide comprising a second Fc domain monomer to form an Fc domain dimer.

### Chimeric Polypeptides/Fusion Proteins

In some embodiments, embodiment, the IL2 ortholog may comprise a functional domain of a chimeric polypeptide. IL2 ortholog fusion proteins of the present disclosure may be readily produced by recombinant DNA methodology by techniques known in the art by constructing a recombinant vector comprising a nucleic acid sequence comprising a nucleic acid sequence encoding the IL2 ortholog in frame with a nucleic acid sequence encoding the fusion partner either at the N-terminus or C-terminus of the IL2 ortholog, the sequence optionally further comprising a nucleic acid sequence in frame encoding a linker or spacer polypeptide.

### Flag Tags

In other embodiments, the IL2 ortholog can be modified to include an additional polypeptide sequence that functions as an antigenic tag, such as a FLAG sequence. FLAG sequences are recognized by biotinylated, highly specific, anti-FLAG antibodies, as described herein (see e.g., Blanar et al. (1992) Science 256:1014 and LeClair, et al. (1992) PNAS-USA 89:8145). In some embodiments, the IL2 ortholog polypeptide further comprises a C-terminal c-myc epitope tag.

### His Tags

In some embodiment, the IL2 orthologs (including fusion proteins of such IL2 orthologs) of the present invention are expressed as a fusion protein with one or more transition metal chelating polypeptide sequences. The incorporation of such a transition metal chelating domain facilitates purification immobilized metal affinity chromatography (IMAC) as described in Smith, et al. U.S. Pat. No. 4,569,794 issued Feb. 11, 1986. Examples of transition metal chelating polypeptides useful in the practice of the present invention are described in Smith, et al. supra and Dobeli, et al. U.S. Pat. No. 5,320,663 issued May 10, 1995, the entire teachings of which are hereby incorporated by reference. Particular transition metal chelating polypeptides useful in the practice of the present invention are peptides comprising 3-6 contiguous histidine residues (SEQ ID NO: 51) such as a six-histidine peptide (His)₆ (SEQ ID NO: 52) and are frequently referred to in the art as “His-tags.”

### Targeted IL2 Ortholog Fusion Proteins:

In some embodiments, the IL2 ortholog is provided as a fusion protein with a polypeptide sequence (“targeting domain”) to facilitate selective binding to particular cell type or tissue expressing a cell surface molecule that specifically binds to such targeting domain, optionally incorporating a linker molecule of from 1-40 (alternatively 2-20, alternatively 5-20, alternatively 10-20) amino acids between the IL2 ortholog sequence and the sequence of the targeting domain of the fusion protein.

In other embodiments, a chimeric polypeptide including a orthogonal IL-2 and an antibody or antigen-binding portion thereof can be generated. The antibody or antigen-binding component of the chimeric protein can serve as a targeting moiety. For example, it can be used to localize the chimeric protein to a particular subset of cells or target molecule. Methods of generating cytokine-antibody chimeric polypeptides are described, for example, in U.S. Pat. No. 6,617,135.

In some embodiments, the targeting domain of the IL2 ortholog fusion protein specifically binds to a cell surface molecule of a tumor cell. In one embodiment wherein the ECD of the CAR of a CAR-T cell specifically binds to CD-19, the IL2 ortholog may be provided as a fusion protein with a CD-19 targeting moiety. For example, in one embodiment wherein the ECD of the CAR of a CAR-T cell is an scFv molecule that provides specific binding to CD-19, the IL2 ortholog is provided as a fusion protein with a CD-19 targeting moiety such as a single chain antibody (e.g., an scFv or VHH) that specifically binds to CD-19.

In some embodiments, the fusion protein comprises an IL-2 mutein and the anti-CD19 sdFv FMC63 (Nicholson, et al. (1997) Mol Immunol 34: 1157-1165). Similarly, in some embodiments wherein the ECD of the CAR of a CAR-T cell specifically binds to BCMA, the IL2 ortholog is provided as a fusion protein with a BCMA targeting moiety, such as antibody comprising the CDRs of anti-BMCA antibodies as described in in Kalled, et al. (U.S. Pat. No. 9,034,324 issued May 9, 2015) or antibodies comprising the CDRs as described in Brogdon, et al., (U.S. Pat. No. 10,174,095 issued Jan. 8, 2019). In some embodiments the IL2 ortholog is provided as a fusion protein with a GD2 targeting moiety, such as an antibody comprising the CDRs of described in Cheung, et al., (U.S. Pat. No. 9,315,585 issued Apr. 19, 2016) or the CDRs derived from ME36.1 (Thurin et al., (1987) Cancer Research 47:1229-1233), 14G2a, 3F8 (Cheung, et al., 1985 Cancer Research 45:2642-2649), hu14.18, 8B6, 2E12, or ic9.

In an alternative embodiment, the targeted IL2 orthologs of the present disclosure may be administered in combination with CAR-T cell therapy to provide targeted delivery of the IL2 ortholog to the CAR-T cell based on an extracellular receptor of the CAR-T cell such as by and anti-FMC63 antibody to target the IL2 activity to the CAR-T cells and rejuvenate exhausted CAR-T cells in vivo. Consequently, embodiments of the present disclosure include targeted delivery of IL2 orthologs by conjugation of such IL2 orthologs to antibodies or ligands that are designed to interact with specific cell surface molecules of CAR-T cells. An example of such a molecule would an anti-FMC63-hIL2 ortholog.

In other embodiments, the chimeric polypeptide includes the mutant IL-2 polypeptide and a heterologous polypeptide that functions to enhance expression or direct cellular localization of the mutant IL-2 polypeptide, such as the Aga2p agglutinin subunit (see, e.g., Boder and Wittrup, Nature Biotechnol. 15:553-7, 1997).

### Protein Transduction Domain Fusion Proteins:

In some embodiments, the IL2 ortholog further comprises a “Protein Transduction Domain” or “PTD.” A PTD is a polypeptide, polynucleotide, carbohydrate, or organic or inorganic molecule that facilitates traversing a lipid bilayer, micelle, cell membrane, organelle membrane, or vesicle membrane. The incorporation of a PTD into an IL2 ortholog facilitates the molecule traversing a membrane. In some embodiments, a PTD is covalently linked to the amino or carboxy terminus of an IL2 ortholog. In some embodiments, the PTD is incorporated as part of an PTD-IL2 ortholog fusion protein, either at the N or C terminus of the molecule.

Exemplary protein transduction domains include, but are not limited to, a minimal decapeptide protein transduction domain (corresponding to residues 47-57 of HIV-1 TAT); a polyarginine sequence comprising a number of arginine residues sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008), Transportan (as described in Wierzbicki, et al., (2014) Folio Histomchemica et Cytobiologica 52(4): 270-280 and Pooga, et a (1998) FASEB J 12(1) 67-77 and commercially available from AnaSpec as Catalog No. AS-61256); KALA (as described in Wyman et al., (1997) Biochemistry 36(10) 3008-3017 and commercially available from AnaSpec as Catalog No. AS-65459); Antennapedia Peptide (as described in Pieterse et al., (2001) Vaccine 19:1397 and commercially available from AnaSpec as Catalog No. AS-61032); TAT 47-57 (commercially available from AnaSpec as Catalog No. AS-60023).

In some embodiments, the IL-2 conjugate comprises a plasma half-life in a human subject of greater than 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 10 days, 14 days, or 30 days.

In some embodiments, when the IL2 ortholog is to be administered in the format of an Fc fusion, particularly in those situations when the polypeptide chains conjugated to each subunit of the Fc dimer are different, the Fc fusion may be engineered to possess a “knob-into-hole modification.” The knob-into-hole modification is more fully described in Ridgway, et al. (1996) Protein Engineering 9(7):617-621 and U.S. Pat. No. 5,731,168, issued Mar. 24, 1998. The knob-into-hole modification refers to a modification at the interface between two immunoglobulin heavy chains in the CH3 domain, wherein: i) in a CH3 domain of a first heavy chain, an amino acid residue is replaced with an amino acid residue having a larger side chain (e.g. tyrosine or tryptophan) creating a projection from the surface (“knob”) and ii) in the CH3 domain of a second heavy chain, an amino acid residue is replaced with an amino acid residue having a smaller side chain (e.g. alanine or threonine), thereby generating a cavity (“hole”) within at interface in the second CH3 domain within which the protruding side chain of the first CH3 domain (“knob”) is received by the cavity in the second CH3 domain. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. Furthermore, the Fc domains may be modified by the introduction of cysteine residues at positions 5354 and Y349 which results in a stabilizing disulfide bridge between the two antibody heavy chains in the Fe region (Carter, et al. (2001) Immunol Methods 248, 7-15). The knob-into-hole format is used to facilitate the expression of a first polypeptide (e.g. an IL2 ortholog) on a first Fc monomer with a “knob” modification and a second polypeptide on the second Fc monomer possessing a “hole” modification to facilitate the expression of heterodimeric polypeptide conjugates.

### Synthesis of IL2 Orthologs

The IL2 orthologs of the present disclosure may be produced by conventional methodology for the construction of polypeptides including recombinant or solid phase syntheses.

### Solid Phase Chemical Synthesis:

In addition to generating mutant polypeptides via expression of nucleic acid molecules that have been altered by recombinant molecular biological techniques, subject IL2 orthologs can be chemically synthesized. Chemically synthesized polypeptides are routinely generated by those of skill in the art. Chemical synthesis includes direct synthesis of a peptide by chemical means of the protein sequence encoding for an IL2 ortholog exhibiting the properties described.

In some embodiments, the IL2 orthologs of the present disclosure may be prepared by chemical synthesis. The chemical synthesis of the IL2 orthologs of may proceed via liquid-phase or solid-phase. Solid-phase peptide synthesis (SPPS) allows the incorporation of unnatural amino acids and/or peptide/protein backbone modification. Various forms of SPPS are available for synthesizing IL2 orthologs of the present disclosure are known in the art (e.g., Ganesan A. (2006) Mini Rev. Med. Chem. 6:3-10; and Camarero J. A. et al., (2005) Protein Pept Lett. 12:723-8). In the course of chemical synthesis, the alpha functions and any reactive side chains may be protected with acid-labile or base-labile groups that are stable under the conditions for linking amide bonds but can readily be cleaved without impairing the peptide chain that has formed.

In the solid phase synthesis, either the N-terminal or C-terminal amino acid may be coupled to a suitable support material. Suitable support materials are those which are inert towards the reagents and reaction conditions for the stepwise condensation and cleavage reactions of the synthesis process and which do not dissolve in the reaction media being used. Examples of commercially available support materials include styrene/divinylbenzene copolymers which have been modified with reactive groups and/or polyethylene glycol; chloromethylated styrene/divinylbenzene copolymers; hydroxymethylated or aminomethylated styrene/divinylbenzene copolymers; and the like. The successive coupling of the protected amino acids can be carried out according to conventional methods in peptide synthesis, typically in an automated peptide synthesizer.

At the end of the solid phase synthesis, the peptide is cleaved from the support material while simultaneously cleaving the side chain protecting groups. The peptide obtained can be purified by various chromatographic methods including but not limited to hydrophobic adsorption chromatography, ion exchange chromatography, distribution chromatography, high pressure liquid chromatography (HPLC) and reversed-phase HPLC.

### Recombinant Production:

Alternatively, the IL2 orthologs of the present disclosure are produced by recombinant DNA technology. In the typical practice of recombinant production of polypeptides, a nucleic acid sequence encoding the desired polypeptide is incorporated into an expression vector suitable for the host cell in which expression will be accomplish, the nucleic acid sequence being operably linked to one or more expression control sequences encoding by the vector and functional in the target host cell. The recombinant protein may be recovered through disruption of the host cell or from the cell medium if a secretion leader sequence (signal peptide) is incorporated into the polypeptide. The recombinant protein may be purified and concentrated for further use including incorporation. The process for the recombinant production of IL2 polypeptides is known in the art and described in Fernandes and Taforo, U.S. Pat. No. 4,604,377 issued Aug. 5, 1986 and IL2 orthologs in Mark, et al., U.S. Pat. No. 4,512,584 issued May 21, 1985, Gillis, U.S. Pat. No. 4,401,756 issued Aug. 30, 1983 the entire teachings of which are herein incorporated by reference.

### Construction of Nucleic Acid Sequences Encoding the IL2 Ortholog

In some embodiments, the IL2 ortholog is produced by recombinant methods using a nucleic acid sequence encoding the IL2 ortholog (or fusion protein comprising the IL2 ortholog). The nucleic acid sequence encoding the desired IL2 ortholog can be synthesized by chemical means using an oligonucleotide synthesizer.

The nucleic acid molecules are not limited to sequences that encode polypeptides; some or all of the non-coding sequences that lie upstream or downstream from a coding sequence (e.g., the coding sequence of IL2) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

The nucleic acid molecules encoding the IL2 ortholog (and fusions thereof) may contain naturally occurring sequences or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

Nucleic acid sequences encoding the IL2 ortholog may be obtained from various commercial sources that provide custom made nucleic acid sequences. Amino acid sequence variants of the IL2 polypeptides to the produce the IL2 orthologs of the present disclosure are prepared by introducing appropriate nucleotide changes into the coding sequence based on the genetic code which is well known in the art. Such variants represent insertions, substitutions, and/or specified deletions of, residues as noted. Any combination of insertion, substitution, and/or specified deletion is made to arrive at the final construct, provided that the final construct possesses the desired biological activity as defined herein.

Methods for constructing a DNA sequence encoding the IL2 orthologs and expressing those sequences in a suitably transformed host include, but are not limited to, using a PCR-assisted mutagenesis technique. Mutations that consist of deletions or additions of amino acid residues to an IL2 polypeptide can also be made with standard recombinant techniques. In the event of a deletion or addition, the nucleic acid molecule encoding IL2 is optionally digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

An IL2 ortholog of the present disclosure may be produced recombinantly not only directly, but also as a fusion polypeptide with a heterologous polypeptide, e.g. a signal sequence or other polypeptide having a specific cleavage site at the N-terminus or C-terminus of the mature IL2 ortholog. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. In some embodiments, the signal sequence is the signal sequence that is natively associated with the IL2 ortholog (i.e. the human IL2 signal sequence). The inclusion of a signal sequence depends on whether it is desired to secrete the IL2 ortholog from the recombinant cells in which it is made. If the chosen cells are prokaryotic, it generally is preferred that the DNA sequence not encode a signal sequence. If the chosen cells are eukaryotic, it generally is preferred that a signal sequence be encoded and most preferably that the wild type IL2 signal sequence be used. Alternatively, heterologous mammalian signal sequences may be suitable, such as signal sequences from secreted polypeptides of the same or related species, as well as viral secretory leaders, for example, the herpes simplex gD signal. When the recombinant host cell is a yeast cell such as Saccharomyces cerevisiae, the alpha mating factor secretion signal sequence may be employed to achieve extracellular secretion of the IL2 ortholog into the culture medium as described in Singh, U.S. Pat. No. 7,198,919 B1 issued Apr. 3, 2007.

In the event the IL2 ortholog to be expressed is to be expressed as a chimera (e.g., a fusion protein comprising an IL2 ortholog and a heterologous polypeptide sequence), the chimeric protein can be encoded by a hybrid nucleic acid molecule comprising a first sequence that encodes all or part of the IL2 ortholog and a second sequence that encodes all or part of the heterologous polypeptide. For example, subject IL2 orthologs described herein may be fused to a hexa-histidine tag (SEQ ID NO: 52) to facilitate purification of bacterially expressed protein, or to a hemagglutinin tag to facilitate purification of protein expressed in eukaryotic cells. By first and second, it should not be understood as limiting to the orientation of the elements of the fusion protein and a heterologous polypeptide can be linked at either the N-terminus and/or C-terminus of the IL2 ortholog. For example, the N-terminus may be linked to a targeting domain and the C-terminus linked to a hexa-histidine tag (SEQ ID NO: 52) purification handle.

The complete amino acid sequence of the polypeptide (or fusion/chimera) to be expressed can be used to construct a back-translated gene. A DNA oligomer containing a nucleotide sequence coding for IL2 ortholog can be synthesized. For example, several small oligonucleotides coding for portions of the desired polypeptide can be synthesized and then ligated. The individual oligonucleotides typically contain 5′ or 3′ overhangs for complementary assembly.

### Codon Optimization:

In some embodiments, the nucleic acid sequence encoding the recombinant protein (IL2 ortholog, orthogonal CD122, or CAR) may be “codon optimized” to facilitate expression in a particular host cell type. Techniques for codon optimization in a wide variety of expression systems, including mammalian, yeast and bacterial host cells, are well known in the and there are online tools to provide for a codon optimized sequences for expression in a variety of host cell types. See e.g. Hawash, et al., (2017) 9:46-53 and Mauro and Chappell in Recombinant Protein Expression in Mammalian Cells: Methods and Protocols, edited by David Hacker (Human Press New York). Additionally, there are a variety of web based online software packages that are freely available to assist in the preparation of codon optimized nucleic acid sequences.

### Expression Vectors:

Once assembled (by synthesis, site-directed mutagenesis or another method), the nucleic acid sequence encoding an IL2 ortholog will be inserted into an expression vector. A variety of expression vectors for uses in various host cells are available and are typically selected based on the host cell for expression. An expression vector typically includes, but is not limited to, one or more of the following: an origin of replication, one or more marker genes, an enhancer element, a promoter, and a transcription termination sequence. Vectors include viral vectors, plasmid vectors, integrating vectors, and the like. Plasmids are examples of non-viral vectors.

To facilitate efficient expression of the recombinant polypeptide, the nucleic acid sequence encoding the polypeptide sequence to be expressed is operably linked to transcriptional and translational regulatory control sequences that are functional in the chosen expression host.

### Selectable Marker

Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

### Regulatory Control Sequences:

Expression vectors for IL2 orthologs of the present disclosure contain a regulatory sequence that is recognized by the host organism and is operably linked to nucleic acid sequence encoding the IL2 ortholog. The terms “regulatory control sequence,” “regulatory sequence” or “expression control sequence” are used interchangeably herein to refer to promoters, enhancers, and other expression control elements (e.g., polyadenylation signals). See, for example, Goeddel (1990) in Gene Expression Technology: Methods in Enzymology 185 (Academic Press, San Diego Calif. USA Regulatory sequences include those that direct constitute expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. In selecting an expression control sequence, a variety of factors understood by one of skill in the art are to be considered. These include, for example, the relative strength of the sequence, its controllability, and its compatibility with the actual DNA sequence encoding the subject IL2 ortholog, particularly as regards potential secondary structures.

### Promoters

In some embodiments, the regulatory sequence is a promoter, which is selected based on, for example, the cell type in which expression is sought. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

A T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

Transcription from vectors in mammalian host cells may be controlled, for example, by promoters obtained from the genomes of viruses such as polyoma virus, fowlpox virus, adenovirus (such as human adenovirus serotype 5), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication.

### Enhancers

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence but is preferably located at a site 5′ from the promoter. Expression vectors used in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

In addition to sequences that facilitate transcription of the inserted nucleic acid molecule, vectors can contain origins of replication, and other genes that encode a selectable marker. For example, the neomycin-resistance (neoR) gene imparts G418 resistance to cells in which it is expressed, and thus permits phenotypic selection of the transfected cells. Additional examples of marker or reporter genes include beta-lactamase, chloramphenicol acetyltransferase (CAT), adenosine deaminase (ADA), dihydrofolate reductase (DHFR), hygromycin-B-phosphotransferase (HPH), thymidine kinase (TK), lacZ (encoding beta-galactosidase), and xanthine guanine phosphoribosyltransferase (XGPRT). Those of skill in the art can readily determine whether a given regulatory element or selectable marker is suitable for use in a particular experimental context.

Proper assembly of the expression vector can be confirmed by nucleotide sequencing, restriction mapping, and expression of a biologically active polypeptide in a suitable host.

### Host Cells for Production of IL2 Orthologs

The present disclosure further provides prokaryotic or eukaryotic cells that contain and express a nucleic acid molecule that encodes a IL2 ortholog. A cell of the present disclosure is a transfected cell, i.e., a cell into which a nucleic acid molecule, for example a nucleic acid molecule encoding a mutant IL2 polypeptide, has been introduced by means of recombinant DNA techniques. The progeny of such a cell are also considered within the scope of the present disclosure.

Host cells are typically selected in accordance with their compatibility with the chosen expression vector, the toxicity of the product coded for by the DNA sequences of this invention, their secretion characteristics, their ability to fold the polypeptides correctly, their fermentation or culture requirements, and the ease of purification of the products coded for by the DNA sequences. Suitable host cells for cloning or expressing the DNA in the vectors herein are the prokaryote, yeast, or higher eukaryote cells.

In some embodiments the recombinant IL2 orthologs or biologically active variants thereof can also be made in eukaryotes, such as yeast or human cells. Suitable eukaryotic host cells include insect cells (examples of Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39)); yeast cells (examples of vectors for expression in yeast S. cerenvisiae include pYepSecl (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and pPicZ (Invitrogen Corporation, San Diego, Calif.)); or mammalian cells (mammalian expression vectors include pCDM8 (Seed (1987) Nature 329:840) and pMT2PC (Kaufman et al. (1987) EMBO J. 6:187:195)).

Examples of useful mammalian host cell lines are mouse L cells (L-M[TK-], ATCC #CRL-2648), monkey kidney CV1 line transformed by SV40 (COS-7, ATCC CRL 1651); human embryonic kidney line (HEK293 or HEK293 cells subcloned for growth in suspension culture; baby hamster kidney cells (BHK, ATCC CCL 10); Chinese hamster ovary cells/-DHFR (CHO); mouse sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2). In mammalian cells, the expression vector's control functions are often provided by viral regulatory elements. For example, commonly used promoters are derived from polyoma, Adenovirus 2, cytomegalovirus, and Simian Virus 40.

The IL2 ortholog can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

In some embodiments, IL2 orthologs obtained will be glycosylated or unglycosylated depending on the host organism used to produce the mutein. If bacteria are chosen as the host then the IL2 ortholog produced will be unglycosylated. Eukaryotic cells, on the other hand, will glycosylate the IL2 orthologs, although perhaps not in the same way as native-IL2 is glycosylated.

For other additional expression systems for both prokaryotic and eukaryotic cells, see Chapters 16 and 17 of Sambrook et al. (1989) Molecular Cloning: A Laboratory Manual (2nd ed., Cold Spring Harbor Laboratory Press, Plainview, N.Y.). See, Goeddel (1990) in Gene Expression Technology: Methods in Enzymology 185 (Academic Press, San Diego, Calif.).

### Transfection:

The expression constructs of the can be introduced into host cells to thereby produce the IL2 orthologs disclosed herein or to produce biologically active muteins thereof. Vector DNA can be introduced into prokaryotic or eukaryotic cells via conventional transformation or transfection techniques. Suitable methods for transforming or transfecting host cells can be found in Sambrook et al. (1989) Molecular Cloning: A Laboratory Manual (2d ed., Cold Spring Harbor Laboratory Press, Plainview, N.Y.) and other standard molecular biology laboratory manuals.

In order to facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, and magnetic fields (electroporation).

### Cell Culture:

Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression and will be apparent to the ordinarily skilled artisan.

Recovery of Recombinant Proteins: Recombinantly produced IL2 ortholog polypeptides can be recovered from the culture medium as a secreted polypeptide if a secretion leader sequence is employed. Alternatively, the IL2 ortholog polypeptides can also be recovered from host cell lysates. A protease inhibitor, such as phenyl methyl sulfonyl fluoride (PMSF) may be employed during the recovery phase from cell lysates to inhibit proteolytic degradation during purification, and antibiotics may be included to prevent the growth of adventitious contaminants.

Purification: Various purification steps are known in the art and find use, e.g. affinity chromatography. Affinity chromatography makes use of the highly specific binding sites usually present in biological macromolecules, separating molecules on their ability to bind a particular ligand. Covalent bonds attach the ligand to an insoluble, porous support medium in a manner that overtly presents the ligand to the protein sample, thereby using natural specific binding of one molecular species to separate and purify a second species from a mixture. Antibodies are commonly used in affinity chromatography. Size selection steps may also be used, e.g. gel filtration chromatography (also known as size-exclusion chromatography or molecular sieve chromatography) is used to separate proteins according to their size. In gel filtration, a protein solution is passed through a column that is packed with semipermeable porous resin. The semipermeable resin has a range of pore sizes that determines the size of proteins that can be separated with the column.

The IL2 ortholog produced by the transformed host can be purified according to any suitable method. Various methods are known for purifying IL2. See, e.g. Current Protocols in Protein Science, Vol 2. Eds: John E. Coligan, Ben M. Dunn, Hidde L. Ploehg, David W. Speicher, Paul T. Wingfield, Unit 6.5 (Copyright 1997, John Wiley and Sons, Inc. IL2 orthologs can be isolated from inclusion bodies generated in E. coli, or from conditioned medium from either mammalian or yeast cultures producing a given mutein using cation exchange, gel filtration, and or reverse phase liquid chromatography.

The substantially purified forms of the recombinant polypeptides can be purified from the expression system using routine biochemical procedures, and can be used, e.g., as therapeutic agents, as described herein.

The biological activity of the IL2 orthologs can be assayed by any suitable method known in the art and may be evaluated as substantially purified forms or as part of the cell lysate or cell medium when secretion leader sequences are employed for expression. Such activity assays include CTLL-2 proliferation, induction of phospho-STAT5 (pSTAT5) activity in T cells, PHA-blast proliferation and NK cell proliferation.

### Routes of Administration of IL2 Orthologs:

In embodiments of the therapeutic methods of the present disclosure involve the administration of a pharmaceutical formulation comprising an IL2 ortholog (and/or nucleic acids encoding the IL2 ortholog) to a subject in need of treatment. Administration to the subject may be achieved by intravenous, as a bolus or by continuous infusion over a period of time. Alternative routes of administration include intramuscular, intraperitoneal, intra-cerobrospinal, subcutaneous, intra-articular, intrasynovial, intrathecal, oral, topical, or inhalation routes. The IL2 orthologs also are suitably administered by intratumoral, peritumoral, intralesional, intranodal or perilesional routes or to the lymph, to exert local as well as systemic therapeutic effects.

In some embodiments, subject IL2 orthologs (and/or nucleic acids encoding the IL2 ortholog) can be incorporated into compositions, including pharmaceutical compositions. Such compositions typically include the polypeptide or nucleic acid molecule and a pharmaceutically acceptable carrier. A pharmaceutical composition is formulated to be compatible with its intended route of administration and is compatible with the therapeutic use for which the IL2 ortholog is to be administered to the subject in need of treatment or prophyaxis.

### Formulations of IL2 Orthologs

The IL2 orthologs (or nucleic acids encoding same) of the present disclosure may be administered to a subject in a pharmaceutically acceptable dosage form. The preferred formulation depends on the intended mode of administration and therapeutic application.

Parenteral Formulations: In some embodiments, the methods of the present disclosure involve the parental administration of an IL2 ortholog. Examples of parenteral routes of administration include, for example, intravenous, intradermal, subcutaneous, transdermal (topical), transmucosal, and rectal administration. Parenteral formulations comprise solutions or suspensions used for parenteral application can include vehicles the carriers and buffers. Pharmaceutical formulations for parenteral administration include sterile aqueous solutions (where water soluble) or dispersions and sterile powders for the extemporaneous preparation of sterile injectable solutions or dispersion. The parenteral preparation can be enclosed in ampoules, disposable syringes or multiple dose vials made of glass or plastic. In one embodiment, the formulation is provided in a prefilled syringe for parenteral administration

Oral Formulations: Oral compositions, if used, generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

Inhalation Formulations: In the event of administration by inhalation, subject IL2 orthologs, or the nucleic acids encoding them, are delivered in the form of an aerosol spray from pressured container or dispenser which contains a suitable propellant, e.g., a gas such as carbon dioxide, or a nebulizer. Such methods include those described in U.S. Pat. No. 6,468,798.

Mucosal and Transdermal: Systemic administration of the subject IL2 orthologs or nucleic acids can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories suppositories (e.g., with conventional suppository bases such as cocoa butter and other glycerides) or retention enemas for rectal delivery. For transdermal administration, the active compounds are formulated into ointments, salves, gels, or creams as generally known in the art and may incorporate permeation enhancers such as ethanol or lanolin.

Extended Release and Depot Formulations: In some embodiments of the method of the present disclosure, the IL2 ortholog is administered to a subject in need of treatment in a formulation to provide extended release of the IL2 ortholog agent. Examples of extended release formulations of the injectable compositions can be brought about by including in the composition an agent which delays absorption, for example, aluminum monostearate and gelatin. In one embodiment, the subject IL2 orthologs or nucleic acids are prepared with carriers that will protect the mutant IL-2 polypeptides against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In one embodiment, the IL2 ortholog formulation is provided in accordance with the teaching of Fernandes and Taforo, U.S. Pat. No. 4,604,377 issued Aug. 5, 1986 the teaching of which is herein incorporated by reference. And Yasui, et al., U.S. Pat. No. 4,645,830.

### Administration of Nucleic Acids Encoding the Ortholog:

Alternative to the administration to a subject of a IL2 ortholog protein pharmaceutical formulation comprising an IL2 ortholog, the IL2 ortholog may be provided to a subject by the administration of pharmaceutically acceptable formulation of a nucleic acid construct encoding the IL2 ortholog to the subject to achieve continuous exposure of the subject to the selective IL2 ortholog. The administration of a recombinant vector encoding the IL2 ortholog provides for extended delivery of the IL2 ortholog to the subject and prolonged activation of the corresponding cells engineered to express the cognate orthogonal receptor associated with such IL2 ortholog. In some embodiments of the method of the present disclosure, nucleic acids encoding the IL2 ortholog is administered to the subject by transfection or infection using methods known in the art, including but not limited to the methods described in McCaffrey et al. (Nature 418:6893, 2002), Xia et al. (Nature Biotechnol. 20: 1006-1010, 2002), or Putnam (Am. J. Health Syst. Pharm. 53: 151-160, 1996, erratum at Am. J. Health Syst. Pharm. 53:325, 1996

Non-Viral Vectors Encoding the Ortholog: In one embodiment, the IL2 ortholog may be administered to a subject in the form of nucleic acid expression construct for the IL2 ortholog in a non-viral vector may be provided in a non-viral delivery system. Non-viral delivery systems are typically complexes to facilitate transduction of the target cell with a nucleic acid cargo wherein the nucleic acid is complexed with agents such as cationic lipids (DOTAP, DOTMA), surfactants, biologicals (gelatin, chitosan), metals (gold, magnetic iron) and synthetic polymers (PLG, PEI, PAMAM). Numerous embodiments of non-viral delivery systems are well known in the art including lipidic vector systems (Lee et al. (1997) Critical Reviews of Therapeutic Drug Carrier Systems 14:173-206); polymer coated liposomes (Marin et al., U.S. Pat. No. 5,213,804, issued May 25, 1993; Woodle, et al., U.S. Pat. No. 5,013,556, issued May 7, 1991); cationic liposomes (Epand et al., U.S. Pat. No. 5,283,185, issued Feb. 1, 1994; Jessee, J. A., U.S. Pat. No. 5,578,475, issued Nov. 26, 1996; Rose et al, U.S. Pat. No. 5,279,833, issued Jan. 18, 1994; Gebeyehu et al., U.S. Pat. No. 5,334,761, issued Aug. 2, 1994). In one embodiment, the nucleic acid sequence in the non-viral vector system encoding the IL2 receptor is under control of a regulatable promoter, inducible promoter, tissue specific or tumor specific promoter, or temporally regulated promoter.

Viral Vectors Encoding the Ortholog: In another embodiment, IL2 ortholog may be administered to a subject in the form of nucleic acid expression construct in viral vector encoding the IL2 ortholog. The terms “viral vector” and “virus” are used interchangeably herein to refer to any of the obligate intracellular parasites having no protein-synthesizing or energy generating mechanism. The viral genome may be RNA or DNA contained with a coated structure of protein of a lipid membrane, The terms virus(es) and viral vectors) are used interchangeably herein. The viruses useful in the practice of the present invention include recombinantly modified enveloped or nonenveloped DNA and RNA viruses, preferably selected from baculoviridiae, parvoviridiae, picomoviridiae, herpesviridiae, poxviridae, or adenoviridiae. The viruses are modified by recombinant DNA techniques to include expression of exogenous transgenes (e.g. a nucleic acid sequence encoding the IL2 ortholog) and may be engineered to be replication deficient, conditionally replicating or replication competent. Minimal vector systems in which the viral backbone contains only the sequences need for packaging of the viral vector and may optionally include a transgene expression cassette may also be employed. The term “replication deficient” refers to vectors that are highly attenuated for replication in a wild type mammalian cell. in order to produce such vectors in quantity, a producer cell line is generally created by co-transfection with a helper virus or genomically modified to complement the missing functions. The term “replication competent viral vectors” refers to a viral vector that is capable of infection, DNA replication, packaging and lysis of an infected cell. The term “conditionally replicating viral vectors” is used herein to refer to replication competent vectors that are designed to achieve selective expression in particular cell types. Such conditional replication may be achieved by operably linking tissue specific, tumor specific or cell type specific or other selectively induced regulatory control sequences to early genes (e.g., the E1 gene of adenoviral vectors). Infection of the subject with the recombinant virus or non-viral vector can provide for long term expression of the IL2 ortholog in the subject and provide continuous selective maintenance of the engineered T cells expressing the CD122 orthogonal receptor. In one embodiment, the nucleic acid sequence in the viral vector system encoding the IL2 receptor is under control of a regulatable promoter, inducible promoter, tissue specific or tumor specific promoter, or temporally regulated promoter.

### Orthogonal Receptors

In some embodiments, the orthogonal receptor is a chimeric receptor wherein the extracellular domain comprises an orthogonal extracellular domain of a first receptor protein operably linked (e.g. as a fusion protein) to the transmembrane (TM) domain and/or intracellular domain (ICD) of a second receptor protein such that the binding of the orthogonal ligand to the orthogonal ECD results in intracellular signaling characteristic of the second receptor protein. The orthogonal hCD122 is a variant of hCD122 that comprises one or more amino acid modifications (e.g., deletions or substitutions) at those positions involved in the binding of native cytokine (i.e. wild-type hIL2) to wild-type hCD122 so as to disrupt the binding of the native cytokine (i.e. wt-hIL2) to the orthogonal hCD122. Amino acids involved in the binding of the hIL2 to hCD122 include but are not limited to amino acids R41, R42, Q70, K71, T73, T74, V75, 5132, H133, Y134, F135, E136, and/or Q214. In some embodiments, the orthogonal CD122 comprises a one or more substitutions or deletions of amino acids R41, R42, Q70, K71, T73, T74, V75, 5132, H133, Y134, F135, E136, and/or Q214. In some embodiments, the orthogonal CD122 comprises one or more substitutions or deletions of the amino acids Q70, T73, H133, and/or Y134. In some embodiments, the orthogonal CD122 comprises one or more substitutions or deletions of the amino acids H133 and/or Y134. In some embodiments, the orthogonal CD122 comprises one or more substitutions or deletions of the amino acids H133 and/or Y134. In some embodiments, the orthogonal CD122 comprises one or more substitutions or deletions of the amino acids H133 and Y134.

A receptor polypeptide comprising an extracellular domain, a transmembrane domain and an intracellular domain, the extracellular domain of said polypeptide comprising an amino acid sequence of the following structure:

(SEQ ID NO: 53)
Ala-Val-Asn-Gly-Thr-Ser-Gln-Phe-Thr-Cys-Phe-Tyr-
Asn-Ser-Arg-Ala-Asn-Ile-Ser-Cys-Val-Trp-Ser-Gln-
Asp-Gly-Ala-Leu-Gln-Asp-Thr-Ser-Cys-Gln-Val-His-
Ala-Trp-Pro-Asp-Arg-Arg-Arg-Trp-Asn-Gln-Thr-Cys-
Glu-Leu-Leu-Pro-Val-Ser-Gln-Ala-Ser-Trp-Ala-Cys-
Asn-Leu-Ile-Leu-Gly-Ala-Pro-Asp-Ser-AA70-Lys-Leu-
AA73-Thr-Val-Asp-Ile-Val-Thr-Leu-Arg-Val-Leu-Cys-
Arg-Glu-Gly-Val-Arg-Trp-Arg-Val-Met-Ala-Ile-Gln-
Asp-Phe-Lys-Pro-Phe-Glu-Asn-Leu-Arg-Leu-Met-Ala-
Pro-Ile-Ser-Leu-Gln-Val-Val-His-Val-Glu-Thr-His-
Arg-Cys-Asn-Ile-Ser-Trp-Glu-Ile-S er-Gln-Ala-Ser-
AA133-AA134-Phe-Glu-Arg-His-Leu-Glu-Phe-Glu-Ala-
Arg-Thr-Leu-Ser-Pro-Gly-His-Thr-Trp-Glu-Glu-Ala-
Pro-Leu-Leu-Thr-Leu-Lys-Gln-Lys-Gln-Glu-Trp-Ile-
Cys-Leu-Glu-Thr-Leu-Thr-Pro-Asp-Thr-Gln-Tyr-Glu-
Phe-Gln-Val-Arg-Val-Lys-Pro-Leu-Gln-Gly-Glu-Phe-
Thr-Thr-Trp-Ser-Pro-Trp-Ser-Gln-Pro-Leu-Ala-Phe-
Arg-Thr-Lys-Pro-Ala-Ala-Leu-Gly-Lys-Asp-Thr

wherein:

AA70=Gln or Tyr;

AA73=Thr, Asp or Tyr;

AA133=His, Asp, Glu or Lys; and/or

AA134=Tyr, Phe, Glu or Arg.

The ECD of the hCD122 receptor comprises several secondary structural features as summarized in the Table 3 below:

TABLE 3
Secondary Structural Features of hCD122 ECD

| | Position In Precursor | Position In Mature |
|---|---|---|
| | form of hCD122 | form of hCD122 |
| | (including 26AA | (excluding signal |
| Feature | signal sequence) | sequence) |
| N-linked glycosylation site | Asn29 | Asn3 |
| Disulfide bond | Cys36-Cys46 | Cys10-Cys20 |
| N-linked glycosylation site | Asn43 | Asn17 |
| Disulfide bond | Cys59-Cys110 | Cys33-Cys84 |
| N-linked glycosylation site | Asn71 | Asn45 |
| Disulfide bond | Cys74-Cys86 | Cys48-Cys60 |
| N-linked glycosylation site | Asn149 | Asn123 |

When making modifications in the ECD sequence of hCD122, it some embodiments, the amino acids involved in the formation of secondary structural features are retained to maintain the secondary structure of the protein. In general, maintenance of disulfide bonds is desirable. In some embodiments deletion of glycosylation sites may be desired. Consequently, one or more conservative amino acid substitutions of asparagine (for example with alanine or isoleucine) at or more of positions 3, 17, 42 and/or 123 of the ECD mature of hCD122 may be incorporated to eliminate one or more these N-linked glycosylation sites.

In one embodiment, the orthogonal CD122 is human CD122 comprising amino acid modifications at as positions 133 and 134 of numbered in accordance with the naturally occurring form of mature human CD122 (SEQ ID NO: 1). In some embodiments, the orthogonal CD122 is a hCD122 molecule comprising the amino acid substitutions H133D and Y134. In one embodiment, the orthogonal receptor is a modified human CD122 wherein the amino acid sequence of the ECD is a 214 amino acid polypeptide of the sequence:

(SEQ ID NO: 6)
AVNGTSQFTC FYNSRANISC VWSQDGALQD TSCQVHAWPD
RRRWNQTCEL LPVSQASWAC NLILGAPDSQ KLTTVDIVTL
RVLCREGVRW RVMAIQDFKP FENLRLMAPI SLQVVHVETH
RCNISWEISQ ASDFFERHLE FEARTLSPGH TWEEAPLLTL
KQKQEWICLE TLTPDTQYEF QVRVKPLQGE FTTWSPWSQP
LAFRTKPAAL GKDT

In one embodiment, the orthogonal receptor is a modified human CD122 having the amino acid sequence (less the signal peptide) of the ECD of hCD122 having substitutions H133D and Y134F and the transmembrane (TM) and intracellular domain (ICD) of the wild-type hCD122 molecule having the amino acid sequence:

(SEQ ID NO: 7)
AVNGTSQFTC FYNSRANISC VWSQDGALQD TSCQVHAWPD
RRRWNQTCEL LPVSQASWAC NLILGAPDSQ KLTTVDIVTL
RVLCREGVRW RVMAIQDFKP FENLRLMAPI SLQVVHVETH
RCNISWEISQ ASDFFERHLE FEARTLSPGH TWEEAPLLTL
KQKQEWICLE TLTPDTQYEF QVRVKPLQGE FTTWSPWSQP
LAFRTKPAAL GKDTIPWLGH LLVGLSGAFG FIILVYLLIN
CRNTGPWLKK VLKCNTPDPS KFFSQLSSEH GGDVQKWLSS
PFPSSSFSPG GLAPEISPLE VLERDKVTQL LLQQDKVPEP
ASLSSNHSLT SCFTNQGYFF FHLPDALEIE ACQVYFTYDP
YSEEDPDEGV AGAPTGSSPQ PLQPLSGEDD AYCTFPSRDD
LLLFSPSLLG GPSPPSTAPG GSGAGEERMP PSLQERVPRD
WDPQPLGPPT PGVPDLVDFQ PPPELVLREA GEEVPDAGPR
EGVSFPWSRP PGQGEFRALN ARLPLNTDAY LSLQELQGQD
PTHL

“hoCD122” or “hoIL2Rb” are used interchangeably to refers to a variant of hCD122 comprising amino acid substitutions at positions histidine 133 (H133) and tyrosine 134 (Y134) in the ECD of the hCD122 polypeptide.

In one embodiment, the orthogonal receptor comprises a variant of naturally occurring mature CD122 ECD that comprises one or more amino acid modifications (e.g., deletions or substitutions) at those positions involved in the binding of native cytokine (i.e. wild-type hIL2) to the ECD of the wild-type hCD122 so as to disrupt the binding of the native cytokine (i.e. wt-hIL2) to the ECD of the orthogonal hCD122. Amino acids involved in the binding of the hIL2 to hCD122 ECD include but are not limited to amino acids R41, R42, Q70, K71, T73, T74, V75, 5132, H133, Y134, F135, E136, and/or Q214. In some embodiments, the orthogonal receptor comprises a CD122 ECD comprising one or more substitutions or deletions of amino acids R41, R42, Q70, K71, T73, T74, V75, 5132, H133, Y134, F135, E136, and/or Q214. In some embodiments, the orthogonal CD122 ECD (or hoCD122 receptor) comprises one or more substitutions or deletions of the amino acids Q70, T73, H133, and/or Y134. In some embodiments, the orthogonal CD122 comprises one or more substitutions or deletions of the amino acids H133 and/or Y134. In some embodiments, the orthogonal CD122 comprises one or more substitutions or deletions of the amino acids H133 and/or Y134. In some embodiments, the orthogonal CD122 comprises one or more substitutions or deletions of the amino acids H133 and Y134.

In some embodiments the orthogonal receptor comprises an hoCD122 ECD having amino acid substitutions at position 133 from histidine to aspartic acid (H133D), glutamic acid (H133E) or lysine (H133K) and/or amino acid substitutions at position 134 to from tyrosine to phenylalanine (Y134F), glutamic acid (Y134E), or arginine (Y134R). In one embodiment, the orthogonal receptor is a polypeptide wherein the ECD comprises the amino acid sequence of SEQ ID NO: 6. In one embodiment, the orthogonal CD122 ortho is a hCD122 molecule having amino acid substitutions H133D and Y134F. In one embodiment, the hoCD122 receptor is a polypeptide comprising the amino acid sequence of SEQ ID NO: 7)

In one embodiment, the orthogonal receptor is a fusion protein comprising the ECD of hoCD122 (e.g. SEQ ID NO. 6) and the transmembrane and intracellular domains of a second receptor in the IL2 common gamma chain family of receptors (e.g. IL4 receptor Type II receptor subunit a (hIL4Ra UniProt P24394), IL-7 receptor subunit a (hIL7Ra UniProt 16871), IL9 receptor (hIL9R UniProt Q01113), and the IL21 receptor (hIL21R UniProt Q9HBE5). The amino acid sequences and nucleic acid sequences of these common gamma-chain receptor family members are well known in the literature in addition to the locations of the signal, extracellular, transmembrane and intracellular domains. Consequently, the ordinarily skilled artisan would be readily able to prepare fusion proteins comprising the orthogonal CD122 ECD with the transmembrane and/or intraceulluar domains of these alternative common gamma chain family members. Examples of the orthogonal chimeric fusion receptors along with sequence information is provided in Table 5 below:

TABLE 4
Chimeric Orthogonal Common Gamma Chain Receptors

| Fusion | | | |
|---|---|---|---|
| Domain | ECD | TM | ICD |
| hIL4RA | SEQ ID NO: 6 | LLLGVSV | KIKKEWWDQIPNPARSRLVAIIIQDAQGSQWEKRSRGQEPAKCPH |
| | | SCIVILA | WKNCLTKLLPCFLEHNMKRDEDPHKAAKEMPFQGSGKSAWCPVEI |
| | | VCLLCYV | SKTVLWPESISVVRCVELFEAPVECEEEEEVEEEKGSFCASPESS |
| | | SIT | RDDFQEGREGIVARLTESLFLDLLGEENGGFCQQDMGESCLLPPS |
| | | (SEQ ID | GSTSAHMPWDEFPSAGPKRAPPWGKEQPLHLEPSPPASPTQSPDN |
| | | NO: 8) | LTCTETPLVIAGNPAYRSFSNSLSQSPCPRELGPDPLLARHLEEV |
| | | | EPEMPCVPQLSEPTTVPQPEPETWEQILRRNVLQHGAAAAPVSAP |
| | | | TSGYQEFVHAVEQGGTQASAVVGLGPPGEAGYKAFSSLLASSAVS |
| | | | PEKCGFGASSGEEGYKPFQDLIPGCPGDPAPVPVPLFTFGLDREP |
| | | | PRSPQSSHLPSSSPEHLGLEPGEKVEDMPKPPLPQEQATDPLVDS |
| | | | LGSGIVYSALTCHLCGHLKQCHGQEDGGQTPVMASPCCGCCCGDR |
| | | | SSPPTTPLRAPDPSPGGVPLEASLCPASLAPSGISEKSKSSSSFH |
| | | | PAPGNAQSSSQTPKIVNFVSVGPTYMRVS (SEQ ID NO: 9) |
| hIl7Ra | SEQ ID NO: 6 | PILLTIS | KKRIKPIVWPSLPDHKKTLEHLCKKPRKNLNVSFNPESFLDCQIH |
| | | ILSFFSV | RVDDIQARDEVEGFLQDTFPQQLEESEKQRLGGDVQSPNCPSEDV |
| | | ALLVILA | VITPESFGRDSSLTCLAGNVSACDAPILSSSRSLDCRESGKNGPH |
| | | CVLW | VYQDLLLSLGTTNSTLPPPFSLQSGILTLNPVAQGQPILTSLGSN |
| | | (SEQ ID | QEEAYVTMSSFYQNQ (SEQ ID NO: 11) |
| | | NO: 10) | |
| hIL9R | SEQ ID NO: 6 | GNTLVAV | KLSPRVKRIFYQNVPSPAMFFQPLYSVHNGNFQTWMGAHGAGVLL |
| | | SIFLLLT | SQDCAGTPQGALEPCVQEATALLTCGPARPWKSVALEEEQEGPGT |
| | | GPTYLLF | RLPGNLSSEDVLPAGCTEWRVQTLAYLPQEDWAPTSLTRPAPPDS |
| | | (SEQ ID | EGSRSSSSSSSSNNNNYCALGCYGGWHLSALPGNTQSSGPIPALA |
| | | NO: 12) | CGLSCDHQGLETQQGVAWVLAGHCQRPGLHEDLQGMLLPSVLSKA |
| | | | RSWTF (SEQ ID NO: 13) |
| hIL21R | SEQ ID NO: 6 | GWNPHLL | SLKTHPLWRLWKKIWAVPSPERFFMPLYKGCSGDFKKWVGAPFTG |
| | | LLLLLVI | SSLELGPWSPEVPSTLEVYSCHPPRSPAKRLQLTELQEPAELVES |
| | | VFIPAFW | DGVPKPSFWPTAQNSGGSAYSEERDRPYGLVSIDTVTVLDAEGPC |
| | | (SEQ ID | TWPCSCEDDGYPALDLDAGLEPSPGLEDPLLDAGTTVLSCGCVSA |
| | | NO: 14) | GSPGLGGPLGSLLDRLKPPLADGEDWAGGLPWGGRSPGGVSESEA |
| | | | GSPLAGLDMDTFDSGFVGSDCSSPVECDFTSPGDEGPPRSYLRQW |
| | | | VVIPPPLSSPGPQAS(SEQ ID NO:15) |

Orthogonal CD122 Receptors with STAT3 Motif

The present disclosure provides an orthogonal CD122 comprising, in addition to a native STAT5 recognition motif, one or more STAT3 binding motifs. The additional STAT3 binding motifs boosts the signaling and also stabilizes the IL2 response.

STAT proteins act as transcriptional activators upon phosphorylation of a conserved tyrosine residue at the C terminus followed by translocation into the nucleus, where they bind to DNA and activate target gene transcription. Hennighausen and Robinson (2008) Genes Dev. 2008; 22:711-21. Seven STAT proteins have been identified in the STAT family: STAT1, STAT2, STAT3, STAT4, STAT5A, STAT5B and STAT6, and they have functions in a variety of pathways, from innate and acquired immunity to cell proliferation, differentiation and survival. Basham et al., (2008) Nucleic Acids Res. 2008 June; 36(11): 3802-3818.

STATs binding motifs are typically present in cytokine receptors and binding of their respective cytokine ligand activates the tyrosine kinases in the Janus kinase (JAK) families, which phosphorylate certain tyrosine residues in the intracellular domains. The phosphorylated receptor recruits STATs to STAT recognition motifs on the receptor and the STAT becomes phosphorylated. The phosphorylated STATs dimerize and translocate to the nucleus wherein they initiate transcription of a variety of genes. Hennighausen, supra.

Of these STAT proteins, STAT5 is activated by the binding of cytokines including IL2, IL-4, IL-7, IL-9, IL-15, and IL21 to their cognate receptors. Lara E. Kallal & Christine A. Biron (2013) Changing partners at the dance, JAK-STAT, 2:1, e23504, DOI: 10.4161/jkst.23504, Page 2, Col. 2. For example, CD122 and orthogonal CD122 contain a STATS recognition motif and can recruit and activate STAT5. Activated STAT5 results in the activation of transcription of genes such as Cis, spi2.1, and Socs-1. Basham et al., Nucleic Acids Res supra.

The STAT5 binding motif has a sequence of YX₁X₂L ((SEQ ID NO: 19). X₁ and X₂ can be any natural amino acid. In some cases, X₁ and X₂ are the same amino acid residues. In some cases, X₁ and X₂ are different amino acid residues. In one embodiment, the STAT5 motif has a sequence of YLSL (SEQ ID NO: 20).

The STAT3 binding motif is not present in the naturally occurring form (wild-type) human CD122. It is typically present in other cytokine receptors that bind to IL-6, IL-10, IL21, IFNα, IFNβ, IFNγ, and IFN λ. Upon activation, STAT3 targets Bcl-XL, survivin, cyclin D1, and activating c-myc. Kallal, et al, supra. STAT3 can be activated through tyrosine phosphorylation by a variety of cytokines whose receptors share the gp130 chain, including IL-6 and IL21, oncostatin M (OSM) and leukemia inhibitory factor (LIF). STAT3 has roles in a variety of biological functions including oncogenesis, angiogenesis and tumor metastasis, and, anti-apoptosis. See e.g. Sun et al. (2006) FEBS Lett. 580(25):5880-4 and Fukada, et al. (1996) Immunity 5(5): 449-460. The incorporation of one or more functional STAT3 signaling motifs in the intracellular domain of the orthogonal CD122 upregulates anti-apoptotic factors in the modified cells expressing the orthogonal IL2 in response to binding of a cognate IL2 ortholog to the ECD of such STAT3 modified orthogonal CD122. Consequently, orthogonal cells which express an orthogonal CD122 comprising an intracellular domain incorporating one or more functional STAT3 domains have an enhanced survival and longer life and therefore a longer duration of action in vivo.

In some embodiments, the present disclosure provides a human orthogonal CD122 (comprising the intact STAT5 motif) has been modified to introduce one or more STAT3 binding motifs and the modified human CD122 so produced retains STAT5 recognition motif and gains one or more STAT3 binding motifs.

In some embodiments, the modified orthogonal CD122 may comprise one, two, three, or more STAT3 binding motifs. In some embodiments, the STAT3 recognition motif has an amino acid sequence of YX₁X₂Q (SEQ ID NO: 21). In some embodiments, X₁ is selected from the group consisting of L, R, F, M, and X₂ is selected from the group consisting of R, K, H, and P. In some embodiments, the STAT3 recognition motif has an amino acid sequence selected from the group consisting of: YLRQ (SEQ ID NO:24); YLKQ (SEQ ID NO: 25); YRHQ (SEQ ID NO: 26); YLRQ (SEQ ID NO: 24); YFKQ (SEQ ID NO: 28); YLPQ (SEQ ID NO: 16); YMPQ (SEQ ID NO: 17), and YDKPH (SEQ ID NO: 18).

In some embodiments, the one or more STAT3 binding motifs may be incorporated at the C-terminus of the orthogonal CD122 ICD or as an internal sequence of the ICD of the orthogonal CD122

In some cases, the modified human CD122 comprises one or more STAT3 binding motifs fused to the C-terminus of the intracellular domain of a human CD122. In some representative embodiments, the orthogonal CD122 comprises the addition of C-terminal STAT3 recognition domains resulting in orthogonal CD122 polypeptides of the structures:

| | |
|---|---|
| Ortho-CD122-GGYLRQ | (″GGYLRQ″ disclosed as SEQ ID NO: 54); |
| Ortho-CD122-GGYLKQ | (″GGYLKQ″ disclosed as SEQ ID NO: 55); |
| Ortho-CD122-GGYRHQ | (″GGYRHQ″ disclosed as SEQ ID NO: 56); |
| Ortho-CD122-GGYLRQ | (″GGYLRQ″ disclosed as SEQ ID NO: 54); |
| Ortho-CD122-GGYFKQ | (″GGYFKQ″ disclosed as SEQ ID NO: 58); |
| Ortho-CD122-GGYLPQ | (″GGYLPQ″ disclosed as SEQ ID NO: 59); |
| Ortho-CD122-GGYMPQ | (″GGYMPQ″ disclosed as SEQ ID NO: 60); |
| | and |
| Ortho-CD122-GGYDKPH | (″GGYDKPH″ disclosed as SEQ ID NO: 61). |

In some cases, the orthogonal CD122 comprises a STAT3 binding motif that is connected to the human CD122 through a linker. Linkers can be derived from naturally-occurring proteins or synthetic sequences. Methods for designing linkers are well-known in the art, for example, as disclosed in Chen et al. (2013) Adv. Drug. Deliv. Rev. 65(10):1357-1369, the relevant portion thereof is herein incorporated by reference. Suitable linkers can be readily selected and can be of any suitable length, such as 1 amino acid (e.g., Gly), 2, 3, 4, 5, 6, 7, 8, 9, 10, 10-20, or 20-30 amino acids. Examples of flexible linkers include glycine polymers (G)ₙ, glycine-serine polymers, glycine-alanine polymers, alanine-serine polymers, and other flexible linkers. Glycine and glycine-serine polymers are relatively unstructured, and therefore can serve as a neutral tether between components. Further examples of flexible linkers include glycine polymers (G)ₙ, glycine-alanine polymers, alanine-serine polymers, glycine-serine polymers. Glycine and glycine-serine polymers are relatively unstructured, and therefore may serve as a neutral tether between components. The modified orthogonal CD122 may comprise one or more STAT3 binding motifs and one or more linker sequences. Said linker sequences may connect the human CD122 and one of the STAT3 binding motifs or connect individual STAT3 binding motifs. The one or more linker sequence may have the same or different sequences.

In some embodiments, one or more STAT3 binding motifs are present as an internal (i.e., at neither C nor N terminus) sequence of the orthogonal CD122. A modified orthogonal CD122 of this configuration can be produced by identifying a suitable region within the orthogonal CD122 ICD amino acid sequence that can be mutated to create a STAT3 binding motif. In one embodiment, the naturally occurring ICD of the CD122 (which is typically present in the orthogonal CD122) possess sequences similar to the STAT3 binding motif that may readily be modified to create a STAT3 binding motif with minimal modification. For example a region comprising a four-nucleotide sequence that begins with a tyrosine residue. One such region in the native human CD122 encodes a sequence of YFTYDPYSEE (SEQ ID NO: 62), which is located between position s355 and position 364 of the native human CD122 protein. In some embodiments, one or two of the YFTY (SEQ ID NO: 63), YDPY (SEQ ID NO: 64), or YSEE (SEQ ID NO: 65) comprised in this region are substituted with a STAT3 recognition motif to produce a modified human CD122 disclosed herein.

Such modified orthogonal CD122s are able to induce STAT3 and STAT5 signaling upon binding to a cognate IL2 ligand and the ability can be confirmed by e.g., monitoring the levels of phosphorylated STAT3 and STAT5 in response to contacting a cell expressing the orthogonal CD122 having the STAT modified ICD with a cognate IL2 ortholog. For example, the modified human CD122 can be introduced and expressed in T cells and antibodies that are specific to phospho-STAT5 and phosphor-STAT3 are used to detect the phosphorylation of STAT3 and STAT5. One exemplary method for detecting a recombinant protein's ability to induce STAT3 and STAT5 signaling is described in Kagoya et al. (2018) Nat Med. 24(3):352-359.

In some embodiments, this disclosure provides a method of stimulating an engineered cell expressing a modified human orthogonal CD122 comprising one or more STAT3 binding motifs, the method comprising contacting the engineered cell with a human IL2 ortholog which is a cognate ligand of the modified human orthogonal CD122 thereby stimulating the engineered cells. In some embodiments, this disclosure provides a method of increasing the intracellular levels of STAT3 and STAT5 in an engineered cell expressing a modified human orthogonal CD122 comprising one or more STAT3 binding motifs, the method comprising contacting the engineered cell with a human IL2 ortholog which is a cognate ligand of the modified human orthogonal CD122 such that the intracellular levels of STAT3 and STAT5 are increased in the engineered cell.

### Method of Selective Activation of STAT5 STAT3

In some embodiments, this disclosure provides a method of stimulating an engineered T cell expressing a modified human orthogonal CD122 comprising one or more STAT3 binding motifs, the method comprising contacting the engineered T cell with a human IL2 ortholog which is a cognate ligand of the modified human orthogonal CD122 thereby stimulating the engineered T cells. In some embodiments, this disclosure provides a method of increasing the intracellular levels of STAT3 and STAT5 in an engineered T cell expressing a modified human orthogonal CD122 comprising one or more STAT3 binding motifs, the method comprising contacting the engineered T cell with a human IL2 ortholog which is a cognate ligand of the modified human orthogonal CD122 such that the intracellular levels of STAT3 and STAT5 are increased in said cell.

In some embodiments, this disclosure provides a method of stimulating an CAR-T cell expressing a modified human orthogonal CD122 comprising one or more STAT3 binding motifs, the method comprising contacting the CAR-T cell with a human IL2 ortholog which is a cognate ligand of the modified human orthogonal CD122 thereby stimulating the engineered T cells. In some embodiments, this disclosure provides a method of increasing the intracellular levels of STAT3 and STAT5 in an CAR-T cell expressing a modified human orthogonal CD122 comprising one or more STAT3 binding motifs, the method comprising contacting the CAR-T cell with a human IL2 ortholog which is a cognate ligand of the modified human orthogonal CD122 such that the intracellular levels of STAT3 and STAT5 are increased in said CAR-T cell.

### Orthogonal Engineered Cells

The preparation of orthogonal immune cells useful in the practice of the present invention is achieved by transforming isolated immune cells with an expression vector comprising a nucleic acid sequence encoding an CD122 orthogonal receptor. The IL2 orthologs of the present disclosure are employed in methods of selectively expanding such engineered T cells (e.g., human T-cells) which have been engineered to express a corresponding orthogonal CD122 receptor.

Cells useful for engineering with the constructs described herein include naïve T-cells, central memory T-cells, effector memory T-cells or combination thereof. T cells for engineering as described above are collected from a subject or a donor may be separated from a mixture of cells by techniques that enrich for desired cells or may be engineered and cultured without separation. Alternatively, the T cells for engineering may be separated from other cells. Techniques providing accurate separation include fluorescence activated cell sorters. The cells may be selected against dead cells by employing dyes associated with dead cells (e.g., propidium iodide). The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum (FCS). The collected and optionally enriched cell population may be used immediately for genetic modification or may be frozen at liquid nitrogen temperatures and stored, being thawed and capable of being reused. The cells will usually be stored in 10% DMSO, 50% FCS, 40% RPMI 1640 medium.

In some embodiments, the engineered cells comprise a complex mixture of immune cells, e.g., tumor infiltrating lymphocytes (TILs) isolated from an individual in need of treatment. See, for example, Yang and Rosenberg (2016) Adv Immunol. 130:279-94, “Adoptive T Cell Therapy for Cancer; Feldman et al (2015) Seminars in Oncol. 42(4):626-39 “Adoptive Cell Therapy-Tumor-Infiltrating Lymphocytes, T-Cell Receptors, and Chimeric Antigen Receptors”; Clinical Trial NCT01174121, “Immunotherapy Using Tumor Infiltrating Lymphocytes for Patients With Metastatic Cancer”; Tran et al. (2014) Science 344(6184)641-645, “Cancer immunotherapy based on mutation-specific CD4+ T cells in a patient with epithelial cancer”.

### CAR-T Cells

In one embodiment of the invention the T-cell expressing the orthogonal receptor is a T-cell (e.g., human T-cell) which has been modified to surface express a chimeric antigen receptor (a ‘CAR-T’ cell). In one embodiment, the modified T cell is an allogenic CAR-T cell. In an allogenic CAR-In some embodiments, the allogenic CAR-T is modified to remove the endogenous TCRa and TCRb functions.

As defined herein “CAR” are refer to a chimeric polypeptide comprising multiple functional domains arranged from amino to carboxy terminus in the sequence: (a) an extracellular domain (ECD) comprising an antigen binding domain (ABD), and optionally comprising a “hinge” domain, (b) a transmembrane domain (TD); and (c) one or more cytoplasmic signaling domains (CSDs) wherein the foregoing domains may optionally be linked by one or more spacer domains. The CAR may also further comprise a signal peptide sequence which is conventionally removed during post-translational processing and presentation of the CAR on the cell surface of a cell transformed with an expression vector comprising a nucleic acid sequence encoding the CAR. CARs may be prepared in accordance with principles well known in the art. See e.g., Eshhar, et al. (U.S. Pat. No. 7,741,465 B1 issued Jun. 22, 2010); Sadelain, et al. (2013) Cancer Discovery 3(4):388-398; Campana and Imai (U.S. Pat. No. 8,399,645 issued Mar. 19, 2013) Jensen and Riddell (2015) Current Opinions in Immunology 33:9-15; Gross, et al. (1989) PNAS (USA) 86(24):10024-10028; Curran, et al. (2012) J Gene Med 14(6):405-15; Brogdon, et al. (U.S. Pat. No. 10,174,095 issued Jan. 8, 2019) Guedan, et al. (2019) Engineering and Design of Chimeric Antigen Receptors Molecular Therapy: Methods & Clinical Development Vol. 12: 145-156.

CARs useful in the practice of the present invention are prepared in accordance with principles well known in the art. See e.g., Eshhaar et al. U.S. Pat. No. 7,741,465 B1 issued Jun. 22, 2010; Sadelain, et al (2013) Cancer Discovery 3(4):388-398 (The basic principles of chimeric antigen receptor (CAR) design); Jensen and Riddell (2015) Current Opinions in Immunology 33:9-15 (Designing chimeric antigen receptors to effectively and safely target tumors); Gross, et al. (1989) PNAS (USA) 86(24):10024-10028 (Expression of immunoglobulin-T-cell receptor chimeric molecules as functional receptors with antibody-type specificity); Curran, et al. (2012) J Gene Med 14(6):405-15. Considerations regarding the construction of the CAR and of the functional domains thereof in the context of the present invention are discussed below.

### Signal Sequence

The CAR of the disclosure may comprises a signal peptide. In the practice of the present invention any eukaryotic signal peptide sequence may be employed. The signal peptide may be derived from native signal peptides of surface expressed proteins. In one embodiment of the invention, the signal peptide of the CAR is the signal peptide selected from the group consisting of human serum albumin signal peptide, prolactin albumin signal peptide, the human IL2 signal peptide, human trypsinogen-2, human CD-5, the human immunoglobulin kappa light chain, human azurocidin, Gaussia luciferase and functional derivatives thereof. Particular amino acid substitutions to increase secretion efficiency using signal peptides are described in Stern, et al. (2007) Trends in Cell and Molecular Biology 2:1-17 and Kober, et al. (2013) Biotechnol Bioeng. 1110(4):1164-73. Alternatively, the signal peptide may be a synthetic sequence prepared in accordance established principles. See e.g., Nielsen, et al. (1997) Protein Engineering 10(1):1-6 (Identification of prokaryotic and eukaryotic signal peptides and prediction of their cleavage sites); Bendtsen, et al (2004) J. Mol. Biol 340(4):783-795 (Improved Prediction of Signal Peptides SignalP 3.0); Petersen, et al (2011) Nature Methods 8:785-796 (Signal P 4.0; discriminating signal peptides from transmembrane regions).

### Extracellular Antigen Binding Domain

As used herein, the term antigen binding domain (ABD) refers to a polypeptide that contains at least one binding domain that specifically binds to at least one antigen expressed on the surface of a target cell. In some embodiments, the ABD comprises a polypeptide with two binding domains that selectively bind to the same antigen or two different antigens on the surface of the target cells. The ABD may be any polypeptide that specifically binds to one or more antigens expressed on the surface of a target cell. The ABD is a polypeptide that

The ABD of the CAR may be monovalent or multivalent and comprise one or multiple (e.g. 1, 2, or 3) polypeptide sequence (e.g. scFv, VHH, ligand) that specifically bind to a cell surface tumor antigen. In some embodiments, tumor antigens and CARs comprising ABDs that selectively bind to such cell surface tumor are known in the art (see, e.g., Dotti, et al., Immunol Rev. 2014 January; 257(1). The methods and compositions of the present disclosure are useful in conjunction with CAR therapy wherein the ABD of the CAR specifically binds a tumor antigen including but not limited to CD123, CD19, CD20, BCMA, CD22, CD30, CD70, Lewis Y, GD3, GD3, mesothelin, ROR CD44, CD171, EGP2, EphA2, ErbB2, ErbB3/4, FAP, FAR IL11Ra, PSCA, PSMA, NCAM, HER2, NY-ESO-1, MUC1, CD123, FLT3, B7-H3, CD33, IL1RAP, CLL1 (CLEC12A)PSA, CEA, VEGF, VEGF-R2, CD22, ROR1, mesothelin, c-Met, Glycolipid F77, FAP, EGFRvIII, MAGE A3, 5T4, WT1, KG2D ligand, a folate receptor (FRa), and Wnt1 antigens. Antibodies reactive with these targets are well known in the literature and one of skill in the art is capable of isolating the CDRs from such antibodies for the construction of polypeptide sequences of single chain antibodies (e.g. scFvs, CDR grafted VHHs and the like) that may be incorporated into the ABD of the CAR.

In one embodiment, the ABD is a single chain Fv (ScFv). An ScFv is a polypeptide comprised of the variable regions of the immunoglobulin heavy and light chain of an antibody covalently connected by a peptide linker (Bird, et al. (1988) Science 242:423-426; Huston, et al. (1988) PNAS (USA) 85:5879-5883; S-z Hu, et al. (1996) Cancer Research, 56, 3055-3061; Ladner, U.S. Pat. No. 4,946,778 issued Aug. 7, 1990). The preparation of an anti-targeting antigen ScFv involves the identification of a monoclonal antibody against the targeting antigen for from which the anti-targeting antigen ScFv is derived. The generation of monoclonal antibodies and isolation of hybridomas is a technique well known to those of skill in the art. See e.g. Monoclonal Antibodies: A Laboratory Manual, Second Edition, Chapter 7 (E. Greenfield, Ed. 2014 Cold Spring Harbor Press). Immune response may be enhanced through co-administration of adjuvants well known in the art such as alum, aluminum salts, or Freund's, SP-21, etc. Antibodies generated may be optimized to select for antibodies possessing particular desirable characteristics through techniques well known in the art such as phage display and directed evolution. See, e.g. Barbas, et al. (1991) PNAS (USA) 88:7978-82; Ladner, et al. U.S. Pat. No. 5,223,409 issued Jun. 29, 1993; Stemmer, W. (1994) Nature 370:389-91; Garrard U.S. Pat. No. 5,821,047 issued Oct. 13, 1998; Camps, et al. (2003) PNAS (USA) 100(17): 9727-32; Dulbecco U.S. Pat. No. 4,593,002 issued Jun. 3, 1986; McCafferty U.S. Pat. No. 6,806,079 issued Oct. 19, 2004; McCafferty, U.S. Pat. No. 7,635,666 issued Dec. 22, 2009; McCafferty, U.S. Pat. No. 7,662,557 issued Feb. 16, 2010; McCafferty, U.S. Pat. No. 7,723,271 issued May 25, 2010; and/or McCafferty U.S. Pat. No. 7,732,377. The generation of ScFvs based on monoclonal antibody sequences is well known in the art. See, e.g. The Protein Protocols Handbook, John M. Walker, Ed. (2002) Humana Press Section 150 “Bacterial Expression, Purification and Characterization of Single-Chain Antibodies” Kipriyanov, S.

In another embodiment, the ABD is a single domain antibody obtained through immunization of a camel or llama with a targeting antigen. Muyldermans, S. (2001) Reviews in Molecular Biotechnology 74: 277-302.

Alternatively, the ABD may be generated wholly synthetically through the generation of peptide libraries and isolating compounds having the desired target cell antigen binding properties. Such techniques are well known in the scientific literature. See, e.g. Wigler, et al. U.S. Pat. No. 6,303,313 B1 issued Nov. 12, 1999; Knappik, et al., U.S. Pat. No. 6,696,248 B1 issued Feb. 24, 2004, Binz, et al (2005) Nature Biotechnology 23:1257-1268; Bradbury, et al. (2011) Nature Biotechnology 29:245-254.

In addition to the ABD having affinity for the target cell expressed antigen, the ARD may also have affinity for additional molecules. For example, an ARD of the present invention may be bi-specific, i.e. have capable of providing for specific binding to a first target cell expressed antigen and a second target cell expressed antigen. Examples of bivalent single chain polypeptides are known in the art. See, e.g. Thirion, et al. (1996) European J. of Cancer Prevention 5(6):507-511; DeKruif and Logenberg (1996) J. Biol. Chem 271(13) 7630-7634; and Kay, et al. United States Patent Application Publication Number 2015/0315566 published Nov. 5, 2015.

The ABD may have affinity for more than one target antigen. For example, an ABD of the present invention may comprise chimeric bispecific binding members, i.e. have capable of providing for specific binding to a first target cell expressed antigen and a second target cell expressed antigen. Non-limiting examples of chimeric bispecific binding members include bispecific antibodies, bispecific conjugated monoclonal antibodies (mab)₂, bispecific antibody fragments (e.g., F(ab)₂, bispecific scFv, bispecific diabodies, single chain bispecific diabodies, etc.), bispecific T cell engagers (BiTE), bispecific conjugated single domain antibodies, micabodies and mutants thereof, and the like. Non-limiting examples of chimeric bispecific binding members also include those chimeric bispecific agents described in Kontermann (2012) MAbs. 4(2): 182-197; Stamova et al. (2012) Antibodies, 1(2), 172-198; Farhadfar et al. (2016) Leuk Res. 49:13-21; Benjamin et al. Ther Adv Hematol. (2016) 7(3):142-56; Kiefer et al. Immunol Rev. (2016) 270(1):178-92; Fan et al. (2015) J Hematol Oncol. 8:130; May et al. (2016) Am J Health Syst Pharm. 73(1):e6-e13. In some embodiments, the chimeric bispecific binding member is a bivalent single chain polypeptides. See, e.g. Thirion, et al. (1996) European J. of Cancer Prevention 5(6):507-511; DeKruif and Logenberg (1996) J. Biol. Chem 271(13)7630-7634; and Kay, et al. United States Patent Application Publication Number 2015/0315566 published Nov. 5, 2015.

In some instances, a chimeric bispecific binding member may be a CAR T cell adapter. As used herein, by “CAR T cell adapter” is meant an expressed bispecific polypeptide that binds the antigen recognition domain of a CAR and redirects the CAR to a second antigen. Generally, a CAR T cell adapter will have to binding regions, one specific for an epitope on the CAR to which it is directed and a second epitope directed to a binding partner which, when bound, transduces the binding signal activating the CAR. Useful CAR T cell adapters include but are not limited to e.g., those described in Kim et al. (2015) J Am Chem Soc. 137(8):2832-5; Ma et al. (2016) Proc Natl Acad Sci USA. 113(4):E450-8 and Cao et al. (2016) Angew Chem Int Ed Engl. 55(26):7520-4

In some embodiments, an antigen binding domain against GD2 is an antigen binding portion of an antibody selected from mAb 14.18, 14G2a, ch14.18, hu14.18, 3F8, hu3F8, 3G6, 8B6, 60C3, 10B8, ME36.1, and 8H9, see e.g., WO2012033885, WO2013040371, WO2013192294, WO2013061273, WO2013123061, WO2013074916, and WO201385552. In some embodiments, an antigen binding domain against GD2 is an antigen binding portion of an antibody described in US Publication No.: 20100150910 or PCT Publication No.: WO 2011160119. Another antibody is S58 (anti-GD2, neuroblastoma). Cotara™ [Perregrince Pharmaceuticals] is a monoclonal antibody described for treatment of recurrent glioblastoma.

In some embodiments the ABD of the CAR comprises the scFvFMC-63 and humanize variants thereof

Linkers/Hinge

CARs useful in the practice of the present invention may optionally include one or more polypeptide spacers linking the domains of the CAR, in particular the linkage between the ARD to the transmembrane spanning domain of the CAR. Although not an essential element of the CAR structure, the inclusion of a spacer domain is generally considered desirable to facilitate antigen recognition by the ARD. Moritz and Groner (1995) Gene Therapy 2(8) 539-546. As used in conjunction with the CAR-T T cell technology described herein, the terms “linker”, “linker domain” and “linker region” refer to an oligo- or polypeptide region from about 1 to 100 amino acids in length, which links together any of the domains/regions of the CAR of the disclosure. Linkers may be composed of flexible residues like glycine and serine so that the adjacent protein domains are free to move relative to one another. Certain embodiments comprise the use of linkers of longer length when it is desirable to ensure that two adjacent domains do not sterically interfere with each another.

In some embodiments, the linkers are non-cleavable, while in others they are cleavable (e.g., 2A linkers (for example T2A)), 2A-like linkers or functional equivalents thereof, and combinations of the foregoing. There is no particular sequence of amino acids that is necessary to achieve the spacer function but the typical properties of the spacer are flexibility to enable freedom of movement of the ARD to facilitate targeting antigen recognition. Similarly, it has been found that there is there is substantial leniency in spacer length while retaining CAR function. Jensen and Riddell (2014) Immunol. Review 257(1) 127-144. Sequences useful as spacers in the construction of CARs useful in the practice of the present invention include but are not limited to the hinge region of IgG1, the immunoglobulin1CH2-CH3 region, IgG4 hinge-CH2-CH3, IgG4 hinge-CH3, and the IgG4 hinge. The hinge and transmembrane domains may be derived from the same molecule such as the hinge and transmembrane domains of CD8-alpha. Imai, et al. (2004) Leukemia 18(4):676-684. Embodiments of the present disclosure are contemplated wherein the linkers include the picornaviral 2A-like linker, CHYSEL sequences of porcine teschovirus (P2A), Thosea asigna virus (T2A), or combinations, variants and functional equivalents thereof. In still further embodiments, the linker sequences comprise Asp-Val/Ile-Glu-X-Asn-Pro-Gly^{(2A)}-pro^{(2B)} motif, which results in cleavage between the 2A glycine and the 2B proline.

### Transmembrane Domain

CARs can further comprise a transmembrane domain joining the ABD (or linker, if employed) to the intracellular cytoplasmic domain of the CAR. The transmembrane domain is comprised of any polypeptide sequence which is thermodynamically stable in a eukaryotic cell membrane. The transmembrane spanning domain may be derived from the transmembrane domain of a naturally occurring membrane spanning protein or may be synthetic. In designing synthetic transmembrane domains, amino acids favoring alpha-helical structures are preferred. Transmembrane domains useful in construction of CARs are comprised of approximately 10, 11, 12, 13, 14, 15, 16, 17, 18, 19, 20, 21, 22, 22, 23, or 24 amino acids favoring the formation having an alpha-helical secondary structure. Amino acids having a to favor alpha-helical conformations are well known in the art. See, e.g Pace, et al. (1998) Biophysical Journal 75: 422-427. Amino acids that are particularly favored in alpha helical conformations include methionine, alanine, leucine, glutamate, and lysine. In some embodiments, the CAR transmembrane domain may be derived from the transmembrane domain from type I membrane spanning proteins, such as CD3, CD4, CD8, CD28, etc.

### Intracellular Signaling Domain

The cytoplasmic domain of the CAR polypeptide comprises one or more intracellular signal domains. In one embodiment, the intracellular signal domains comprise the cytoplasmic sequences of the T-cell receptor (TCR) and co-receptors that initiate signal transduction following antigen receptor engagement and functional derivatives and sub-fragments thereof. A cytoplasmic signaling domain, such as those derived from the T cell receptor zeta-chain, is employed as part of the CAR in order to produce stimulatory signals for T lymphocyte proliferation and effector function following engagement of the chimeric receptor with the target antigen. Examples of cytoplasmic signaling domains include but are not limited to the cytoplasmic domain of CD27, the cytoplasmic domain S of CD28, the cytoplasmic domain of CD137 (also referred to as 4-1BB and TNFRSF9), the cytoplasmic domain of CD278 (also referred to as ICOS), p110α, β, or δ catalytic subunit of PI3 kinase, the human CD3 ζ-chain, cytoplasmic domain of CD134 (also referred to as OX40 and TNFRSF4), FcεR1γ and β chains, MB1 (Igα) chain, B29 (Igβ) chain, etc.), CD3 polypeptides (δ, Δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28.

### Co-Stimulatory Domain

In some embodiments, the CAR may also provide a co-stimulatory domain. The term “co-stimulatory domain”, refers to a stimulatory domain, typically an endodomain, of a CAR that provides a secondary non-specific activation mechanism through which a primary specific stimulation is propagated. The co-stimulatory domain refers to the portion of the CAR which enhances the proliferation, survival or development of memory cells. Examples of co-stimulation include antigen nonspecific T cell co-stimulation following antigen specific signaling through the T cell receptor and antigen nonspecific B cell co-stimulation following signaling through the B cell receptor. Co-stimulation, e.g., T cell co-stimulation, and the factors involved have been described in Chen & Flies. (2013) Nat Rev Immunol 13(4):227-42. In some embodiments of the present disclosure, the CSD comprises one or more of members of the TNFR superfamily, CD28, CD137 (4-1BB), CD134 (OX40), Dap10, CD27, CD2, CD5, ICAM-1, LFA-1 (CD11a/CD18), Lck, TNFR-I, TNFR-II, Fas, CD30, CD40 or combinations thereof.

CARs are often referred to as first, second, third or fourth generation. The term first-generation CAR refers to a CAR wherein the cytoplasmic domain transmits the signal from antigen binding through only a single signaling domain, for example a signaling domain derived from the high-affinity receptor for IgE FcεR1γ or the CD3ζ chain. The domain contains one or three immunoreceptor tyrosine-based activating motif(s) [ITAM(s)] for antigen-dependent T-cell activation. The ITAM-based activating signal endows T-cells with the ability to lyse the target tumor cells and secret cytokines in response to antigen binding. Second-generation CARs include a co-stimulatory signal in addition to the CD3 signal. Coincidental delivery of the delivered co-stimulatory signal enhances cytokine secretion and antitumor activity induced by CAR-transduced T-cells. The co-stimulatory domain is usually be membrane proximal relative to the CD3ζ domain. Third-generation CARs include a tripartite signaling domain, comprising for example a CD28, CD3ζ, OX40 or 4-1BB signaling region. In fourth generation, or “armored car” CAR T-cells are further modified to express or block molecules and/or receptors to enhance immune activity such as the expression of IL-12, IL-18, IL-7, and/or IL-10; 4-1BB ligand, CD-40 ligand.

Examples of intracellular signaling domains comprising may be incorporated into the CAR of the present invention include (amino to carboxy): CD3ζ; CD28-41BB-CD3ζ; CD28-OX40-CD3ζ; CD28-41BB-CD3ζ; 41BB-CD-28-CD3ζ and 41BB-CD3ζ.

Examples of CAR architectures useful in the practice of the present invention include but are not limited to the following examples which illustrate the ECD targeting domain(s) and the architecture of the ICD of the CAR include but are not limited to:
anti-CD20-CD3ζ
anti-CD20-CD28-41BB-CD3,
anti-CD20-CD28-CD3ζ
anti-CD20-CD28-OX40-CD3ζ
anti-CD20-CD28-41BB-CD3ζ
anti-CD20-OX40-CD3ζ
anti-CD20-OX40-CD28-CD3ζ
anti-CD20-41BB-CD3ζ
anti-CD20-ICOS-CD3ζ
anti-CD20-ICOS-41BB-CD3ζ
anti-CD20-41BB-ICOS-CD3ζ
anti-CD20-41BB-OX40-CD3ζ,
anti-CD20-41BB-CD28-CD3ζ.
anti-HER2-CD3ζ
anti-HER2-CD28-41BB-CD3ζ,
anti-HER2-CD28-CD3ζ
anti-HER2-CD28-OX40-CD3ζ
anti-HER2-CD28-41BB-CD3ζ
anti-HER2-OX40-CD3ζ
anti-HER2-OX40-CD28-CD3ζ
anti-HER2-41BB-CD3ζ
anti-HER2-ICOS-CD3ζ
anti-HER2-ICOS-41BB-CD3ζ
anti-HER2-41BB-ICOS-CD3ζ
anti-HER2-41BB-OX40-CD3ζ,
anti-HER2-41BB-CD28-CD3ζ.
anti-CEA-CD3ζ
anti-CEA-CD28-41BB-CD3ζ,
anti-CEA-CD28-CD3ζ
anti-CEA-CD28-OX40-CD3ζ
anti-CEA-CD28-41BB-CD3ζ
anti-CEA-OX40-CD3ζ
anti-CEA-OX40-CD28-CD3ζ
anti-CEA-41BB-CD3ζ
anti-CEA-ICOS-CD3ζ
anti-CEA-ICOS-41BB-CD3ζ
anti-CEA-41BB-ICOS-CD3ζ
anti-CEA-41BB-OX40-CD3ζ,
anti-CEA-41BB-CD28-CD3ζ.
anti-VEGF-CD3ζ
anti-VEGF-CD28-41BB-CD3ζ,
anti-VEGF-CD28-CD3ζ
anti-VEGF-CD28-OX40-CD3ζ
anti-VEGF-CD28-41BB-CD3ζ
anti-VEGF-OX40-CD3ζ
anti-VEGF-OX40-CD28-CD3ζ
anti-VEGF-41BB-CD3ζ
anti-VEGF-ICOS-CD3ζ
anti-VEGF-ICOS-41BB-CD3ζ
anti-VEGF-41BB-ICOS-CD3ζ
anti-VEGF-41BB-OX40-CD3ζ,
anti-VEGF-41BB-CD28-CD3ζ.
anti-CD19-CD3ζ
anti-CD19-CD28-41BB-CD3ζ,
anti-CD19-CD28-CD3ζ
anti-CD19-CD28-OX40-CD3ζ
anti-CD19-CD28-41BB-CD3ζ
anti-CD19-OX40-CD3ζ
anti-CD19-OX40-CD28-CD3ζ
anti-CD19-41BB-CD3ζ
anti-CD19-ICOS-CD3ζ
anti-CD19-ICOS-41BB-CD3ζ
anti-CD19-41BB-ICOS-CD3ζ
anti-CD19-41BB-OX40-CD3ζ,
anti-CD19-41BB-CD28-CD3ζ.
anti-EGFR-CD3ζ
anti-EGFR-CD28-41BB-CD3ζ,
anti-EGFR-CD28-CD3ζ
anti-EGFR-CD28-OX40-CD3ζ
anti-EGFR-CD28-41BB-CD3ζ
anti-EGFR-OX40-CD3ζ
anti-EGFR-OX40-CD28-CD3ζ
anti-EGFR-41BB-CD3ζ
anti-EGFR-ICOS-CD3ζ
anti-EGFR-ICOS-41BB-CD3ζ
anti-EGFR-41BB-ICOS-CD3ζ
anti-EGFR-41BB-OX40-CD3ζ, and
anti-EGFR-41BB-CD28-CD3ζ.
anti-CD19-CD3ζ
anti-CD19-CD28-41BB-CD3ζ,
anti-CD19-CD28-CD3ζ
anti-CD19-CD28-OX40-CD3ζ
anti-CD19-CD28-41BB-CD3ζ
anti-CD19-OX40-CD3ζ
anti-CD19-OX40-CD28-CD3ζ
anti-CD19-41BB-CD3ζ
anti-CD19-ICOS-CD3ζ
anti-CD19-ICOS-41BB-CD3ζ
anti-CD19-41BB-ICOS-CD3ζ
anti-CD19-41BB-OX40-CD3ζ,
anti-CD19-41BB-CD28-CD3ζ.
anti-PSMA-CD3ζ
anti-PSMA-CD28-41BB-CD3ζ,
anti-PSMA-CD28-CD3ζ
anti-PSMA-CD28-OX40-CD3ζ
anti-PSMA-CD28-41BB-CD3ζ
anti-PSMA-OX40-CD3ζ
anti-PSMA-OX40-CD28-CD3ζ
anti-PSMA-41BB-CD3ζ
anti-PSMA-ICOS-CD3ζ
anti-PSMA-ICOS-41BB-CD3ζ
anti-PSMA-41BB-ICOS-CD3ζ
anti-PSMA-41BB-OX40-CD3ζ,
anti-PSMA-41BB-CD28-CD3ζ.
anti-BCMA-CD3ζ
anti-BCMA-CD28-41BB-CD3ζ,
anti-BCMA-CD28-CD3ζ
anti-BCMA-CD28-OX40-CD3ζ
anti-BCMA-CD28-41BB-CD3ζ
anti-BCMA-OX40-CD3ζ
anti-BCMA-OX40-CD28-CD3ζ
anti-BCMA-41BB-CD3ζ
anti-BCMA-ICOS-CD3ζ
anti-BCMA-ICOS-41BB-CD3ζ
anti-BCMA-41BB-ICOS-CD3ζ
anti-BCMA-41BB-OX40-CD3ζ,
anti-BCMA-41BB-CD28-CD3ζ.
anti-mesothelin-CD3ζ
anti-mesothelin-CD28-41BB-CD3ζ,
anti-mesothelin-CD28-CD3ζ
anti-mesothelin-CD28-OX40-CD3ζ
anti-mesothelin-CD28-41BB-CD3ζ
anti-mesothelin-OX40-CD3ζ
anti-mesothelin-OX40-CD28-CD3ζ
anti-mesothelin-41BB-CD3ζ
anti-mesothelin-ICOS-CD3ζ
anti-mesothelin-ICOS-41BB-CD3ζ
anti-mesothelin-41BB-ICOS-CD3ζ
anti-mesothelin-41BB-OX40-CD3ζ,
anti-mesothelin-41BB-CD28-CD3ζ.
[anti-CD19 & anti-CD20]-CD3ζ
[anti-CD19 & anti-CD20]-CD28-41BB-CD3ζ,
[anti-CD19 & anti-CD20]-CD28-CD3ζ
[anti-CD19 & anti-CD20]-CD28-OX40-CD3ζ
[anti-CD19 & anti-CD20]-CD28-41BB-CD3ζ
[anti-CD19 & anti-CD20]-OX40-CD3ζ
[[anti-CD19 & anti-CD20]-OX40-CD28-CD3ζ
[anti-CD19 & anti-CD20]-41BB-CD3ζ
[anti-CD19 & anti-CD20]-ICOS-CD3ζ
[anti-CD19 & anti-CD20]-ICOS-41BB-CD3ζ
[anti-CD19 & anti-CD20]-41BB-ICOS-CD3ζ
[anti-CD19 & anti-CD20]-41BB-OX40-CD3ζ,
[anti-CD19 & anti-CD20]-41BB-CD28-CD3ζ.
anti-EGFR-CD3ζ
anti-EGFR-CD28-41BB-CD3ζ,
anti-EGFR-CD28-CD3ζ
anti-EGFR-CD28-OX40-CD3ζ
anti-EGFR-CD28-41BB-CD3ζ
anti-EGFR-OX40-CD3ζ
anti-EGFR-OX40-CD28-CD3ζ
anti-EGFR-41BB-CD3ζ
anti-EGFR-ICOS-CD3ζ
anti-EGFR-ICOS-41BB-CD3ζ
anti-EGFR-41BB-ICOS-CD3ζ
anti-EGFR-41BB-OX40-CD3ζ, and
anti-EGFR-41BB-CD28-CD3ζ.
[anti-CD19 & anti-CD22]-CD3ζ
[anti-CD19 & anti-CD22]-CD28-41BB-CD3ζ,
[anti-CD19 & anti-CD22]-CD28-CD3ζ
[anti-CD19 & anti-CD22]-CD28-OX40-CD3ζ
[anti-CD19 & anti-CD22]-CD28-41BB-CD3ζ
[anti-CD19 & anti-CD22]-OX40-CD3ζ
[anti-CD19 & anti-CD22]-OX40-CD28-CD3ζ
[anti-CD19 & anti-CD22]-41BB-CD3ζ
[anti-CD19 & anti-CD22]-ICOS-CD3ζ
[anti-CD19 & anti-CD22]-ICOS-41BB-CD3ζ
[anti-CD19 & anti-CD22]-41BB-ICOS-CD3ζ
[anti-CD19 & anti-CD22]-41BB-OX40-CD3ζ, and
[anti-CD19 & anti-CD22]-41BB-CD28-CD3.

Furthermore, in addition to the more conventional first and second generation CARS, the term CAR includes CAR variants including but not limited split CARs, ON-switch CARS, bispecific or tandem CARs, inhibitory CARs (iCARs) and induced pluripotent stem (iPS) CAR-T cells.

The term “Split CARs” refers to CARs wherein the extracellular portion, the ABD and the cytoplasmic signaling domain of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR molecules and derivatives thereof (i.e., CAR variants) are described, e.g., in PCT Application Nos. US2014/016527, US1996/017060, US2013/063083; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

The term “bispecific or tandem CARs” refers to CARs which include a secondary CAR binding domain that can either amplify or inhibit the activity of a primary CAR.

The term “inhibitory chimeric antigen receptors” or “iCARs” are used interchangeably herein to refer to a CAR where binding iCARs use the dual antigen targeting to shut down the activation of an active CAR through the engagement of a second suppressive receptor equipped with inhibitory signaling domains of a secondary CAR binding domain results in inhibition of primary CAR activation. T cells with specificity for both tumor and off target tissues can be restricted to tumor only by using an antigen-specific: WAR introduced into the T cells to protect the off-target tissue (Fedorov, et al., (2013). Science Translational Medicine, 5:215)Inhibitory CARs (iCARs) are designed to regulate CAR-T cells activity through inhibitory receptors signaling modules activation. This approach combines the activity of two CARs, one of which generates dominant negative signals limiting the responses of CAR-T cells activated by the activating receptor. iCARs can switch off the response of the counteracting activator CAR when bound to a specific antigen expressed only by normal tissues. In this way, iCARs-T cells can distinguish cancer cells from healthy ones, and reversibly block functionalities of transduced T cells in an antigen-selective fashion. CTLA-4 or PD-1 intracellular domains in iCARs trigger inhibitory signals on T lymphocytes, leading to less cytokine production, less efficient target cell lysis, and altered lymphocyte motility. In some embodiments, the iCAR comprises an single chain antibody (e.g. scFv, VIM, etc) that specifically binds to an inhibitory antigen, one or more intracellular derived from the ICDs immunoinhibitory receptors (including but not limited to CTLA-4, PD-1, LAG-3, 2B4 (CD244), BMA (CD272), MR, TIM-3, TG beta receptor dominant negative analog etc.) via a transmembrane region that inhibits T cell function specifically upon antigen recognition.

The term “tandem CAR” or “TanCAR” refers to CARs which mediate bispecific activation of T cells through the engagement of two chimeric receptors designed to deliver stimulatory or costimulatory signals in response to an independent engagement of two different tumor associated antigens.

Typically, the chimeric antigen receptor T-cells (CAR-T cells) are T-cells which have been recombinantly modified by transduction with an expression vector encoding a CAR in substantial accordance with the teaching above.

In some embodiments, an engineered T cell is allogeneic with respect to the individual that is treated. Graham et al. (2018) Cell 7(10) E155. In some embodiments an allogeneic engineered T cell is fully HLA matched. However not all patients have a fully matched donor and a cellular product suitable for all patients independent of HLA type provides an alternative.

Because the cell product may consist of a subject's own T-cells, the population of the cells to be administered is to the subject is necessarily variable. Consequently identifying the optimal concentration of the Additionally, since the CAR-T cell agent is variable, the response to such agents can vary and thus involves the ongoing monitoring and management of therapy related toxicities which are managed with a course of pharmacologic immunosuppression or B cell depletion prior to the administration of the CAR-T cell treatment. Usually, at least 1×10⁶ cells/kg will be administered, at least 1×10⁷ cells/kg, at least 1×10⁸ cells/kg, at least 1×10⁹ cells/kg, at least 1×10¹⁰ cells/kg, or more, usually being limited by the number of T cells that are obtained during collection. The engineered cells may be infused to the subject in any physiologically acceptable medium by any convenient route of administration, normally intravascularly, although they may also be introduced by other routes, where the cells may find an appropriate site for growth

If the T cells used in the practice of the present invention are allogeneic T cells, such cells may be modified to reduce graft versus host disease. For example, the engineered cells of the present invention may be TCRαβ receptor knock-outs achieved by gene editing techniques. TCRαβ is a heterodimer and both alpha and beta chains need to be present for it to be expressed. A single gene codes for the alpha chain (TRAC), whereas there are 2 genes coding for the beta chain, therefore TRAC loci KO has been deleted for this purpose. A number of different approaches have been used to accomplish this deletion, e.g. CRISPR/Cas9; meganuclease; engineered I-CreI homing endonuclease, etc. See, for example, Eyquem et al. (2017) Nature 543:113-117, in which the TRAC coding sequence is replaced by a CAR coding sequence; and Georgiadis et al. (2018) Mol. Ther. 26:1215-1227, which linked CAR expression with TRAC disruption by clustered regularly interspaced short palindromic repeats (CRISPR)/Cas9 without directly incorporating the CAR into the TRAC loci. An alternative strategy to prevent GVHD modifies T cells to express an inhibitor of TCRαβ signaling, for example using a truncated form of CD3 as a TCR inhibitory molecule.

### Vectors Encoding the CAR:

The preparation of CAR T-cells useful in the practice of the present invention is achieved by transforming isolated T-cells with an expression vector comprising a nucleic acid sequence encoding the CAR polyprotein described above. The vector may be a nonviral vector comprising either RNA or DNA or a viral vector.

Expression vectors to effect expression of the CD122 and/or CAR. In some embodiments, the vectors may be viral vectors or non-viral vectors. The term “non-viral vector” refers to an autonomously replicating, extrachromosomal circular DNA molecule, distinct from the normal genome and nonessential for cell survival under nonselective conditions capable of effecting the expression of a coding sequence in the target cell. Plasmids are examples of non-viral vectors. In order to facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, magnetic fields (electroporation)

### Expression Cassette:

The recombinant expression vector comprises one or more expression cassettes to direct the expression of the orthogonal CD122 and/or CAR. The term “expression cassette refers” to a recombinant (or synthetic) nucleic acid construct encoding a desired polypeptide operably linked to suitable genetic control elements that are capable of effecting expression of the polypeptide in the host cell to be transformed with the expression vector. The term “operably linked” refers to a linkage of polynucleotide elements in a functional relationship. A nucleic acid sequence is “operably linked” when it is placed into a functional relationship with another nucleic acid sequence. For example, a promoter is operably linked to a coding sequence if it controls the transcription of the polypeptide; a ribosome binding site is operably linked to a coding sequence if it is positioned to permit translation, a nucleic acid encoding signal peptide is operably linked to a nucleic acid sequence encoding such polypeptide if it is expressed as a fusion protein and participates in directing the fusion protein to the cell membrane or in secretion of the polypeptide. Typically, nucleotide sequences that are operably linked are contiguous. However, as enhancers generally function when separated from the promoter by several kilobases and intronic sequences may be of variable lengths, some polynucleotide elements may be operably linked yet physically distant and may even function in trans from a different allele or chromosome.

### Control Elements

The specific type of control elements necessary to effect expression will depend upon the cell type to be transformed. In the practice of the present invention, the cell to be transformed is a mammalian T-cell. The term control elements refers collectively to promoter sequences, polyadenylation signals, transcription termination sequences, upstream regulatory domains, origins of replication, internal ribosome entry sites (“IRES”), enhancers, transcription enhancers to elevate the level of mRNA expression, a sequence that encodes a suitable ribosome binding site, and sequences that terminate transcription and translation which affect the replication, transcription and translation of the polypeptide coding sequence in a recipient cell. Expression vectors also usually contain an origin of replication that allows the vector to replicate independently of the host cell.

In one embodiment of the expression cassette, the nucleic acid sequence to be expressed (e.g. encoding the orthogonal CD122 and/or CAR) is operably linked to a promoter sequence. The term “promoter” is used in its conventional sense to refer to a nucleotide sequence at which the initiation and rate of transcription of a coding sequence is controlled. The promoter contains the site at which RNA polymerase binds and also contains sites for the binding of regulatory factors (such as repressors or transcription factors). Promoters can be naturally occurring or synthetic. The promoter can be constitutively active, activated in response to external stimuli (inducible), active in particular cell type or cell state (tissue specific or tumor specific) promoters, and/or regulatable promoters. The term “inducible promoter” refers to promoters that facilitate transcription of the Bioactive polypeptide preferably (or solely) under certain conditions and/or in response to external chemical or other stimuli. Examples of inducible promoters are known in the scientific literature (see, e.g., Yoshida et al., Biochem. Biophys. Res. Comm., 230:426-430 (1997); Iida et al., J. Virol., 70(9): 6054-6059 (1996); Hwang et al., J. Virol., 71(9): 7128-7131 (1997); Lee et al., Mol. Cell. Biol., 17(9): 5097-5105 (1997); and Dreher et al., J. Biol. Chem., 272(46): 29364-29371 (1997). Examples of radiation inducible promoters include the EGR-1 promoter. Boothman et al., volume 138, supplement pages S68-S71 (1994). In some embodiments the promoter is a tissue specific promoter. In some embodiments the promoter is a tumor specific promoter. Tissue specific promoters and tumor specific promoters are well known in the art, e.g., pancreas specific promoters (Palmiter et al., Cell, 50:435 (1987)), liver specific promoters (Rovet et al., J. Biol. Chem., 267:20765 (1992); Lemaigne et al., J. Biol. Chem., 268:19896 (1993); Nitsch et al., Mol. Cell. Biol., 13:4494 (1993)), stomach specific promoters (Kovarik et al., J. Biol. Chem., 268:9917 (1993)), pituitary specific promoters (Rhodes et al., Genes Dev., 7:913 (1993)), and prostate specific promoters (Henderson et. al., U.S. Pat. No. 5,698,443, issued Dec. 16, 1997). In one embodiment the promoter is the phosphoglycerase kinase (PGK) promoter. In one embodiment the promoter is the elongase factor 1 alpha (EF1a) promoter. In one embodiment the promoter is the myoproliferative sarcoma virus (enhancer negative control region deleted dl587rev primer binding site substituted) (“MND”) promoter.

When expressing multiple polypeptides (e.g., a CAR and orthogonal CD122 polypeptides) as in the practice of the present invention, each polypeptide may be operably linked to an expression control sequence (monocistronic) or multiple polypeptides may be encoded by a polycistronic construct where multiple polypeptides are expressed under the control of a single expression control sequence. Examples of elements which may be employed to facilitate polycistronic expression internal ribosome entry site (IRES) elements or the foot and mouth disease virus protein 2A (FMVD2A) system or a T2A peptide A wide variety of IRES sites are known (see e.g. Doudna J A, Sarnow P. Translation initiation by viral internal ribosome entry sites. In: Translational Control in Biology and Medicine; Mathews et al, Ed. Cold Spring Harbor, N.Y.: Cold Spring Harbor Laboratory Press; 2007. pp. 129-154; http://www.IRESite.org). Examples of IRES elements include the picornavirus IRES of poliovirus, rhinovirus, encepahlomyocardits virus, the aphthovirus IRES of foot and mouth disease virus, the IRES cricket paralysis virus (CrPV) the hepatitis A IRES of hepatitis A virus, the hepatitis C IRES of hepatitis C virus, the pestivirus IRES of swine fever or bovine diarrhea viruses, the cripavirus IRES, and mammalian IRES elements such as the fibroblast growth factor-1 IRES, the fibroblast growth factor-2 IRES, PDGF IRES, VEGF IRES, IGF-2 IRES. The use of IRES elements typically results in significantly lower expression of the second protein of the polycistronic message. The use of the FMDV2A system results in more efficient production of the downstream proteins as the multiple proteins are first expressed as a fusion protein which contains the autoproteolytic FMDV2A domain which cleaves the polyprotein into functional subunits. Ryan and Drew (1994) EMBO J. 13(4):928-933. Depending on the construction of the polycistronic coding sequence, especially to facilitate restriction endonuclease sites, the use of the FMDV2A system frequently may in the addition of a small number amino acids to the carboxy terminus of the upstream protein.

### Optional Encoded Proteins: Rescue Gene/Drug Resistance:

The expression vector encoding the CAR and/or orthogonal CD122 may optionally provide one or more expression cassettes comprising a nucleic acid sequence encoding a “rescue” gene. A “rescue gene” is a nucleic acid sequence, the expression of which in the transduced cell renders the cell susceptible to killing by external factors or causes a toxic condition in the cell such that the cell is killed. Providing a rescue gene enables selective cell killing of transduced cells. Thus the rescue gene provides an additional safety precaution when said constructs are incorporated into the cells of a mammalian subject to prevent undesirable spreading of transduced cells or the effects of replication competent vector systems. In one embodiment, the rescue gene is the thymidine kinase (TK) gene (see e.g. Woo, et al. U.S. Pat. No. 5,631,236 issued May 20, 1997 and Freeman, et al. U.S. Pat. No. 5,601,818 issued Feb. 11, 1997) in which the cells expressing the TK gene product are susceptible to selective killing by the administration of gancyclovir. Alternatively, an inducible promoter may be operably linked to a proapototic gene to facilitate targeted cell killing by the administration of an exogenous agent that induces expression from the inducible promoter.

The expression vector may optionally provide additional genes, such as those encoding drug resistance, can be included to allow selection or screening for the presence of the recombinant vector. Such additional genes can include, for example, genes encoding neomycin resistance, multi-drug resistance, thymidine kinase, beta-galactosidase, dihydrofolate reductase (DHFR), and chloramphenicol acetyl transferase.

In one embodiment, a non-viral vector may be provided in a non-viral delivery system. Non-viral delivery systems are typically complexes to facilitate transduction of the target cell with a nucleic acid cargo wherein the nucleic acid is complexed with agents such as cationic lipids (DOTAP, DOTMA), surfactants, biologicals (gelatin, chitosan), metals (gold, magnetic iron) and synthetic polymers (PLG, PEI, PAMAM). Numerous embodiments of non-viral delivery systems are well known in the art including lipidic vector systems (Lee et al. (1997) Crit Rev Ther Drug Carrier Syst. 14:173-206); polymer coated liposomes (Marin et al., U.S. Pat. No. 5,213,804, issued May 25, 1993; Woodle, et al., U.S. Pat. No. 5,013,556, issued May 7, 1991); cationic liposomes (Epand et al., U.S. Pat. No. 5,283,185, issued Feb. 1, 1994; Jessee, J. A., U.S. Pat. No. 5,578,475, issued Nov. 26, 1996; Rose et al, U.S. Pat. No. 5,279,833, issued Jan. 18, 1994; Gebeyehu et al., U.S. Pat. No. 5,334,761, issued Aug. 2, 1994). The efficiency of expression CAR sequences in T-cells with non-viral vectors can be considerably increased by the use of transposon/transposase systems such as the so-called Sleeping Beauty (SB) transposon system (See. e.g., Geurts, et al. (2003) Mol Ther 8(1):108-117) and the piggyBac system (See, e.g. Manuri, et al. (2010) Human Gene Therapy 21(4):427-437) can be used to stably introduce non-viral vectors (e.g. plasmids) comprising nucleic acid sequences encoding anti-targeting antigen CAR into human T-cells.

In alternative procedure for non-viral gene delivery to create the CAR is achieved by transfection of mRNA vector encoding the CAR in substantial accordance with the teaching of Rabinovich, et al (U.S. Pat. No. 10,155,038BS issued Dec. 18, 2018, the entire teaching of which is herein incorporated by reference. In some embodiments when the vector encoding the CAR is an RNA vector, optionally the RNA vector may encode one or more additional biologically active molecules. For example, when the vector is an RNA vector, the vector can encode one or more RNA(s) that reprogram the cells to prevent expression of one or more antigens. For example, as discussed in more detail below, the RNA may be an interfering RNA that prevents expression of an mRNA encoding antigens as CTLA-4 or PD-1. This method can be used to prepare universal donor cells. RNAs used to alter the expression of allogenic antigens may be used alone or in combination with RNAs that result in de-differentiation of the target cell. In some embodiments, the biologically active RNA is a short Interfering RNA (siRNA). siRNA is a double-stranded RNA that can induce sequence-specific post-transcriptional gene silencing, thereby decreasing or even inhibiting gene expression. In one example, a siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs.

In another embodiment, the expression vector for the CAR and/or orthogonal receptor may be a viral vector. As used herein, the term viral vector is used in its conventional sense to refer to any of the obligate intracellular parasites having no protein-synthesizing or energy-generating mechanism and generally refers to any of the enveloped or non-enveloped animal viruses commonly employed to deliver exogenous transgenes to mammalian cells. A viral vector may be replication competent (e.g., substantially wild-type), conditionally replicating (recombinantly engineered to replicate under certain conditions) or replication deficient (substantially incapable of replication in the absence of a cell line capable of complementing the deleted functions of the virus). The viral vector can possess certain modifications to make it “selectively replicating,” i.e. that it replicates preferentially in certain cell types or phenotypic cell states, e.g., cancerous. Viral vector systems useful in the practice of the instant invention include, for example, naturally occurring or recombinant viral vector systems. Examples of viruses useful in the practice of the present invention include recombinantly modified enveloped or non-enveloped DNA and RNA viruses. For example, viral vectors can be derived from the genome of human or bovine adenoviruses, vaccinia virus, lentivirus, herpes virus, adeno-associated virus, human immunodeficiency virus, sindbis virus, and retroviruses (including but not limited to Rous sarcoma virus), and hepatitis B virus. Typically, genes of interest are inserted into such vectors to allow packaging of the gene construct, typically with accompanying viral genomic sequences, followed by infection of a sensitive host cell resulting in expression of the gene of interest (e.g. a targeting antigen). Additionally, the expression vector encoding the Anti-targeting antigen CAR may also be an mRNA vector. When a viral vector system is to be employed for transfection, retroviral or lentiviral expression vectors are preferred to transfect T-cells due to an enhanced efficacy of gene transfer to T-cells using these systems resulting in a decreased time for culture of significant quantities of T-cells for clinical applications. In particular, gamma retroviruses a particularly preferred for the genetic modification of clinical grade T-cells and have been shown to have therapeutic effect. Pule, et al. (2008) Nature Medicine 14(11):1264-1270. Similarly, self-inactivating lentiviral vectors are also useful as they have been demonstrated to integrate into quiescent T-cells. June, et al. (2009) Nat Rev Immunol 9(10):704-716.

The expression vector encoding the CAR and/or orthogonal receptor may encode one or more polypeptides in addition to the CAR and/or orthogonal receptor. When expressing multiple polypeptides as in the practice of the present invention, each polypeptide may be operably linked to an expression control sequence (monocistronic) or multiple polypeptides may be encoded by a polycistronic construct where multiple polypeptides are expressed under the control of a single expression control sequence. In one embodiment, the expression vector comprises a polycistronic expression cassette comprising a nucleic acid sequence encoding a CAR and orthogonal CD122.

In one embodiment, the expression vector encoding the CAR and/or orthogonal receptor may optionally further encode one or more polypeptide supplementary agents as described herein. In some embodiments, expression vector encoding the targeting antigen may optionally further encode one or more polypeptide supplementary agents as described herein. the immunological modulators. Examples of immunological modulators useful in the practice of the present invention include but are not limited to cytokines. Examples of such cytokines are interleukins including but not limited to one more or of IL-1, IL-2, IL-3, IL-4, IL-10, IL-12, TNF-alpha, interferon alpha, interferon alpha-2b, interferon-beta, interferon-gamma, GM-CSF, MIP1-alpha, MIP1-beta, MIP3-alpha, TGF-beta and other suitable cytokines capable of modulating immune response. The expressed cytokines can be directed for intracellular expression or expressed with a signal sequence for extracellular presentation or secretion. The co-administration of a CAR with IL-12 has reportedly resulted in enhanced antitumor efficacy (See Yeku, et al Scientific Reports Vol. 7, Article number: 10541(2017) Published online: 5 Sep. 2017

Alternative to the use of multiple expression cassettes, the nucleic acid sequences encoding the CAR and orthogonal CD122 polypeptide may be encoded by a polycistronic construct, the expression cassette comprising the nucleic acid sequences CAR and orthogonal

CD122 polypeptide employing an internal ribosome entry site (IRES) element or the foot and mouth disease virus protein 2A (FMVD2A) to facilitate co-expression in the target cell. In one embodiment, the expression vector comprises two expression cassettes, a first expression cassette comprising a nucleic acid sequence encoding the CAR operably linked to an expression control sequence and a second expression cassette comprising a nucleic acid sequence encoding the orthogonal CD122 operably linked to an expression control sequence, in each case the expression control sequence being functional in the cell type (e.g. T cell) to be used to host expression of the CAR and orthogonal CD122. In one embodiment, the expression vector comprises a polycistronic expression cassette comprising a nucleic acid sequence encoding a CAR and orthogonal CD122.

The expression vector may optionally provide an additional expression cassette comprising a nucleic acid sequence encoding a “rescue” gene. A “rescue gene” is a nucleic acid sequence, the expression of which renders the cell susceptible to killing by external factors or causes a toxic condition in the cell such that the cell is killed. Providing a rescue gene enables selective cell killing of transduced cells. Thus, the rescue gene provides an additional safety precaution when said constructs are incorporated into the cells of a mammalian subject to prevent undesirable spreading of transduced cells or the effects of replication competent vector systems. In one embodiment, the rescue gene is the thymidine kinase (TK) gene (see e.g. Woo, et al. U.S. Pat. No. 5,631,236 issued May 20, 1997 and Freeman, et al. U.S. Pat. No. 5,601,818 issued Feb. 11, 1997) in which the cells expressing the TK gene product are susceptible to selective killing by the administration of gancyclovir.

Transforming T-Cells with an Expression Vector Encoding the Anti-Targeting Antigen CAR

An prerequisite to transforming T-cells with an expression vector encoding the anti-targeting antigen CAR is a source of T-cells. T-cells may be obtained from the mammalian subject to be treated or may be any of a variety of T cell lines available in the art. T-cells for transformation are typically obtained from the mammalian subject to be treated. T cells can be obtained from a number of sources of the mammalian subject, including peripheral blood mononuclear cells, bone marrow, lymph node tissue, cord blood, thymus tissue, spleen tissue, and tumors. In one embodiment, T-cells are obtained by apheresis. In another embodiment, T cells are isolated from peripheral blood and particular T cells (such as CD3⁺, CD28⁺, CD4⁺, CD8⁺, CD45RA⁺, and CD45RO⁺T cells) can be isolate by selection techniques well known in the art such is incubation with anti-CD3/anti-CD28 conjugated beads.

The population of selected T-cells is transformed with an expression vector encoding the Anti-targeting antigen CAR in substantial accordance with teachings hereinabove. Following transformation, T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 2006/0121005. Generally, the T cells of the invention are expanded by culturing the cells in contact with a surface providing an agent that stimulates a CD3 TCR complex associated signal (e.g., an anti-CD3 antibody) and an agent that stimulates a co-stimulatory molecule on the surface of the T cells (e.g an anti-CD28 antibody). Conditions appropriate for T cell culture are well known in the art Lin, et al. (2009) Cytotherapy 11(7):912-922 (Optimization and validation of a robust human T-cell culture method for monitoring phenotypic and polyfunctional antigen-specific CD4 and CD8 T-cell responses); Smith, et al. (2015) Clinical & Translational Immunology 4:e31 published online 16 Jan. 2015 (“Ex vivo expansion of human T cells for adoptive immunotherapy using the novel Xeno-free CTS Immune Cell Serum Replacement”). The target cells are maintained under conditions necessary to support growth, for example, an appropriate temperature (e.g., 37° C.) and atmosphere (e.g., air plus 5% CO₂).

### Genetic Modification of the Immune Cell to Express the Orthogonal Receptor

In an alternative to the expression of the orthogonal receptor from a vector, the genome of the cell may be modified to express the orthogonal receptor using techniques known in the art. In some embodiments, the compositions and methods of the present disclosure comprise the step of genetically modifying a human immune cell by using at least one endonuclease to facilitate incorporate the modifications of to the ECD of the orthogonal hCD122 of Formula 1 into the genomic sequence of the human immune cell. As used herein, the term “endonuclease” is used to refer to a wild-type or variant enzyme capable of catalyzing the cleavage of bonds between nucleic acids within a DNA or RNA molecule, preferably a DNA molecule. The endonucleases of the present disclosure are sequence specific in that they recognize and cleave the nucleic acid molecules a specific “target” sequences. Endonucleases are often categorized with respect to the degree of specificity and sequence identity characteristic of the target sequences. Endonucleases are referred to as “rare-cutting” endonucleases when such endonucleases have a polynucleotide recognition site greater than about 12 base pairs (bp) in length, more preferably of 14-55 bp. Rare-cutting endonucleases can be used for inactivating genes at a locus or to integrate transgenes by homologous recombination (HR) i.e. by inducing DNA double-strand breaks (DSBs) at a locus and insertion of exogenous DNA at this locus by gene repair mechanism. Examples of rare-cutting endonucleases include homing endonucleases (Grizot, et al (2009) Nucleic Acids Research 37(16):5405-5419), chimeric Zinc-Finger nucleases (ZEN) resulting from the fusion of engineered zinc-finger domains (Porteus NI and Carroll D., Gene targeting using zinc finger nucleases (2005) Nature Biotechnology 23(3):967-973, a TALE-nuclease, a Cas9 endonuclease from CRISPR system as or a modified restriction endonuclease to extended sequence specificity (Eisenschmidt, et al. 2005; 33(22): 7039-7047). In some embodiments of the invention, the immune cell (e.g. a CAR-T expressing the orthogonal receptor CCD of Formula 1) is modified to reduce alloreactivity through inactivation of one more components of the T-cell receptor (TCR). Methods for such modification of T cells is described in Galetto, et al. United States Patent Application Publication No. US 2013/015884A1 published Nov. 28, 2013 and methods for TCRalpha deficient T-cells by expressing pTalpha resulting in restoration of a functional CD3 complex as described in Galetto, et al. U.S. Pat. No. 10,426,795B2 issued Oct. 21, 2019. the teaching of which is herein incorporated by reference.

### Use of Ortho CAR-T Cells with Ortho Ligand

In one embodiment, the present disclosure provides a method of selectively expanding a population of engineered cells expressing an orthogonal CD122 receptor from a mixed cell population, the method comprising contacting the mixed cell population with an IL2 ortholog of the present disclosure under conditions that facilitate the proliferation of the engineered cell. In one embodiment when the orthogonal CD122 receptor expressing CAR-T cell, the orthogonal receptor expressing CAR-T cells may also be selectively expanded from the background or mixed population of transduced and non-transduced cells through the use of the IL2 orthologs described herein. Expansion of the T cells for therapeutic applications typically involves culturing the cells in contact with a surface providing an agent that stimulates a CD3 TCR complex associated signal and an agent that stimulates a co-stimulatory molecule on the surface of the T-cell. In conventional practice, engineered T-cells are stimulated prior to administration of the cell therapy product by contacting with CD3/D28, particularly in the preparation of CAR-T cells for use in clinical applications. A variety of commercially available products are available to facilitate bead-based activation of T-cells including but not limited to the Invitrogen® CTS Dynabeads® CD3/28 (Life Technologies, Inc. Carlsbad Calif.) or Miltenyi MACS® GMP ExpAct Treg beads or Miltenyi MACS GMP TransAct™ CD3/28 beads (Miltenyi Biotec, Inc.). Conditions appropriate for T-cell culture are well known in the art. Lin, et al. (2009) Cytotherapy 11(7):912-922; Smith, et al. (2015) Clinical & Translational Immunology 4:e31 published online 16 Jan. 2015. The target cells are maintained under conditions necessary to support growth, for example, an appropriate temperature (e.g., 37° C.) and atmosphere (e.g., air plus 5% CO₂). Wherein the mixed cell population containing engineered T cells expressing the CD122 orthogonal receptor is cultured in the presence of a concentration of the IL2 ortholog. In some embodiments for at least 2 hours, alternatively at least 3 hours, alternatively at least 4 hours, alternatively at least 6 hours, alternatively at least 8 hours, alternatively at least 12 hours, alternatively at least 24 hours, alternatively at least 48 hours, alternatively at least 72 hours, or more. The concentration of the IL2 ortholog in such ex vivo situations is sufficient to induce cellular proliferation in the cell population. T cell proliferation can be readily assessed by microscopic methods and the determination of the optimal concentration of the IL2 ortholog will depend upon the relative activity of the IL2 ortholog for the orthogonal CD122 receptor.

Where the cells are contacted with the IL2 ortholog in vitro, the cytokine is added to the engineered cells in a dose and for a period of time sufficient to activate signaling from the receptor, which may utilize the native cellular machinery, e.g. accessory proteins, co-receptors, and the like. Any suitable culture medium may be used. The cells thus activated may be used for any desired purpose, including experimental purposes relating to determination of antigen specificity, cytokine profiling, and the like, and for delivery in vivo.

Where the contacting is performed in vivo, an effective dose of engineered cells, including without limitation CAR-T cells modified to express an orthogonal CD122 receptor, are infused to the recipient, in combination with or prior to administration of the orthogonal cytokine, e.g. IL2 and allowed to contact T cells in their native environment, e.g. in lymph nodes, etc. Dosage and frequency may vary depending on the agent; mode of administration; nature of the IL2 ortholog, and the like. It will be understood by one of skill in the art that such guidelines will be adjusted for the individual circumstances. The dosage may also be varied for route of administration, e.g. intramuscular, intraperitoneal, intradermal, subcutaneous, intravenous infusion and the like. Generally at least about 10⁴ engineered cells/kg are administered, at least about 10⁵ engineered cells/kg; at least about 10⁶ engineered cells/kg, at least about 10⁷ engineered cells/kg, or more.

Where the engineered cells are T cells, an enhanced immune response may be manifest as an increase in the cytolytic response of T cells towards the target cells present in the recipient, e.g. towards elimination of tumor cells, infected cells; decrease in symptoms of autoimmune disease; and the like. In some embodiments when the engineered T cell population is to be administered to a subject, the subject is provided with immunosuppressive course of therapy prior to or in combination with the administration of the engineered T cell population. Examples of such immunosuppressive regimens include but are not limited to systemic corticosteroids (e.g., methylprednisolone). Therapies for B cell depletion include intravenous immunoglobulin (IVIG) by established clinical dosing guidelines to restore normal levels of serum immunoglobulin levels. In some embodiments, prior to administration of the CAR-T cell therapy of the present invention, the subject may optionally be subjected to a lymphodepleting regimen. One example of a such lymphodepleting regimen consists of the administration to the subject of fludarabine (30 mg/m² intravenous daily for 4 days) and cyclophosphamide (500 mg/m² IV daily for 2 days starting with the first dose of fludarabine).

Engineered T cells can be provided in pharmaceutical compositions suitable for therapeutic use, e.g. for human treatment. Therapeutic formulations comprising such cells can be frozen, or prepared for administration with physiologically acceptable carriers, excipients or stabilizers (Remington's Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The cells can be administered by any suitable means, usually parenteral. Parenteral infusions include intramuscular, intravenous (bolus or slow infusion), intraarterial, intraperitoneal, intrathecal or subcutaneous administration. In the typical practice, the engineered T cells are infused to the subject in a physiologically acceptable medium, normally intravascularly, although they may also be introduced into any other convenient site, where the cells may find an appropriate site for growth. Usually, at least 1×10⁵ cells/kg will be administered, at least 1×10⁶ cells/kg, at least 1×10⁷ cells/kg, at least 1×10⁸ cells/kg, at least 1×10⁹ cells/kg, or more, usually being limited by the number of T cells that are obtained during collection.

For example, typical ranges for the administration of hoRb cells for use in the practice of the present invention range from about 1×10⁵ to 5×10⁸ viable cells per kg of subject body weight per course of therapy. Consequently, adjusted for body weight, typical ranges for the administration of viable cells in human subjects ranges from approximately 1×10⁶ to approximately 1×10¹³ viable cells, alternatively from approximately 5×10⁶ to approximately 5×10¹² viable cells, alternatively from approximately 1×10⁷ to approximately 1×10¹² viable cells, alternatively from approximately 5×10⁷ to approximately 1×10¹² viable cells, alternatively from approximately 1×10⁸ to approximately 1×10¹² viable cells, alternatively from approximately 5×10⁸ to approximately 1×10¹² viable cells, alternatively from approximately 1×10⁹ to approximately 1×10¹² viable cells per course of therapy. In one embodiment, the dose of the cells is in the range of 2.5-5×10⁹ viable cells per course of therapy.

A course of therapy may be a single dose or in multiple doses over a period of time. In some embodiments, the cells are administered in a single dose. In some embodiments, the cells are administered in two or more split doses administered over a period of 1, 2, 3, 4, 5, 6, 7, 8, 9, 10, 11, 12, 13, 14, 21, 28, 30, 60, 90, 120 or 180 days. The quantity of engineered cells administered in such split dosing protocols may be the same in each administration or may be provided at different levels. Multi-day dosing protocols over time periods may be provided by the skilled artisan (e.g. physician) monitoring the administration of the cells taking into account the response of the subject to the treatment including adverse effects of the treatment and their modulation as discussed above.

### Absence of Lymphodepletion

The compositions and methods of the present disclosure also provide a method for the treatment of a subject with a T cell therapy (especially CART cell therapy) in the absence of prior lymphodepletion. Lymphodepletion is typically performed in a subject in conjunction with CAR T cell therapy because the subsequent administration of the mixed cell population and the administration of non-specific agents (e.g. IL2) to expand the engineered cell population in the subject in combination with the administration of the cell therapy product acts results in significant systemic toxicity (including cytokine release syndrome or “cytokine storm”) arising from the widespread proliferation and activation of immune cells by administration of agents that result in widespread activation as well as the presence of a substantial fraction of non-engineered cells in the cell therapy product itself. The methods and compositions of the present disclosure obviate this significant hurdle by both (or either) providing a substantially purified population of engineered cells largely devoid of contamination by non-engineered cells when the foregoing ex vivo method is employed and/or the selective activation and expansion of the engineered T cells with the IL2 orthologs which provide substantially reduced off-target effects of non-specific proliferative agents such as IL2.

For example, in the current clinical practice of CAR-T cell therapy, CAR-T cells are commonly administered in combination with lymphodepletion (e.g. by administration of Alemtuzumab (monoclonal anti-CD52), purine analogs, and the like) to facilitate expansion of the CAR-T cells to prior to host immune recovery. In some embodiments, the CAR-T cells may be modified for resistance to Alemtuzumab. In one aspect of the invention, the lymphodepletion currently employed in association with CAR-T therapy may be obviated or reduced by the orthogonal ligand expressing CAR-Ts. As noted above, the lymphodepletion is commonly employed to enable expansion of the CAR-T cells. However, the lymphodepletion is also associated with major side effects of CAR-T cell therapy. Because the orthogonal ligand provides a means to selectively expand a particular T-cell population, the need for lymphodepletion prior to administration of the orthogonal ligand expressing CAR-Ts may be reduced. The present invention enables the practice of CAR-T cell therapy without or with reduced lymphodepletion prior to administration of the orthogonal ligand expressing CAR-Ts.

In one embodiment, the present disclosure provides a method of treating a subject suffering from a disease, disorder or condition amendable to treatment with CAR-T cell therapy (e.g. cancer) by the administration of a orthogonal ligand expressing CAR-Ts in the absence of lymphodepletion prior to administration of the orthogonal ligand CAR-Ts. In one embodiment, the present disclosure provides for a method of treatment of a mammalian subject suffering from a disease, disorder associated with the presence of an aberrant population of cells (e.g. a tumor) said population of cells characterized by the expression of one or more surface antigens (e.g. tumor antigen(s)), the method comprising the steps of (a) obtaining a biological sample comprising T-cells from the individual; (b) enriching the biological sample for the presence of T-cells; (c) transfecting the T-cells with one or more expression vectors comprising a nucleic acid sequence encoding a CAR and a nucleic acid sequence encoding an orthogonal CD122 receptor, the antigen targeting domain of the CAR being capable of binding to at least one antigen present on the aberrant population of cells; (d) expanding the population of the orthogonal receptor expressing CAR-T cells ex vivo with an IL2 ortholog; (e) administering a pharmaceutically effective amount of the orthogonal receptor expressing CAR-T cells to the mammal; and (f) modulating the growth of the orthogonal CD122 receptor expressing CAR-T cells by the administration of a therapeutically effective amount of an IL2 ortholog that binds selectively to the orthogonal CD122 receptor expressed on the CAR-T cell. In one embodiment, the foregoing method is associated with lymphodepletion or immunosuppression of the mammal prior to the initiation of the course of CAR-T cell therapy. In another embodiment, the foregoing method is practiced in the absence of lymphodepletion and/or immunosuppression of the mammal.

### Maintaining Threshold Levels of Orthogonal Immune Cells Over Time Using Ortho Ligand:

In one embodiment in the practice of the currently disclosed methods of using orthogonal immune cells in combination with an orthogonal ligand, the orthogonal ligand is administered to the subject in an amount to maintain a therapeutically effective circulating level of orthogonal cells in the circulation of an human subject wherein the therapeutically effective circulating level of orthogonal is about 10,000 to about 1,000,000 orthogonal cells, alternatively from about 20,000 to about 500,000 orthogonal cells, alternatively from about 30,000 to about 300,000 orthogonal cells, alternatively from about 30,000 to about 200,000 orthogonal cells, alternatively from about 20,000 to about 150,000 orthogonal cells, alternatively from about 50,000 to about 150,000 orthogonal cells per kg of subject body weight, is maintained for a period of at least one week, alternatively at least two weeks, alternatively at least 3 weeks, alternatively at least one month, alternatively at least two months, alternatively at least 3 months alternatively at least 6 months alternatively at least 9 months alternatively at least 12 months by the periodic administration of an orthogonal ligand, e.g., an orthogonal ligand of Formula 1. Quantitation of the engineered cells may readily be determined by conventional antibody based methods. Excessive levels of CRP and Ferritin are used clinically to evaluate for the onset of CRS and will be typically be monitored by the attending physician to determine an optimum tolerable and efficacious levels of the orthogonal cells and the levels of orthogonal ligand necessary to maintain such level as the response of human subjects can be variable. Identification of an optimal circulating concentration of orthogonal cells for an individual patient is within the skill of the ordinary practitioner of cell therapies.

As previously noted, the present disclosure provides a method of treating a subject who exhibits evidence of significant clinical response to the initial orthogonal cell therapy treatment to prevent relapse or recurrence by the administration of a maintenance regimen wherein a lower dose of the orthogonal ligand is periodically administered to subject to maintain a lower circulating level of orthogonal cells of about 10,000 to about 500,000 orthogonal cells, alternatively from about 10,000 to about 200,000 orthogonal cells, alternatively from about 10,000 to about 100,000 orthogonal cells, per kg of subject body weight is maintained for a maintenance phase period of at least one week, alternatively at least two weeks, alternatively at least 3 weeks, alternatively at least one month, alternatively at least two months, alternatively at least 3 months alternatively at least 6 months alternatively at least 9 months alternatively at least 12 months by the periodic administration of an orthogonal ligand, e.g., an orthogonal ligand of Formula 1.

### Use of Ortho CAR-T Cells with Ortho Ligand

In one embodiment, the present disclosure provides a method of selectively expanding a population of engineered cells expressing an orthogonal CD122 receptor from a mixed cell population, the method comprising contacting the mixed cell population with an IL2 ortholog of the present disclosure under conditions that facilitate the proliferation of the engineered cell. In one embodiment when the orthogonal CD122 receptor expressing CAR-T cell, the orthogonal receptor expressing CAR-T cells may also be selectively expanded from the background or mixed population of transduced and non-transduced cells through the use of the IL2 orthologs described herein. Expansion of the T cells for therapeutic applications typically involves culturing the cells in contact with a surface providing an agent that stimulates a CD3 TCR complex associated signal and an agent that stimulates a co-stimulatory molecule on the surface of the T-cell. In conventional practice, engineered T-cells are stimulated prior to administration of the cell therapy product by contacting with CD3/D28, particularly in the preparation of CAR-T cells for use in clinical applications. A variety of commercially available products are available to facilitate bead-based activation of T-cells including but not limited to the Invitrogen® CTS Dynabeads® CD3/28 (Life Technologies, Inc. Carlsbad Calif.) or Miltenyi MACS® GMP ExpAct Treg beads or Miltenyi MACS GMP TransAct™ CD3/28 beads (Miltenyi Biotec, Inc.). Conditions appropriate for T-cell culture are well known in the art. Lin, et al. (2009) Cytotherapy 11(7):912-922; Smith, et al. (2015) Clinical & Translational Immunology 4:e31 published online 16 Jan. 2015. The target cells are maintained under conditions necessary to support growth, for example, an appropriate temperature (e.g., 37° C.) and atmosphere (e.g., air plus 5% CO₂). Wherein the mixed cell population containing engineered T cells expressing the CD122 orthogonal receptor is cultured in the presence of a concentration of the IL2 ortholog. In some embodiments for at least 2 hours, alternatively at least 3 hours, alternatively at least 4 hours, alternatively at least 6 hours, alternatively at least 8 hours, alternatively at least 12 hours, alternatively at least 24 hours, alternatively at least 48 hours, alternatively at least 72 hours, or more. The concentration of the IL2 ortholog in such ex vivo situations is sufficient to induce cellular proliferation in the cell population. T cell proliferation can be readily assessed by microscopic methods and the determination of the optimal concentration of the IL2 ortholog will depend upon the relative activity of the IL2 ortholog for the orthogonal CD122 receptor.

Where the cells are contacted with the IL2 ortholog in vitro, the cytokine is added to the engineered cells in a dose and for a period of time sufficient to activate signaling from the receptor, which may utilize the native cellular machinery, e.g. accessory proteins, co-receptors, and the like. Any suitable culture medium may be used. The cells thus activated may be used for any desired purpose, including experimental purposes relating to determination of antigen specificity, cytokine profiling, and the like, and for delivery in vivo.

Where the contacting is performed in vivo, an effective dose of engineered cells, including without limitation CAR-T cells modified to express an orthogonal CD122 receptor, are infused to the recipient, in combination with or prior to administration of the orthogonal cytokine, e.g. IL2 and allowed to contact T cells in their native environment, e.g. in lymph nodes, etc. Dosage and frequency may vary depending on the agent; mode of administration; nature of the IL2 ortholog, and the like. It will be understood by one of skill in the art that such guidelines will be adjusted for the individual circumstances. The dosage may also be varied for route of administration, e.g. intramuscular, intraperitoneal, intradermal, subcutaneous, intravenous infusion and the like. Generally at least about 10⁴ engineered cells/kg are administered, at least about 10⁵ engineered cells/kg; at least about 10⁶ engineered cells/kg, at least about 10⁷ engineered cells/kg, or more.

Where the engineered cells are T cells, an enhanced immune response may be manifest as an increase in the cytolytic response of T cells towards the target cells present in the recipient, e.g. towards elimination of tumor cells, infected cells; decrease in symptoms of autoimmune disease; and the like. In some embodiments when the engineered T cell population is to be administered to a subject, the subject is provided with immunosuppressive course of therapy prior to or in combination with the administration of the engineered T cell population. Examples of such immunosuppressive regimens include but are not limited to systemic corticosteroids (e.g., methylprednisolone). Therapies for B cell depletion include intravenous immunoglobulin (IVIG) by established clinical dosing guidelines to restore normal levels of serum immunoglobulin levels. In some embodiments, prior to administration of the CAR-T cell therapy of the present invention, the subject may optionally be subjected to a lymphodepleting regimen. One example of a such lymphodepleting regimen consists of the administration to the subject of fludarabine (30 mg/m² intravenous daily for 4 days) and cyclophosphamide (500 mg/m² IV daily for 2 days starting with the first dose of fludarabine).

Engineered T cells can be provided in pharmaceutical compositions suitable for therapeutic use, e.g. for human treatment. Therapeutic formulations comprising such cells can be frozen, or prepared for administration with physiologically acceptable carriers, excipients or stabilizers (Remington's Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The cells can be administered by any suitable means, usually parenteral. Parenteral infusions include intramuscular, intravenous (bolus or slow infusion), intraarterial, intraperitoneal, intrathecal or subcutaneous administration. In the typical practice, the engineered T cells are infused to the subject in a physiologically acceptable medium, normally intravascularly, although they may also be introduced into any other convenient site, where the cells may find an appropriate site for growth. Usually, at least 1×10⁵ cells/kg will be administered, at least 1×10⁶ cells/kg, at least 1×10⁷ cells/kg, at least 1×10⁸ cells/kg, at least 1×10⁹ cells/kg, or more, usually being limited by the number of T cells that are obtained during collection.

For example, typical ranges for the administration of hoRb cells for use in the practice of the present invention range from about 1×10⁵ to 5×10⁸ viable cells per kg of subject body weight per course of therapy. Consequently, adjusted for body weight, typical ranges for the administration of viable cells in human subjects ranges from approximately 1×10⁶ to approximately 1×10¹³ viable cells, alternatively from approximately 5×10⁶ to approximately 5×10¹² viable cells, alternatively from approximately 1×10⁷ to approximately 1×10¹² viable cells, alternatively from approximately 5×10⁷ to approximately 1×10¹² viable cells, alternatively from approximately 1×10⁸ to approximately 1×10¹² viable cells, alternatively from approximately 5×10⁸ to approximately 1×10¹² viable cells, alternatively from approximately 1×10⁹ to approximately 1×10¹² viable cells per course of therapy. In one embodiment, the dose of the cells is in the range of 2.5-5×10⁹ viable cells per course of therapy.

A course of therapy may be a single dose or in multiple doses over a period of time. In some embodiments, the cells are administered in a single dose. In some embodiments, the cells are administered in two or more split doses administered over a period of 1, 2, 3, 4, 5, 6, 7, 8, 9, 10, 11, 12, 13, 14, 21, 28, 30, 60, 90, 120 or 180 days. The quantity of engineered cells administered in such split dosing protocols may be the same in each administration or may be provided at different levels. Multi-day dosing protocols over time periods may be provided by the skilled artisan (e.g. physician) monitoring the administration of the cells taking into account the response of the subject to the treatment including adverse effects of the treatment and their modulation as discussed above.

### Absence of Lymphodepletion

The compositions and methods of the present disclosure also provide a method for the treatment of a subject with a T cell therapy (especially CART cell therapy) in the absence of prior lymphodepletion. Lymphodepletion is typically performed in a subject in conjunction with CAR T cell therapy because the subsequent administration of the mixed cell population and the administration of non-specific agents (e.g. IL2) to expand the engineered cell population in the subject in combination with the administration of the cell therapy product acts results in significant systemic toxicity (including cytokine release syndrome or “cytokine storm”) arising from the widespread proliferation and activation of immune cells by administration of agents that result in widespread activation as well as the presence of a substantial fraction of non-engineered cells in the cell therapy product itself. The methods and compositions of the present disclosure obviate this significant hurdle by both (or either) providing a substantially purified population of engineered cells largely devoid of contamination by non-engineered cells when the foregoing ex vivo method is employed and/or the selective activation and expansion of the engineered T cells with the IL2 orthologs which provide substantially reduced off-target effects of non-specific proliferative agents such as IL2.

For example, in the current clinical practice of CAR-T cell therapy, CAR-T cells are commonly administered in combination with lymphodepletion (e.g. by administration of Alemtuzumab (monoclonal anti-CD52), purine analogs, and the like) to facilitate expansion of the CAR-T cells to prior to host immune recovery. In some embodiments, the CAR-T cells may be modified for resistance to Alemtuzumab. In one aspect of the invention, the lymphodepletion currently employed in association with CAR-T therapy may be obviated or reduced by the orthogonal ligand expressing CAR-Ts. As noted above, the lymphodepletion is commonly employed to enable expansion of the CAR-T cells. However, the lymphodepletion is also associated with major side effects of CAR-T cell therapy. Because the orthogonal ligand provides a means to selectively expand a particular T-cell population, the need for lymphodepletion prior to administration of the orthogonal ligand expressing CAR-Ts may be reduced. The present invention enables the practice of CAR-T cell therapy without or with reduced lymphodepletion prior to administration of the orthogonal ligand expressing CAR-Ts.

In one embodiment, the present disclosure provides a method of treating a subject suffering from a disease, disorder or condition amendable to treatment with CAR-T cell therapy (e.g. cancer) by the administration of a orthogonal ligand expressing CAR-Ts in the absence of lymphodepletion prior to administration of the orthogonal ligand CAR-Ts. In one embodiment, the present disclosure provides for a method of treatment of a mammalian subject suffering from a disease, disorder associated with the presence of an aberrant population of cells (e.g. a tumor) said population of cells characterized by the expression of one or more surface antigens (e.g. tumor antigen(s)), the method comprising the steps of (a) obtaining a biological sample comprising T-cells from the individual; (b) enriching the biological sample for the presence of T-cells; (c) transfecting the T-cells with one or more expression vectors comprising a nucleic acid sequence encoding a CAR and a nucleic acid sequence encoding an orthogonal CD122 receptor, the antigen targeting domain of the CAR being capable of binding to at least one antigen present on the aberrant population of cells; (d) expanding the population of the orthogonal receptor expressing CAR-T cells ex vivo with an IL2 ortholog; (e) administering a pharmaceutically effective amount of the orthogonal receptor expressing CAR-T cells to the mammal; and (0 modulating the growth of the orthogonal CD122 receptor expressing CAR-T cells by the administration of a therapeutically effective amount of an IL2 ortholog that binds selectively to the orthogonal CD122 receptor expressed on the CAR-T cell. In one embodiment, the foregoing method is associated with lymphodepletion or immunosuppression of the mammal prior to the initiation of the course of CAR-T cell therapy. In another embodiment, the foregoing method is practiced in the absence of lymphodepletion and/or immunosuppression of the mammal.

### Maintaining Threshold Levels of Orthogonal Immune Cells Over Time Using Ortho Ligand:

In one embodiment in the practice of the currently disclosed methods of using orthogonal immune cells in combination with an orthogonal ligand, the orthogonal ligand is administered to the subject in an amount to maintain a therapeutically effective circulating level of orthogonal cells in the circulation of an human subject wherein the therapeutically effective circulating level of orthogonal is about 10,000 to about 1,000,000 orthogonal cells, alternatively from about 20,000 to about 500,000 orthogonal cells, alternatively from about 30,000 to about 300,000 orthogonal cells, alternatively from about 30,000 to about 200,000 orthogonal cells, alternatively from about 20,000 to about 150,000 orthogonal cells, alternatively from about 50,000 to about 150,000 orthogonal cells per kg of subject body weight, is maintained for a period of at least one week, alternatively at least two weeks, alternatively at least 3 weeks, alternatively at least one month, alternatively at least two months, alternatively at least 3 months alternatively at least 6 months alternatively at least 9 months alternatively at least 12 months by the periodic administration of an orthogonal ligand, e.g an orthogonal ligand of Formula 1. Quantitation of the engineered cells may readily be determined by conventional antibody based methods. Excessive levels of CRP and Ferritin are used clinically to evaluate for the onset of CRS and will be typically be monitored by the attending physician to determine an optimum tolerable and efficacious levels of the orthogonal cells and the levels of orthogonal ligand necessary to maintain such level as the response of human subjects can be variable. Identification of an optimal circulating concentration of orthogonal cells for an individual patient is within the skill of the ordinary practitioner of cell therapies.

As previously noted, the present disclosure provides a method of treating a subject who exhibits evidence of significant clinical response to the initial orthogonal cell therapy treatment to prevent relapse or recurrence by the administration of a maintenance regimen wherein a lower dose of the orthogonal ligand is periodically administered to subject to maintain a lower circulating level of orthogonal cells of about 10,000 to about 500,000 orthogonal cells, alternatively from about 10,000 to about 200,000 orthogonal cells, alternatively from about 10,000 to about 100,000 orthogonal cells, per kg of subject body weight is maintained for a maintenance phase period of at least one week, alternatively at least two weeks, alternatively at least 3 weeks, alternatively at least one month, alternatively at least two months, alternatively at least 3 months alternatively at least 6 months alternatively at least 9 months alternatively at least 12 months by the periodic administration of an orthogonal ligand, e.g an orthogonal ligand of Formula 1.

### EXAMPLES

The following examples are presented in order to more fully illustrate the preferred embodiments of the invention. They should in no way, however, be construed as limiting the broad scope of the invention.

### Example 1. Generation of the Human IL2 Expression Vector pcDNA3.1/Hygro(+)-huIL2

The human IL2 DNA ORF (Genbank NM_000586.3) was synthesized (Life Technologies GeneArt Service, Carlsbad, Calif.), and amplified via PCR using Platinum SuperFi II DNA polymerase kit (item #12361050, ThermoFisher) following the manufacturer's protocol, and using primers:

(SEQ ID NO: 66)
5′ TATAGTCAGCGCCACcCATGTACAGGATGCAACTCCTGTC 3′

which incorporates and NheI restriction site, and

(SEQ ID NO: 67)
5′ TATAGGGCCCTATCAAGTCAGTGTTGAGATG 3′

which incorporates an ApaI restriction site. The PCR fragment was visualized on a 1% agarose gel (item #54803, Lonza, Rockland, Me.), excised from the gel and purified using a QIAquick PCR Purification kit (item #28106, Qiagen, Germany) according to the manufacturer's protocol.

The purified PCR fragment and mammalian expression vector pcDNA 3.1/Hygro(+) (#V87020, ThermoFisher) were digested with NheI and ApaI (#R0111S and #R0114L, New England Biolabs, Ipswich, Mass.) restriction enzymes. The expression vector was further treated with a Quick Dephosphorylation kit (#M0508L, New England Biolabs) according to the manufacturer's protocol. The PCR fragment was ligated into pcDNA 3.1/Hygro(+) using the Rapid DNA Ligation Kit (#11635379001, Sigma Aldrich, St. Louis, Mo.) following the manufacturer's protocol, transformed into One Shot TOP10 Chemically Competent E. coli (#C404006, Life Technologies, Carlsbad, Calif.), plated onto LB Agar plates containing 100 ug/ml carbenicillin (#L1010, Teknova, Hollister, Calif.), and grown overnight at 37 C.

The following day individual bacterial colonies were picked and used to start a 3 ml bacterial culture in LB Broth (#10855-001, Life Technologies) with 100 ug/ml ampicillin (#A9626, Teknova). The cultures were grown overnight at 37 C.

The following day the E. coli were pelleted (6,000 rpm, 10 minutes, tabletop centrifuge #5424, Eppendorf, Hauppauge, N.Y.), and the DNA expression vector isolated using QIAprep Spin Miniprep Kit (#27106, Qiagen). The plasmid DNA was sequence verified (MCLab, South San Francisco, Calif.).

### Example 2. Generation of the Human IL2 ORTHO Expression Vector pcDNA3.1/Hygro(+)-huIL2-ORTHO

An expression vector which introduced six mutations into the human IL2 ORF (E35S, H36Q, L39V, D40L, Q42K and M43A; all numbering is based on the full length human IL2 ORF NM_000586.3 numbering) was assembled in substantial accordance with the teaching of Example 1 for the human IL2 expression vector in pcDNA3.1/Hygro(+), with the following exceptions: The initial template DNA used for PCR was synthesized with the E35S, H36Q, L39V, D40L, Q42K and M43A mutations.

### Example 3. Introduction of Mutations into pcDNA3.1/Hygro(+)-huIL2 or Back-Mutations into pcDNA3.1/Hygro(+)-huIL2 IRTHO Expression Vectors

All mutations or back-mutations (reverting a mutation in pcDNA3.1/hygro(+)-huIL2-ORTHO back to match the wild type human IL2 ORF) were introduced into the pcDNA3.1/Hygro(±)-huIL2 or pcDNA3.1/Hygro(+)-huIL2-ORTHO expression vectors using a Quik Change II Site Directed Mutagenesis Kit (#200524, Agilent Technologies, Santa Clara, Calif.) in substantial accordance with the manufacturer's protocol.

Table 5 lists the mutations generated, the template into which the mutation was introduced, and the primer sets used to introduce the mutation. The transformation of the Quik Change PCR reactions into E. coli, as well as the isolation and sequence analysis of the plasmid DNA, was performed using substantially the same protocol as in the generation of the pcDNA3.1/Hygro-huIL2 expression vector.

TABLE 7
QuikChange Mutagenesis and
Sequence Information Regarding Mutations

| | Peptide Name | | | | | | | |
|---|---|---|---|---|---|---|---|---|
| | 35 | 36 | 39 | 40 | 42 | 43 | *Templates | |
| | Mature Peptide # | | | | | | IL2: | pcDNA3,1/hygro(+)-huIL2 |
| human | 15 | 16 | 19 | 20 | 22 | 23 | IL2 ORTHO: | pcDNA3.1/Hygro(+)-huIL2 |

| IL2 | Wild Type | | | | | | ORTHO | |
|---|---|---|---|---|---|---|---|---|
| Residue | E | H | L | D | Q | M | Primer Set (5' → 3') | Template* |
| -QVLKA | E | Q | V | L | K | A | CAAAGAAAACACAGCTACAACTGGAGC | IL2 |
| | | | | | | | AGTTACTGGTGCTCTTAAAGGC | ORTHO |
| | | | | | | | (SEQ ID NO: 68) | |
| | | | | | | | GCCTTTAAGAGCACCAGTAACTGCTCCA | |
| | | | | | | | GTTGTAGCTGTGTTTTCTTTG | |
| | | | | | | | (SEQ ID NO: 69) | |
| S-VLKA | S | H | V | L | K | A | CAGCTACAACTGAGCCATTTACTGGTGC | IL2 |
| | | | | | | | TCTTAAA (SEQ ID NO: 70) | ORTHO |
| | | | | | | | TTTAAGAGCACCAGTAAATGGCTCAGTT | |
| | | | | | | | GTAGCTG (SEQ ID NO: 71) | |
| SQ-LKA | S | Q | L | L | K | A | CTGAGCCAGTTACTGCTGCTCTTAAAGG | IL2 |
| | | | | | | | CC (SEQ ID NO: 72) | ORTHO |
| | | | | | | | GGCCTTTAAGAGCAGCAGTAACTGGCTC | |
| | | | | | | | AG (SEQ ID NO: 73) | |
| SQV-KA | S | Q | V | D | K | A | AACTGAGCCAGTTACTGGTGGATTTAAA | IL2 |
| | | | | | | | GGCCATTTTGAATG (SEQ ID NO: 74) | ORTHO |
| | | | | | | | CATTCAAAATGGCCTTTAAATCCACCAG | |
| | | | | | | | TAACTGGCTCAGTT (SEQ ID NO: 75) | |
| SQVL-A | S | Q | V | L | Q | A | TGAGCCAGTTACTGGTGCTCTTACAGGC | IL2 |
| | | | | | | | CATTTTGA (SEQ ID NO: 76) | ORTHO |
| | | | | | | | TCAAAATGGCCTGTAAGAGCACCAGTA | |
| | | | | | | | ACTGGCTCA (SEQ ID NO: 77) | |
| SQVLK- | S | Q | V | L | K | M | CTGGTGCTCTTAAAGATGATTTTGAATG | IL2 |
| | | | | | | | GAATTAA (SEQ ID NO: 78) | ORTHO |
| | | | | | | | TTAATTCCATTCAAAATCATCTTTAAGA | |
| | | | | | | | GCACCAG (SEQ ID NO: 79) | |
| S----- | S | H | L | D | Q | M | CACAGCTACAACTGTCGCATTTACTGCT | |
| | | | | | | | GG (SEQ ID NO: 80) | IL2 |
| | | | | | | | CCAGCAGTAAATGCGACAGTTGTAGCT | |
| | | | | | | | GTG (SEQ ID NO: 81) | |
| -Q----- | E | Q | L | D | Q | M | GCTACAACTGGAGCAGTTACTGCTGGAT | IL2 |
| | | | | | | | TTAC (SEQ ID NO: 82) | |
| | | | | | | | GTAAATCCAGCAGTAACTGCTCCAGTTG | |
| | | | | | | | TAGC (SEQ ID NO: 83) | |
| --V--- | E | H | V | D | Q | M | CTGGAGCATTTACTGGTGGATTTACAGA | IL2 |
| | | | | | | | TGATTTTG (SEQ ID NO: 84) | |
| | | | | | | | CAAAATCATCTGTAAATCCACCAGTAAA | |
| | | | | | | | TGCTCCAG (SEQ ID NO: 85) | |
| ---L-- | E | H | L | L | Q | M | GAGCATTTACTGCTGCTATTACAGATGA | IL2 |
| | | | | | | | TTTTG (SEQ ID NO: 86) | |
| | | | | | | | CAAAATCATCTGTAATAGCAGCAGTAA | |
| | | | | | | | ATGCTC (SEQ ID NO: 87) | |
| ----K- | E | H | L | D | K | M | CATTTACTGCTGGATTTAAAGATGATTT | IL2 |
| | | | | | | | TGAATGG (SEQ ID NO: 88) | |
| | | | | | | | CCATTCAAAATCATCTTTAAATCCAGCA | |
| | | | | | | | GTAAATG (SEQ ID NO: 89) | |
| -----A | E | H | L | D | Q | A | CTGGAGCATTTACTGCTGGATTTACAGG | IL2 |
| | | | | | | | CGATTTTGAATGGAATTAATAATTACA | |
| | | | | | | | (SEQ ID NO: 90) | |
| | | | | | | | TGTAATTATTAATTCCATTCAAAATCGC | |
| | | | | | | | CTGTAAATCCAGCAGTAAATGCTCCAG | |
| | | | | | | | (SEQ ID NO: 91) | |
| SQ---- | S | Q | L | D | Q | M | CAAAGAAAACACAGCTACAACTGAGCC | IL2 |
| | | | | | | | AGTTACTGCTGGATTTACAGATG | |
| | | | | | | | (SEQ ID NO: 92) | |
| | | | | | | | CATCTGTAAATCCAGCAGTAACTGGCTC | |
| | | | | | | | AGTTGTAGCTGTGTTTTCTTTG | |
| | | | | | | | (SEQ ID NO: 93) | |
| SQVL-- | S | Q | V | L | Q | M | CCATTCAAAATCATCTGTAAGAGCACCA | IL2 |
| | | | | | | | GTAACTGGCTCAGTTGTAGCTG | |
| | | | | | | | (SEQ ID NO: 27) | |
| | | | | | | | CAACTGAGCCAGTTACTGGTGCTCTTAA | |
| | | | | | | | AGGCCATTTTGAATGGAATTAATAATTA | |
| | | | | | | | CAAG (SEQ ID NO: 57) | |

### Example 4. Transient Transfections in HEK293 Cells

All expression vectors were transiently transfected into HEK293 cells (#CRL-1573, ATCC, Manassas, Va.). ˜1E6 HEK293 cells were plated into each well of a 6 well tissue culture plate in 2 ml of DMEM (#10569044, Life Technologies) supplemented with 10% Fetal Bovine serum (#SH30071.03, Fisher Scientific, Chicago, Ill.), and grown overnight at 37 C and 5% CO₂.

The next day the cells were transfected using Lipofectamine 3000 Reagent (#L3000150, Life Technologies) following the manufacturer's protocol, using 2.5 ug DNA, 5 ul P3000 reagent, and 7.5 ul Lipofectamine 3000 per transfection. The transfected cells were grown at 37 C, 5% CO2 for 48-72 hours and then the conditioned media was harvested.

### Example 5. Analysis of Protein Expression

Protein expression was measured by ELISA using the Human IL2 V-PLEX ELISA kit (#K151QQD-4, Mesoscale Diagnostics, Baltimore, Md.) following the manufacturer's protocol (transfected media was diluted 1:4 initially, then 1:2 serially). The plate was read on a Meso Quickplex SQ120 (Mesoscale Diagnostics) using the manufacture's preprogrammed setting for this ELISA kit. The human IL2 standard in the kit was used to compute an approximate expression level in the conditioned media samples. Table 8 below details the approximate expression levels for the proteins expressed.

TABLE 8
Expression Levels of Human IL2 Orthologs

| | Full ORF # | | | | | | |
|---|---|---|---|---|---|---|---|
| | 35 | 36 | 39 | 40 | 42 | 43 | |
| | Mature Peptide # | | | | | | |
| | 15 | 16 | 19 | 20 | 22 | 23 | |
| | Wild Type | | | | | | Expression |
| | human IL2 Residue | | | | | | Level in Transient |
| | E | H | L | D | Q | M | Transfection (ng/ml) |
| WT IL2 | E | H | L | D | Q | M | 1000 |
| SQVLKA | S | Q | V | L | K | A | 325 |
| -QVLKA | E | Q | V | L | K | A | 75 |
| S-VLKA | S | H | V | L | K | A | 325 |
| SQ-LKA | S | Q | L | L | K | A | 350 |
| SQV-KA | S | Q | V | D | K | A | 325 |
| SQVL-A | S | Q | V | L | Q | A | 225 |
| SQVLK- | S | Q | V | L | K | M | 325 |
| S----- | S | H | L | D | Q | M | 650 |
| -Q----- | E | Q | L | D | Q | M | 1000 |
| --V--- | E | H | V | D | Q | M | 650 |
| ---L-- | E | H | L | L | Q | M | 650 |
| ----K- | E | H | L | D | K | M | 900 |
| -------A | E | H | L | D | Q | A | 1400 |
| SQ---- | S | Q | L | D | Q | M | 625 |
| SQVL-- | S | Q | V | L | Q | M | 225 |

### Example 6. Evaluation of Activity of Orthologs in Cell Lines Expressing hoCD122

The IL2 orthologs were evaluated for activity in NKL cells (Robertson, et al (1996) Experimental Hematology 24(3):406-15). To generate the cell line that expresses the human orthogonal CD122 (hoNKL hoRB), NKL cells were infected with a retrovirus encoding the hoRB CD122 and co-expressing YFP (MSCV-hoRb-IRES-YFP) in accordance with procedures known in the art.

NKL and NKL hoRB cells were contacted with supernatants from 293T cells transfected with IL2 orthologs as follows: Cells were seeded in growth medium consisting of RPMI 1640 (ThermoFisher), 10 percent fetal bovine serum (ThermoFisher), 1 percent penicillin/streptomycin (ThermoFisher), 1 percent glutamax (ThermoFisher) at 0.5 million cells per ml. After two days of culture, cells were seeded into 96-well plates (Falcon) at 25 thousand cells per well in 100 μl growth medium. Two-fold serial dilutions of transfected 293T cell supernatants were made in growth medium and 100 μl of each dilution was added in duplicate to plates of NKL and NKL hoRB cells at final titrations ranging from 1:2 to 1:256. Plates were transferred to a humidified incubator (ThermoFisher) and incubated at 37 degrees centigrade, 5 percent carbon dioxide for three days.

Plates were removed from the incubator and kept at room temperature for 30 minutes. Plates were centrifuged 5 minutes at 400×g and supernatants discarded. Cells were lysed by adding 50 μl per well of a 1:1 dilution of Celltiterglo (Promega) in PBS. Cell lysates were mixed on an orbital shaker (VWR Scientific) for two minutes at 600 rpm then held at room temperature for 10 minutes. Lysates were transferred to black, clear bottom 96 well plates (Costar) and luminescence for NKL cell lysates (FIG. 1) and NKL hoRB cell lysates (FIG. 2) were read as counts per second in an Envision 2103 Multilabel Plate Reader (Perkin Elmer).

To compare the effect of each IL2 variant upon NKL cell and NKL hoRB cell proliferation, celltiterglo values for cells treated with the supernatants were compared to those obtained for control cells treated with growth medium alone, with 293T supernatant from empty-vector transfection, wild-type IL2 transfection, or supernatant from human orthogonal IL2 transfection. The data from these experiments is presented in FIGS. 2 and 3 of the accompanying drawings.

### Example 7 Efficacy of orthoCAR T Cells in Disseminated RAJI-Luc Lymphoma Mouse Model

The efficacy of orthogonal CAR-T cells in combination with a cognate orthogonal ligand was evaluated in a mouse leukemia model as follows. The CD19 chimeric antigen receptor protein used in this study (hereinafter referred to as “CD19_28 z”) had the following structure: a GMCSF receptor signal peptide, the FMC63 anti-CD19 scFv, an AAA linker, a CD28 hinge/transmembrane/costimulatory domain and a CD3zeta. The amino acid sequence of the CD19_28z is as follows:

(SEQ ID NO: 30)
MLLLVTSLLLCELPHPAFLLIPDIQMTQTTSSLSASLGDRVTISCRAS
QDISKYLNWYQQKPDGTVKLLIYHTSRLHSGVPSRFSGSGSGTDYSLT
ISNLEQEDIATYFCQQGNTLPYTFGGGTKLEITGSTSGSGKPGSGEGS
TKGEVKLQESGPGLVAPSQSLSVTCTVSGVSLPDYGVSWIRQPPRKGL
EWLGVIWGSETTYYNSALKSRLTIIKDNSKSQVFLKMNSLQTDDTAIY
YCAKHYYYGGSYAMDYWGQGTSVTVSSAAAIEVMYPPPYLDNEKSNGT
IIHVKGKHLCPSPLFPGPSKPFWVLVVVGGVLACYSLLVTVAFIIFWV
RSKRSRLLHSDYMNMTPRRPGPTRKHYQPYAPPRDFAAYRSRVKFSRS
ADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDA
LHMQALPPR

The orthogonal T cells used in the conduct of this study were generated by techniques known in the art by transfecting isolated T cells with a lentiviral vector the foregoing CD19_28 z CAR, T2A linker polypeptide and human orthogonal CD122 orthogonal receptor (hoCD122) having the amino acid sequence:

(SEQ ID NO: 31)
MLLLVTSLLLCELPHPAFLLIPDIQMTQTTSSLSASLGDRVTIS
CRASQDISKYLNWYQQKPDGTVKLLIYHTSRLHSGVPSRFSGSG
SGTDYSLTISNLEQEDIATYFCQQGNTLPYTFGGGTKLEITGST
SGSGKPGSGEGSTKGEVKLQESGPGLVAPSQSLSVTCTVSGVSL
PDYGVSWIRQPPRKGLEWLGVIWGSETTYYNSALKSRLTIIKDN
SKSQVFLKMNSLQTDDTAIYYCAKHYYYGGSYAMDYWGQGTSVT
VSSAAAIEVMYPPPYLDNEKSNGTIIHVKGKHLCPSPLFPGPSK
PFWVLVVVGGVLACYSLLVTVAFIIFWVRSKRSRLLHSDYMNMT
PRRPGPTRKHYQPYAPPRDFAAYRSRVKFSRSADAPAYQQGQNQ
LYNELNLGRREEYDVLDKRRGRDPEMGGKPRRKNPQEGLYNELQ
KDKMAEAYSEIGMKGERRRGKGHDGLYQGLSTATKDTYDALHMQ
ALPPGSGEGRGSLLTCGDVEENPGPMAAPALSWRLPLLILLLPL
ATSWASAAVNGTSQFTCFYNSRANISCVWSQDGALQDTSCQVHA
WPDRRRWNQTCELLPVSQASWACNLILGAPDSQKLTTVDIVTLR
VLCREGVRWRVMAIQDFKPFENLRLMAPISLQVVHVETHRCNIS
WEISQASDFFERHLEFEARTLSPGHTWEEAPLLTLKQKQEWICL
ETLTPDTQYEFQVRVKPLQGEFTTWSPWSQPLAFRTKPAALGKD
TIPWLGHLLVGLSGAFGFIILVYLLINCRNTGPWLKKVLKCNTP
DPSKFFSQLSSEHGGDVQKWLSSPFPSSSFSPGGLAPEISPLEV
LERDKVTQLLLQQDKVPEPASLSSNHSLTSCFTNQGYFFFHLPD
ALEIEACQVYFTYDPYSEEDPDEGVAGAPTGSSPQPLQPLSGED
DAYCTFPSRDDLLLFSPSLLGGPSPPSTAPGGSGAGEERMPPSL
QERVPRDWDPQPLGPPTPGVPDLVDFQPPPELVLREAGEEVPDA
GPREGVSFPWSRPPGQGEFRALNARLPLNTDAYLSLQELQGQDP
THLV

allowing the use of a single lentiviral plasmid to generate the orthogonal CAR-T cells. The resulting orthogonal CAR-T cells were designated CD19_28z orthoCAR T cells also referred to SYNCAR-001.

Healthy donor primary blood mononuclear cells (PBMCs) were isolated from leukopaks via Ficoll-Paque separation (Global Life Sciences Solutions). CD4 and CD8 T cells were stained with CD4 and CD8 microbeads and isolated using MACS magnetic separation columns (Miltenyi Biotec). Cells were stimulated with anti-CD3 (clone OKT3, Miltenyi Biotec) and anti-CD28 antibody (clone CD28.2, BD Biosciences). 48h post-stimulation, cells were transduced with lentivirus and maintained in complete OpTmizer T cell media (Gibco) with wild type IL-2 (Miltenyi Biotec). 48 hours post-transduction, cells were washed and either maintained in WT IL-2 containing media or switched to STK-009 containing media. Cells were expanded and maintained at 1E6 cells/ml during the remainder of manufacturing. Cell counts and viability were performed using a Vi-Cell XR (Beckman Coulter). On day 14, cells were frozen down in CryoStor CS10 cell freezing medium (STEMCELL).

The orthogonal cognate ligand for the hoCD122 receptor used in these studies was a compound of the Formula 1 described as STK-009 described in the specification comprising a monopegylated 40 kD branched (2×20 kD) PEG molecule with an aldehyde linker a 40 kDa 2-arm branched PEG-aldehyde the 40 kDA PEG-aldehyde comprising two 20kDA linear PEG molecules (Sunbright® GL2-400AL3, NOF America Corporation, One North Broadway, White Plains, N.Y. 10601 USA.

Female NOD scid gamma (NSG) mice aged 6 to 8 weeks were obtained from The Jackson Laboratory, 600 Main Street, Bar Harbor, Me. USA 04609). Prior to initiation of the study, animals were weighed and given a clinical examination to ensure that they were in good health. Weights were then tracked through the duration of the study. Animals were housed 5 per cage and acclimated for 9 days. Animals were stratified into 5 groups for treatment with the combination of CD19 28z orthoCAR T cells (2E6 total T cells, 1E6 CAR+ T cells) and STK-009 to evaluate the potential for combinatorial efficacy in vivo. STK-009 sub cutaneous dosing is performed every other day. Details of the conduct of the study plan are provided in the following Table 9.

TABLE 9
Study Design
Efficacy of STK-009 and CD19 orthoCAR T cells
in a disseminated RAJI NSG model

| Group | Treatment | # Mice |
|---|---|---|
| 1 | (1) PBS | 8 |
| 2 | (1) CD19 orthoCAR T cells (1E6 CARP⁺ T cells) | 8 |
| | (2) PBS | |
| 3 | (1) CD19 orthoCAR T cells (1E6 CARP⁺ T cells) | 8 |
| | (2) STK-009 (10 μg) | |
| 4 | (1) PBS CD19 orthoCAR T cells (1E6 CAR⁺ | 8 |
| | T cells) | |
| | (2) STK-009 (2 μg) | |
| 5 | (1) CD19 orthoCAR T cells (1E6 CARP⁺ T cells) | 8 |
| | (2) STK-009 (1 μg) | |
| | Total mice | 40 |

Tumor cell numbers were quantified twice weekly using an IVIS imager (Perkin Elmer). Mice were intraperitoneally injected with 100 ul of D-luciferin (15 mg/ml D-luciferin in PBS). Mice were put under anesthesia via controlled low-flow isoflurane exposure (Kent Scientific Somnosuite). A region of interest (ROI) was drawn around each individual mouse and total flux (photons/second) directly measuring luminescence was measured using Living Image software (Perkin Elmer). Mice were imaged on study day 5 and twice weekly thereafter. Mice were taken down when hind limb paralysis was detected (Day 21 in the PBS cohort).

On study day 0, the mice were implanted with 5×10⁵ Raji-fluc-puro tumor cells were injected intravenously in the tail vein and allowed to grow for 5 days. After tumor implantation, treatment began after 5 days. On study day 5, mice were dosed intraperitoneally with Vehicle (PBS) or CD19_28 z orthoCAR T cells in combination with subcutaneous doses of Vehicle (PBS), 1 μg of STK-009, 2 μg of STK-009, or 10 μg of STK-009. PBS and STK-009 dosing were performed every other day until Day 17 (10 μg) or Day 19 (1 μg, 2 μg). CD19_28 z orthoCAR T cells were stored at −80 degrees. On the day of injection, CD19_28 z orthoCAR T cells were thawed, spun down, and resuspended at a concentration of 2E6 total T cells/200 microliters PBS. Mice were injected intraperitoneally with orthoCAR T cells (2E6 total T cells, 1E6 CAR⁺ T cells). Eight mice/cohort were administered PBS, CD19_28 z orthoCAR T cells and PBS/STK-009 subcutaneously. Vehicle or STK-009 were administered every other day until Day 17 (10 μg) or Day 19 (1 μg, 2 μg). Blood was collected from all animals by mandibular cheek bleeds and the plasma isolated from blood via centrifugation in plasma collection tubes. Blood samples were immediately analyzed by FACS analysis and plasma was frozen at −80 degrees for future analysis. Spleen, kidneys, liver, and hind limbs were fixed in formaldehyde for IHC processing.

The results of the experiment are provided in FIG. 3 of the attached drawings. Bioluminescence data is provided in the following Table 10.

### Table 10. Bioluminescence Data from Disseminated Raji-Luc Study

TABLE 10
Bioluminescence Data From Disseminated Raji-Luc Study

| | | | | | | | | |
|---|---|---|---|---|---|---|---|---|
| Time (days) | 1 | 2 | 3 | 4 | 5 | 6 | 7 | 8 |

PBS

| | | | | | | | | |
|---|---|---|---|---|---|---|---|---|
| 5 | 118590000 | 3120000 | 24477000 | 24477000 | 65320000 | 8114000 | 1268000 | 36800000 |
| 8 | 17300000000 | 354000000 | 557000000 | 557000000 | 142000000 | 67900000 | 109000000 | 2080000000 |
| 12 | 357000000000 | 7340000000 | 16900000000 | 16900000000 | 993000000 | 967000000 | 3160000000 | 31700000000 |
| 15 | 560000000000 | 18000000000 | 35500000000 | 35500000000 | 23000000 | 3600000000 | 4410000000 | 47200000000 |
| 19 | 1.52E+11 | 65700000000 | 85800000000 | 79300000000 | 1.29E+11 | 15400000000 | 1.37E+11 | 1.17E+11 |

OD19_28z orthoCAR + PBS

| | | | | | | | | |
|---|---|---|---|---|---|---|---|---|
| 5 | 92240000 | 20745000 | 21660000 | 4180000 | 88120000 | 21325000 | 17273000 | 15667000 |
| 8 | 1100000000 | 757000000 | 1480000000 | 337000000 | 111000000 | 195000000 | 267800000 | 166000000 |
| 12 | 2770000000 | 3950000000 | 2460000000 | 1920000000 | 750000000 | 249000000 | 774000000 | 255000000 |
| 15 | 631000000 | 1810000000 | 183000000 | 326000000 | 265000000 | 68800000 | 23100000 | 2860000 |
| 19 | 49900000 | 43900000 | 3470000 | 8220000 | 216000000 | 4510000 | 2310000 | 2800000 |
| 22 | 7530000 | 46100000 | 2910000 | 8520000 | 837000000 | 13500000 | 5350000 | 9510000 |
| 26 | 5340000 | 48000000 | 3130000 | 15300000 | 3060000000 | 37000000 | 1210000 | 75000000 |
| 33 | 1930000 | 50400000 | 2330000 | 52400000 | 9380000000 | 2690000 | 2080000 | 277000000 |

OD19_28z_T2A_hORB + STK-009 10 ug

| | | | | | | | | |
|---|---|---|---|---|---|---|---|---|
| 5 | 98430000 | 3818000 | 36780000 | 20342000 | 63490000 | 27710000 | 10792000 | 12658000 |
| 8 | 1410000000 | 503000000 | 791000000 | 274000000 | 306000000 | 458000000 | 23151000 | 28280000 |
| 12 | 9170000000 | 533000000 | 2040000000 | 713000000 | | 15500000000 | | |
| 15 | 5700000000 | 344000000 | 615000000 | 56300000 | | 13900000000 | | |
| 19 | 108000000 | | | 2610000 | | 969000000 | | |

OD19_28z_T2A_hORB + STK-009 2 ug

| | | | | | | | | |
|---|---|---|---|---|---|---|---|---|
| 5 | 83520000 | 2259000 | 20424000 | 11066000 | 62070000 | 8927000 | 2016100 | 15603000 |
| 8 | 1080000000 | 213000000 | 1030000000 | 98850000 | 1230000000 | 109000000 | 21400000 | 163000000 |
| 12 | 8990000000 | 1290000000 | 9210000000 | 476000000 | 6120000000 | 910000000 | 227000000 | 859000000 |
| 15 | 139000000 | 688000000 | 1450000000 | 30900000 | 1470000000 | 735000000 | 461000000 | 564000000 |
| 19 | 2480000 | 5180000 | 21700000 | 1200000 | 13300000 | 6720000 | 136000000 | 8350000 |
| 22 | 2820000 | 4810000 | 889000 | 2040000 | 4310000 | 1640000 | 16400000 | 44440000 |
| 26 | 2130000 | | 1310000 | 3730000 | 3450000 | 2320000 | 2320000 | |
| 33 | 502000 | | 609000 | | 2960000 | 2310000 | 2310000 | |

OD19_28z_T2A_hORB + STK-009 1 ug

| | | | | | | | | |
|---|---|---|---|---|---|---|---|---|
| 5 | 76820000 | 366800 | 25897000 | 13226000 | 49250000 | 4102500 | 18859000 | 14926000 |
| 8 | 165000000 | 550000000 | 626000000 | 140000000 | 83000000 | 14106000 | 239000000 | 188000000 |
| 12 | 1280000000 | 1770000000 | 1490000000 | 159000000 | 329000000 | 62700000 | 731000000 | 1250000000 |
| 15 | 157000000 | 749000000 | 88400000 | 37500000 | 6530000 | 3820000 | 310000000 | 209000000 |
| 19 | 28800000 | 5250000 | 5930000 | 3690000 | 3770000 | | 2410000 | 8590000 |
| 22 | 1300000 | 2260000 | 1360000 | 2100000 | 4350000 | 3650000 | 2870000 | 3160000 |
| 26 | 2740000 | 1340000 | 1510000 | 2660000 | 656000 | 1170000 | 820000 | 1290000 |
| 33 | 987000 | 1530000 | 222000 | 584000 | 153000 | 11900 | 142000 | 752000 |

As demonstrated by the data provided in FIG. 3 and the above table the administration of PBS failed to control the tumor burden FIG. 3, Panel A, Group 1 and FIG. 3, Panel B, upper left) resulting the animals needing to be sacrificed due to toxicity on approximately day 21 of the study. The administration of CD19_28 z orthoCAR T cells led to an antitumor response in 4/8 mice (FIG. 3, Panel A, Group 2 and FIG. 3, Panel B, upper right). The combined treatment of both CD19_28 z orthoCAR T cells with STK-009 at both dose levels (FIG. 3, Panel A, 1 μg (Group 3, and FIG. 3, Panel B lower right) and 2 μg (Group 4 and FIG. 3, Panel B lower left) provided additional anti-tumor function compared to CD19_28 z orthoCAR T cell treatment alone.

As previously noted, the bodyweights of the animals were determined throughout the course of the foregoing study. Significant loss of bodyweight is an established measure of toxicity. The bodyweights of the animals in the foregoing study in the treatment groups with the orthogonal CAR-T cells with and without the addition of the orthogonal ligand was generally well tolerated and there was little evidence of systemic toxicity associated with the oCD19 CART or the STK-009 ligand.

### Example 8. Subcutaneous Raji Lymphoma Solid Tumor Model

For the subcutaneous lymphoma models, 6-8 week old NSG and NSG MHC I/II KO mice (Jackson laboratories) were injected subcutaneously with a 50:50 ratio of 5E5 Raji-fluc-puro cells and phenol-red free Matrigel (Corning). After 4 days, mice were imaged as described, randomized and injected subcutaneously with either PBS or STK-009. On day 5, mice were treated with either PBS, SYNCAR T cells, and either PBS or STK-009, subcutaneously. Mice received subcutaneous injections of either PBS or STK-009 every other day and paused once body weight dropped below 90% of their weight at the beginning of the study. Tumor caliper measurements were made twice a week, mice were bled once a week for subsequent cytokine and flow cytometric analysis, while body weights were taken up to 5 times a week.

### Example 9. Histology Analysis and Immunohistochemistry

Immunohistochemistry analysis was performed on tissues for anti-human CD4, CD8, GranzymeB, CD3 and anti-mouse CD11b. FFPE blocks were sectioned at 4 um thickness. Slides were de-paraffinized in a series of Xylenes and progressively diluted alcohols to water. Slides were treated with Citrate based low pH antigen retrieval before commencing IHC staining on Dako autostainer at room temperature. Endogenous peroxidase was quenched with 3% hydrogen peroxide for 5 minutes, followed by primary antibody incubation for 1 hr at room temperature. Tissues were subsequently incubated with HRP-conjugated polymer secondary reagent for 30 minutes, followed by DAB or TSA-conjugated Alexa fluor 488, 594 or 647 to visualize the signal. Human CD3, mouse CD11b were stained with DAB for chromogenic detection and analysis. Anti-human CD4, CD8 and GranzymeB were multiplexed for quantification using fluorescence detection. For dual labeling, staining was performed in sequential manner by including second antigen retrieval step between the two procedures to elute out any unbound reagents from the first marker. Appropriate cross-reactivity controls were included for quality check. Upon completion of fluorescence staining, tissues were counterstained with DAPI and cover slipped using Prolong Gold aqueous mounting medium. Upon completion of chromogenic staining, samples were counterstained with Hematoxylin and cover slipped for imaging.

Whole slide scanning and signal quantification was performed using Akoya Vectra multi-spectral imaging system and Akoya Vectra InForm analysis software suite. The Primary antibodies used were: CD4: R&D Systems, AF-379-NA, 5 ug/ml for 1 hr, Goat polyclonal; CD8: Dako, M7103, Clone C8/144B, 1:500 for 1 hr, Mouse monoclonal; GranzymeB: Cell Signaling, 46890s, Clone D6E9W, 1:500 for 1 hr, Rabbit monoclonal; CD3: Lab Vision, RM-9107-S, Clone SP7, 1:200 for 1 hr, Rabbit monoclonal; CD11b: Abcam, ab216445, Clone EPR1344, 1:3000 for 1 hr, Rabbit monoclonal and the Secondary antibodies use are Leica Powervision Anti-Rabbit-HRP, Leica Powervision Anti-Mouse-HRP, Vector Immpress Anti-Goat-HRP

TABLE 11
Bodyweight Data From Disseminated Raji-Luc Study (g/mouse)

| | | | | | | | | |
|---|---|---|---|---|---|---|---|---|
| Time (days) | 1 | 2 | 3 | 4 | 5 | 6 | 7 | 8 |

PBS

| | | | | | | | | |
|---|---|---|---|---|---|---|---|---|
| 7 | 22.73 | 25.41 | 24.31 | 24.30 | 17.95 | 21.54 | 25.22 | 24.97 |
| 8 | 22.71 | 25.91 | 24.53 | 24.32 | 18.19 | 21.47 | 24.73 | 23.51 |
| 11 | 23.25 | 26.45 | 24.08 | 24.21 | 19.65 | 22.25 | 25.02 | 24.73 |
| 13 | 22.85 | 25.43 | 24.05 | 24.05 | 18.45 | 21.99 | 24.63 | 23.55 |
| 14 | 22.72 | 25.46 | 24.53 | 24.28 | 18.24 | 21.67 | 25.25 | 24.23 |
| 15 | 23.07 | 25.48 | 24.94 | 24.62 | 17.74 | 21.93 | 25.2 | 24.42 |
| 18 | 22.79 | 26.29 | 24.36 | 24.19 | 15.69 | 22.74 | 23.51 | 23.63 |
| 19 | 21.26 | 26.44 | 23.54 | 22.82 | 17.83 | 22.09 | 21.75 | 21.09 |

OD19_28z orthoCAR + PBS

| | | | | | | | | |
|---|---|---|---|---|---|---|---|---|
| 7 | 23.27 | 24.25 | 22.37 | 22.2 | 24.16 | 22.03 | 21.26 | 22.64 |
| 8 | 23.32 | 24.36 | 22.73 | 22.26 | 24.32 | 22.04 | 21.81 | 23.19 |
| 11 | 23.67 | 24.22 | 23.32 | 22.95 | 24.99 | 22.62 | 22.22 | 23.13 |
| 13 | 22.94 | 23.92 | 22.59 | 22.56 | 24.45 | 22.08 | 21.84 | 22.92 |
| 14 | 23.89 | 24.43 | 23.02 | 22.52 | 24.67 | 22.12 | 21.95 | 23.43 |
| 15 | 23.28 | 24.43 | 23.02 | 22.32 | 24.94 | 22.72 | 22.1 | 23.31 |
| 18 | 24.42 | 24.07 | 23.49 | 23.49 | 25.78 | 23.33 | 22.72 | 23.81 |
| 19 | 24.17 | 24.36 | 23.51 | 23.51 | 25.64 | 23.91 | 23.05 | 23.85 |
| 22 | 24.81 | 23.97 | 23.66 | 23.66 | 25.9 | 24.27 | 22.47 | 24.99 |
| 26 | 25.42 | 24.84 | 23.43 | 23.43 | 25.47 | 23.98 | 23.6 | 24.77 |
| 32 | 24.51 | 27.31 | 24.45 | 24.45 | 25.62 | 24.69 | 24.59 | 26.35 |
| 33 | 24.32 | 26.72 | 25.26 | 25.26 | 23.83 | 24.55 | 24.29 | 25.59 |

OD19_28z_T2A_hORB + STK-009 10 ug

| | | | | | | | | |
|---|---|---|---|---|---|---|---|---|
| 7 | 22.83 | 20.25 | 21.96 | 23.17 | 23.66 | 25.17 | 22.65 | 21.77 |
| 8 | 23.01 | 20.77 | 22.25 | 23.03 | 24.25 | 24.99 | 22.87 | 21.52 |
| 11 | 24.71 | 21.11 | 22.79 | 23.62 | | 25.7 | | |
| 13 | 24.22 | 21.25 | 22.02 | 24.05 | | 25.25 | | |
| 14 | 22.75 | 20.69 | 21.5 | 23.09 | | 23.64 | | |
| 15 | 21.36 | 19.02 | 19.75 | 21.4 | | 22.01 | | |
| 18 | 19.36 | 17.14 | 17.74 | 19.29 | | 18.88 | | |
| 19 | 19.25 | | 17.93 | 19.14 | | 18.36 | | |

OD19_28z_T2A_hORB + STK-D09 2 ug

| | | | | | | | | |
|---|---|---|---|---|---|---|---|---|
| 7 | 23.49 | 22.03 | 20.83 | 20.93 | 24.61 | 23.53 | 22.14 | 23.35 |
| 8 | 23.53 | 22.16 | 20.4 | 20.03 | 25.15 | 24.24 | 22.24 | 23.71 |
| 11 | 24.29 | 22.46 | 20.5 | 21.56 | 25.31 | 24.79 | 23.02 | 24.26 |
| 13 | 23.65 | 22.48 | 20.37 | 20.56 | 24.97 | 24.28 | 22.55 | 23.72 |
| 14 | 23.11 | 22.69 | 19.64 | 21.19 | 24.45 | 24.59 | 22.6 | 23.78 |
| 15 | 21.83 | 22.25 | 19.2 | 21.55 | 22.96 | 24.61 | 22.58 | 23.02 |
| 18 | 19.06 | 19.56 | 17.2 | 18.67 | 21.23 | 23.58 | 19.27 | 21.55 |
| 19 | 19.44 | 18.79 | 56.75 | 17.72 | 20.58 | 21.78 | 19.02 | 20.51 |
| 22 | | 18.01 | | 18.23 | 18.09 | 21.36 | 20.95 | 21.68 |
| 26 | | 21.58 | | 20.73 | 21.64 | 23.17 | 22.4 | 23.73 |
| 32 | | 26.06 | | 23.31 | 27.19 | 26.3 | 24.98 | 24.2 |
| 33 | | 25.74 | | 23.39 | | 26.69 | 25.04 | 23.05 |

OD19_28z_T2A_hORB + STK-D09 1 ug

| | | | | | | | | |
|---|---|---|---|---|---|---|---|---|
| 7 | 21.58 | 21.71 | 24.55 | 21.95 | 20.31 | 22.81 | 21.77 | 21.15 |
| 8 | 21.02 | 21.73 | 24.56 | 21.68 | 20.56 | 22.91 | 21.45 | 21.16 |
| 11 | 21.1 | 22.79 | 25.67 | 22.77 | 21.13 | 23.1 | 22.24 | 21.32 |
| 13 | 21.12 | 22.76 | 24.96 | 22.32 | 21.06 | 22.43 | 21.95 | 21.06 |
| 14 | 20.53 | 23.4 | 24.5 | 22.59 | 21.05 | 22.91 | 22.21 | 21.55 |
| 15 | 19.44 | 23.95 | 24.06 | 22.47 | 20.48 | 23.43 | 22.42 | 21.84 |
| 18 | 27.33 | 24.54 | 21.55 | 20.22 | 15.44 | 23.41 | 22.58 | 21.57 |
| 19 | 16.58 | 24.3 | 20.55 | 19.42 | 17.69 | 23.49 | 20.94 | 20.02 |
| 22 | 18.1 | 23.38 | 23.41 | 21.02 | 19.68 | 23.25 | 22.74 | 20.36 |
| 26 | 22.36 | 23.12 | 23.9 | 23.72 | 22.03 | 23.75 | 22.6 | 22.27 |
| 32 | 25.55 | 26.57 | 24.53 | 26.32 | 23.06 | 24.07 | 25.02 | 23.65 |
| 33 | 25.03 | 24.84 | 26.05 | 25.75 | 22.65 | 22.96 | 24.83 | 23.57 |

### Example 9: RAJI-Luc Lymphoma Rechallenge Mouse Model

The following experiment is a continuation of the study described in Example 8 above. On day 34, mice effectively cured from the initial phase of treatment (CD19 orthoCAR T cell +1 ug STK-009 treated mice, (Group 3 in FIG. 3 Panel A)were split into two groups and were continuously dosed with either PBS or STK-009 every other day. The schedule of dosing is presented in FIG. 5 Panel A of the attached drawings. (FIG. 4A). The inverted triangles represent the administration of the dose of STK-009. It should be noted that no additional CD19 orthoCAR T cells were administered, only the STK-009 orthogonal ligand.

On day 43, the treatment group mice and PBS group mice were subcutaneously injected with 5E5 RAJI-luc cells and 50% matrigel into the right rear flanks. Tumor burden was assessed biweekly as previously described. PBS group were euthanized 21 days post-injection (day 64) due to excessive tumor burden (FIG. 4B, first panel), 2/4 mice treated with PBS were able to control growth of the tumor (FIG. 4B, second panel), while all mice (4/4) treated with STK-009 were able to control tumor growth (FIG. 2B, third panel).

These data demonstrate that STK009 redosing is capable of restoring the anti-tumor activity of CART cells even a prolonged period of no antigen or tumor ligand exposure.

### Example 10. STK-009 Treatment of RAJI-Luc Lymphoma after Relapse

The following experiment is a continuation of the study described in Example 8 above. On day 34, those mice that were treated with CD19 orthoCAR T cells and PBS (i.e. naïve to treatment with the STK-009 orthogonal ligand) and relapsed (Group 2 in FIG. 3, Panel A, n=4) were isolated for further treatment. As noted in FIG. 5, panel B these mice exhibited relapse on approximately day 20 of the study. All mice were treated by the subcutaneous administration of 1 ug pf STK-009 every other day for 16 days (8 doses total, FIG. 5 panel A). Tumor burden was assessed biweekly as previously described.

As noted in the data provided in FIG. 5, Panel B, all four mice in the study showed signs of tumor regression after dosing with STK-009, again without additional administration of CAR-T cells. This data demonstrates that the administration of STK-009 alone is capable of effectuating anti-tumor activity of CAR-T cells in animal that have relapsed from a prior course of therapy.

### Example 11. Evaluation Phenotype of Cells

The following is an evaluation of T-cell quantity (Y-axis) and phenotype (shading as provided in the Figure legend) in the treatment groups of the study described in Example 8 above. Briefly, samples obtained on study days 20, 25 and 32 (X-axis). Cells were quantified by FACS analysis the data is provided in the histogram of FIG. 6 of the attached drawings. As demonstrated from the data provide in FIG. 6, an orthogonal ligand (STK-009) is capable of expanding the orthogonal CAR-T cells and retains the stem cell memory CAR-T cell population. Longevity of CAR-T cells is limited in vivo by differentiation to an effector T cell fate in a progression from stem cell memory (SCM) phenotype, to central memory (CM) phenotype, to effector memory (EM) phenotype, to effector memory CD45+ RA (EMRA) phenotype. As demonstrated from the data provided on FIG. 6 of the attached drawings, upon cessation of treatment, approximately 60% of the orthogonal CAR-T cells treated with the orthogonal ligand (STK-009) retained the SCM phenotype.

### Example 12. Evaluation of Effect of Orthogonal CAR-T/Ligand System in a Subcutaneous Solid Tumor Model

The following study was performed to evaluate the in vivo efficacy of STK-009 (prepared in substantial accordance with Example 8), in combination with CD19_28z orthoCAR T cells (prepared in substantial accordance with Example 8), in the control of subcutaneous solid tumor RAJI tumor growth in NSG mice. Mice were obtained and treated as provided in Example 8. The study design is provided in the Table 12 Below.

TABLE 12
Study Design
Efficacy of STK-009 and CD19 orthoCAR T cells in
a subcutaneous RAJI NSG model

| Group | Treatment | # Mice |
|---|---|---|
| 1 | (2) PBS | 8 |
| 2 | (3) CD19 orthoCAR T cells (1E6 CAR⁺ T cells) | 8 |
| | (4) PBS | |
| 3 | (3) CD19 orthoCAR T cells (1E6 CAR⁺ T cells) | 8 |
| | (4) STK-009 (10 μg) | |
| 4 | (3) PBS CD19 orthoCAR T cells (1E6 CAR⁺ | 8 |
| | T cells) | |
| | (4) STK-009 (1 μg); changed to 10 μg at Day 50 | |
| | Total mice | 32 |

5E5 Raji-fluc-puro tumor cells (Imanis Life Sciences #CL-161) were subcutaneously injected in a volume of 100 microliters PBS+100 microliters Matrigel (Corning) into the right flank. After tumor implantation, treatment began after 6 days. On day 6, mice were dosed intraperitoneally with Vehicle (PBS) or CD19_28 z orthoCAR T cells in combination with subcutaneous doses of Vehicle (PBS), 1 μg of STK-009 or 10 μg of STK-009. PBS and STK-009 dosing were performed every other day. Mice were injected intraperitoneally with CD19_28 z orthoCAR T cells (2E6 total T cells, 1E6 CAR^{T} T cells). CD19_28 z orthoCAR T cells were stored at −80 degrees. On the day of injection, CD19_28 z orthoCAR T cells were thawed, spun down, and resuspended at a concentration of 2E6 total T cells/200 microliters PBS. STK-009 was aliquoted and stored at −80 degree and further diluted with vehicle (PBS) at time of dosing. Vehicle or STK-009 were administered every other day until day 75. On day 50, treatment of mice in the CD19_28 z orthoCAR T cell +STK-009 1 μg group was increased to 10 μg.

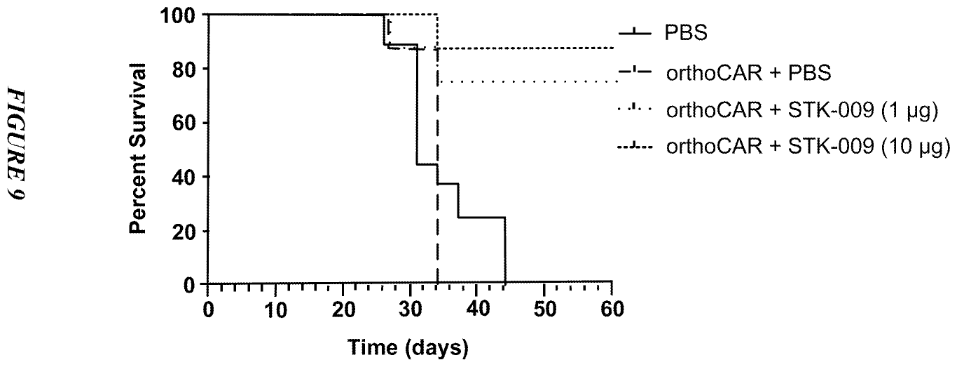

Mice were imaged and tumor caliper measurements were made weekly. Tumor size and cell numbers were measured weekly using calipers and an IVIS imager (Perkin Elmer), respectively. For imaging, mice were intraperitoneally injected with 100 ul of D-luciferin (15 mg/ml D-luciferin in PBS). Mice were put under anesthesia via controlled low-flow isoflurane exposure (Kent Scientific Somnosuite). A region of interest (ROI) was drawn around each individual mouse and total flux (photons/second) directly measuring luminescence was measured using Living Image software (Perkin Elmer). Caliper measurements were taken of the length and width of the tumor. Tumor volumes were calculated using the formula 1/2 L×W². Caliper measurements were stopped on Day 60 since IVIS imaging was more sensitive. Blood was collected from all animals by mandibular cheek bleeds and the plasma isolated from blood via centrifugation in plasma collection tubes. Blood samples were immediately analyzed by FACS analysis and plasma was frozen at −80 degrees for future analysis. Tumors, spleen, kidneys, liver, lung, and hind limbs were fixed in formaldehyde for IHC processing. All samples were stored appropriately for analysis. Mice were taken down if tumor volume exceeded >2000 mm³ The data is provided in FIGS. 9-12 of the attached drawings.

As shown in FIGS. 7-10, administration of CD19_28 z orthoCAR T cells at this dose (4E5 CAR⁺ T cells, a dose that would typically considered a sub-efficacious dose) nevertheless demonstrated significant anti-tumor efficacy.

FIG. 7 Panel A, provides the results of tumor volume in this subcutaneous study as measured with a calipers in the four treatment conditions. FIG. 7 Panel B, provides the results of tumor volume in this subcutaneous study as measured with a calipers in the four treatment conditions indicating that STK-009 expends CAR-T cells in vivo and expands SCM phenotype CAR-T cells (FIG. 7 Panel C). The combined treatment of both CD19_28 z orthoCAR T cells and STK-009 (1 μg, 10 μg) provided significant anti-tumor function compared to CD19_28 z orthoCAR T cell treatment alone in a dose dependent manner

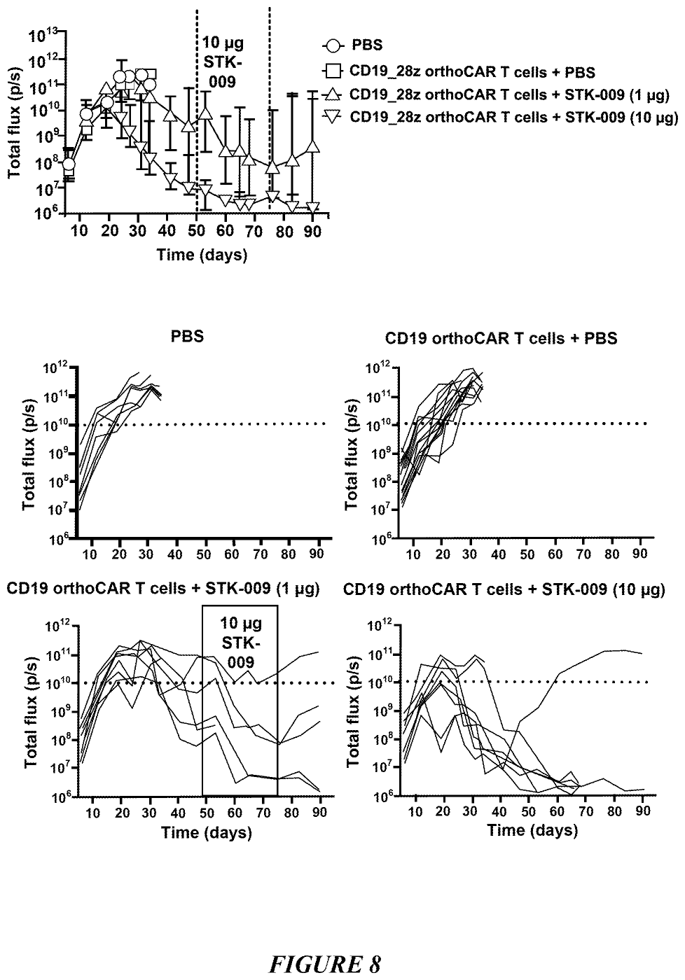

FIG. 8, Panel A provides the results of tumor volume in this subcutaneous study as measured by median tumor luminescence in the four treatment conditions indicated in the Figure legend. FIG. 8, Panel B provides the results of tumor volume in this subcutaneous study as measured by median tumor luminescence in the four treatment conditions indicated in the four treatment groups as indicated. To address whether increasing the dosage of STK-009 can enhance anti-tumor efficacy in mice already receiving 1 μg of STK-009, dosing was increased to 10 μg on day 50 (FIG. 8, Panel B, lower left). A significant decrease in tumor burden was observed after increasing the dose from 1 μg to 10 μg.

FIG. 9 is a Kaplan-Meier survival plot of the survival of animals in each treatment group as indicated. As demonstrated by this data, the combinatorial treatment of CD19_28 z orthoCAR T cells and STK-009 significantly extended mouse survival compared to PBS and CD19_28 z orthoCAR T cell treatment alone.

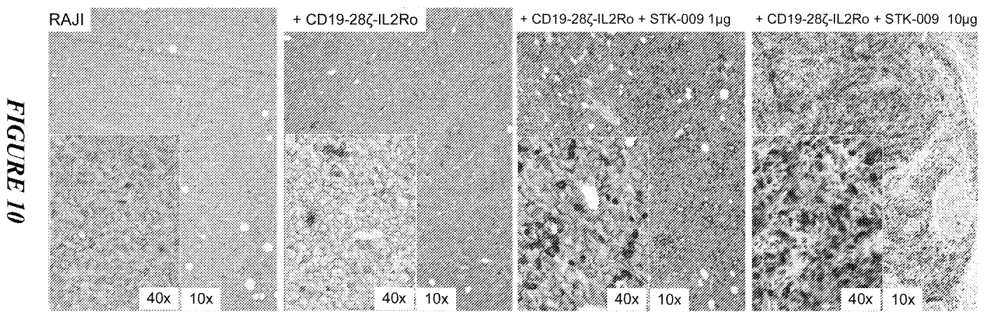

FIG. 10 is a 10× (inset 40×) photomicrograph of an immunohistochemical analysis of the CAR-T infiltration in the subcutaneous RAJI solid tumors. As illustrated from these slides, STK-009 induces CAR-T infiltration and tumor rejection of SC RAJI tumors. The analysis indicated no viable RAJI cells in high dose STK-009 treated tumors.

What is claimed:

1. A method of treating or preventing a disease, disorder, or condition in a mammalian subject in need of treatment or prevention, the method comprising the steps of:
(a) Isolating a quantity of immune cells from the subject;
(b) Contacting said isolated quantity of isolated immune cells with a nucleic acid sequence under conditions for the uptake of said nucleic acid sequence by the isolated immune cells, said nucleic acid sequence encoding a transmembrane receptor, said transmembrane receptor comprising an intracellular signaling domain in operable communication with an extracellular domain, said extracellular domain of said receptor comprising the ECD of a an orthogonal hCD122 or a functional fragment thereof;
(c) Contacting the isolated quantity of cells from step (b) ex vivo with a quantity of a orthogonal ligand sufficient to induce proliferation of cells transduced by the contacting of step (b), said contacting being applied for a period of time to such that the transduced cells comprise at least 20% of the cells of the population;
(d) Administering a therapeutically effective quantity of the cells of the cell population produced from step (c) to the mammalian subject in combination with the administration of a therapeutically effective dose of a orthogonal ligand.

2. The method of claim 1 wherein the population comprises one or more of species human immune cells selected from the group consisting myeloid cells, lymphocytes, peripheral blood mononuclear cells (PBMCs), tumor infiltrating lymphocytes (TILs), T cells, CD8+ T cells, CD25+CD8+ T cells, CAR-T cells, NK cells, CD4+ T cells, and Tregs.

3. The method of claim 1 wherein after step (a) but prior to step (b), the population of cells is manipulated ex vivo to enrich said population for activated immune cells or antigen experienced T cells.

4. The method of claim 1 wherein the orthogonal hCD122 or functional fragment thereof comprises an amino acid sequence with an amino acid substitution at position 133 and/or 134 numbered in accordance with wild-type hCD122.

5. The method of any one of claims 1-4 wherein the contacting of step (b) further comprises the uptake of a nucleic acid sequence encoding a chimeric antigen receptor (CAR).

6. The method of claim 5 wherein the nucleic acid sequence encoding the CAR and the nucleic acid sequence encoding the receptor are provided on separate vectors, each nucleic acid sequence operably linked to an expression control sequence operatable in a mammalian immune cell.

7. The method of claim 5 wherein the nucleic acid sequence encoding the CAR and the nucleic acid sequence encoding the receptor are provided on a single vector.

8. The method of claim 7 wherein the nucleic acid sequences are operably linked to the same expression control element.

9. The method of claim 8 wherein the vector comprises the two nucleic acid sequences are separated by an IRES element of T2A coding sequence.

10. The method of claim 9 wherein the vector is a viral vector.

11. The method of claim 10 wherein the vector is a lentiviral vector or retroviral vector.

12. The method of any of claims 1-11 wherein the orthogonal ligand employed ex vivo in step (b) is different than the orthogonal ligand used in vivo in step (c).

13. The method of anyone of claims 1-12 wherein prior to step (d) the subject is treated with a lymphodepleting regiment.

14. The method of can one of claims 1-13 wherein the initial dose administered in step (d) is between 100,000 and 1,000,000 activate immune cells per kg of bodyweight of the subject.

15. The method of any one of claims 1-14 wherein the administration of the orthogonal ligand is administered periodically to the subject to maintain a level of between 100,000 and 1,000,000 activate immune cells per kg of bodyweight of the subject for a period of time of at least two weeks

16. The method of any one of claims 1-15 wherein the orthogonal ligand is administered until a point where there is no substantial sign of remaining tumor at which time the dose of the orthogonal ligand is reduced to a level sufficient to maintain a low circulating level of orthogonal immune cells of approximately 10,000 to 100,000 cells per kg of bodyweight for a period of time of at least 3 months following the observation

17. The method of any one of claims 1-15 wherein the orthogonal ligand is administered until a point where there is no substantial sign of remaining tumor at which time the dose of the orthogonal ligand terminated.

18. The method of claim 17 wherein if the patient relapses from the initial course of immune cell therapy; the method further comprising the step of administering to the patient in relapse a therapeutically effective amount of an orthogonal ligand in the absence of additional dose of the orthogonal engineered cell such that the orthogonal ligand induces the activation and/or proliferation of the previously administered orthogonal cell, the orthogonal ligand being applied to the subject for a period of time until remission of the relapsed tumor is observed.

19. The method of any one of claims 1-18 wherein the disease, disorder of condition is a neoplastic disease.

20. The method of any one of claims 1-19 wherein the disease, disorder of condition is a chronic viral disease.

21. The method of any one of claims 1-19 wherein the disease, disorder of condition is a inflammatory disease.

22. A cell product substantially enriched for a population of activated orthogonal immune cells the product obtained by a process comprising the steps of:
(a) Isolating a quantity of immune cells from a mammalian subject;
(b) Contacting said isolated quantity of isolated immune cells with a nucleic acid sequence under conditions for the uptake of said nucleic acid sequence by the isolated immune cells, said nucleic acid sequence encoding a transmembrane receptor, said transmembrane receptor comprising an intracellular signaling domain in operable communication with an extracellular domain, said extracellular domain of said receptor comprising the ECD of a an orthogonal hCD122 or a functional fragment thereof;
(c) Contacting the isolated quantity of cells from step (b) ex vivo with a quantity of a orthogonal ligand sufficient to induce proliferation of cells transduced by the contacting of step (b), said contacting being applied for a period of time to such that the transduced cells comprise at least 20% of the cells of the population.

23. The cell product of claim 22 wherein the cell product comprises one or more of species human immune cells selected from the group consisting myeloid cells, lymphocytes, peripheral blood mononuclear cells (PBMCs), tumor infiltrating lymphocytes (TILs), T cells, CD8+ T cells, CD25+CD8+ T cells, CAR-T cells, NK cells, CD4+ T cells, and Tregs.

24. The composition of claim 23 wherein the cell product is further manipulated to deleted the endogenous TCR domain of said cell.
